SEQUENCE LISTING (396 sequences)

SEQ ID NO: 1 - DNA, 30 bp, Artificial Sequence
  source: /note=“Description of Artificial Sequence Synthetic oligonucleotide”
cttcagcttcccgatatccgacggtagtgt

SEQ ID NO: 2 - DNA, 21 bp, Artificial Sequence
  source: /note=“Description of Artificial Sequence Synthetic oligonucleotide”
aggagtaagtctgccagcatt

SEQ ID NO: 3 - DNA, 24 bp, Artificial Sequence
  source: /note=“Description of Artificial Sequence Synthetic oligonucleotide”
aaatgtcttgtgaaacaaaatgct

SEQ ID NO: 4 - DNA, 105 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
aggagtaagtctgccagcattnnnnnnnnnncttcagcttcccgattacgggtacgatccgacggtagtgtnnnnnnnnnnaaatgtcttgtgaaacaaaatgct

SEQ ID NO: 5 - DNA, 21 bp, Artificial Sequence
  source: /note=“Description of Artificial Sequence Synthetic oligonucleotide”
accacctcccatatgtccaga

SEQ ID NO: 6 - DNA, 20 bp, Artificial Sequence
  source: /note=“Description of Artificial Sequence Synthetic oligonucleotide”
accagtctgggcaacatagc

SEQ ID NO: 7 - DNA, 21 bp, Artificial Sequence
  source: /note=“Description of Artificial Sequence Synthetic oligonucleotide”
gtctgaatcaaatgccaaagt

SEQ ID NO: 8 - DNA, 21 bp, Artificial Sequence
  source: /note=“Description of Artificial Sequence Synthetic oligonucleotide”
tcccctgtgtgagagaaaaga

SEQ ID NO: 9 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gtctgaatcaaatgccaaagtnnnnnnnnnncttcagcttcccgattacgggtacgatccgacggtagtgtnnnnnnnnnntcccctgtgtgagagaaaaga

SEQ ID NO: 10 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tccgaaggtaattgcctcccnnnnnnnnnncttcagcttcccgattacgggtacgatccgacggtagtgtnnnnnnnnnntacttcttcccaccaaagca

SEQ ID NO: 11 - DNA, 106 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
acgtttagtttgtgacaagctcannnnnnnnnncttcagcttcccgattacgggtacgatccgacggtagtgtnnnnnnnnnntgtttttaagcctactggagcaa

SEQ ID NO: 12 - DNA, 103 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
agtcctctacttcttcccaccannnnnnnnnncttcagcttcccgattacgggtacgatccgacggtagtgtnnnnnnnnnntgcttctttgcaaactactgt

SEQ ID NO: 13 - DNA, 109 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
caaaatggactatgtacctgtgtnnnnnnnnnncttcagcttcccgattacgggtacgatccgacggtagtgtnnnnnnnnnntgcattttagatgaaagagaagatgt

SEQ ID NO: 14 - DNA, 110 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
actttccattatgatgtgttagtgtnnnnnnnnnncttcagcttcccgattacgggtacgatccgacggtagtgtnnnnnnnnnnaccttagaaaattgtgcatttaccc

SEQ ID NO: 15 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tgtgcatttacccattttgtgannnnnnnnnncttcagcttcccgattacgggtacgatccgacggtagtgtnnnnnnnnnnatttccagatttgcacagct

SEQ ID NO: 16 - DNA, 105 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
atgaaagagaagatgttcaaaagaannnnnnnnnncttcagcttcccgattacgggtacgatccgacggtagtgtnnnnnnnnnnccccaaaccagcatcactca

SEQ ID NO: 17 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgacctacaggatgggaggcnnnnnnnnnncttcagcttcccgattacgggtacgatccgacggtagtgtnnnnnnnnnntcggcagattaattatgcac

SEQ ID NO: 18 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
acaaagcacacttccaatgatacannnnnnnnnncttcagcttcccgattacgggtacgatccgacggtagtgtnnnnnnnnnnccagtttttgccctgtcagg

SEQ ID NO: 19 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
caggccttcgaggaggtctannnnnnnnnncttcagcttcccgattacgggtacgatccgacggtagtgtnnnnnnnnnnacgaggttgctttactaagga

SEQ ID NO: 20 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tcagaccagaaactgacaacannnnnnnnnncttcagcttcccgattacgggtacgatccgacggtagtgtnnnnnnnnnntcagtgacctacaggatggga

SEQ ID NO: 21 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggtctggatgctgtgacacannnnnnnnnncttcagcttcccgattacgggtacgatccgacggtagtgtnnnnnnnnnnctctgctggtcagtgaacact

SEQ ID NO: 22 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aacgaacagagcctgtgaggnnnnnnnnnncttcagcttcccgattacgggtacgatccgacggtagtgtnnnnnnnnnnggcatgaactcttgtggatcc

SEQ ID NO: 23 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cgcagtgccttgttgacattnnnnnnnnnncttcagcttcccgattacgggtacgatccgacggtagtgtnnnnnnnnnntttctctgcatttggggcca

SEQ ID NO: 24 - DNA, 110 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
cactgaccagcagagagaccgacaannnnnnnnnncttcagcttcccgattacgggtacgatccgacggtagtgtnnnnnnnnnncaaagccctcactcaaacatgaagc

SEQ ID NO: 25 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
acccttgacgtgtgaaacaannnnnnnnnncttcagcttcccgattacgggtacgatccgacggtagtgtnnnnnnnnnnacccctttctttaacaggttga

SEQ ID NO: 26 - DNA, 105 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
accaagagtcagtttatgatttccannnnnnnnnncttcagcttcccgattacgggtacgatccgacggtagtgtnnnnnnnnnnaagcagcactatggagcagg

SEQ ID NO: 27 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ataatcctccactggcaggtnnnnnnnnnncttcagcttcccgattacgggtacgatccgacggtagtgtnnnnnnnnnnagctaaatgcaattaccttcacgt

SEQ ID NO: 28 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
cgtgaaggtaattgcatttagctnnnnnnnnnncttcagcttcccgattacgggtacgatccgacggtagtgtnnnnnnnnnnacctgccagtggaggattat

SEQ ID NO: 29 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tcatggctggattgcaacaannnnnnnnnncttcagcttcccgattacgggtacgatccgacggtagtgtnnnnnnnnnntgtctcattactaattggccct

SEQ ID NO: 30 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tccttgagcaagaaccatgcannnnnnnnnncttcagcttcccgattacgggtacgatccgacggtagtgtnnnnnnnnnnccagctggtggtgaagttga

SEQ ID NO: 31 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gattctcctgagctgggtccnnnnnnnnnncttcagcttcccgattacgggtacgatccgacggtagtgtnnnnnnnnnngtttgcatggttcttgctca

SEQ ID NO: 32 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
acgagttgattgtcggacccnnnnnnnnnncttcagcttcccgattacgggtacgatccgacggtagtgtnnnnnnnnnntgatctggaaccatactgggg

SEQ ID NO: 33 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcctggctttgaatgctctcnnnnnnnnnncttcagcttcccgattacgggtacgatccgacggtagtgtnnnnnnnnnnggctggattgcaacaaacca

SEQ ID NO: 34 - DNA, 118 bp, Artificial Sequence
  source: /note=“Description of Artificial Sequence Synthetic polynucleotide”
  modified_base (28)..(37): a, c, t, g, unknown or other
  modified_base (78)..(87): a, c, t, g, unknown or other
ttcattacatttttgacctacatgtggnnnnnnnnnncttcagcttcccgattacgggtacgatccgacggtagtgtnnnnnnnnnngtctcagtaatcttcttacctatgactatgg

SEQ ID NO: 35 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
acatgcattcaacatcgccannnnnnnnnncttcagcttcccgattacgggtacgatccgacggtagtgtnnnnnnnnnngactatgggcattggttgtcaa

SEQ ID NO: 36 - DNA, 111 bp, Artificial Sequence
  source: /note=“Description of Artificial Sequence Synthetic polynucleotide”
  modified_base (31)..(40): a, c, t, g, unknown or other
  modified_base (81)..(90): a, c, t, g, unknown or other
accctttaaaatatttctatttaaacaagtnnnnnnnnnncttcagcttcccgattacgggtacgatccgacggtagtgtnnnnnnnnnnttccagtcaaataggtctggc

SEQ ID NO: 37 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ccagtcaaataggtctggccnnnnnnnnnncttcagcttcccgattacgggtacgatccgacggtagtgtnnnnnnnnnnaaaagcagtggtagtccaga

SEQ ID NO: 38 - DNA, 108 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
ggatcgagtagtttctctatgccnnnnnnnnnncttcagcttcccgattacgggtacgatccgacggtagtgtnnnnnnnnnntcttcactgcaattttagatactgg

SEQ ID NO: 39 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
tctgagacttgtcatttctacacannnnnnnnnncttcagcttcccgattacgggtacgatccgacggtagtgtnnnnnnnnnnagtcagccacacaacgactg

SEQ ID NO: 40 - DNA, 106 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tgtccatgaatgtcctccagagnnnnnnnnnncttcagcttcccgattacgggtacgatccgacggtagtgtnnnnnnnnnnggacttcttatctggataggtggt

SEQ ID NO: 41 - DNA, 103 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cactttaggtggccttggcannnnnnnnnncttcagcttcccgattacgggtacgatccgacggtagtgtnnnnnnnnnnaggctttgtatatatacacgtgt

SEQ ID NO: 42 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gaagccatccaggaagtggannnnnnnnnncttcagcttcccgattacgggtacgatccgacggtagtgtnnnnnnnnnntgatgtgtagtgttaatgtgct

SEQ ID NO: 43 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
ggacttcttatctggataggtggtnnnnnnnnnncttcagcttcccgattacgggtacgatccgacggtagtgtnnnnnnnnnntcactttaggtggccttggc

SEQ ID NO: 44 - DNA, 105 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
tgcatttttaggtattacgtgcacannnnnnnnnncttcagcttcccgattacgggtacgatccgacggtagtgtnnnnnnnnnnagcattgaagccatccagga

SEQ ID NO: 45 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aggagggggaaaaaccataannnnnnnnnncttcagcttcccgattacgggtacgatccgacggtagtgtnnnnnnnnnncgtgtagggtcagaggtggt

SEQ ID NO: 46 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cggagcccatttccttcacannnnnnnnnncttcagcttcccgattacgggtacgatccgacggtagtgtnnnnnnnnnngtcagtctagcacagggatatg

SEQ ID NO: 47 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aggtggtgacataagcagccnnnnnnnnnncttcagcttcccgattacgggtacgatccgacggtagtgtnnnnnnnnnncaaaccagctcttcacgagg

SEQ ID NO: 48 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
caaaccagagaactgcttccannnnnnnnnncttcagcttcccgattacgggtacgatccgacggtagtgtnnnnnnnnnnccctaagcctcgattcaaga

SEQ ID NO: 49 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agagaagggtttgggggagtnnnnnnnnnncttcagcttcccgattacgggtacgatccgacggtagtgtnnnnnnnnnnggtggtgacataagcagcct

SEQ ID NO: 50 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
gatgtggaagtggtgaaagaccnnnnnnnnnncttcagcttcccgattacgggtacgatccgacggtagtgtnnnnnnnnnnttgtgcagcatttggaagct

SEQ ID NO: 51 - DNA, 103 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tcagcagaaagaagccacgatnnnnnnnnnncttcagcttcccgattacgggtacgatccgacggtagtgtnnnnnnnnnngaggaaaaaggatgacttgcca

SEQ ID NO: 52 - DNA, 112 bp, Artificial Sequence
  source: /note=“Description of Artificial Sequence Synthetic polynucleotide”
  modified_base (31)..(40): a, c, t, g, unknown or other
  modified_base (81)..(90): a, c, t, g, unknown or other
gattgttccagtacattaaatgatgaatcgnnnnnnnnnncttcagcttcccgattacgggtacgatccgacggtagtgtnnnnnnnnnnactctccatcaatgaactgcca

SEQ ID NO: 53 - DNA, 103 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
ctatgatgtgcttgggattccannnnnnnnnncttcagcttcccgattacgggtacgatccgacggtagtgtnnnnnnnnnnatgtggaagtggtgaaagacc

SEQ ID NO: 54 - DNA, 106 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
tttgatgtggtttgatggttaagnnnnnnnnnncttcagcttcccgattacgggtacgatccgacggtagtgtnnnnnnnnnnctcctaaattcaagatgggaatg

SEQ ID NO: 55 - DNA, 103 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gggccgggttggtaatattctnnnnnnnnnncttcagcttcccgattacgggtacgatccgacggtagtgtnnnnnnnnnngggccacaagtttaaaactgca

SEQ ID NO: 56 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
accctgaggcattcccatctnnnnnnnnnncttcagcttcccgattacgggtacgatccgacggtagtgtnnnnnnnnnnaagaaagctgtgtgccttgg

SEQ ID NO: 57 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
acccctgacaaagaaggaagttnnnnnnnnnncttcagcttcccgattacgggtacgatccgacggtagtgtnnnnnnnnnnatgctagctaccctgaggca

SEQ ID NO: 58 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgcagaatcccaaaaccactnnnnnnnnnncttcagcttcccgattacgggtacgatccgacggtagtgtnnnnnnnnnntgggctgtcaaatccatcatgt

SEQ ID NO: 59 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
ggaaaaacaaagcaagtaagtccnnnnnnnnnncttcagcttcccgattacgggtacgatccgacggtagtgtnnnnnnnnnncagggccgggttggtaatat

SEQ ID NO: 60 - DNA, 103 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tcgcatttgggggcatctatnnnnnnnnnncttcagcttcccgattacgggtacgatccgacggtagtgtnnnnnnnnnngccagtcattcaactctttcagt

SEQ ID NO: 61 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gaagagcctcttggacctgannnnnnnnnncttcagcttcccgattacgggtacgatccgacggtagtgtnnnnnnnnnnagttgctttcaaagaggtca

SEQ ID NO: 62 - DNA, 110 bp, Artificial Sequence
  source: /note=“Description of Artificial Sequence Synthetic polynucleotide”
  modified_base (28)..(37): a, c, t, g, unknown or other
  modified_base (78)..(87): a, c, t, g, unknown or other
cctatacacagtaacacagatgacatgnnnnnnnnnncttcagcttcccgattacgggtacgatccgacggtagtgtnnnnnnnnnncttgaagacctaaaacgccaagt

SEQ ID NO: 63 - DNA, 106 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
gccagtcattcaactctttcagtnnnnnnnnnncttcagcttcccgattacgggtacgatccgacggtagtgtnnnnnnnnnnaagcacgcaacataagatacacc

SEQ ID NO: 64 - DNA, 107 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agtggagatcacgcaactgcnnnnnnnnnncttcagcttcccgattacgggtacgatccgacggtagtgtnnnnnnnnnngcaaatcatttcaacacacatgtaaga

SEQ ID NO: 65 - DNA, 109 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ccaccaccatgtgagtgagannnnnnnnnncttcagcttcccgattacgggtacgatccgacggtagtgtnnnnnnnnnnttttcaagttatagttcttttaaaggaca

SEQ ID NO: 66 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tctgctacatctcaggtactccnnnnnnnnnncttcagcttcccgattacgggtacgatccgacggtagtgtnnnnnnnnnnaccaccaccatgtgagtgag

SEQ ID NO: 67 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
acacacactcataatcagctgaannnnnnnnnncttcagcttcccgattacgggtacgatccgacggtagtgtnnnnnnnnnntggagatcacgcaactgctg

SEQ ID NO: 68 - DNA, 106 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
ccttggaattctttaatgtcttgcnnnnnnnnnncttcagcttcccgattacgggtacgatccgacggtagtgtnnnnnnnnnnccgctgggttcttttacaagac

SEQ ID NO: 69 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aatggcatgaataatttgccnnnnnnnnnncttcagcttcccgattacgggtacgatccgacggtagtgtnnnnnnnnnncgttgccatttgagaaggat

SEQ ID NO: 70 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
cgctagaagttggaagggacannnnnnnnnncttcagcttcccgattacgggtacgatccgacggtagtgtnnnnnnnnnntttgcccatcgatctcccaa

SEQ ID NO: 71 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agctgtaaaagacacgggggnnnnnnnnnncttcagcttcccgattacgggtacgatccgacggtagtgtnnnnnnnnnntgctgatgctgtgcttgattg

SEQ ID NO: 72 - DNA, 105 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
aagccatgcactaaaaaggcannnnnnnnnncttcagcttcccgattacgggtacgatccgacggtagtgtnnnnnnnnnntgaaagctagaaagtacatacggc

SEQ ID NO: 73 - DNA, 106 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
agccagttgtgtgaatcttgtnnnnnnnnnncttcagcttcccgattacgggtacgatccgacggtagtgtnnnnnnnnnncccactttaattcagaaaagtagca

SEQ ID NO: 74 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
acaagattcacacaactggctttnnnnnnnnnncttcagcttcccgattacgggtacgatccgacggtagtgtnnnnnnnnnnagctgtaaaagacacggggg

SEQ ID NO: 75 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
acagcacaggttagtgataccaannnnnnnnnncttcagcttcccgattacgggtacgatccgacggtagtgtnnnnnnnnnngcaatccatgggcaaactgt

SEQ ID NO: 76 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
taagcctgggttgcattccannnnnnnnnncttcagcttcccgattacgggtacgatccgacggtagtgtnnnnnnnnnnttatcccaacaccgggcaaa

SEQ ID NO: 77 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
aagcaatccatgggcaaactgnnnnnnnnnncttcagcttcccgattacgggtacgatccgacggtagtgtnnnnnnnnnnttttgatcctttgcgggcac

SEQ ID NO: 78 - DNA, 103 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tatccagccatgcttccgtcnnnnnnnnnncttcagcttcccgattacgggtacgatccgacggtagtgtnnnnnnnnnnagggcaaaaactaatctggttgc

SEQ ID NO: 79 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tgctcaagaggaacttccaccnnnnnnnnnncttcagcttcccgattacgggtacgatccgacggtagtgtnnnnnnnnnntgccactccaagcagtcttt

SEQ ID NO: 80 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgcctcttcttttggggaggnnnnnnnnnncttcagcttcccgattacgggtacgatccgacggtagtgtnnnnnnnnnncaggtacccgaggattctgg

SEQ ID NO: 81 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
gcttgttggtagattgaccttcagnnnnnnnnnncttcagcttcccgattacgggtacgatccgacggtagtgtnnnnnnnnnngatggctgagtggtggtgac

SEQ ID NO: 82 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agcagttttgttggtggtgtnnnnnnnnnncttcagcttcccgattacgggtacgatccgacggtagtgtnnnnnnnnnntacggtgaccacaagggaac

SEQ ID NO: 83 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggtggtgacagcctgtgaaannnnnnnnnncttcagcttcccgattacgggtacgatccgacggtagtgtnnnnnnnnnntgcctcttcttttggggagg

SEQ ID NO: 84 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgcagagtcctgaatttgcannnnnnnnnncttcagcttcccgattacgggtacgatccgacggtagtgtnnnnnnnnnngtcaggcaggagtctcagat

SEQ ID NO: 85 - DNA, 108 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tgagcgagtaatccagctgtgnnnnnnnnnncttcagcttcccgattacgggtacgatccgacggtagtgtnnnnnnnnnnactagtagaatcacagataacaaagca

SEQ ID NO: 86 - DNA, 110 bp, Artificial Sequence
  source: /note=“Description of Artificial Sequence Synthetic polynucleotide”
  modified_base (27)..(36): a, c, t, g, unknown or other
  modified_base (77)..(86): a, c, t, g, unknown or other
agatagcaagcaaaatcaaagtttagnnnnnnnnnncttcagcttcccgattacgggtacgatccgacggtagtgtnnnnnnnnnnggcaacttctcagacttaaaagaa

SEQ ID NO: 87 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agcagcactattttccctgtnnnnnnnnnncttcagcttcccgattacgggtacgatccgacggtagtgtnnnnnnnnnntccagctgtgaagttcagtt

SEQ ID NO: 88 - DNA, 106 bp, Artificial Sequence
  source: /note=“Description of Artificial Sequence Synthetic polynucleotide”
  modified_base (27)..(36): a, c, t, g, unknown or other
  modified_base (77)..(86): a, c, t, g, unknown or other
ggtgaatggtaattacacgagttgatnnnnnnnnnncttcagcttcccgattacgggtacgatccgacggtagtgtnnnnnnnnnnctctcatgctgcaggccata

SEQ ID NO: 89 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tctacttgccctttcaagcctnnnnnnnnnncttcagcttcccgattacgggtacgatccgacggtagtgtnnnnnnnnnnctgatctgctggcatcttgc

SEQ ID NO: 90 - DNA, 105 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
atctgctggcatcttgcagtnnnnnnnnnncttcagcttcccgattacgggtacgatccgacggtagtgtnnnnnnnnnnagtgcttgtctgatataattcagct

SEQ ID NO: 91 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tgtcatctgctccaattgttgnnnnnnnnnncttcagcttcccgattacgggtacgatccgacggtagtgtnnnnnnnnnnttatgctccaaatggaaggag

SEQ ID NO: 92 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
accggctgttcagttgttctnnnnnnnnnncttcagcttcccgattacgggtacgatccgacggtagtgtnnnnnnnnnnacttttaattgctgttggctctga

SEQ ID NO: 93 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
gccagttgctaagtgagagactnnnnnnnnnncttcagcttcccgattacgggtacgatccgacggtagtgtnnnnnnnnnnttcagtctgtgggttcaggg

SEQ ID NO: 94 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
tggcaatttccaagaagacagtannnnnnnnnncttcagcttcccgattacgggtacgatccgacggtagtgtnnnnnnnnnnaaaatccaacccaccacccc

SEQ ID NO: 95 - DNA, 105 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
accacatgaatgatttcaaaccagannnnnnnnnncttcagcttcccgattacgggtacgatccgacggtagtgtnnnnnnnnnnaccggctgttcagttgttct

SEQ ID NO: 96 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttctgatgtgcaggccagagnnnnnnnnnncttcagcttcccgattacgggtacgatccgacggtagtgtnnnnnnnnnngcacaggatgaagtcaaccg

SEQ ID NO: 97 - DNA, 103 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
agcagtaaggcaagtttagctnnnnnnnnnncttcagcttcccgattacgggtacgatccgacggtagtgtnnnnnnnnnnaacatgggtccttgtcctttct

SEQ ID NO: 98 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggaacatgggtccttgtcctnnnnnnnnnncttcagcttcccgattacgggtacgatccgacggtagtgtnnnnnnnnnnaccttctggatttccccaca

SEQ ID NO: 99 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
accattctccctacaacctgtnnnnnnnnnncttcagcttcccgattacgggtacgatccgacggtagtgtnnnnnnnnnncaggccagagagaaagagct

SEQ ID NO: 100 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttggtggcaaagtgtcaaaannnnnnnnnncttcagcttcccgattacgggtacgatccgacggtagtgtnnnnnnnnnngcttgataagcgtgctttattg

SEQ ID NO: 101 - DNA, 103 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
agtcggtgacactaagttgaggnnnnnnnnnncttcagcttcccgattacgggtacgatccgacggtagtgtnnnnnnnnnnttgctcaatgggcaaactacc

SEQ ID NO: 102 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
ttcacactttgccatgttttcctnnnnnnnnnncttcagcttcccgattacgggtacgatccgacggtagtgtnnnnnnnnnntggtttctgactgctggacc

SEQ ID NO: 103 - DNA, 105 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgacactttgccaccaatgcnnnnnnnnnncttcagcttcccgattacgggtacgatccgacggtagtgtnnnnnnnnnnagcaggaatgtatcttcataatcat

SEQ ID NO: 104 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggggaattgcaggtctgtgannnnnnnnnncttcagcttcccgattacgggtacgatccgacggtagtgtnnnnnnnnnntgcgctatcaggagaccatg

SEQ ID NO: 105 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
aggagcaaatgaataaactccgannnnnnnnnncttcagcttcccgattacgggtacgatccgacggtagtgtnnnnnnnnnngagatgtcgaagaaagcgcc

SEQ ID NO: 106 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggccactttgttgctcttgcnnnnnnnnnncttcagcttcccgattacgggtacgatccgacggtagtgtnnnnnnnnnntcttccagcgtccctcaatt

SEQ ID NO: 107 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gctgggaggagagcttcttcnnnnnnnnnncttcagcttcccgattacgggtacgatccgacggtagtgtnnnnnnnnnnagatgctgaaggtcaaatgctt

SEQ ID NO: 108 - DNA, 106 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gccctctgaaattagccggannnnnnnnnncttcagcttcccgattacgggtacgatccgacggtagtgtnnnnnnnnnnagatttcaagtacagttaatttcact

SEQ ID NO: 109 - DNA, 108 bp, Artificial Sequence
  source: /note=“Description of Artificial Sequence Synthetic polynucleotide”
  modified_base (29)..(38): a, c, t, g, unknown or other
  modified_base (79)..(88): a, c, t, g, unknown or other
tctatcagttataaacttctagtggtaannnnnnnnnncttcagcttcccgattacgggtacgatccgacggtagtgtnnnnnnnnnnggccactttgttgctcttgc

SEQ ID NO: 110 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
caggcccaaaaacaattcccannnnnnnnnncttcagcttcccgattacgggtacgatccgacggtagtgtnnnnnnnnnncaggccattcctccttcagaa

SEQ ID NO: 111 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggccattcctccttcagaaannnnnnnnnncttcagcttcccgattacgggtacgatccgacggtagtgtnnnnnnnnnnaggagagcaaaatccacccc

SEQ ID NO: 112 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cagctgaaacagtgcagagtnnnnnnnnnncttcagcttcccgattacgggtacgatccgacggtagtgtnnnnnnnnnntcagcacaccagtaatgcctt

SEQ ID NO: 113 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgggactgatggcattgcatnnnnnnnnnncttcagcttcccgattacgggtacgatccgacggtagtgtnnnnnnnnnnctgcccaccttcattgacact

SEQ ID NO: 114 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cctaatgtctcccttcaccgnnnnnnnnnncttcagcttcccgattacgggtacgatccgacggtagtgtnnnnnnnnnngccagagtttgcttcgagac

SEQ ID NO: 115 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tcagtgggatcacatgtgccnnnnnnnnnncttcagcttcccgattacgggtacgatccgacggtagtgtnnnnnnnnnntcagacaattcagcccagtc

SEQ ID NO: 116 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gaagcaaactctggctctgcnnnnnnnnnncttcagcttcccgattacgggtacgatccgacggtagtgtnnnnnnnnnnaagtacgttgaggcaagcca

SEQ ID NO: 117 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
ggtgggcagaagataaagaatgnnnnnnnnnncttcagcttcccgattacgggtacgatccgacggtagtgtnnnnnnnnnngccatcagtcccaattttac

SEQ ID NO: 118 - DNA, 106 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
ccacaaaacaaacaaacaaaacacgnnnnnnnnnncttcagcttcccgattacgggtacgatccgacggtagtgtnnnnnnnnnngcttgtgtcatccattcgtgc

SEQ ID NO: 119 - DNA, 106 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tgcacgaatggatgacacaagnnnnnnnnnncttcagcttcccgattacgggtacgatccgacggtagtgtnnnnnnnnnncgtgttttgtttgtttgttttgtgg

SEQ ID NO: 120 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
catggggatcagatacactcaannnnnnnnnncttcagcttcccgattacgggtacgatccgacggtagtgtnnnnnnnnnnttcaaggcctcctttctggc

SEQ ID NO: 121 - DNA, 103 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
cctcctttctggcatagacctnnnnnnnnnncttcagcttcccgattacgggtacgatccgacggtagtgtnnnnnnnnnnaccttcatctcttcaactgctt

SEQ ID NO: 122 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcagttgaagagatgaaggtnnnnnnnnnncttcagcttcccgattacgggtacgatccgacggtagtgtnnnnnnnnnngccagaaaggaggccttgaa

SEQ ID NO: 123 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gccagaaaggaggccttgaannnnnnnnnncttcagcttcccgattacgggtacgatccgacggtagtgtnnnnnnnnnnttgagtgtatctgatccccatgag

SEQ ID NO: 124 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
gaaagaaatgcaacaatgcttgnnnnnnnnnncttcagcttcccgattacgggtacgatccgacggtagtgtnnnnnnnnnncgaatggatgacacaagctg

SEQ ID NO: 125 - DNA, 104 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
gggccatttgcttaacttgtgtnnnnnnnnnncttcagcttcccgattacgggtacgatccgacggtagtgtnnnnnnnnnngctgaatgggaaatgcaagact

SEQ ID NO: 126 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgaactccagtctcttccatnnnnnnnnnncttcagcttcccgattacgggtacgatccgacggtagtgtnnnnnnnnnngcttctttttgttgggcctct

SEQ ID NO: 127 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
tggtcatatgtgaggcatagtggnnnnnnnnnncttcagcttcccgattacgggtacgatccgacggtagtgtnnnnnnnnnnctcaagctccacctgtagca

SEQ ID NO: 128 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttcccattcagcctagtgcannnnnnnnnncttcagcttcccgattacgggtacgatccgacggtagtgtnnnnnnnnnngccaaagttgttttgcactgg

SEQ ID NO: 129 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gggcctcttctttagctctctnnnnnnnnnncttcagcttcccgattacgggtacgatccgacggtagtgtnnnnnnnnnngtgcagagccactggtagtt

SEQ ID NO: 130 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ctcaagctccacctgtagcannnnnnnnnncttcagcttcccgattacgggtacgatccgacggtagtgtnnnnnnnnnnactgggatgttgtgagaaag

SEQ ID NO: 131 - DNA, 105 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
ctagcacctcagagatttcctcannnnnnnnnncttcagcttcccgattacgggtacgatccgacggtagtgtnnnnnnnnnnaaggttattagggggaacaaag

SEQ ID NO: 132 - DNA, 120 bp, Artificial Sequence
  source: /note=“Description of Artificial Sequence Synthetic polynucleotide”
  modified_base (31)..(40): a, c, t, g, unknown or other
  modified_base (81)..(90): a, c, t, g, unknown or other
cagtattaaaagaggtcaagtaccaaatagnnnnnnnnnncttcagcttcccgattacgggtacgatccgacggtagtgtnnnnnnnnnntagaatttaaacttaaaaccactgaaaaca

SEQ ID NO: 133 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
ggtcacaagattttgcaaaggnnnnnnnnnncttcagcttcccgattacgggtacgatccgacggtagtgtnnnnnnnnnngcaaacaagtggctaaatgaa

SEQ ID NO: 134 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
gcagctagacagtttcatcatctnnnnnnnnnncttcagcttcccgattacgggtacgatccgacggtagtgtnnnnnnnnnntgccaacatgcccaaacttc

SEQ ID NO: 135 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ccaacatgcccaaacttcctnnnnnnnnnncttcagcttcccgattacgggtacgatccgacggtagtgtnnnnnnnnnnagcacctcagagatttcctca

SEQ ID NO: 136 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggagaaagcaaacaagtggcnnnnnnnnnncttcagcttcccgattacgggtacgatccgacggtagtgtnnnnnnnnnnacctgctacaaagtaaaggtg

SEQ ID NO: 137 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agggtctgtgccaatatgcgnnnnnnnnnncttcagcttcccgattacgggtacgatccgacggtagtgtnnnnnnnnnnatctgagaggcctgtatctgc

SEQ ID NO: 138 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcggagtcatggatgagctannnnnnnnnncttcagcttcccgattacgggtacgatccgacggtagtgtnnnnnnnnnntcagaagatactgagcatttgc

SEQ ID NO: 139 - DNA, 103 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tggattatcagcaaatgctcannnnnnnnnncttcagcttcccgattacgggtacgatccgacggtagtgtnnnnnnnnnntccctccaacgagaattaaatg

SEQ ID NO: 140 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gtagttccctccaacgagaatnnnnnnnnnncttcagcttcccgattacgggtacgatccgacggtagtgtnnnnnnnnnncagtgtctggcattggattgt

SEQ ID NO: 141 - DNA, 103 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
acaccaaggagcatttttgctnnnnnnnnnncttcagcttcccgattacgggtacgatccgacggtagtgtnnnnnnnnnntcctctgaatgtcgcatcaaat

SEQ ID NO: 142 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gctcagctttcaggtttcagannnnnnnnnncttcagcttcccgattacgggtacgatccgacggtagtgtnnnnnnnnnnggcggagtcatggatgagct

SEQ ID NO: 143 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
agacagatttcgcagcttcctnnnnnnnnnncttcagcttcccgattacgggtacgatccgacggtagtgtnnnnnnnnnnttcagtctcctgggcagact

SEQ ID NO: 144 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
gcaagtacatctgggaatcagcnnnnnnnnnncttcagcttcccgattacgggtacgatccgacggtagtgtnnnnnnnnnnaacagagcatccagtctgcc

SEQ ID NO: 145 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gcttgaacagagcatccagtcnnnnnnnnnncttcagcttcccgattacgggtacgatccgacggtagtgtnnnnnnnnnngagctgaatgagtgccagga

SEQ ID NO: 146 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
acttttgcctccttacagcctnnnnnnnnnncttcagcttcccgattacgggtacgatccgacggtagtgtnnnnnnnnnngcttcctgaggcatttgagc

SEQ ID NO: 147 - DNA, 107 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cattgacaagcagttggcagnnnnnnnnnncttcagcttcccgattacgggtacgatccgacggtagtgtnnnnnnnnnnacatttaactgatacactcttattcct

SEQ ID NO: 148 - DNA, 105 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
cgtccaccttgtctgcaatataagnnnnnnnnnncttcagcttcccgattacgggtacgatccgacggtagtgtnnnnnnnnnnagacccccttttcttcctacc

SEQ ID NO: 149 - DNA, 103 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
ccacctctaccatgtagcttccnnnnnnnnnncttcagcttcccgattacgggtacgatccgacggtagtgtnnnnnnnnnngcctccttcccctgattatgt

SEQ ID NO: 150 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
actctttgggcagcctccttnnnnnnnnnncttcagcttcccgattacgggtacgatccgacggtagtgtnnnnnnnnnntgtcctcaaatccaatcttgcc

SEQ ID NO: 151 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
cgttgggcattatactccagtctnnnnnnnnnncttcagcttcccgattacgggtacgatccgacggtagtgtnnnnnnnnnntcctcccaacagaaaatcca

SEQ ID NO: 152 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agacgctgctcaaaattggcnnnnnnnnnncttcagcttcccgattacgggtacgatccgacggtagtgtnnnnnnnnnnggtacctgcgtatttgccac

SEQ ID NO: 153 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
agatctgcctttatttctgaagannnnnnnnnncttcagcttcccgattacgggtacgatccgacggtagtgtnnnnnnnnnngctgctcaaaattggctggt

SEQ ID NO: 154 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
ggacagtgtaaaaaggcactgannnnnnnnnncttcagcttcccgattacgggtacgatccgacggtagtgtnnnnnnnnnngtttccaatgcaggcaagtg

SEQ ID NO: 155 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
caggtaccccttgactttccnnnnnnnnnncttcagcttcccgattacgggtacgatccgacggtagtgtnnnnnnnnnntccagaaaccagccaatttt

SEQ ID NO: 156 - DNA, 114 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
tttgcctttcaaacaataactggtcnnnnnnnnnncttcagcttcccgattacgggtacgatccgacggtagtgtnnnnnnnnnnttgccaccagaaatacataccacacaatg

SEQ ID NO: 157 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcacttgcctgcattggaaannnnnnnnnncttcagcttcccgattacgggtacgatccgacggtagtgtnnnnnnnnnnaggaccagttattgtttgaaaggc

SEQ ID NO: 158 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tctttgtttccaatgcaggcnnnnnnnnnncttcagcttcccgattacgggtacgatccgacggtagtgtnnnnnnnnnngccacaatacatgtgccaat

SEQ ID NO: 159 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tctttgggattttccgtctgcnnnnnnnnnncttcagcttcccgattacgggtacgatccgacggtagtgtnnnnnnnnnnttgcccgttgctttacaattt

SEQ ID NO: 160 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
tccacttcagacttcacttcactnnnnnnnnnncttcagcttcccgattacgggtacgatccgacggtagtgtnnnnnnnnnnacctttgctcccagctcatt

SEQ ID NO: 161 - DNA, 104 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
actggacgtcagattgtacagannnnnnnnnncttcagcttcccgattacgggtacgatccgacggtagtgtnnnnnnnnnnacatggaatagcaattaagggg

SEQ ID NO: 162 - DNA, 108 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
gtggtcaatatctagcttttgcattnnnnnnnnnncttcagcttcccgattacgggtacgatccgacggtagtgtnnnnnnnnnntccacttcagacttcacttcact

SEQ ID NO: 163 - DNA, 103 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gctgagaccacaaacacttctnnnnnnnnnncttcagcttcccgattacgggtacgatccgacggtagtgtnnnnnnnnnntggtgataaagactggacgtca

SEQ ID NO: 164 - DNA, 117 bp, Artificial Sequence
  source: /note=“Description of Artificial Sequence Synthetic polynucleotide”
  modified_base (30)..(39): a, c, t, g, unknown or other
  modified_base (80)..(89): a, c, t, g, unknown or other
ttctccaactgttgctttctttctgttacnnnnnnnnnncttcagcttcccgattacgggtacgatccgacggtagtgtnnnnnnnnnnctttccccaggcaacttcagaatccaaa

SEQ ID NO: 165 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cagcagttgaaggaatgcctnnnnnnnnnncttcagcttcccgattacgggtacgatccgacggtagtgtnnnnnnnnnnagcaacagttggagaaatgct

SEQ ID NO: 166 - DNA, 105 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
tgaaggttattttgaacatacgtgannnnnnnnnncttcagcttcccgattacgggtacgatccgacggtagtgtnnnnnnnnnnagaatggctggcagctacag

SEQ ID NO: 167 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tttccccaggcaacttcagannnnnnnnnncttcagcttcccgattacgggtacgatccgacggtagtgtnnnnnnnnnntcatggtcctgaaaagcacaga

SEQ ID NO: 168 - DNA, 109 bp, Artificial Sequence
  source: /note=“Description of Artificial Sequence Synthetic polynucleotide”
  modified_base (29)..(38): a, c, t, g, unknown or other
  modified_base (79)..(88): a, c, t, g, unknown or other
cacttatttggaacttttatatttctgtnnnnnnnnnncttcagcttcccgattacgggtacgatccgacggtagtgtnnnnnnnnnntcctttcgcatcttacgggac

SEQ ID NO: 169 - DNA, 108 bp, Artificial Sequence
  source: /note=“Description of Artificial Sequence Synthetic polynucleotide”
  modified_base (27)..(36): a, c, t, g, unknown or other
  modified_base (77)..(86): a, c, t, g, unknown or other
gaacatacgtgaaaacacataatatgnnnnnnnnnncttcagcttcccgattacgggtacgatccgacggtagtgtnnnnnnnnnntttcaggtaacagaaagaaagc

SEQ ID NO: 170 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cctttcgcatcttacgggacnnnnnnnnnncttcagcttcccgattacgggtacgatccgacggtagtgtnnnnnnnnnnggttttacctttccccaggc

SEQ ID NO: 171 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggcctctcctacctctgtgannnnnnnnnncttcagcttcccgattacgggtacgatccgacggtagtgtnnnnnnnnnnttaaccactcttctgctcggg

SEQ ID NO: 172 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
caagaaggagacgttggtggannnnnnnnnncttcagcttcccgattacgggtacgatccgacggtagtgtnnnnnnnnnntgctctccttttcacaggct

SEQ ID NO: 173 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
acacccttctctgtcacgagnnnnnnnnnncttcagcttcccgattacgggtacgatccgacggtagtgtnnnnnnnnnncaagaaggagacgttggtgga

SEQ ID NO: 174 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgaaacggctttctgtatggnnnnnnnnnncttcagcttcccgattacgggtacgatccgacggtagtgtnnnnnnnnnnaggcctctcctacctctgtg

SEQ ID NO: 175 - DNA, 103 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tgtacagagacataccatggcannnnnnnnnncttcagcttcccgattacgggtacgatccgacggtagtgtnnnnnnnnnnagcacgtcttctttttgctgg

SEQ ID NO: 176 - DNA, 111 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
caggctgacacacttttggannnnnnnnnncttcagcttcccgattacgggtacgatccgacggtagtgtnnnnnnnnnnttctttaagaatattgtctaaccaataatgc

SEQ ID NO: 177 - DNA, 107 bp, Artificial Sequence
  source: /note=“Description of Artificial Sequence Synthetic polynucleotide”
  modified_base (27)..(36): a, c, t, g, unknown or other
  modified_base (77)..(86): a, c, t, g, unknown or other
accagttacttcaatcatctttgtccnnnnnnnnnncttcagcttcccgattacgggtacgatccgacggtagtgtnnnnnnnnnncacaaagtggatcattcaggc

SEQ ID NO: 178 - DNA, 108 bp, Artificial Sequence
  source: /note=“Description of Artificial Sequence Synthetic polynucleotide”
  modified_base (28)..(37): a, c, t, g, unknown or other
  modified_base (78)..(87): a, c, t, g, unknown or other
gtggtattttcatatagaatattgcgtnnnnnnnnnncttcagcttcccgattacgggtacgatccgacggtagtgtnnnnnnnnnntgtggtccacattctggtcaa

SEQ ID NO: 179 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
cacgtcttctttttgctggggnnnnnnnnnncttcagcttcccgattacgggtacgatccgacggtagtgtnnnnnnnnnntccattcaaagggggaagga

SEQ ID NO: 180 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgagagcaagcacatgcagannnnnnnnnncttcagcttcccgattacgggtacgatccgacggtagtgtnnnnnnnnnngcgtatgtcattcagttctgcc

SEQ ID NO: 181 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cggtgaccactgcaggaaatnnnnnnnnnncttcagcttcccgattacgggtacgatccgacggtagtgtnnnnnnnnnnctcgctctgtttggctctct

SEQ ID NO: 182 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgagctctgagatttggggcnnnnnnnnnncttcagcttcccgattacgggtacgatccgacggtagtgtnnnnnnnnnntgaaaaccttgctgtggggt

SEQ ID NO: 183 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcagtactctgaaaggggcannnnnnnnnncttcagcttcccgattacgggtacgatccgacggtagtgtnnnnnnnnnnagcaaacttgatggcaaacc

SEQ ID NO: 184 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggtcacgtgtagagtccaccnnnnnnnnnncttcagcttcccgattacgggtacgatccgacggtagtgtnnnnnnnnnncgcaagagaccatttagcaca

SEQ ID NO: 185 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cctctttcagattcacccccnnnnnnnnnncttcagcttcccgattacgggtacgatccgacggtagtgtnnnnnnnnnnaaggccaagaatattctgcat

SEQ ID NO: 186 - DNA, 105 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
tggaaagaacttagataagtctccannnnnnnnnncttcagcttcccgattacgggtacgatccgacggtagtgtnnnnnnnnnncttgaaccactggaggctga

SEQ ID NO: 187 - DNA, 103 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cttcaaaggaatggaggcctnnnnnnnnnncttcagcttcccgattacgggtacgatccgacggtagtgtnnnnnnnnnntttccactcctagttcattcaca

SEQ ID NO: 188 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttgcttgaaccactggaggcnnnnnnnnnncttcagcttcccgattacgggtacgatccgacggtagtgtnnnnnnnnnntgtgattagtttagcaacaggagg

SEQ ID NO: 189 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
catttattcaacctcctgttgcnnnnnnnnnncttcagcttcccgattacgggtacgatccgacggtagtgtnnnnnnnnnntttcagattcaccccctgct

SEQ ID NO: 190 - DNA, 107 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
agatgagagaaagcgagaggannnnnnnnnncttcagcttcccgattacgggtacgatccgacggtagtgtnnnnnnnnnnaccaaaatgaagactgtacttgttgt

SEQ ID NO: 191 - DNA, 106 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttgtctgtaacagctgctgtnnnnnnnnnncttcagcttcccgattacgggtacgatccgacggtagtgtnnnnnnnnnngaacagaaaaagtgagtttctgatga

SEQ ID NO: 192 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
tgagtggtatttgattttgaacgnnnnnnnnnncttcagcttcccgattacgggtacgatccgacggtagtgtnnnnnnnnnntgagagaaagcgagaggaaa

SEQ ID NO: 193 - DNA, 109 bp, Artificial Sequence
  source: /note=“Description of Artificial Sequence Synthetic polynucleotide”
  modified_base (27)..(36): a, c, t, g, unknown or other
  modified_base (77)..(86): a, c, t, g, unknown or other
gctcatagcctttcttttacatttggnnnnnnnnnncttcagcttcccgattacgggtacgatccgacggtagtgtnnnnnnnnnnacagtaccctcattgtcttcatt

SEQ ID NO: 194 - DNA, 107 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
ccctcattgtcttcattctgatcannnnnnnnnncttcagcttcccgattacgggtacgatccgacggtagtgtnnnnnnnnnntgttttgtctgtaacagctgctg

SEQ ID NO: 195 - DNA, 108 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
ttgttgcaaagaggagacaactnnnnnnnnnncttcagcttcccgattacgggtacgatccgacggtagtgtnnnnnnnnnnagcattccatgaaagttttaaattgg

SEQ ID NO: 196 - DNA, 105 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
ttgatgttcttgtttctattaacgtnnnnnnnnnncttcagcttcccgattacgggtacgatccgacggtagtgtnnnnnnnnnngaggcaggctgatgatctcc

SEQ ID NO: 197 - DNA, 111 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
cctcaaatcctgttcatggtgcnnnnnnnnnncttcagcttcccgattacgggtacgatccgacggtagtgtnnnnnnnnnntggtattgacattctaaaacaacattacc

SEQ ID NO: 198 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tcagtacaagaggcaggctgnnnnnnnnnncttcagcttcccgattacgggtacgatccgacggtagtgtnnnnnnnnnntaactgcagccagaagtgca

SEQ ID NO: 199 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gctcaggtaggctggctaatnnnnnnnnnncttcagcttcccgattacgggtacgatccgacggtagtgtnnnnnnnnnnacaacacacaatacaaggaaatgc

SEQ ID NO: 200 - DNA, 107 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgtcatccaagcatttcaggnnnnnnnnnncttcagcttcccgattacgggtacgatccgacggtagtgtnnnnnnnnnncaacattttaaatatgatcttcacagg

SEQ ID NO: 201 - DNA, 105 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
ttgtgcaaagttgagtcttcgannnnnnnnnncttcagcttcccgattacgggtacgatccgacggtagtgtnnnnnnnnnnagtgttacagaagcccaaagtga

SEQ ID NO: 202 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gagctggatctgagttggctnnnnnnnnnncttcagcttcccgattacgggtacgatccgacggtagtgtnnnnnnnnnnaaacacatacgtgggtttgc

SEQ ID NO: 203 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tttgctctcaatttcccgccnnnnnnnnnncttcagcttcccgattacgggtacgatccgacggtagtgtnnnnnnnnnnccactcactttcagaatgtaca

SEQ ID NO: 204 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ctggcaaacccacgtatgtgnnnnnnnnnncttcagcttcccgattacgggtacgatccgacggtagtgtnnnnnnnnnngagctgaatgcagtgcgtag

SEQ ID NO: 205 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcagtggagccaactcagatnnnnnnnnnncttcagcttcccgattacgggtacgatccgacggtagtgtnnnnnnnnnntgcttgcaagtcggttgatg

SEQ ID NO: 206 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ccagggcagttagctaaccannnnnnnnnncttcagcttcccgattacgggtacgatccgacggtagtgtnnnnnnnnnnttgctctcaatttcccgcca

SEQ ID NO: 207 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tcaaaggctgttgtccctttnnnnnnnnnncttcagcttcccgattacgggtacgatccgacggtagtgtnnnnnnnnnnccatcctcagacaagccctc

SEQ ID NO: 208 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aatgctcctgacctctgtgcnnnnnnnnnncttcagcttcccgattacgggtacgatccgacggtagtgtnnnnnnnnnntaccagcacactgtccgtga

SEQ ID NO: 209 - DNA, 117 bp, Artificial Sequence
  source: /note=“Description of Artificial Sequence Synthetic polynucleotide”
  modified_base (29)..(38): a, c, t, g, unknown or other
  modified_base (79)..(88): a, c, t, g, unknown or other
ccatcatcgtttcttcacggacagtgtgnnnnnnnnnncttcagcttcccgattacgggtacgatccgacggtagtgtnnnnnnnnnncttcagagactcctcttgcttaaagagat

SEQ ID NO: 210 - DNA, 104 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
cctaacagtgaaacctcctccatnnnnnnnnnncttcagcttcccgattacgggtacgatccgacggtagtgtnnnnnnnnnngggcttgtgagacatgagtga

SEQ ID NO: 211 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
tgcatcatgatggcattttgactnnnnnnnnnncttcagcttcccgattacgggtacgatccgacggtagtgtnnnnnnnnnngctcctgacctctgtgctaa

SEQ ID NO: 212 - DNA, 105 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gggcttgtgagacatgagtgannnnnnnnnncttcagcttcccgattacgggtacgatccgacggtagtgtnnnnnnnnnngtgctttggttttaccttcagaga

SEQ ID NO: 213 - DNA, 108 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tctacaacaaagctcaggtcggnnnnnnnnnncttcagcttcccgattacgggtacgatccgacggtagtgtnnnnnnnnnngtcaataattaagaattgcaacacca

SEQ ID NO: 214 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
acaaatcccaaaggtagcaaatggnnnnnnnnnncttcagcttcccgattacgggtacgatccgacggtagtgtnnnnnnnnnnttccacaggcgttgcacttt

SEQ ID NO: 215 - DNA, 110 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gggagagagcttcctgtagcnnnnnnnnnncttcagcttcccgattacgggtacgatccgacggtagtgtnnnnnnnnnnctgaaataaattctacagttccctgaaaac

SEQ ID NO: 216 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
ggaccgacaagggtaggtaacnnnnnnnnnncttcagcttcccgattacgggtacgatccgacggtagtgtnnnnnnnnnnacaacaaagctcaggtcgga

SEQ ID NO: 217 - DNA, 103 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
actgttcagcttctgttagccannnnnnnnnncttcagcttcccgattacgggtacgatccgacggtagtgtnnnnnnnnnntccatcacccttcagaacctg

SEQ ID NO: 218 - DNA, 109 bp, Artificial Sequence
  source: /note=“Description of Artificial Sequence Synthetic polynucleotide”
  modified_base (28)..(37): a, c, t, g, unknown or other
  modified_base (78)..(87): a, c, t, g, unknown or other
ggatcaagaaaaatagatggattatgtnnnnnnnnnncttcagcttcccgattacgggtacgatccgacggtagtgtnnnnnnnnnncccaattctcaggaatttgtgt

SEQ ID NO: 219 - DNA, 107 bp, Artificial Sequence
  source: /note=“Description of Artificial Sequence Synthetic polynucleotide”
  modified_base (28)..(37): a, c, t, g, unknown or other
  modified_base (78)..(87): a, c, t, g, unknown or other
ggttatactgacaaagatatcactctgnnnnnnnnnncttcagcttcccgattacgggtacgatccgacggtagtgtnnnnnnnnnnagatctgtcaaatcgcctgc

SEQ ID NO: 220 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
ttcctgagaattgggaacatgcnnnnnnnnnncttcagcttcccgattacgggtacgatccgacggtagtgtnnnnnnnnnnatgcttttacctgcaggcga

SEQ ID NO: 221 - DNA, 109 bp, Artificial Sequence; identical to SEQ ID NO: 218
  source: /note=“Description of Artificial Sequence Synthetic polynucleotide”
  modified_base (28)..(37): a, c, t, g, unknown or other
  modified_base (78)..(87): a, c, t, g, unknown or other

SEQ ID NO: 222 - DNA, 110 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
tgcaggtaaaagcatatggatcaagnnnnnnnnnncttcagcttcccgattacgggtacgatccgacggtagtgtnnnnnnnnnntccatcacccttcagaacctgatct

SEQ ID NO: 223 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttgggaagcctgaatctgcgnnnnnnnnnncttcagcttcccgattacgggtacgatccgacggtagtgtnnnnnnnnnnggggcttcatttttgttttgcc

SEQ ID NO: 224 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cccaatgccatcctggagttnnnnnnnnnncttcagcttcccgattacgggtacgatccgacggtagtgtnnnnnnnnnntctgtctgacagctgtttgca

SEQ ID NO: 225 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
caaaaatgaagccccatgtcnnnnnnnnnncttcagcttcccgattacgggtacgatccgacggtagtgtnnnnnnnnnntttcttccccagttgcattc

SEQ ID NO: 226 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tgacatgcccatatccaaaggannnnnnnnnncttcagcttcccgattacgggtacgatccgacggtagtgtnnnnnnnnnnccaatgccatcctggagttc

SEQ ID NO: 227 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgacagctgtttgcagacctnnnnnnnnnncttcagcttcccgattacgggtacgatccgacggtagtgtnnnnnnnnnngttagtgcctttcaccctgc

SEQ ID NO: 228 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agaggtagggcgacagatctnnnnnnnnnncttcagcttcccgattacgggtacgatccgacggtagtgtnnnnnnnnnnggcaaactgttgtcagaaca

SEQ ID NO: 229 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
agcaatgttatctgcttcctccannnnnnnnnncttcagcttcccgattacgggtacgatccgacggtagtgtnnnnnnnnnnctttatgcaagcaggccctg

SEQ ID NO: 230 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ctgggacacaaacatggcaannnnnnnnnncttcagcttcccgattacgggtacgatccgacggtagtgtnnnnnnnnnntgttatctgcttcctccaacca

SEQ ID NO: 231 - DNA, 103 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
acctggaaaagagcagcaacnnnnnnnnnncttcagcttcccgattacgggtacgatccgacggtagtgtnnnnnnnnnntctttctccaggctagaagaaca

SEQ ID NO: 232 - DNA, 114 bp, Artificial Sequence
  source: /note=“Description of Artificial Sequence Synthetic polynucleotide”
  modified_base (28)..(37): a, c, t, g, unknown or other
  modified_base (78)..(87): a, c, t, g, unknown or other
gacaagatattcttttgttcttctagcnnnnnnnnnncttcagcttcccgattacgggtacgatccgacggtagtgtnnnnnnnnnncttgacttgctcaagcttttcttttag

SEQ ID NO: 233 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gtttgagaattccctggcgcnnnnnnnnnncttcagcttcccgattacgggtacgatccgacggtagtgtnnnnnnnnnnacacatgtgacggaagagatgg

SEQ ID NO: 234 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
ggaggctggtatgtggattgtnnnnnnnnnncttcagcttcccgattacgggtacgatccgacggtagtgtnnnnnnnnnngtgctcccataagcccagaa

SEQ ID NO: 235 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
ggcccagtggtacctcaaatannnnnnnnnncttcagcttcccgattacgggtacgatccgacggtagtgtnnnnnnnnnngggcaactcttccaccagtaa

SEQ ID NO: 236 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aggacccgtgcttgtaagtgnnnnnnnnnncttcagcttcccgattacgggtacgatccgacggtagtgtnnnnnnnnnnctcggtcaagtcgcttcatt

SEQ ID NO: 237 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tggagatttgtctgcttgagctnnnnnnnnnncttcagcttcccgattacgggtacgatccgacggtagtgtnnnnnnnnnntagccaaagcaaacggtcag

SEQ ID NO: 238 - DNA, 115 bp, Artificial Sequence
  source: /note=“Description of Artificial Sequence Synthetic polynucleotide”
  modified_base (29)..(38): a, c, t, g, unknown or other
  modified_base (79)..(88): a, c, t, g, unknown or other
gtaactgaaacagacaaatgcaacaacgnnnnnnnnnncttcagcttcccgattacgggtacgatccgacggtagtgtnnnnnnnnnngtctaacctttatccactggagatttg

SEQ ID NO: 239 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgctgctgtggttatctcctnnnnnnnnnncttcagcttcccgattacgggtacgatccgacggtagtgtnnnnnnnnnnttcctttcaggtttccagagct

SEQ ID NO: 240 - DNA, 109 bp, Artificial Sequence
  source: /note=“Description of Artificial Sequence Synthetic polynucleotide”
  modified_base (29)..(38): a, c, t, g, unknown or other
  modified_base (79)..(88): a, c, t, g, unknown or other
ggcaatatcactgaattttctcatttggnnnnnnnnnncttcagcttcccgattacgggtacgatccgacggtagtgtnnnnnnnnnnctgctgctgtggttatctcct

SEQ ID NO: 241 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tttcaagctgcccaaggtctnnnnnnnnnncttcagcttcccgattacgggtacgatccgacggtagtgtnnnnnnnnnnaacgtcaaatggtccttcttgg

SEQ ID NO: 242 - DNA, 106 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
ggtaaataattctcaaggcataagcnnnnnnnnnncttcagcttcccgattacgggtacgatccgacggtagtgtnnnnnnnnnntttcaagctgcccaaggtctt

SEQ ID NO: 243 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tctcttccacatccggttgtnnnnnnnnnncttcagcttcccgattacgggtacgatccgacggtagtgtnnnnnnnnnngtccacgtcaatggcaaatgt

SEQ ID NO: 244 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttcctggggaaaagaacccannnnnnnnnncttcagcttcccgattacgggtacgatccgacggtagtgtnnnnnnnnnntgcttcattaccttcactggct

SEQ ID NO: 245 - DNA, 103 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gggcagcatttgtacaaggannnnnnnnnncttcagcttcccgattacgggtacgatccgacggtagtgtnnnnnnnnnntctgcaatacatgtggagtctcc

SEQ ID NO: 246 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcctggtacataagggcacannnnnnnnnncttcagcttcccgattacgggtacgatccgacggtagtgtnnnnnnnnnntccacatccggttgtttagct

SEQ ID NO: 247 - DNA, 103 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
agcagttcaagctaaacaaccgnnnnnnnnnncttcagcttcccgattacgggtacgatccgacggtagtgtnnnnnnnnnnttctctgcaccaaaagctaca

SEQ ID NO: 248 - DNA, 105 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tggatcccattctctttggctnnnnnnnnnncttcagcttcccgattacgggtacgatccgacggtagtgtnnnnnnnnnnagaggaagttagaagatctgagct

SEQ ID NO: 249 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
agtgggtagaatttcttttaaaggnnnnnnnnnncttcagcttcccgattacgggtacgatccgacggtagtgtnnnnnnnnnnggtttaccgccttccactca

SEQ ID NO: 250 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
acttcaagagctgagggcaannnnnnnnnncttcagcttcccgattacgggtacgatccgacggtagtgtnnnnnnnnnnttcaccaaatggattaagatgttc

SEQ ID NO: 251 - DNA, 116 bp, Artificial Sequence
  source: /note=“Description of Artificial Sequence Synthetic polynucleotide”
  modified_base (29)..(38): a, c, t, g, unknown or other
  modified_base (79)..(88): a, c, t, g, unknown or other
attcatgaacatcttaatccatttggtgnnnnnnnnnncttcagcttcccgattacgggtacgatccgacggtagtgtnnnnnnnnnntctctctcacccagtcatcacttcatag

SEQ ID NO: 252 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agtccaggagctaggtcaggnnnnnnnnnncttcagcttcccgattacgggtacgatccgacggtagtgtnnnnnnnnnntctctctcacccagtcatcac

SEQ ID NO: 253 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcagatttcaaccgggcttgnnnnnnnnnncttcagcttcccgattacgggtacgatccgacggtagtgtnnnnnnnnnntttcctttttgcaaaaaccca

SEQ ID NO: 254 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
agccaaactcttattcatgacannnnnnnnnncttcagcttcccgattacgggtacgatccgacggtagtgtnnnnnnnnnnaccacaggttgtgtcaccag

SEQ ID NO: 255 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gtcacccaccatcaccctctnnnnnnnnnncttcagcttcccgattacgggtacgatccgacggtagtgtnnnnnnnnnnagttgcctaagaactggtggg

SEQ ID NO: 256 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
aatgaagattttccaccaatcacnnnnnnnnnncttcagcttcccgattacgggtacgatccgacggtagtgtnnnnnnnnnntaccgactggctttctctgc

SEQ ID NO: 257 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
tgtgtcaccagagtaacagtctgnnnnnnnnnncttcagcttcccgattacgggtacgatccgacggtagtgtnnnnnnnnnnaagcagagaaagccagtcgg

SEQ ID NO: 258 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
cgagatgatcatcaagcagaaggnnnnnnnnnncttcagcttcccgattacgggtacgatccgacggtagtgtnnnnnnnnnngttggaggtacctgctctgg

SEQ ID NO: 259 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttgggcagcggtaatgagttnnnnnnnnnncttcagcttcccgattacgggtacgatccgacggtagtgtnnnnnnnnnntgaaacttgtcatgcatcttgc

SEQ ID NO: 260 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tgtgagaccagccaaaacactnnnnnnnnnncttcagcttcccgattacgggtacgatccgacggtagtgtnnnnnnnnnnttcaaattttgggcagcggt

SEQ ID NO: 261 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agaccagcaatcaagaggctnnnnnnnnnncttcagcttcccgattacgggtacgatccgacggtagtgtnnnnnnnnnncacaacgctgaagaaccctg

SEQ ID NO: 262 - DNA, 116 bp, Artificial Sequence
  source: /note=“Description of Artificial Sequence Synthetic polynucleotide”
  modified_base (28)..(37): a, c, t, g, unknown or other
  modified_base (78)..(87): a, c, t, g, unknown or other
catcccactgattctgaattctttcaannnnnnnnnncttcagcttcccgattacgggtacgatccgacggtagtgtnnnnnnnnnncttggtttctgtgattttcttttggattg

SEQ ID NO: 263 - DNA, 106 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
atagggaccctccttccatgannnnnnnnnncttcagcttcccgattacgggtacgatccgacggtagtgtnnnnnnnnnnactgttcatttcagctttaacgtga

SEQ ID NO: 264 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aaatgctagtctggaggagannnnnnnnnncttcagcttcccgattacgggtacgatccgacggtagtgtnnnnnnnnnngcctgtcctaagacctgctc

SEQ ID NO: 265 - DNA, 106 bp, Artificial Sequence
  source: /note=“Description of Artificial Sequence Synthetic polynucleotide”
  modified_base (27)..(36): a, c, t, g, unknown or other
  modified_base (77)..(86): a, c, t, g, unknown or other
ccaaaagaaaatcacagaaaccaaggnnnnnnnnnncttcagcttcccgattacgggtacgatccgacggtagtgtnnnnnnnnnngaaccggaggcaacagttga

SEQ ID NO: 266 - DNA, 105 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
ggctaggatgatgaacaacaggnnnnnnnnnncttcagcttcccgattacgggtacgatccgacggtagtgtnnnnnnnnnnggtgttcttgtacttcatcccac

SEQ ID NO: 267 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
accggaggcaacagttgaatnnnnnnnnnncttcagcttcccgattacgggtacgatccgacggtagtgtnnnnnnnnnnagcaacataaatgtgagataacgt

SEQ ID NO: 268 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tggtgaaactggatggaccannnnnnnnnncttcagcttcccgattacgggtacgatccgacggtagtgtnnnnnnnnnnttggccctgaaacttctccg

SEQ ID NO: 269 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
atgtggcaaatgacttggccnnnnnnnnnncttcagcttcccgattacgggtacgatccgacggtagtgtnnnnnnnnnntgaggattcagaagctgtttacga

SEQ ID NO: 270 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
aggtctttggccaactgctatnnnnnnnnnncttcagcttcccgattacgggtacgatccgacggtagtgtnnnnnnnnnnatgaatgcttctccaagagg

SEQ ID NO: 271 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tgaatgcttctccaagaggcannnnnnnnnncttcagcttcccgattacgggtacgatccgacggtagtgtnnnnnnnnnnagaagtctgagccaagtccg

SEQ ID NO: 272 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tacgggtagcatcctgtaggannnnnnnnnncttcagcttcccgattacgggtacgatccgacggtagtgtnnnnnnnnnntttgtccctggcttgtcagt

SEQ ID NO: 273 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
caccctgcaaaggaccaaatgnnnnnnnnnncttcagcttcccgattacgggtacgatccgacggtagtgtnnnnnnnnnngcctttccttacgggtagca

SEQ ID NO: 274 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gggtgagttgttgctacagcnnnnnnnnnncttcagcttcccgattacgggtacgatccgacggtagtgtnnnnnnnnnntcttccaaagcagcctctcg

SEQ ID NO: 275 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cccctggacctggaaaagttnnnnnnnnnncttcagcttcccgattacgggtacgatccgacggtagtgtnnnnnnnnnntggagttcactaggtgcacc

SEQ ID NO: 276 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tcaggcatttccgctttagcnnnnnnnnnncttcagcttcccgattacgggtacgatccgacggtagtgtnnnnnnnnnntactgcaacagttccccctg

SEQ ID NO: 277 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tcaagtggagtgaacttcggannnnnnnnnncttcagcttcccgattacgggtacgatccgacggtagtgtnnnnnnnnnnttcttcttcctgctgtcctgt

SEQ ID NO: 278 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
atgtggagcaaaaaggccacnnnnnnnnnncttcagcttcccgattacgggtacgatccgacggtagtgtnnnnnnnnnntcctgagatccctggaaggt

SEQ ID NO: 279 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tcctacaggacagcaggaagannnnnnnnnncttcagcttcccgattacgggtacgatccgacggtagtgtnnnnnnnnnnaacaggactgcatcatcgga

SEQ ID NO: 280 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
cgatgaatgtgaatttggagaannnnnnnnnncttcagcttcccgattacgggtacgatccgacggtagtgtnnnnnnnnnnttggctgttttcatccaggt

SEQ ID NO: 281 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aacaggactgcatcatcggannnnnnnnnncttcagcttcccgattacgggtacgatccgacggtagtgtnnnnnnnnnntgtgagataccagttacttgtgct

SEQ ID NO: 282 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
caaatcccttttcttggcgtnnnnnnnnnncttcagcttcccgattacgggtacgatccgacggtagtgtnnnnnnnnnnagcttcaatttcaccttggagg

SEQ ID NO: 283 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tgagagccacaaaacagaggatnnnnnnnnnncttcagcttcccgattacgggtacgatccgacggtagtgtnnnnnnnnnnttccactggtcagaactggc

SEQ ID NO: 284 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agccacaccagaagttcctgnnnnnnnnnncttcagcttcccgattacgggtacgatccgacggtagtgtnnnnnnnnnntgtgcttaacatgtgcaaggc

SEQ ID NO: 285 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gaggcgactttccagcagttnnnnnnnnnncttcagcttcccgattacgggtacgatccgacggtagtgtnnnnnnnnnntctgacatggtacgctgctg

SEQ ID NO: 286 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ctcttctcacccaagggtcannnnnnnnnncttcagcttcccgattacgggtacgatccgacggtagtgtnnnnnnnnnntccagcagttcagaagcaga

SEQ ID NO: 287 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ccctcttgaaggcctgtgaannnnnnnnnncttcagcttcccgattacgggtacgatccgacggtagtgtnnnnnnnnnnctgctccgtcaccactgatc

SEQ ID NO: 288 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
accaggagcccagaggtaatnnnnnnnnnncttcagcttcccgattacgggtacgatccgacggtagtgtnnnnnnnnnntgagaagaatgccacaagcca

SEQ ID NO: 289 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
cctgggtgctcagaacttgttnnnnnnnnnncttcagcttcccgattacgggtacgatccgacggtagtgtnnnnnnnnnntccaaaggctgctctgtcag

SEQ ID NO: 290 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cagggtctggatagctctcannnnnnnnnncttcagcttcccgattacgggtacgatccgacggtagtgtnnnnnnnnnngaaactctaccaggagcccag

SEQ ID NO: 291 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tcaatgaggagatcgcccacnnnnnnnnnncttcagcttcccgattacgggtacgatccgacggtagtgtnnnnnnnnnntgtgaaagacggactgatttctct

SEQ ID NO: 292 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agggccctttgagagactcannnnnnnnnncttcagcttcccgattacgggtacgatccgacggtagtgtnnnnnnnnnntgagacccttgaaagactcc

SEQ ID NO: 293 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aagctgaggtgatcaagggannnnnnnnnncttcagcttcccgattacgggtacgatccgacggtagtgtnnnnnnnnnnagagcccagaatgtcactcg

SEQ ID NO: 294 - DNA, 105 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
ggcataaattttgatacagcccagannnnnnnnnncttcagcttcccgattacgggtacgatccgacggtagtgtnnnnnnnnnnttctgggctctctcctcagg

SEQ ID NO: 295 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttctgggctctctcctcaggnnnnnnnnnncttcagcttcccgattacgggtacgatccgacggtagtgtnnnnnnnnnncagcttgaggtccagctcat

SEQ ID NO: 296 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aaattgaacctgcactccgcnnnnnnnnnncttcagcttcccgattacgggtacgatccgacggtagtgtnnnnnnnnnntgtggcctaaaaccttgtca

SEQ ID NO: 297 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tcgaagtgcctgtgtgcaatnnnnnnnnnncttcagcttcccgattacgggtacgatccgacggtagtgtnnnnnnnnnntgcagaagcttccatctggt

SEQ ID NO: 298 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
tgttcatggtaatatttgtgaggannnnnnnnnncttcagcttcccgattacgggtacgatccgacggtagtgtnnnnnnnnnntctggaagacctgaacacca

SEQ ID NO: 299 - DNA, 105 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
agcacattgtaaacattgttgtcctnnnnnnnnnncttcagcttcccgattacgggtacgatccgacggtagtgtnnnnnnnnnncacgtcaatgaccttgctcg

SEQ ID NO: 300 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cacgtcaatgaccttgctcgnnnnnnnnnncttcagcttcccgattacgggtacgatccgacggtagtgtnnnnnnnnnnagcaaacattactggcactgc

SEQ ID NO: 301 - DNA, 105 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
tggttgataagttgagaaggttaggnnnnnnnnnncttcagcttcccgattacgggtacgatccgacggtagtgtnnnnnnnnnnatgaagcccacagggacttt

SEQ ID NO: 302 - DNA, 108 bp, Artificial Sequence
  source: /note=“Description of Artificial Sequence Synthetic polynucleotide”
  modified_base (29)..(38): a, c, t, g, unknown or other
  modified_base (79)..(88): a, c, t, g, unknown or other
ccagtaagtcattttcagcttttatcacnnnnnnnnnncttcagcttcccgattacgggtacgatccgacggtagtgtnnnnnnnnnnctccttttcctcccaggtgg

SEQ ID NO: 303 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgctgagatgctggaccaaannnnnnnnnncttcagcttcccgattacgggtacgatccgacggtagtgtnnnnnnnnnncaggatgatttatgcttctactgc

SEQ ID NO: 304 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
tccaagactgagaacactaaagcannnnnnnnnncttcagcttcccgattacgggtacgatccgacggtagtgtnnnnnnnnnnttcatgcagctgcctgactc

SEQ ID NO: 305 - DNA, 107 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
tcaagtaagttggaagtatcacattnnnnnnnnnncttcagcttcccgattacgggtacgatccgacggtagtgtnnnnnnnnnnagcaaacagaccaatatcagtg

SEQ ID NO: 306 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gccaaacaaagtgccctactnnnnnnnnnncttcagcttcccgattacgggtacgatccgacggtagtgtnnnnnnnnnntgtcttcatgggcagctgag

SEQ ID NO: 307 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ccctggacagacgctgaaaannnnnnnnnncttcagcttcccgattacgggtacgatccgacggtagtgtnnnnnnnnnnacaggtattgtaggccaggc

SEQ ID NO: 308 - DNA, 105 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
catcgcaaacaggaaagacannnnnnnnnncttcagcttcccgattacgggtacgatccgacggtagtgtnnnnnnnnnnacaggttagtcacaataaatgctct

SEQ ID NO: 309 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcttttgaaccattcggaatnnnnnnnnnncttcagcttcccgattacgggtacgatccgacggtagtgtnnnnnnnnnngctctgtcattttgggatgg

SEQ ID NO: 310 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tgcagtgtgaaagttacttgctnnnnnnnnnncttcagcttcccgattacgggtacgatccgacggtagtgtnnnnnnnnnntgtgttttagccacgagact

SEQ ID NO: 311 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggatggtcccagcaagttgtnnnnnnnnnncttcagcttcccgattacgggtacgatccgacggtagtgtnnnnnnnnnntggataggaaggtgccactg

SEQ ID NO: 312 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gctgtcacaattcctgttgcannnnnnnnnncttcagcttcccgattacgggtacgatccgacggtagtgtnnnnnnnnnnaggactgccatgaaactccg

SEQ ID NO: 313 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aggactgccatgaaactccgnnnnnnnnnncttcagcttcccgattacgggtacgatccgacggtagtgtnnnnnnnnnntattggcaaatcactgggcg

SEQ ID NO: 314 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aaagggccttctgcagtcttnnnnnnnnnncttcagcttcccgattacgggtacgatccgacggtagtgtnnnnnnnnnnaggcaaactctaggccaagg

SEQ ID NO: 315 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aggtcagctgaaaagagggannnnnnnnnncttcagcttcccgattacgggtacgatccgacggtagtgtnnnnnnnnnntacattgcaacaggaattgtg

SEQ ID NO: 316 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ataacagacaacccacccccnnnnnnnnnncttcagcttcccgattacgggtacgatccgacggtagtgtnnnnnnnnnnacttacagcaaagggccttct

SEQ ID NO: 317 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
accttcctttcagtgtccttnnnnnnnnnncttcagcttcccgattacgggtacgatccgacggtagtgtnnnnnnnnnncttgctccaggcggtcataa

SEQ ID NO: 318 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
accacactctctttgaaaggtgtnnnnnnnnnncttcagcttcccgattacgggtacgatccgacggtagtgtnnnnnnnnnncagctgacaggctcaagaga

SEQ ID NO: 319 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcccatggatatcctgcagannnnnnnnnncttcagcttcccgattacgggtacgatccgacggtagtgtnnnnnnnnnnagggtatgagagagtcctagct

SEQ ID NO: 320 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttcagcagccagttcagacannnnnnnnnncttcagcttcccgattacgggtacgatccgacggtagtgtnnnnnnnnnncttccagggccctgttgtaa

SEQ ID NO: 321 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
acaggaggcttagcgtacagnnnnnnnnnncttcagcttcccgattacgggtacgatccgacggtagtgtnnnnnnnnnnttatgaccgcctggagcaag

SEQ ID NO: 322 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ttgaggttgtgctggtccaannnnnnnnnncttcagcttcccgattacgggtacgatccgacggtagtgtnnnnnnnnnnttcagcagccagttcagaca

SEQ ID NO: 323 - DNA, 104 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cctccctgttcgtcccctatnnnnnnnnnncttcagcttcccgattacgggtacgatccgacggtagtgtnnnnnnnnnnaagaacagtctgtcatttcccatc

SEQ ID NO: 324 - DNA, 106 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
atctgtacttgtcttccaaatgtgcnnnnnnnnnncttcagcttcccgattacgggtacgatccgacggtagtgtnnnnnnnnnntgacaaggaatggcacaaacc

SEQ ID NO: 325 - DNA, 103 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
actggcatcatttccctgtgtnnnnnnnnnncttcagcttcccgattacgggtacgatccgacggtagtgtnnnnnnnnnnagagttcacacatcattgagca

SEQ ID NO: 326 - DNA, 104 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tcataaaatttggtttgtgccannnnnnnnnncttcagcttcccgattacgggtacgatccgacggtagtgtnnnnnnnnnnttcataataggggacgaacagg

SEQ ID NO: 327 - DNA, 107 bp, Artificial Sequence
  source: /note=“Description of Artificial Sequence Synthetic polynucleotide”
  modified_base (28)..(37): a, c, t, g, unknown or other
  modified_base (78)..(87): a, c, t, g, unknown or other
accactgttttattaagattgttttgannnnnnnnnncttcagcttcccgattacgggtacgatccgacggtagtgtnnnnnnnnnngacacggatcctccctgttc

SEQ ID NO: 328 - DNA, 106 bp, Artificial Sequence
  source: /note=“Description of Artificial Sequence Synthetic polynucleotide”
  modified_base (26)..(35): a, c, t, g, unknown or other
  modified_base (76)..(85): a, c, t, g, unknown or other
acagcagattcctcatgtaagatgtnnnnnnnnnncttcagcttcccgattacgggtacgatccgacggtagtgtnnnnnnnnnnactggcatcatttccctgtgt

SEQ ID NO: 329 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
acccacagagcttcgttttctnnnnnnnnnncttcagcttcccgattacgggtacgatccgacggtagtgtnnnnnnnnnntgggcctccttctgcatgat

SEQ ID NO: 330 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gggcctccttctgcatgattnnnnnnnnnncttcagcttcccgattacgggtacgatccgacggtagtgtnnnnnnnnnnactggctactcttgagaattgc

SEQ ID NO: 331 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aaattggaagcagctccggannnnnnnnnncttcagcttcccgattacgggtacgatccgacggtagtgtnnnnnnnnnnaacctagagttccagaagctgc

SEQ ID NO: 332 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgaacttgccacttgcttgannnnnnnnnncttcagcttcccgattacgggtacgatccgacggtagtgtnnnnnnnnnnctccggacacttggctcaat

SEQ ID NO: 333 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
gtggggttacttctaatttgtgctnnnnnnnnnncttcagcttcccgattacgggtacgatccgacggtagtgtnnnnnnnnnngcgctggtcacaaaatcctg

SEQ ID NO: 334 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ccagcagaacctgacatccannnnnnnnnncttcagcttcccgattacgggtacgatccgacggtagtgtnnnnnnnnnncccccaaaggatgcaacttc

SEQ ID NO: 335 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gctggcttttcacagcttgtnnnnnnnnnncttcagcttcccgattacgggtacgatccgacggtagtgtnnnnnnnnnnccgcttcgatctctggctta

SEQ ID NO: 336 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggagagagaaggagggcaaannnnnnnnnncttcagcttcccgattacgggtacgatccgacggtagtgtnnnnnnnnnncatttggcctgatgcttggc

SEQ ID NO: 337 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
atccagtctaggaagagggccnnnnnnnnnncttcagcttcccgattacgggtacgatccgacggtagtgtnnnnnnnnnntggacactctttgcagatgtt

SEQ ID NO: 338 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
gccagttgctgttagttcgtacnnnnnnnnnncttcagcttcccgattacgggtacgatccgacggtagtgtnnnnnnnnnncagagtggctgctgcagaaa

SEQ ID NO: 339 - DNA, 103 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
caatgattggacactctttgcannnnnnnnnncttcagcttcccgattacgggtacgatccgacggtagtgtnnnnnnnnnnggagggtgacaggaatgatcg

SEQ ID NO: 340 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tggatgagactggaaccccannnnnnnnnncttcagcttcccgattacgggtacgatccgacggtagtgtnnnnnnnnnncacctcctttgccatcttgc

SEQ ID NO: 341 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
atgacatctgccaaagctgcnnnnnnnnnncttcagcttcccgattacgggtacgatccgacggtagtgtnnnnnnnnnntgtgggactaatgaacattgct

SEQ ID NO: 342 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcactatcccatggtggaatnnnnnnnnnncttcagcttcccgattacgggtacgatccgacggtagtgtnnnnnnnnnnttgggaatttgattcgaaga

SEQ ID NO: 343 - DNA, 103 bp, Artificial Sequence
  source: /note=“Description of Artificial Sequence Synthetic polynucleotide”
  modified_base (24)..(33): a, c, t, g, unknown or other
  modified_base (74)..(83): a, c, t, g, unknown or other
gtgctttagactcctgtacctgannnnnnnnnncttcagcttcccgattacgggtacgatccgacggtagtgtnnnnnnnnnntcaggctggcgtcaaactta

SEQ ID NO: 344 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gccttttgcaactcgaccagnnnnnnnnnncttcagcttcccgattacgggtacgatccgacggtagtgtnnnnnnnnnntgagagccactttagctggg

SEQ ID NO: 345 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
gtgagagttagttcacctgggannnnnnnnnncttcagcttcccgattacgggtacgatccgacggtagtgtnnnnnnnnnnatgacatctgccaaagctgc

SEQ ID NO: 346 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgtccagttgccactttcccnnnnnnnnnncttcagcttcccgattacgggtacgatccgacggtagtgtnnnnnnnnnnagagggggacaacatggaaa

SEQ ID NO: 347 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ccttggcaaagtctcgaacannnnnnnnnncttcagcttcccgattacgggtacgatccgacggtagtgtnnnnnnnnnngggtgttcagctgagaggag

SEQ ID NO: 348 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tggaatcagacaaatggggcnnnnnnnnnncttcagcttcccgattacgggtacgatccgacggtagtgtnnnnnnnnnnaccttggcaaagtctcgaac

SEQ ID NO: 349 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
acgtttccatgttgtcccccnnnnnnnnnncttcagcttcccgattacgggtacgatccgacggtagtgtnnnnnnnnnngacgtgggaaagtggcaact

SEQ ID NO: 350 - DNA, 106 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
agcagaacacactcttgtttgannnnnnnnnncttcagcttcccgattacgggtacgatccgacggtagtgtnnnnnnnnnntctcccttttagactacatcagga

SEQ ID NO: 351 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
attttgcgaagcatccccgannnnnnnnnncttcagcttcccgattacgggtacgatccgacggtagtgtnnnnnnnnnnaacaagtgtcatggggcaga

SEQ ID NO: 352 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tctggccagtagattctgcgnnnnnnnnnncttcagcttcccgattacgggtacgatccgacggtagtgtnnnnnnnnnnacaccttggtttggctattgc

SEQ ID NO: 353 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tttgctgaagggtgctgctannnnnnnnnncttcagcttcccgattacgggtacgatccgacggtagtgtnnnnnnnnnntttttgcggctgagtttgcg

SEQ ID NO: 354 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gcaatagccaaaccaaggtgtnnnnnnnnnncttcagcttcccgattacgggtacgatccgacggtagtgtnnnnnnnnnnacgcagaatctactggccag

SEQ ID NO: 355 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
aggagacacacgcaaactcannnnnnnnnncttcagcttcccgattacgggtacgatccgacggtagtgtnnnnnnnnnnaaagagaaccaagcgagcga

SEQ ID NO: 356 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cctcgtcccctcagctttcannnnnnnnnncttcagcttcccgattacgggtacgatccgacggtagtgtnnnnnnnnnnagaataaaagcattctaggcca

SEQ ID NO: 357 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
aacccaccacacagttatgttnnnnnnnnnncttcagcttcccgattacgggtacgatccgacggtagtgtnnnnnnnnnntgcctggcatacaactagtct

SEQ ID NO: 358 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
tgcgtgaatgagtatcatcgtgnnnnnnnnnncttcagcttcccgattacgggtacgatccgacggtagtgtnnnnnnnnnngaacggcatgcacgttagag

SEQ ID NO: 359 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
ccccaaacttgtctgattcctnnnnnnnnnncttcagcttcccgattacgggtacgatccgacggtagtgtnnnnnnnnnncttataggcctgcctcgtcc

SEQ ID NO: 360 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ccatttgaggcagtgtgtggnnnnnnnnnncttcagcttcccgattacgggtacgatccgacggtagtgtnnnnnnnnnngctgttttccatttctgctagc

SEQ ID NO: 361 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
ttccatttctgctagcctgatnnnnnnnnnncttcagcttcccgattacgggtacgatccgacggtagtgtnnnnnnnnnntcctgtgctatcctacctct

SEQ ID NO: 362 - DNA, 102 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tgagagcatgtaagtatcccannnnnnnnnncttcagcttcccgattacgggtacgatccgacggtagtgtnnnnnnnnnntcctttctcttcttgccatga

SEQ ID NO: 363 - DNA, 102 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gctcccctctttcctcactcnnnnnnnnnncttcagcttcccgattacgggtacgatccgacggtagtgtnnnnnnnnnncctggcacttttctatgtgtgc

SEQ ID NO: 364 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
ggaaagaggggagctagagagnnnnnnnnnncttcagcttcccgattacgggtacgatccgacggtagtgtnnnnnnnnnnacccccaaagcaaaataagg

SEQ ID NO: 365 - DNA, 103 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
aagtttgaaccaggactccccnnnnnnnnnncttcagcttcccgattacgggtacgatccgacggtagtgtnnnnnnnnnntcaaatacactcctgagtccct

SEQ ID NO: 366 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
ccccttattttgctttgggggnnnnnnnnnncttcagcttcccgattacgggtacgatccgacggtagtgtnnnnnnnnnnagctcccctctttcctcact

SEQ ID NO: 367 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tgtcattggtatgcagagtgcnnnnnnnnnncttcagcttcccgattacgggtacgatccgacggtagtgtnnnnnnnnnncctcgtagtcctgcccagat

SEQ ID NO: 368 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gcttgcagattcctattggcnnnnnnnnnncttcagcttcccgattacgggtacgatccgacggtagtgtnnnnnnnnnnctcagcaatgagctcagcat

SEQ ID NO: 369 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gcaagtgaggagagagatgggnnnnnnnnnncttcagcttcccgattacgggtacgatccgacggtagtgtnnnnnnnnnnccctcctgaaatgatgccca

SEQ ID NO: 370 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
gtggggacaggcctttatgtnnnnnnnnnncttcagcttcccgattacgggtacgatccgacggtagtgtnnnnnnnnnngcctgtgtaactgtgactcca

SEQ ID NO: 371 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tgctgctgctttagacggtcnnnnnnnnnncttcagcttcccgattacgggtacgatccgacggtagtgtnnnnnnnnnntgtggtcttccaggatttgca

SEQ ID NO: 372 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
aacctcagagagcactttttatagnnnnnnnnnncttcagcttcccgattacgggtacgatccgacggtagtgtnnnnnnnnnnccaagctactgcgtcaacac

SEQ ID NO: 373 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
agcctgtgtaactgtgactccnnnnnnnnnncttcagcttcccgattacgggtacgatccgacggtagtgtnnnnnnnnnncactttgcaggcacatacca

SEQ ID NO: 374 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
catctgactgccaccgaagannnnnnnnnncttcagcttcccgattacgggtacgatccgacggtagtgtnnnnnnnnnntggggacaggcctttatgttc

SEQ ID NO: 375 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggacatgaatatttggccgtnnnnnnnnnncttcagcttcccgattacgggtacgatccgacggtagtgtnnnnnnnnnntccgacagcagtcagcctat

SEQ ID NO: 376 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
tggccgtaagtgtttgactcannnnnnnnnncttcagcttcccgattacgggtacgatccgacggtagtgtnnnnnnnnnncacaacggtgtcctctcctt

SEQ ID NO: 377 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
acaacggtgtcctctccttcnnnnnnnnnncttcagcttcccgattacgggtacgatccgacggtagtgtnnnnnnnnnnacaatctttgggagggcttct

SEQ ID NO: 378 - DNA, 107 bp, Artificial Sequence
  source: /note=“Description of Artificial Sequence Synthetic polynucleotide”
  modified_base (28)..(37): a, c, t, g, unknown or other
  modified_base (78)..(87): a, c, t, g, unknown or other
gggatatttcactgttgatataatccannnnnnnnnncttcagcttcccgattacgggtacgatccgacggtagtgtnnnnnnnnnnccattcactttggcctctgc

SEQ ID NO: 379 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agtccgaagtttgactgccannnnnnnnnncttcagcttcccgattacgggtacgatccgacggtagtgtnnnnnnnnnntcagtggctccctgatacca

SEQ ID NO: 380 - DNA, 101 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
cctggggctaagtcatccaaannnnnnnnnncttcagcttcccgattacgggtacgatccgacggtagtgtnnnnnnnnnngtttgactgccaaccactcg

SEQ ID NO: 381 - DNA, 102 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
aacaaagaaaaccctcaagcttnnnnnnnnnncttcagcttcccgattacgggtacgatccgacggtagtgtnnnnnnnnnncacctcctctaaccctgtgc

SEQ ID NO: 382 - DNA, 106 bp, Artificial Sequence
  source: /note=“Description of Artificial Sequence Synthetic polynucleotide”
  modified_base (23)..(32): a, c, t, g, unknown or other
  modified_base (73)..(82): a, c, t, g, unknown or other
ggaagatcttctcagtcctcccnnnnnnnnnncttcagcttcccgattacgggtacgatccgacggtagtgtnnnnnnnnnntccctttaaagaattacttcctca

SEQ ID NO: 383 - DNA, 106 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
tgaggaagtaattctttaaagggannnnnnnnnncttcagcttcccgattacgggtacgatccgacggtagtgtnnnnnnnnnntggggaggactgagaagatctt

SEQ ID NO: 384 - DNA, 106 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
gaaaacagatattaaagggccatgnnnnnnnnnncttcagcttcccgattacgggtacgatccgacggtagtgtnnnnnnnnnnggaaggagttgttgagttgctc

SEQ ID NO: 385 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggaagccaacacgcagtatcnnnnnnnnnncttcagcttcccgattacgggtacgatccgacggtagtgtnnnnnnnnnntcttctcagtcctccccagg

SEQ ID NO: 386 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
cctggggaggactgagaagannnnnnnnnncttcagcttcccgattacgggtacgatccgacggtagtgtnnnnnnnnnntggcctgatcccagcaaatc

SEQ ID NO: 387 - DNA, 103 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
agttgctccatcacctcctcnnnnnnnnnncttcagcttcccgattacgggtacgatccgacggtagtgtnnnnnnnnnncaaatcttttcaccatggaccca

SEQ ID NO: 388 - DNA, 105 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ggaggtgatggagcaactcannnnnnnnnncttcagcttcccgattacgggtacgatccgacggtagtgtnnnnnnnnnnggtgttaaaaatgtaatcatggccc

SEQ ID NO: 389 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
acgcgcatgtgtgtattacannnnnnnnnncttcagcttcccgattacgggtacgatccgacggtagtgtnnnnnnnnnntctctgcctcttcctctctct

SEQ ID NO: 390 - DNA, 104 bp, Artificial Sequence
  source: /note=“Description of Artificial Sequence Synthetic polynucleotide”
  modified_base (25)..(34): a, c, t, g, unknown or other
  modified_base (75)..(84): a, c, t, g, unknown or other
agatgaccatttattctctgctggnnnnnnnnnncttcagcttcccgattacgggtacgatccgacggtagtgtnnnnnnnnnnctcattggctttccaggggt

SEQ ID NO: 391 - DNA, 101 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
ctcattggctttccaggggtnnnnnnnnnncttcagcttcccgattacgggtacgatccgacggtagtgtnnnnnnnnnntgttcctcatgagctgcaagt

SEQ ID NO: 392 - DNA, 100 bp, Artificial Sequence
  source: /note=“Description of Artificial Sequence Synthetic polynucleotide”
  modified_base (21)..(30): a, c, t, g, unknown or other
  modified_base (71)..(80): a, c, t, g, unknown or other
tccacatggcagatgatttgnnnnnnnnnncttcagcttcccgattacgggtacgatccgacggtagtgtnnnnnnnnnncgatgcagcttctgtgttgt

SEQ ID NO: 393 - DNA, 105 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
ctgtttctttgccatttgggannnnnnnnnncttcagcttcccgattacgggtacgatccgacggtagtgtnnnnnnnnnnaacatttattctgctccttcttca

SEQ ID NO: 394 - DNA, 103 bp, Artificial Sequence
  source: /note=“Description of Artificial Sequence Synthetic polynucleotide”
  modified_base (22)..(31): a, c, t, g, unknown or other
  modified_base (72)..(81): a, c, t, g, unknown or other
gcatcactctgtttctttgccnnnnnnnnnncttcagcttcccgattacgggtacgatccgacggtagtgtnnnnnnnnnntctgctccttcttcatctgtca

SEQ ID NO: 395 - DNA, 106 bp, Artificial Sequence
  source: /note=“Description of Artificial Sequence Synthetic polynucleotide”
  modified_base (27)..(36): a, c, t, g, unknown or other
  modified_base (77)..(86): a, c, t, g, unknown or other
ttacaaaaggtgcagatagatagcatnnnnnnnnnncttcagcttcccgattacgggtacgatccgacggtagtgtnnnnnnnnnngcgggaatcaggagttgtaa

SEQ ID NO: 396 - DNA, 91 bp, Artificial Sequence
  source: /note=“Description of Artificial Sequence Synthetic oligonucleotide”
  modified_base (22)..(31): a, c, t, g, unknown or other
  modified_base (62)..(71): a, c, t, g, unknown or other
accacctcccatatgtccagannnnnnnnnncttcagcttcccgatatccgacggtagtgtnnnnnnnnnnaccagtctgggcaacatagc

(19) United States
(12) Patent Application Publication
(10) Pub. No.: US 2019/0024149 A1
(43) Pub. Date: Jan. 24, 2019

(54) SYSTEMS AND METHODS OF GENETIC ANALYSIS

(71) Applicant: Progenity, Inc., San Diego (CA)

(72) Inventors: Heng Wang, Walnut Creek, CA (US); Tobias Mann, Ann Arbor, MI (US); Jeffrey Buis, Ann Arbor, MI (US)

(21) Appl. No.: 15/746,328

(22) PCT Filed: Jul. 29, 2017
(86) PCT No.: PCT/US2016/044915

§ 371 Date: Jan. 19, 2018

Related U.S. Application Data
(60) Provisional application No. 62/198,644, filed on Jul. 29, 2015.

(51) Int. Cl. C12Q 1/6827

(52) U.S. Cl. CPC C12Q 1/6827; C12Q 2600/156

(57) ABSTRACT

Systems and methods for detecting copy number variations, chromosomal abnormalities, exonic deletions or duplications, or other genetic variations using molecular inversion probes and probe capture metrics.

### CROSS REFERENCE TO RELATED APPLICATION

This application claims the benefit of U.S. Provisional Application No. 62/198,644, filed on Jul. 29, 2015, which is hereby incorporated herein by reference in its entirety.

### FIELD OF THE INVENTION

This disclosure relates to systems and methods for determining copy number variations, chromosomal abnormalities or micro-deletions in a subject in need thereof.

### BACKGROUND OF THE INVENTION

Genetic carrier screening is a type of testing that can identify risks of individual subjects, typically prospective parents, at having a child with one of the hereditary diseases that can cause death or disability. A person who has one normal gene and one abnormal gene that can cause a genetic disorder, is called a carrier. A carrier is not affected with the disorder, but they can pass on the abnormal gene to the next generation. For example, genetic carrier screening can determine if a prospective parent is a carrier of a recessive genetic disorder, such as cystic fibrosis, sickle cell disease, thalassemia, Tay-Sachs disease, and spinal muscular atrophy (SMA). If both prospective parents are carriers of a defective gene for a recessive genetic disorder, then they are at risk for having children with that genetic disorder. If neither parent is a carrier, then they can rule out such risk. Therefore, genetic carrier screening is very informative to prospective parents.

Spinal muscular atrophy (SMA) is one of the most common inherited causes of infant death. It affects a person's ability to control their muscles, including those involved in breathing, eating, crawling and walking. SMA has different levels of severity, none of which affects intelligence. However, the most common form of the disorder causes death by age two. About one in every 6,000 to one in every 10,000 babies born in the U.S. has SMA.

SMA is a recessive genetic disorder. It is caused by mutations in the SMN (Survival Motor Neuron) genes, SMN1 and SMN2, that are located on chromosome 5. The SMN gene is composed of 9 exons, with a stop codon near the end of exon 7. Two almost identical SMN genes are present on chromosome 5q13: the telomeric or SMN1 gene, which is the SMA-determining gene, and the centromere or SMN2 gene. The gene sequences of SMN1 and SMN2 differ by only 5 base pairs, and the coding sequence differs by a single nucleotide (840C>T). This single nucleotide difference does not alter an amino acid, but it does affect splicing and causes about 90% of transcripts from SMN2 to lack exon 7. Consequently, in contrast to the SMN1 gene, which produces a full-length SMN protein, the SMN2 gene produces predominantly a shortened, unstable and rapidly degraded isoform.

Individuals having SMA typically have inherited a mutant SMN1 gene from each of their parents. The majority of mutations responsible for SMA are either deletions or gene conversions. A deletion involves partial or complete removal of the SMN1 gene. In a gene conversion, the SMN1 gene is converted into an SMN2-like gene because the “C” in exon 7 is mutated to a “T”. In both cases, SMA patients are missing SMN1 exon 7 and make insufficient amounts of full-length SMN protein. Therefore, a SMA carrier testing can determine whether each parent is a carrier or not based on the copy numbers of the SMN1 and SMN2 genes in the parent.

Current methods for genetic carrier screening, such as SMA carrier testing, are time-consuming or expensive, or require extensive bioinformatics analysis. In addition, current methods for detecting exonic deletions or duplications are also time-consuming or expensive, or require extensive bioinformatics analysis.

Pharmacogenomics testing (also referred as drug-gene testing) refers to the study of how a subject's genes affect the body's response to medications. Pharmacogenomic tests look for changes or variants in one or more genes that may determine whether a medication could be an effective treatment for an individual or whether an individual could have side effects to a specific medication.

Therefore, there is a need for developing cost-effective and efficient tests that have high sensitivities and specificities.

### SUMMARY OF THE INVENTION

Some embodiments of the disclosure are:

1. A method of detecting copy number variation in a subject comprising:

a) obtaining a nucleic acid sample isolated from the subject;

b) capturing one or more target sequences in the nucleic acid sample obtained in step a) by using one or more target populations of targeting molecular inversion probes (MIPs) to produce a plurality of targeting MIPs replicons for each target sequence,

wherein each of the targeting MIPs in each of the target populations comprises in sequence the following components:

first targeting polynucleotide arm—first unique targeting molecular tag—polynucleotide linker—second unique targeting molecular tag—second targeting polynucleotide arm;

wherein the pair of first and second targeting polynucleotide arms in each of the targeting MIPs in each target population are identical, and are substantially complementary to first and second regions in the nucleic acid that, respectively, flank the target sequence that is targeted by the one or more targeting MIPs;

wherein the first and second unique targeting molecular tags in each of the targeting MIPs in each target population are distinct in each of the targeting MIPs, in each member of the target population, and in each of the target populations;

c) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),

wherein each of the control MIPs in each control population comprises in sequence the following components:

first control polynucleotide arm—first unique control molecular tag—polynucleotide linker—second unique control molecular tag—second control polynucleotide arm;

wherein the pair of first and second control polynucleotide arms in each of the control MIPs in each control population are identical, and are substantially complementary to first and second regions in the nucleic acid that, respectively, flank each control sequence;

wherein the first and second unique control molecular tags in each of the control MIPs in each control population are distinct in each of the control MIPs and in each member of the control population, and are different from the unique targeting molecular tags;

d) sequencing the targeting and control MIPs amplicons that are amplified from the targeting and control MIPs replicons obtained in steps b) and c);

e) determining, for each target population, the number of the unique targeting molecular tags present in the targeting MIPs amplicons sequenced in step d);

f) determining, for each control population, the number of the unique control molecular tags present in the control MIPs amplicons sequenced in step d);

g) computing a target probe capture metric, for each of the one or more target sequences, based at least in part on the number of the unique targeting molecular tags determined in step e) and a plurality of control probe capture metrics based at least in part on the numbers of the unique control molecular tags determined in step f);

h) identifying a subset of the control populations of control MIPs that have control probe capture metrics satisfying at least one criterion;

i) normalizing each of the one or more target probe capture metrics by a factor computed from the subset of control probe capture metrics satisfying the at least one criterion, to obtain a test normalized target probe capture metric for each of the one or more target sequences;

j) comparing each test normalized target probe capture metric obtained in step i) to a plurality of reference normalized target probe capture metrics that are computed based on reference nucleic acid samples obtained from reference subjects exhibiting known genotypes using the same target and control sequences, target population, one subset of control populations in steps b)-g) and i); and

k) determining, based on the comparing in step j) and the known genotypes of reference subjects, the copy number variation of each of the one or more target sequences of interest.

2. The method of embodiment 1, wherein the nucleic acid sample is DNA or RNA.

3. The method of embodiment 1 or 2, wherein the nucleic acid sample is genomic DNA.

4. The method of any one of embodiments 1-3, wherein the subject is a carrier screening candidate for one or more diseases or conditions.

5. The method of any one of embodiments 1-3, wherein the subject is a candidate for:

a) a pharmacogenomics test;

b) a targeted tumor test;

c) an exonic deletion test; or

d) an exonic duplication test.

6. The method of any one of embodiments 1-5, wherein the length of each of the targeting polynucleotide arms is between 18 and 35 base pairs.

7. The method of any one of embodiments 1-5, wherein the length of each of the control polynucleotide arms is between 18 and 35 base pairs.

8. The method of any one of embodiments 1-7, wherein each of the targeting polynucleotide arms has a melting temperature between 57° C. and 63° C.

9. The method of any one of embodiments 1-7, wherein each of the control polynucleotide arms has a melting temperature between 57° C. and 63° C.

10. The method of any one of embodiments 1-9, wherein each of the targeting polynucleotide arms has a GC content between 30% and 70%.

11. The method of any one of embodiments 1-9, wherein each of the control polynucleotide arms has a GC content between 30% and 70%.

12. The method of any one of embodiments 1-11, wherein the length of each of the unique targeting molecular tags is between 12 and 20 base pairs.

13. The method of any one of embodiments 1-11, wherein the length of each of the unique control molecular tags is between 12 and 20 base pairs.

14. The method of any one of embodiments 1-13, wherein each of the unique targeting or control molecular tags is not substantially complementary to any genomic region of the subject.

15. The method of any one of embodiments 1-13, wherein the polynucleotide linker is not substantially complementary to any genomic region of the subject.

16. The method of any one of embodiments 1-15, wherein the polynucleotide linker has a length of between 30 and 40 base pairs.

17. The method of any one of embodiments 1-15, wherein the polynucleotide linker has a melting temperature of between 60° C. and 80° C.

18. The method of any one of embodiments 1-15, wherein the polynucleotide linker has a GC content between 30% and 70%.

19. The method of any one of embodiments 1-15, wherein the polynucleotide linker comprises 5′-CTTCAGCTTCCCGATATCCGACGGTAGTGT-3′(SEQ ID NO: 1) 20. The method of any one of embodiments 1-19, wherein the plurality of target population of targeting MIPs and the plurality of control populations of control MIPs are in a probe mixture.

21. The method of embodiment 20, wherein the probe mixture has a concentration between 1-100 pM; 10-100 pM; 50-100 pM; or 10-50 pM.

22. The method of any one of embodiments 1-21, wherein each of the targeting MIPs replicons is a single-stranded circular nucleic acid molecule.

23. The method of embodiment 22, wherein each of the targeting MIPs replicons provided in step b) is produced by:

i) the first and second targeting polynucleotide arms, respectively, hybridizing to the first and second regions in the nucleic acid that, respectively, flank the target sequence; and

ii) after the hybridization, using a ligation/extension mixture to extend and ligate the gap region between the two targeting polynucleotide arms to form single-stranded circular nucleic acid molecules.

24. The method of any one of embodiments 1-23, wherein each of the control MIPs replicons is a single-stranded circular nucleic acid molecule.

25. The method of embodiment 24, wherein each of the control MIPs replicons provided in step b) is produced by:

i) the first and second control polynucleotide arms, respectively, hybridizing to the first and second regions in the nucleic acid that, respectively, flank the control sequence; and

ii) after the hybridization, using a ligation/extension mixture to extend and ligate the gap region between the two control polynucleotide arms to form single-stranded circular nucleic acid molecules.

26. The method of any one of embodiments 1-25, wherein the sequencing step of d) comprises a next-generation sequencing method.

27. The method of embodiment 26, wherein the next-generation sequencing method comprises a massive parallel sequencing method, or a massive parallel short-read sequencing method.

28. The method of any one of embodiments 1-27, wherein the method comprises, before the sequencing step of d), a PCR reaction to amplify the targeting and control MIPs replicons to produce the targeting and control MIPs amplicons for sequencing.

29. The method of embodiment 28, wherein the PCR reaction is an indexing PCR reaction.

30. The method of embodiment 29, wherein the indexing PCR reaction introduces, the following components: a pair of indexing primers, a unique sample barcode and a pair of sequencing adaptors, into each of the targeting or control MIPs replicons to produce barcoded targeting or control MIPs amplicons.

31. The method of embodiment 30, wherein the barcoded targeting MIPs amplicons comprise in sequence the following components:

a first sequencing adaptor—a first sequencing primer—the first unique targeting molecular tag—the first targeting polynucleotide arm—captured target nucleic acid—the second targeting polynucleotide arm—the second unique targeting molecular tag—a unique sample barcode—a second sequencing primer—a second sequencing adaptor; or

wherein the barcoded control MIPs amplicons comprise in sequence the following components:

a first sequencing adaptor—a first sequencing primer—the first unique control molecular tag—the first control polynucleotide arm—captured control nucleic acid—the second control polynucleotide arm—the second unique control molecular tag—a unique sample barcode—a second sequencing primer—a second sequencing adaptor.

32. The method of any one of embodiments 1-31, wherein at least one of the one or more target sequences and at least one of the control sequences are on the same chromosome.

33. The method of any one of embodiments 1-31, wherein at least one of the one or more target sequences and at least one of the control sequences are on different chromosomes.

34. The method of any one of embodiments 1-33, wherein the target sequence is SMN1/SMN2.

35. The method of embodiment 34, wherein the first targeting polynucleotide primer for the target sequence of SMN1/SMN2 comprises the sequence of 5′-AGG AGT AAG TCT GCC AGC ATT-3′ (SEQ ID NO: 2).

36. The method of embodiment 34 or 35, wherein the second targeting polynucleotide primer for the target sequence of SMN1/SMN2 comprises the sequence of 5′-AAA TGT CTT GTG AAA CAA AAT GCT-3′ (SEQ ID NO: 3).

37. The method of any one of embodiments 34-36, wherein the polynucleotide linker comprises 5′-CTT CAG CTT CCC GAT ATC CGA CGG TAG TGT-3′ (SEQ ID NO: 1).

38. The method of any one of embodiments 34-37, wherein the MIP for the target sequence of SMN1/SMN2 comprises the sequence of 5′-AGG AGT AAG TCT GCC AGC ATT NNN NNN NNN NCT TCA GCT TCC CGA TTA CGG GTA CGA TCC GAC GGT AGT GTN NNN NNN NNN AAA TGT CTT GTG AAA CAA AAT GCT-3′ (SEQ ID NO: 4).

39. The method of any one of embodiments 1-38, wherein the control sequences comprise one or more genes or sequences selected from the group consisting of CFTR, HEXA, HFE, HBB, BLM, IDS, IDUA, LCAS, LPL, MEFV, GBA, MPL, PEX6, PCCB, ATM, NBN, FANCC, F8, CBS, CPT1, CPT2, FKTN, G6PD, GALC, ABCC8, ASPA, MCOLN1, SPMD1, CLRN1, NEB, G6PC, TMEM216, BCKDHA, BCKDHB, DLD, IKBKAP, PCDH15, TTN, GAMT, KCNJ11, IL2RG, and GLA.

40. A method of detecting copy number variation in a subject comprising:

a) isolating a genomic DNA sample from the subject;

b) adding the genomic DNA sample into each well of a multi-well plate, wherein each well of the multi-well plate comprises a probe mixture, wherein the probe mixture comprises a plurality of target populations of targeting molecular inversion probes (MIPs), a plurality of control populations of control MIPs and buffer;

wherein each targeting population of targeting MIPs is capable of amplifying a distinct target sequence in the genomic DNA sample obtained in step a),

wherein each of the targeting MIPs in each target population comprises in sequence the following components:

first targeting polynucleotide arm—first unique targeting molecular tag—polynucleotide linker—second unique targeting molecular tag—second targeting polynucleotide arm;

wherein the pair of first and second targeting polynucleotide arms in each of the targeting MIPs in each target population are identical, and are substantially complementary to first and second regions in the genomic DNA that, respectively, flank each target sequence;

wherein the first and second unique targeting molecular tags in each of the targeting MIPs in each target population are distinct in each of the targeting MIPs and in each member of the target population;

wherein each control population of control MIPs is capable of amplifying a distinct control sequence in the genomic DNA sample obtained in step a),

wherein each of the control MIPs in each control population comprises in sequence the following components:

first control polynucleotide arm—first unique control molecular tag—polynucleotide linker—second unique control molecular tag—second control polynucleotide arm;

wherein the pair of first and second control polynucleotide arms in each of the control MIPs in each control population are identical, and are substantially complementary to first and second regions in the genomic DNA that, respectively, flank each control sequence;

wherein the first and second unique control molecular tags in each of the control MIPs in each control population are distinct in each of the control MIPs and in each member of the control population, and are different from the unique targeting molecular tags;

c) incubating the genomic DNA sample with the probe mixture for the targeting MIPs to capture the target sequence and for the control MIPs to capture the control sequences;

d) adding an extension/ligation mixture to the sample of c) for the targeting MIPs and the captured target sequence to form the targeting MIPs replicons and for the control MIPs and the captured control sequences to form the control MIPs replicons, wherein the extension/ligation mixture comprises a polymerase, a plurality of dNTPs, a ligase, and buffer;

e) adding an exonuclease mixture to the targeting and control MIPs replicons to remove excess probes or excess genomic DNA;

f) adding an indexing PCR mixture to the sample of e) to add a pair of indexing primers, a unique sample barcode and a pair of sequencing adaptors to the targeting and control MIPs replicons to produce the targeting and control MIPs amplicons;

g) using a massively parallel sequencing method to determine, for each target population, the number of the unique targeting molecular tags present in the barcoded targeting MIPs amplicons provided in step f);

h) using a massively parallel sequencing method to determine, for each control population, the number of the unique control molecular tags present in the barcoded control MIPs amplicons provided in step f);

i) computing a target probe capture metric for each target sequence based at least in part on the number of the unique targeting molecular tags determined in step g) and a plurality of control probe capture metrics based at least in part on the numbers of the unique control molecular tags determined in step h);

j) identifying a subset of the control populations of control MIPs that have control probe capture metrics satisfying at least one criterion;

k) normalizing each target probe capture metric by a factor computed from the subset of control probe capture metrics satisfying the at least one criterion, to obtain a test normalized target probe capture metric for each target sequence;

l) comparing each test normalized target probe capture metric to a plurality of reference normalized target probe capture metrics that are computed based on reference genomic DNA samples obtained from reference subjects exhibiting known genotypes using the same target and control sequences, target population, one subset of control populations in steps b)-h); and

m) determining, based on the comparing in step l) and the known genotypes of reference subjects, the copy number variation for each target sequence.

41. A nucleic acid molecule comprising the sequence of:

(SEQ ID NO: 4)
5′-AGG AGT AAG TCT GCC AGC ATT NNN NNN NNN NCT
TCA GCT TCC CGA TTA CGG GTA CGA TCC GAC GGT AGT
GTN NNN NNN NNN AAA TGT CTT GTG AAA CAA AAT
GCT-3′.

42. The nucleic acid molecule of embodiment 41, wherein the nucleic acid is 5′ phosphorylated.

43. A method for producing a genotype cluster, the method comprising:

a) receiving sequencing data obtained from a plurality of nucleic acid samples from a plurality of subsets of a plurality of subjects, each sample in the plurality of samples being obtained from a different subject, and each subset being characterized by subjects exhibiting a same known genotype for a gene of interest, wherein the sequencing data for the nucleic acid sample from each subject in the plurality of subsets is obtained by:
i) obtaining a nucleic acid sample isolated from the subject;
ii) capturing one or more target sequences of interest in the nucleic acid sample obtained in step a.i) by using one or more target populations of targeting molecular inversion probes (MIPs) to produce targeting MIPs replicons for each target sequence,
wherein each of the targeting MIPs in each of the target populations comprises in sequence the following components:
first targeting polynucleotide arm—first unique targeting molecular tag—polynucleotide linker—second unique targeting molecular tag—second targeting polynucleotide arm;
wherein the pair of first and second targeting polynucleotide arms in each of the targeting MIPs in each target population are identical, and are substantially complementary to first and second regions in the nucleic acid that, respectively, flank the target sequence of interest that is targeted by the one or more targeting MIPs;
wherein the first and second unique targeting molecular tags in each of the targeting MIPs in each target population are distinct in each of the targeting MIPs and in each member of the target population;
iii) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),
wherein each of the control MIPs in each control population comprises in sequence the following components:
first control polynucleotide arm—first unique control molecular tag—polynucleotide linker—second unique control molecular tag—second control polynucleotide arm;
wherein the pair of first and second control polynucleotide arms in each of the control MIPs in each control population are identical, and are substantially complementary to first and second regions in the nucleic acid that, respectively, flank each control sequence;
wherein the first and second unique control molecular tags in each of the control MIPs in each control population are distinct in each of the control MIPs and in each member of the control population, and are different from the unique targeting molecular tags;
iv) sequencing the targeting and control MIPs amplicons that are amplified from the targeting and control MIPs replicons obtained in steps a.ii) and a.iii);

b) for each respective sample obtained from a subset in the plurality of subsets:
i) determining, for each target population, the number of the unique targeting molecular tags present in the targeting MIPs amplicons sequenced in step a.iv);
ii) determining, for each control population, the number of the unique control molecular tags present in the control MIPs amplicons sequenced in step a.iv);
iii) computing a target probe capture metric, for each target sequence, based at least in part on the number of the unique targeting molecular tags determined in step b.i) and a plurality of control probe capture metrics based at least in part on the numbers of the unique control molecular tags determined in step b.ii);
iv) identifying a subset of the control populations of control MIPs that have control probe capture metrics satisfying at least one criterion;
v) normalizing each target probe capture metric by a factor computed from the control probe capture metrics satisfying the at least one criterion, to obtain a normalized target probe capture metric for each of the one or more target sites; and

c) grouping, across the samples obtained from each subset of subjects, the normalized target probe capture metrics to obtain the genotype cluster for the known genotype.

44. The method of embodiment 43, wherein computing the target probe capture metric at step b.iii) comprises normalizing the number of the unique targeting molecular tags determined in step b.i) by a sum of the number of the unique targeting molecular tags and the numbers of the unique control molecular tags.

45. The method of embodiment 43, wherein computing the plurality of control probe capture metrics at step b.iii) comprises normalizing, for each control population, the number of unique control molecular tags determined in step b.ii) by a sum of the number of the unique targeting molecular tags and the numbers of the unique control molecular tags.

46. The method of any of embodiments 43-45, wherein the target probe capture metric for the target population is indicative of the target population's ability to hybridize to the target sequence of interest, relative to the abilities of the plurality of control populations to hybridize to the distinct control sequences.

47. The method of any of embodiments 43-46, wherein each control probe capture metric for a respective control population is indicative of the respective control population's ability to hybridize to one of the control sequences, relative to the abilities of 1) the target population to hybridize to the target sequence and 2) remaining control populations to hybridize to respective control sequences.

48. The method of any of embodiments 43-47, wherein the target sequence of interest is located on the gene of interest, and the control sequences correspond to one or more reference genes that are different from the gene of interest.

49. The method of any of embodiments 43-48, wherein the gene of interest is a survival of motor neuron 1 (SMN1) gene and/or a survival of motor neuron 2 (SMN2) gene.

50. The method of any of embodiments 43-48, wherein the gene of interest is a BRCA1 gene.

51. The method of any of embodiments 43-48, wherein the gene of interest is a DMD gene.

52. The method of any of embodiments 43-51, wherein the at least one criterion includes a requirement that the control probe capture metric is above a first threshold and below a second threshold.

53. The method of embodiment 52, further comprising determining the first threshold and the second threshold based at least in part on the target probe capture metric computed at step b.iii).

54. The method of embodiment 53, wherein the first threshold and the second threshold are determined further based at least in part on the plurality of control probe capture metrics computed at step b.iii).

55. The method of any of embodiments 43-54, further comprising, for each control population, computing a variability coefficient for the control probe capture metrics computed at step b.iii) across the samples obtained from each subset in the plurality of subsets.

56. The method of embodiment 55, wherein the at least one criterion includes a requirement that the variability coefficient is below a threshold.

57. The method of any of embodiments 43-56, wherein the factor computed at step b.v) is an average of the control probe capture metrics satisfying the at least one criterion.

58. The method of any of embodiments 43-57, wherein a first subset is characterized by subjects exhibiting a known copy count of a survival of motor neuron 1 (SMN1) gene, and a second subset is characterized by subjects exhibiting a known copy count of a survival motor neuron 2 (SMN2) gene.

59. The method of any of embodiments 43-58, wherein the known genotype corresponds to a known copy count of a survival of motor neuron 1 (SMN1) gene or of a survival of motor neuron 2 (SMN2) gene.

60. The method of any of embodiments 43-57, wherein a first subset is characterized by subjects exhibiting a known copy count of exon 11 on a BRCA1 gene.

61. The method of any of embodiments 43-57 and 60, wherein the known genotype corresponds to a known copy count of exon 11 on a BRCA1 gene.

62. The method of any of embodiments 43-57, wherein a first subset is characterized by subjects exhibiting a known copy count of a DMD gene.

63. The method of any of embodiments 43-57 and 62, wherein the known genotype corresponds to a known copy count of a DIVED gene.

64. The method of any of embodiments 43-63, wherein the first and second unique targeting molecular tags and the first and second unique control molecular tags are generated randomly for each MIP in the targeting population of targeting MIPS and in the control populations of control MIPs.

65. A system configured to perform the method of any of embodiments 43-64.

66. A computer program product comprising computer-readable instructions that, when executed in a computerized system comprising at least one processor, cause the processor to carry out one or more steps of the method of any of embodiments 43-64.

67. A method of selecting a genotype for a test subject, the method comprising:

a) receiving sequencing data obtained from a nucleic acid sample from the test subject, wherein the sequencing data for the nucleic acid sample is obtained by:
i) obtaining a nucleic acid sample isolated from the test subject;
ii) capturing one or more target sequences of interest in the nucleic acid sample obtained in step a) by using one or more target populations of targeting molecular inversion probes (MIPs) to produce a plurality of targeting MIPs replicons for each target sequence,
wherein each of the targeting MIPs in the target population comprises in sequence the following components:
first targeting polynucleotide arm—first unique targeting molecular tag—polynucleotide linker—second unique targeting molecular tag—second targeting polynucleotide arm;
wherein the pair of first and second targeting polynucleotide arms in each of the targeting MIPs in each target population are identical, and are substantially complementary to first and second regions in the nucleic acid that, respectively, flank the target sequence of interest that is targeted by the one or more targeting MIPs;
wherein the first and second unique targeting molecular tags in each of the targeting MIPs in each target population are distinct in each of the targeting MIPs and in each member of the target population;
iii) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),
wherein each of the control MIPs in each control population comprises in sequence the following components:
first control polynucleotide arm—first unique control molecular tag—polynucleotide linker—second unique control molecular tag—second control polynucleotide arm;
wherein the pair of first and second control polynucleotide arms in each of the control MIPs in each control population are identical, and are substantially complementary to first and second regions in the nucleic acid that, respectively, flank each control sequence;
wherein the first and second unique control molecular tags in each of the control MIPs in each control population are distinct in each of the control MIPs and in each member of the control population, and are different from the unique targeting molecular tags;
iv) sequencing the targeting and control MIPs amplicons that are amplified from the targeting and control MIPs replicons obtained in steps a.ii) and a.iii);

b) determining, for each target population, the number of the unique targeting molecular tags present in the targeting MIPs amplicons sequenced in step a.iv);

c) determining, for each control population, the number of the unique control molecular tags present in the control MIPs amplicons sequenced in step a.iv);

d) computing a target probe capture metric, for each target site, based at least in part on the number of the unique targeting molecular tags determined in step b) and a plurality of control probe capture metrics based at least in part on the numbers of the unique control molecular tags determined in step c);

e) identifying a subset of the control populations of control MIPs that have control probe capture metrics satisfying at least one criterion;

f) normalizing each of the one or more target probe capture metrics by a factor computed from the control probe capture metrics satisfying the at least one criterion, to obtain a normalized target probe capture metric for each of the one or more target sequences;

g) receiving a group of values corresponding to normalized target probe capture metrics computed from nucleic acid samples from a first plurality of reference subjects exhibiting a same known genotype for a gene of interest;

h) comparing each of the one or more normalized target probe capture metrics obtained in step f) to the group of values received in step g); and

i) determining, based on the comparing in step h), whether the test subject exhibits the same known genotype for the gene of interest in each of the one or more target sequences.

68. The method of embodiment 67, wherein the group of values is a first group of values, the same known genotype is a first copy number of the target sequence of interest, the method further comprising:

j) receiving a second group of values corresponding to normalized target probe capture metrics computed from nucleic acid samples from a second plurality of reference subjects exhibiting a second copy number of the target sequence of interest; and

k) comparing the normalized target probe capture metric obtained in step f) to the second group of values, wherein the determining in step i) comprises selecting between the first copy number and the second copy number for the test subj ect.

69. The method of embodiment 68, wherein:

the comparing in step h) comprises computing a first distance metric between the normalized probe capture metric obtained in step f) and the first group of values;

the comparing in step k) comprises computing a second distance metric between the normalized probe capture metric obtained in step f) and the second group of values; and

the selecting between the first copy number and second copy number comprises selecting the first copy number if the first distance metric is less than the second distance metric, and selecting the second copy number if the first distance metric exceeds the second distance metric.

70. The method of any of embodiments 69, wherein the first group of values and the second group of values are computed by:

repeating steps a-f) for each subject in the first and second pluralities of reference subjects;

grouping the normalized target probe capture metrics for the first plurality of reference subjects to obtain the first group of values; and

grouping the normalized target probe capture metrics for the second plurality of reference subjects to obtain the second group of values.

71. The method of any of embodiments 67-70, wherein the computing the target probe capture metric at step d) comprises normalizing the number of the unique targeting molecular tags determined in step b) by a sum of the number of the unique targeting molecular tags and the numbers of the unique control molecular tags.

72. The method of any of embodiments 67-71, wherein computing the plurality of control probe capture metrics at step d) comprises normalizing, for each control population, the number of the unique control molecular tags determined in step c) by a sum of the unique targeting molecular tags and the numbers of the unique control molecular tags.

73. The method of any of embodiments 67-72, wherein the target probe capture metric for the target population is indicative of the target population's ability to hybridize to the target sequence of interest, relative to the abilities of the plurality of control populations to hybridize to the control sequences.

74. The method of any of embodiments 67-73, wherein the target sequence of interest is on the gene of interest, and the control sequences correspond to one or more reference genes that are different from the gene of interest.

75. The method of any of embodiments 67-74, wherein the gene of interest is a survival of motor neuron 1 (SMN1) gene and/or a survival of motor neuron 2 (SMN2) gene.

76. The method of any of embodiments 67-74, wherein the gene of interest is a BRCA1 gene.

77. The method of any of embodiments 67-74, wherein the gene of interest is a DMD gene.

78. The method of any of embodiments 67-77, wherein the at least one criterion includes a requirement that the control probe capture metric are above a first threshold and below a second threshold.

79. The method of embodiment 78, further comprising determining the first threshold and the second threshold based at least in part on the target probe capture metric computed at step d).

80. The method of embodiment 79, wherein the first threshold and the second threshold are determined further based at least in part on the plurality of control probe capture metrics computed at step d).

81. The method of any of embodiments 67-80, further comprising, for each control population, computing a variability coefficient for the control probe capture metrics computed at step d).

82. The method of embodiment 81, wherein the at least one criterion includes a requirement that the variability coefficient is below a threshold.

83. The method of any of embodiments 67-82, wherein the factor computed at step f) is an average of the control probe capture metrics satisfying the at least one criterion.

84. The method of any of embodiments 67-83, wherein the target sequence of interest is on a survival of motor neuron 1 (SMN1) gene and/or a survival of motor neuron 2 (SMN2) gene.

85. The method of embodiment 84, wherein the same known genotype corresponds to a known copy count of an SMN1 gene or an SMN2 gene.

86. The method of any of embodiments 67-83, wherein the target sequence of interest is on exon 11 of a BRCA1 gene.

87. The method of embodiment 86, wherein the same known genotype corresponds to a known copy count of exon 11 of the BRCA1 gene.

88. The method of any of embodiments 67-83, wherein the target sequence of interest is on a DMD gene.

89. The method of embodiment 88, wherein the same known genotype corresponds to a known copy count of the DMD gene.

90. A system configured to perform the method of any of embodiments 67-89.

91. A computer program product comprising computer-readable instructions that, when executed in a computerized system comprising at least one processor, cause the processor to carry out one or more steps of the method of any of embodiments 67-89.

92. The method of any one of embodiments 1-40, 43-64, and 67-89, wherein the subject or the test subject is a candidate for carrier screening of one or more diseases or conditions.

93. The method of any one of embodiments 1-40, 43-64, and 67-89, wherein the subject or the test subject is a candidate for:

a) a pharmacogenomics test;

b) a targeted tumor test;

c) an exonic deletion test; or

d) an exonic duplication test.

94. The method of any one of embodiments 1-40, 43-64, 67-89, 92, and 93, wherein the method is for detecting a) a single nucleotide polymorphism; or b) an exonic deletion; or c) an exonic duplication.

95. The method of any one of embodiments 1-40, 43-64, 67-89, and 92-94, wherein the one or more target sequences are one or more deleted exons in a gene of interest.

96. The method of any one of embodiments 1-40, 43-64, 67-89, and 92-94, wherein the one or more target sequences are one or more duplicated exons in a gene of interest.

97. The method of embodiment 95 or 96, wherein the gene of interest is a BRCA1 or a BRCA2 gene.

98. The method of embodiment 95 or 96, wherein the gene of interest is a DMD gene.

99. The method of embodiment 97, wherein the targeting MIP comprises the sequence of

(SEQ ID NO: 9)
5′-GTCTGAATCAAATGCCAAAGTNNNNNNNNNNCTTCAGCTTCCCGATT
ACGGGTACGATCCGACGGTAGTGTNNNNNNNNNNTCCCCTGTGTGAGA
GAAAAGA-3′.

100. The method of embodiment 98, wherein the targeting MIPs are selected from Table 3.

101. A nucleic acid molecule comprising the sequences selected from Table 3.

102. A nucleic acid molecule comprising the sequence of

(SEQ ID NO: 9)
5′-GTCTGAATCAAATGCCAAAGTNNNNNNNNNNCTTCAGCTTCCCGATT
ACGGGTACGATCCGACGGTAGTGTNNNNNNNNNNTCCCCTGTGTGAGA
GAAAAGA-3′.

### BRIEF DESCRIPTION OF THE DRAWINGS

FIG. 1 shows the sequence of a molecular inversion probe (MIP) used in some embodiments of the methods of the disclosure (e.g., a specific target site or sequence in SMN1/SMN2). The MIP comprises in sequence the following components: a first targeting polynucleotide arm, a first unique targeting molecular tag, a polynucleotide linker, a second unique targeting molecular tag, and a second targeting polynucleotide arm. The first and second targeting polynucleotide arms in each of the MIP are substantially complementary to first and second regions in the nucleic acid that, respectively, flank a site or sequence of interest (a target site or sequence or control site or sequence). The unique molecular tags are random polynucleotide sequences. In some embodiments, e.g., when the targeting polynucleotide arms hybridize to the first and second regions in the nucleic acid that, respectively, flank a site of interest, “substantially complementary” refers to 0 mismatches in both arms, or at most 1 mismatch in only one arm. In other embodiments, “substantially complementary” refers to at most a small number of mismatches in both arms, such as 1, 2, 3, 3, 5, or any other suitable number.

FIG. 2 is a representative process flow diagram for determining a copy number variant according to some embodiments of the disclosure.

FIG. 3 is a block diagram of a computing device for performing any of the processes described herein.

FIG. 4 is a representative process flow diagram for determining a copy count number for a test subject, according to an illustrative embodiment.

FIG. 5 is a representative process flow diagram for forming a genotype cluster, according to an illustrative embodiment.

FIG. 6 is a plot of six illustrative genotype clusters that are used for comparison to a test metric evaluated from a test subject, according to an illustrative embodiment.

FIG. 7 is a representative process flow diagram for handling the sample and practicing some embodiments of the disclosure.

FIG. 8 is a diagram of a MIP and DNA captured between two targeting polynucleotide arms of the MIP, according to an illustrative embodiment.

FIG. 9 is a diagram of an example MIP and captured DNA, according to an illustrative embodiment.

FIG. 10 is a boxplot of results of an assay for estimating a copy number of the BRCA1 exon 11, according to an illustrative embodiment.

FIGS. 11-14 are plots of averaged probe capture metrics vs. 79 exons in the DMD gene that exhibit duplication or deletion, according to an illustrative embodiment.

### DETAILED DESCRIPTION OF THE INVENTION

This disclosure provides systems and methods for determining, inter alia, copy number variations, chromosomal abnormalities or micro-deletions in a subject in need thereof. In some embodiments, the subject is a candidate for a disease or condition carrier screening. In some embodiments, the subject is a candidate for pharmacogenomics testing. In some embodiments, the subject is a candidate for targeted tumor testing (e.g., targeted tumor sequencing or targeted tumor analysis). In some embodiments, the subject is a candidate for pediatric diagnostic testing, such as for Duchenne's muscular dystrophy.

Embodiments of the disclosure relate to systems and methods that enable accurate and robust copy counting at any particular targeted site or sequence of interest, or targeted gene of interest, or targeted sequence of interest, in a genome using circular capture probes (e.g., molecular inversion probes) and short read sequencing technology. The systems and methods of embodiments of this disclosure allow one to get an accurate representation of how many copies of any targeted site or sequence of interest, or targeted gene of interest, or targeted sequence of interest, exist in the genome. The systems and methods of embodiments of this disclosure are useful for determining the copy count of targeted site or sequence of interest, or targeted gene of interest, or targeted sequence of interest in the context of carrier screening for a variety of diseases (e.g., spinal muscular atrophy) or risk factors.

The systems and methods of embodiments of this disclosure are also useful in other genomic applications where copy count variations or copy number variations are important variables, such as determining exonic deletions, exonic duplications, pharmacogenomics testing, or targeted tumor testing (e.g., sequencing).

The systems and methods of embodiments described herein are useful for examining or determining exonic deletions or duplications in disease-causing genes. For example, the systems and methods of embodiments of this disclosure can be used to determine exonic deletions in BRCA1 and BRCA2, where large exonic deletions account for a significant percentage of all causative variants. The systems and methods of embodiments of this disclosure can also be used to determine or examine exonic deletions or duplications in the DMD gene associated with Duchenne and Beckers Muscular dystrophy.

The systems and methods of embodiments of this disclosure are also applicable to pharmagogenomic testing. For example, The systems and methods of embodiments of this disclosure may be used to determine the copy count of the p450 enzyme CYP2D6, where −5% of the population has a duplication of this gene, causing them to more rapidly metabolize certain drugs such as codeine.

The systems and methods of embodiments of this disclosure are also applicable to targeted tumor testing. For example, The systems and methods of embodiments of this disclosure may be used to determine the duplication of certain genes that are known to be important for tumor progression, such as MYC, MYCN, RET, EGFR etc.

The systems and methods of embodiments of this disclosure offer a simple and cost effective approach for determining copy count in the context of a sequencing assay. Many variants of interest can be jointly and accurately assessed for copy count and sequence variation in a single assay. The systems and methods of embodiments of this disclosure allow for sequencing information to be combined with copy number variation information at a single site or sequence, which results in a simpler and more cost-effective workflow. The systems and methods of embodiments of this disclosure use unique identifiers on each probe (e.g., unique molecular tags) to determine, inter alfa, a maximum likelihood estimate (k), which allows one to estimate probe capture efficiency, thereby increasing accuracy and reducing the need for extraneous sequencing. The systems and methods of embodiments of this disclosure use circular capture probes, which allow for the combination of multiple additional probes in a single, multiplexed assay with minimal interference or cross assay reactions. Combining the information from several probes and their unique reads greatly reduces errors in the system and improves efficiency.

In some embodiments, The systems and methods of embodiments of this disclosure count the number of unique molecular tags and use such counting to estimate a probe capture efficiency and further to determine the copy count of a gene or site or sequence of interest. Counting the number of unique molecular tags provides a more accurate picture of the relative abundance of each sequence in the original nucleic acid sample when compared to counting sequencing reads.

In order that the disclosure herein described may be fully understood, the following detailed description is set forth.

Unless otherwise defined herein, scientific and technical terms used in this application shall have the meanings that are commonly understood by those of ordinary skill in the art to which this disclosure belongs. Generally, nomenclature used in connection with, and techniques of, cell and tissue culture, molecular biology, cell biology, cancer biology, neurobiology, neurochemistry, virology, immunology, microbiology, genetics, protein and nucleic acid chemistry, chemistry, and pharmacology described herein, are those well known and commonly used in the art. Each embodiment described herein may be taken alone or in combination with one or more other embodiments of the disclosure.

The methods and techniques of various embodiments of the present disclosure are generally performed, unless otherwise indicated, according to methods well known in the art and as described in various general and more specific references that are cited and discussed throughout this specification. See, e.g. Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); Gilbert et al., “Developmental Biology, 6th ed.”, Sinauer Associates, Inc., Sunderland, Mass. (2000).

Chemistry terms used herein are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to”. “Including” and “including but not limited to” are used interchangeably.

In order to further define the disclosure, the following terms and definitions are provided herein.

### Definitions

The term “copy number variation,” “CNV,” “a copy number variant,” or “a gene copy number variant,” as used herein, refers to variation in the number of copies of a nucleic acid sequence present in a test sample (e.g., a nucleic acid sample isolated from, or derived from, or obtained from a carrier screening candidate) in comparison with the copy number of the nucleic acid sequence present in a reference sample (e.g., a nucleic acid sample isolated from, or derived from, or obtained from a reference subject exhibiting known genotypes). In some embodiments, the nucleic acid sequence is 1 kb or larger. In some embodiments, the nucleic acid sequence is a whole chromosome or significant portion thereof. In some embodiments, copy number differences are identified by comparison of a sequence of interest in a test sample with an expected level of the sequence of interest. For example, the level of the sequence of interest in the test sample is compared to that present in a reference sample. In some embodiments, copy number variation refers to a form of structural variation of the DNA of a genome that results in a cell having an abnormal or, for certain genes, a normal variation in the number of copies of one or more sections of the DNA.

In some embodiments, copy number variations (“CNVs”) refer to relatively large regions of the genome that have been deleted (fewer than the normal number) or duplicated (more than the normal number) on certain chromosomes. For example, the chromosome that normally has sections in order as A-B-C-D-E might instead have sections A-B-C-C-D-E (a duplication of “C”) or A-B-D-E (a deletion of “C”). This variation accounts for roughly 12% of human genomic DNA and each variation may range from about 500 base pairs (500 nucleotide bases) to several megabases in size (e.g., between 5,000 to 5 million bases). In some embodiments, copy number variations refer to relative small regions of the genome that have been deleted (e.g., micro-deletions) or duplicated on certain chromosomes. In some embodiments, copy number variations refer to genetic variants due to presence of single-nucleotide polymorphisms (SNPs), which affect only one single nucleotide base. In some embodiments, copy number variants/variations include deletions, including micro-deletions, insertions, including micro-insertions, duplications, multiplications, inversions, translocations and complex multi-site variants. In some embodiments, copy number variants/variations encompass chromosomal aneuploidies and partial aneuploidies.

In some embodiments a copy number variation is a fetal copy number variation. Often, a fetal copy number variation is a copy number variation in the genome of a fetus. In some embodiments a copy number variation is a maternal and/or fetal copy number variation. In certain embodiments a maternal and/or fetal copy number variation is a copy number variation within the genome of a pregnant female (e.g., a female subject bearing a fetus), a female subject that gave birth or a female capable of bearing a fetus.

A copy number variation can be a heterozygous copy number variation where the variation (e.g., a duplication or deletion) is present on one allele of a genome. A copy number variation can be a homozygous copy number variation where the variation is present on both alleles of a genome. In some embodiments a copy number variation is a heterozygous or homozygous fetal copy number variation. In some embodiments a copy number variation is a heterozygous or homozygous maternal and/or fetal copy number variation. A copy number variation sometimes is present in a maternal genome and a fetal genome, a maternal genome and not a fetal genome, or a fetal genome and not a maternal genome.

The term “aneuploidy,” as used herein, refers to a chromosomal abnormality characterized by an abnormal variation in chromosome number, e.g., a number of chromosomes that is not an exact multiple of the haploid number of chromosomes. For example, a euploid individual will have a number of chromosomes equaling 2 n, where n is the number of chromosomes in the haploid individual. In humans, the haploid number is 23. Thus, a diploid individual will have 46 chromosomes. An aneuploid individual may contain an extra copy of a chromosome (trisomy of that chromosome) or lack a copy of the chromosome (monosomy of that chromosome). The abnormal variation is with respect to each individual chromosome. Thus, an individual with both a trisomy and a monosomy is aneuploid despite having 46 chromosomes. Examples of aneuploidy diseases or conditions include, but are not limited to, Down syndrome (trisomy of chromosome 21), Edwards syndrome (trisomy of chromosome 18), Patau syndrome (trisomy of chromosome 13), Turner syndrome (monosomy of the X chromosome in a female), and Klinefelter syndrome (an extra copy of the X chromosome in a male). Other, non-aneuploid chromosomal abnormalities include translocation (wherein a segment of a chromosome has been transferred to another chromosome) and deletion (wherein a piece of a chromosome has been lost), and other types of chromosomal damage.

The terms “subject” and “patient”, as used herein, refer to any animal, such as a dog, a cat, a bird, livestock, and particularly a mammal, and preferably a human. The term “reference subject” and “reference patients” refer to any subject or patient that exhibits known genotypes (e.g., known copy number of a site of interest, or a gene of interest, or a sequence of interest). The term “test subject”, “test patients”, or “candidate”, or “candidate subject”, “targeted subject” or “targeted individual” refers to any subject or patient or individual that exhibit known genotypes (e.g., known copy number of a site of interest, or a gene of interest, or a sequence of interest).

The terms “polynucleotide”, “nucleic acid” and “nucleic acid molecules”, as used herein, are used interchangeably and refer to DNA molecules (e.g., cDNA or genomic DNA), RNA molecules (e.g., mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNAs), microRNA (miRNAs), small nucleolar RNA (snoRNAs), ribosomal RNA (rRNA), transfer RNA (tRNA), small interfering RNA (siRNA), heterogeneous nuclear RNAs (hnRNA), or small hairpin RNA (shRNA).

The term “sample”, as used herein, refers to a sample typically derived from a biological fluid, cell, tissue, organ, or organism, comprising a nucleic acid or a mixture of nucleic acids comprising at least one nucleic acid sequence that is to be screened for copy number variation (including aneuploidy or micro-deletions). In some embodiments the sample comprises at least one nucleic acid sequence whose copy number is suspected of having undergone variation. Such samples include, but are not limited to sputum/oral fluid, amniotic fluid, blood, a blood fraction, or fine needle biopsy samples (e.g., surgical biopsy, fine needle biopsy, etc.) urine, peritoneal fluid, pleural fluid, and the like. Although the sample is often taken from a human subject (e.g., a candidate for a disease or condition carrier screening), the assays can be used to detect copy number variations (CNVs) in samples from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, preferably at a concentration proportional to that in an untreated test sample (e.g., namely, a sample that is not subjected to any such pretreatment method(s)). Depending on the type of sample used, additional processing and/or purification steps may be performed to obtain nucleic acid fragments of a desired purity or size, using processing methods including but not limited to sonication, nebulization, gel purification, PCR purification systems, nuclease cleavage, size-specific capture or exclusion, targeted capture or a combination of these methods. Optionally, cell-free DNA may be isolated from, or derived from, or obtained from the sample prior to further analysis. In some embodiments, the sample is from the subject whose copy number variation is to be determined by the systems and methods of embodiments of this disclosure, also referred as “a test sample.”

In some embodiments, the sample is from a subject exhibiting known genome type or copy number variation, also referred as a reference sample. A reference sample refers to a sample comprising a mixture of nucleic acids that are present in a known copy number to which the nucleic acids in a test sample are to be compared. In some embodiments, it is a sample that is normal, i.e. not aneuploid, for the sequence of interest. In some embodiments, it is a sample that is abnormal for the sequence of interest. In some embodiments, reference samples are used for identifying one or more normalizing site or sequences of interest, or genes of interest, or chromosomes of interests.

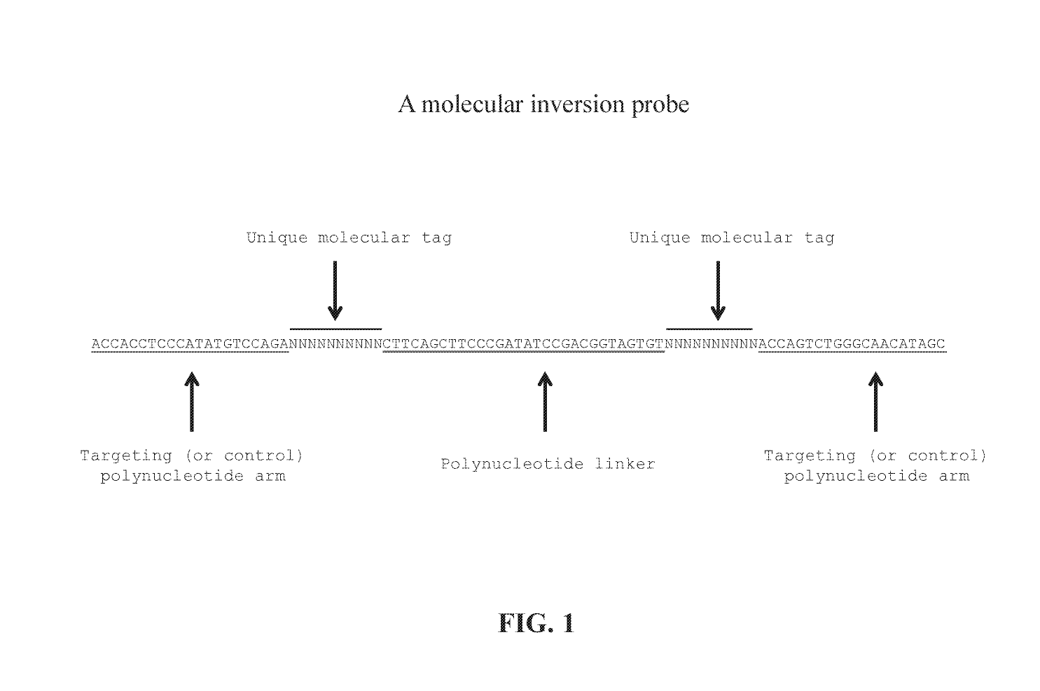

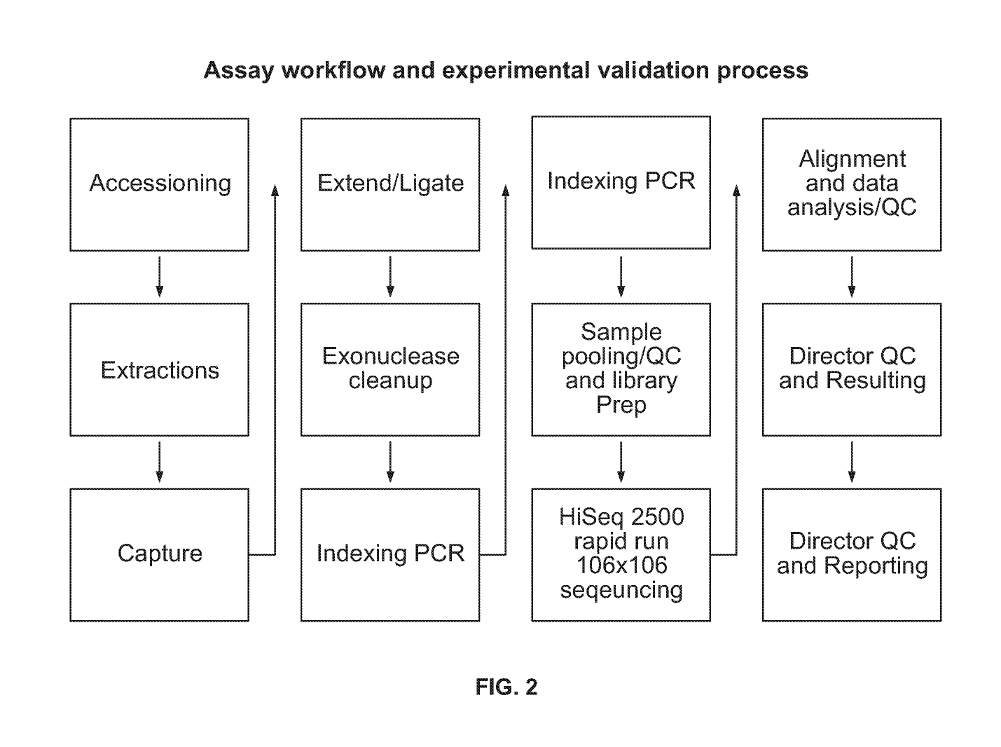

The term “MIP” as used herein, refers to a molecular inversion probe (or a circular capture probe). Molecular inversion probes (or circular capture probes) are nucleic acid molecules that comprise a pair of unique polynucleotide arms, one or more unique molecular tags (or unique molecular identifiers), and a polynucleotide linker (e.g., a universal backbone linker). See, for example, FIG. 1. In some embodiments, a MIP may comprise more than one unique molecular tags, such as, two unique molecular tags, three unique molecular tags, or more. In some embodiments, the unique polynucleotide arms in each MIP are located at the 5′ and 3′ ends of the MIP, while the unique molecular tag(s) and the polynucleotide linker are located internal to the 5′ and 3′ ends of the MIP. For example, the MIPs that are used in some embodiments of this disclosure comprise in sequence the following components: first unique polynucleotide arm—first unique molecular tag—polynucleotide linker—second unique molecular tag—second unique polynucleotide arm. In some embodiments, the MIP is a 5′ phosphorylated single-stranded nucleic acid (e.g., DNA) molecule.

The unique molecular tag may be any tag that is detectable and can be incorporated into or attached to a nucleic acid (e.g., a polynucleotide) and allows detection and/or identification of nucleic acids that comprise the tag. In some embodiments the tag is incorporated into or attached to a nucleic acid during sequencing (e.g., by a polymerase). Non-limiting examples of tags include nucleic acid tags, nucleic acid indexes or barcodes, radiolabels (e.g., isotopes), metallic labels, fluorescent labels, chemiluminescent labels, phosphorescent labels, fluorophore quenchers, dyes, proteins (e.g., enzymes, antibodies or parts thereof, linkers, members of a binding pair), the like or combinations thereof. In some embodiments, particularly sequencing embodiments, the tag (e.g., a molecular tag) is a unique, known and/or identifiable sequence of nucleotides or nucleotide analogues (e.g., nucleotides comprising a nucleic acid analogue, a sugar and one to three phosphate groups). In some embodiments, tags are six or more contiguous nucleotides. A multitude of fluorophore-based tags are available with a variety of different excitation and emission spectra. Any suitable type and/or number of fluorophores can be used as a tag. In some embodiments 1 or more, 2 or more, 3 or more, 4 or more, 5 or more, 6 or more, 7 or more, 8 or more, 9 or more, 10 or more, 20 or more, 30 or more, 50 or more, 100 or more, 500 or more, 1000 or more, 10,000 or more, 100,000 or more different tags are utilized in a method described herein (e.g., a nucleic acid detection and/or sequencing method). In some embodiments, one or two types of tags (e.g., different fluorescent labels) are linked to each nucleic acid in a library. In some embodiments, chromosome-specific tags are used to make chromosomal counting faster or more efficient. Detection and/or quantification of a tag can be performed by a suitable method, machine or apparatus, non-limiting examples of which include flow cytometry, quantitative polymerase chain reaction (qPCR), gel electrophoresis, a luminometer, a fluorometer, a spectrophotometer, a suitable gene- chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In the MIPs, the unique polynucleotide arms are designed to hybridize immediately upstream and downstream of a specific target sequence (or site) in a genomic nucleic acid sample. The unique molecular tags are short nucleotide sequences that are randomly generated. In some embodiments, the unique molecular tags do not hybridize to any sequence or site located on a genomic nucleic acid fragment or in a genomic nucleic acid sample. In some embodiments, the polynucleotide linker (or the backbone linker) in the MIPs are universal in all the MIPs used in embodiments of this disclosure.

In some embodiments, the MIPs are introduced to nucleic acid fragments derived from a test subject (or a reference subject) to perform capture of target sequences or sites (or control sequences or sites) located on a nucleic acid sample (e.g., a genomic DNA). In some embodiments, fragmenting aids in capture of target nucleic acid by molecular inversion probes. In some embodiments, for example, when the nucleic acid sample is comprised of cell free nucleic acid, fragmenting may not be necessary to improve capture of target nucleic acid by molecular inversion probes. As described in greater detail herein, after capture of the target sequence (e.g., locus) of interest, the captured target may be subjected to enzymatic gap-filling and ligation steps, such that a copy of the target sequence is incorporated into a circle-like structure. Capture efficiency of the MIP to the target sequence on the nucleic acid fragment can, in some embodiments, be improved by lengthening the hybridization and gap-filing incubation periods. (See, e.g., Turner E H, et al., Nat Methods. 2009 Apr. 6:1-2.).

In some embodiments, the MIPs that are used according to the disclosure to capture a target site or target sequence comprise in sequence the following components:
first targeting polynucleotide arm—first unique targeting molecular tag—polynucleotide linker—second unique targeting molecular tag—second targeting polynucleotide arm.

In some embodiments, the MIPs that are used in the disclosure to capture a control site or control sequence comprise in sequence the following components:
first control polynucleotide arm—first unique control molecular tag—polynucleotide linker—second unique control molecular tag—second control polynucleotide arm.

MIP technology may be used to detect or amplify particular nucleic acid sequences in complex mixtures. One of the advantages of using the MIP technology is in its capacity for a high degree of multiplexing, which allows thousands of target sequences to be captured in a single reaction containing thousands of MIPs. Various aspects of MIP technology are described in, for example, Hardenbol et al., “Multiplexed genotyping with sequence-tagged molecular inversion probes,” Nature Biotechnology, 21(6): 673-678 (2003); Hardenbol et al., “Highly multiplexed molecular inversion probe genotyping: Over 10,000 targeted SNPs genotyped in a single tube assay,” Genome Research, 15: 269-275 (2005); Burmester et al., “DMET microarray technology for pharmacogenomics-based personalized medicine,” Methods in Molecular Biology, 632: 99-124 (2010); Sissung et al., “Clinical pharmacology and pharmacogenetics in a genomics era: the DMET platform,” Pharmacogenomics, 11(1): 89-103 (2010); Deeken, “The Affymetrix DMET platform and pharmacogenetics in drug development,” Current Opinion in Molecular Therapeutics, 11(3): 260-268 (2009); Wang et al., “High quality copy number and genotype data from FFPE samples using Molecular Inversion Probe (MIP) microarrays,” BMC Medical Genomics, 2:8 (2009); Wang et al., “Analysis of molecular inversion probe performance for allele copy number determination,” Genome Biology, 8(11): R246 (2007); Ji et al., “Molecular inversion probe analysis of gene copy alternations reveals distinct categories of colorectal carcinoma,” Cancer Research, 66(16): 7910-7919 (2006); and Wang et al., “Allele quantification using molecular inversion probes (MIP),” Nucleic Acids Research, 33(21): e183 (2005), each of which is hereby incorporated by reference in its entirety for all purposes. See also in U.S. Pat. Nos. 6,858,412; 5,817,921; 6,558,928; 7,320,860; 7,351,528; 5,866,337; 6,027,889 and 6,852,487, each of which is hereby incorporated by reference in its entirety for all purposes.

MIP technology has previously been successfully applied to other areas of research, including the novel identification and subclassification of biomarkers in cancers. See, e.g., Brewster et al., “Copy number imbalances between screen- and symptom-detected breast cancers and impact on disease-free survival,” Cancer Prevention Research, 4(10): 1609-1616 (2011); Geiersbach et al., “Unknown partner for USP6 and unusual SS18 rearrangement detected by fluorescence in situ hybridization in a solid aneurysmal bone cyst,” Cancer Genetics, 204(4): 195-202 (2011); Schiffman et al., “Oncogenic BRAF mutation with CDKN2A inactivation is characteristic of a subset of pediatric malignant astrocytomas,” Cancer Research, 70(2): 512-519 (2010); Schiffman et al., “Molecular inversion probes reveal patterns of 9p21 deletion and copy number aberrations in childhood leukemia,” Cancer Genetics and Cytogenetics, 193(1): 9-18 (2009); Press et al., “Ovarian carcinomas with genetic and epigenetic BRCA1 loss have distinct molecular abnormalities,” BMC Cancer, 8:17 (2008); and Deeken et al., “A pharmacogenetic study of docetaxel and thalidomide in patients with castration-resistant prostate cancer using the DMET genotyping platform,” Pharmacogenomics, 10(3): 191-199 (2009), ach of which is hereby incorporated by reference in its entirety for all purposes.

MIP technology has also been applied to the identification of new drug- related biomarkers. See, e.g., Caldwell et al., “CYP4F2 genetic variant alters required warfarin dose,” Blood, 111(8): 4106-4112 (2008); and McDonald et al., “CYP4F2 Is a Vitamin K1 Oxidase: An Explanation for Altered Warfarin Dose in Carriers of the V433M Variant,” Molecular Pharmacology, 75: 1337-1346 (2009), each of which is hereby incorporated by reference in its entirety for all purposes. Other MIP applications include drug development and safety research. See, e.g., Mega et al., “Cytochrome P-450 Polymorphisms and Response to Clopidogrel,” New England Journal of Medicine, 360(4): 354-362 (2009); Dumaual et al., “Comprehensive assessment of metabolic enzyme and transporter genes using the Affymetrix Targeted Genotyping System,” Pharmacogenomics, 8(3): 293-305 (2007); and Daly et al., “Multiplex assay for comprehensive genotyping of genes involved in drug metabolism, excretion, and transport,” Clinical Chemistry, 53(7): 1222-1230 (2007), each of which is hereby incorporated by reference in its entirety for all purposes. Further applications of MIP technology include genotype and phenotype databasing. See, e.g., Man et al., “Genetic Variation in Metabolizing Enzyme and Transporter Genes: Comprehensive Assessment in 3 Major East Asian Subpopulations With Comparison to Caucasians and Africans,” Journal of Clinical Pharmacology, 50(8): 929-940 (2010), which is hereby incorporated by reference in its entirety for all purposes.

The term “capture” or “capturing”, as used herein, refers to the binding or hybridization reaction between a molecular inversion probe and its corresponding targeting site. In some embodiments, upon capturing, a circular replicon or a MIP replicon is produced or formed. In some embodiments, the targeting site is a deletion (e.g., partial or full deletion of one or more exons). In some embodiments, a target MIP is designed to bind to or hybridize with a naturally-occurring (e.g., wild-type) genomic region of interest where a target deletion is expected to be located. The target MIP is designed to not bind to a genomic region exhibiting the deletion. In these embodiments, binding or hybridization between a target MIP and the target site of deletion is expected to not occur. The absence of such binding or hybridization indicates the presence of the target deletion. In these embodiments, the phrase “capturing a target site” or the phrase “capturing a target sequence” refers to detection of a target deletion by detecting the absence of such binding or hybridization.

The term “MIP replicon” or “circular replicon”, as used herein, refers to a circular nucleic acid molecule generated via a capturing reaction (e.g., a binding or hybridization reaction between a MIP and its targeted sequence). In some embodiments, the MIP replicon is a single-stranded circular nucleic acid molecule. In some embodiments, a targeting MIP captures or hybridizes to a target sequence or site. After the capturing reaction or hybridization, a ligation/extension mixture is introduced to extend and ligate the gap region between the two targeting polynucleotide arms to form single-stranded circular nucleotide molecules, i.e., a targeting MIP replicon. In some embodiments, a control MIP captures or hybridizes to a control sequence or site. After the capturing reaction or hybridization, a ligation/extension mixture is introduced to extend and ligate the gap region between the two control polynucleotide arms to form single-stranded circular nucleotide molecules, i.e., a control MIP replicon. MIP replicons may be amplified through a polymerase chain reaction (PCR) to produce a plurality of targeting MIP amplicons, which are double-stranded nucleotide molecules.

The term “amplicon”, as used herein, refers to a nucleic acid generated via amplification reaction (e.g., a PCR reaction). In some embodiments, the amplicon is a single-stranded nucleic acid molecule. In some embodiments, the amplicon is a double-stranded nucleic acid molecule. In some embodiments, a targeting MIP replicon is amplified using conventional techniques to produce a plurality of targeting MIP amplicons, which are double-stranded nucleotide molecules. In some embodiments, a control MIP replicon is amplified using conventional techniques to produce a plurality of control MIP amplicons, which are double-stranded nucleotide molecules.

The term “sequencing”, as used herein, is used in a broad sense and may refer to any technique known in the art that allows the order of at least some consecutive nucleotides in at least part of a nucleic acid to be identified, including without limitation at least part of an extension product or a vector insert. In some embodiments, sequencing allows the distinguishing of sequence differences between different target sequences. Exemplary sequencing techniques include targeted sequencing, single molecule real-time sequencing, electron microscopy-based sequencing, transistor-mediated sequencing, direct sequencing, random shotgun sequencing, Sanger dideoxy termination sequencing, targeted sequencing, exon sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, ion semiconductor sequencing, nanoball sequencing, nanopore sequencing, 454 sequencing, Solexa Genome Analyzer sequencing, miSeq (Illumina), HiSeq 2000 (Illumina), HiSeq 2500 (Illumina), Illumina Genome Analyzer (Illumina), Ion Torrent PGMTM (Life Technologies), MinION™ (Oxford Nanopore Technologies), real-time SMIRT™ technology (Pacific Biosciences), the Probe-Anchor Ligation (cPAL™) (Complete Genomics/BGI), SOLiD® sequencing, MS-PET sequencing, mass spectrometry, and a combination thereof. In some embodiments, sequencing comprises detecting the sequencing product using an instrument, for example but not limited to an ABI PRISM® 377 DNA Sequencer, an ABI PRISM® 310, 3100, 3100-Avant, 3730, or 3730xI Genetic Analyzer, an ABI PRISM® 3700 DNA Analyzer, or an Applied Biosystems SOLiD™ System (all from Applied Biosystems), a Genome Sequencer 20 System (Roche Applied Science), or a mass spectrometer. In certain embodiments, sequencing comprises emulsion PCR. In certain embodiments, sequencing comprises a high throughput sequencing technique, for example but not limited to, massively parallel signature sequencing (MPSS).

It will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof

This disclosure will be better understood from the Experimental Details which follow. However, one skilled in the art will readily appreciate that the specific methods and results discussed are merely illustrative of various embodiments of the disclosure as described more fully as follows.

### Methods of the Disclosure

In one aspect, the disclosure provides a method of detecting copy number variation (e.g., single-nucleotide polymorphism, or exonic deletion, or exonic duplication) in a subject in need thereof. In some embodiments, the method comprises:

a) obtaining a nucleic acid sample isolated from the subject;

b) capturing or detecting one or more target sequences (e.g., a genomic region comprising the single nucleotide polymorphism, or one or more deleted exons, or one or more duplicated exons) in the nucleic acid sample obtained in step a) by using one or more target populations of targeting molecular inversion probes (MIPs) to produce a plurality of targeting MIPs replicons for each target sequence,

wherein each of the targeting MIPs in each of the target populations comprises in sequence the following components:

first targeting polynucleotide arm—first unique targeting molecular tag—polynucleotide linker—second unique targeting molecular tag—second targeting polynucleotide arm;

wherein the pair of first and second targeting polynucleotide arms in each of the targeting MIPs in each target population are identical, and are substantially complementary to first and second regions in the nucleic acid that, respectively, flank the target sequence that is targeted by the one or more targeting MIPs;

wherein the first and second unique targeting molecular tags in each of the targeting MIPs in each target population are distinct in each of the targeting MIPs, in each member of the target population, and in each of the target populations;

c) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),

wherein each of the control MIPs in each control population comprises in sequence the following components:

first control polynucleotide arm—first unique control molecular tag—polynucleotide linker—second unique control molecular tag—second control polynucleotide arm;

wherein the pair of first and second control polynucleotide arms in each of the control MIPs in each control population are identical, and are substantially complementary to first and second regions in the nucleic acid that, respectively, flank each control sequence;

wherein the first and second unique control molecular tags in each of the control MIPs in each control population are distinct in each of the control MIPs and in each member of the control population, and are different from the unique targeting molecular tags;

d) sequencing the targeting and control MIPs amplicons that are amplified from the targeting and control MIPs replicons obtained in steps b) and c);

e) determining, for each target population, the number of the unique targeting molecular tags present in the targeting MIPs amplicons sequenced in step d);

f) determining, for each control population, the number of the unique control molecular tags present in the control MIPs amplicons sequenced in step d);

g) computing a target probe capture metric, for each of the one or more target sequences, based at least in part on the number of the unique targeting molecular tags determined in step e) and a plurality of control probe capture metrics based at least in part on the numbers of the unique control molecular tags determined in step f);

h) identifying a subset of the control populations of control MIPs that have control probe capture metrics satisfying at least one criterion;

i) normalizing each of the one or more target probe capture metrics by a factor computed from the subset of control probe capture metrics satisfying the at least one criterion, to obtain a test normalized target probe capture metric for each of the one or more target sequences;

j) comparing each test normalized target probe capture metric obtained in step i) to a plurality of reference normalized target probe capture metrics that are computed based on reference nucleic acid samples obtained from reference subjects exhibiting known genotypes using the same target and control sequences, target population, one subset of control populations in steps b)-g) and i); and

k) determining, based on the comparing in step j) and the known genotypes of reference subjects, the copy number variation of each of the one or more target sequences of interest.

In another aspect, the disclosure provides a method of detecting copy number variation (e.g., single-nucleotide polymorphism, or exonic deletion, or exonic duplication) in a subject in need thereof. In some embodiments, the method comprises:

a) obtaining a nucleic acid sample isolated from the subject;

b) capturing or detecting one or more target sequences (e.g., a genomic region comprising the single nucleotide polymorphism, or one or more deleted exons, or one or more duplicated exons) in the nucleic acid sample obtained in step a) by using one or more target populations of targeting molecular inversion probes (MIPs) to produce a plurality of targeting MIPs replicons for each target sequence,

wherein each of the targeting MIPs in each of the target populations comprises in sequence the following components:

first targeting polynucleotide arm—first unique targeting molecular tag—polynucleotide linker—second unique targeting molecular tag—second targeting polynucleotide arm;

wherein the pair of first and second targeting polynucleotide arms in each of the targeting MIPs in each target population are identical, and are substantially complementary to first and second regions in the nucleic acid that, respectively, flank the target sequence that is targeted by the one or more targeting MIPs;

wherein the first and second unique targeting molecular tags in each of the targeting MIPs in each target population are distinct in each of the targeting MIPs, in each member of the target population, and in each of the target populations;

c) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),

wherein each of the control MIPs in each control population comprises in sequence the following components:

first control polynucleotide arm—first unique control molecular tag—polynucleotide linker—second unique control molecular tag—second control polynucleotide arm;

wherein the pair of first and second control polynucleotide arms in each of the control MIPs in each control population are identical, and are substantially complementary to first and second regions in the nucleic acid that, respectively, flank each control sequence;

wherein the first and second unique control molecular tags in each of the control MIPs in each control population are distinct in each of the control MIPs and in each member of the control population, and are different from the unique targeting molecular tags;

d) sequencing the targeting and control MIPs amplicons that are amplified from the targeting and control MIPs replicons obtained in steps b) and c);

e) determining, for each target population, the number of the target capture events by targeting MIPs based on the number of unique targeting molecular tags present in the targeting MIPs amplicons sequenced in step d);

f) determining, for each control population, the number of the control capture events by control MIPs based on the number of unique control molecular tags present in the control MIPs amplicons sequenced in step d);

g) computing a target probe capture metric, for each of the one or more target sequences, based at least in part on the number of the target capture events determined in step e) and a plurality of control probe capture metrics based at least in part on the numbers of the control capture events determined in step f);

h) identifying a subset of the control populations of control MIPs that have control probe capture metrics satisfying at least one criterion;

i) normalizing each of the one or more target probe capture metrics by a factor computed from the subset of control probe capture metrics satisfying the at least one criterion, to obtain a test normalized target probe capture metric for each of the one or more target sequences;

j) comparing each test normalized target probe capture metric obtained in step i) to a plurality of reference normalized target probe capture metrics that are computed based on reference nucleic acid samples obtained from reference subjects exhibiting known genotypes using the same target and control sequences, target population, one subset of control populations in steps b)-g) and i); and

k) determining, based on the comparing in step j) and the known genotypes of reference subjects, the copy number variation of each of the one or more target sequences of interest.

In another aspect, the disclosure provides a method of detecting copy number variation (e.g., single-nucleotide polymorphism, or exonic deletion, or exonic duplication) in a subject comprising:

a) isolating a genomic DNA sample from the subject;

b) adding the genomic DNA sample into each well of a multi-well plate, wherein each well of the multi-well plate comprises a probe mixture, wherein the probe mixture comprises a plurality of target populations of targeting molecular inversion probes (MIPs), a plurality of control populations of control MIPs and buffer;

wherein each targeting population of targeting MIPs is capable of amplifying (or detecting) a distinct target sequence (e.g., a genomic region comprising the single nucleotide polymorphism, or one or more deleted exons, or one or more duplicated exons) in the genomic DNA sample obtained in step a), wherein each of the targeting MIPs in each target population comprises in sequence the following components:

first targeting polynucleotide arm—first unique targeting molecular tag—polynucleotide linker—second unique targeting molecular tag—second targeting polynucleotide arm;

wherein the pair of first and second targeting polynucleotide arms in each of the targeting MIPs in each target population are identical, and are substantially complementary to first and second regions in the genomic DNA that, respectively, flank each target sequence;

wherein the first and second unique targeting molecular tags in each of the targeting MIPs in each target population are distinct in each of the targeting MIPs and in each member of the target population;

wherein each control population of control MIPs is capable of amplifying a distinct control sequence in the genomic DNA sample obtained in step a),

wherein each of the control MIPs in each control population comprises in sequence the following components:

first control polynucleotide arm—first unique control molecular tag—polynucleotide linker—second unique control molecular tag—second control polynucleotide arm;

wherein the pair of first and second control polynucleotide arms in each of the control MIPs in each control population are identical, and are substantially complementary to first and second regions in the genomic DNA that, respectively, flank each control sequence;

wherein the first and second unique control molecular tags in each of the control MIPs in each control population are distinct in each of the control MIPs and in each member of the control population, and are different from the unique targeting molecular tags;

c) incubating the genomic DNA sample with the probe mixture for the targeting MIPs to capture the target sequence and for the control MIPs to capture the control sequences;

d) adding an extension/ligation mixture to the sample of c) for the targeting MIPs and the captured target sequence to form the targeting MIPs replicons and for the control MIPs and the captured control sequences to form the control MIPs replicons, wherein the extension/ligation mixture comprises a polymerase, a plurality of dNTPs, a ligase, and buffer;

e) adding an exonuclease mixture to the targeting and control MIPs replicons to remove excess probes or excess genomic DNA;

f) adding an indexing PCR mixture to the sample of e) to add a pair of indexing primers, a unique sample barcode and a pair of sequencing adaptors to the targeting and control MIPs replicons to produce the targeting and control MIPs amplicons;

g) using a massively parallel sequencing method to determine, for each target population, the number of the unique targeting molecular tags present in the barcoded targeting MIPs amplicons provided in step f);

h) using a massively parallel sequencing method to determine, for each control population, the number of the unique control molecular tags present in the barcoded control MIPs amplicons provided in step f);

i) computing a target probe capture metric for each target sequence based at least in part on the number of the unique targeting molecular tags determined in step g) and a plurality of control probe capture metrics based at least in part on the numbers of the unique control molecular tags determined in step h);

j) identifying a subset of the control populations of control MIPs that have control probe capture metrics satisfying at least one criterion;

k) normalizing each target probe capture metric by a factor computed from the subset of control probe capture metrics satisfying the at least one criterion, to obtain a test normalized target probe capture metric for each target sequence;

l) comparing each test normalized target probe capture metric to a plurality of reference normalized target probe capture metrics that are computed based on reference genomic DNA samples obtained from reference subjects exhibiting known genotypes using the same target and control sequences, target population, one subset of control populations in steps b)-h); and

m) determining, based on the comparing in step l) and the known genotypes of reference subjects, the copy number variation for each target sequence.

In another aspect, the disclosure provides a method of detecting copy number variation (e.g., single-nucleotide polymorphism, or exonic deletion, or exonic duplication) in a subject comprising:

a) isolating a genomic DNA sample from the subject;

b) adding the genomic DNA sample into each well of a multi-well plate, wherein each well of the multi-well plate comprises a probe mixture, wherein the probe mixture comprises a plurality of target populations of targeting molecular inversion probes (MIPs), a plurality of control populations of control MIPs and buffer;

wherein each targeting population of targeting MIPs is capable of amplifying (or detecting) a distinct target sequence (e.g., a genomic region comprising the single nucleotide polymorphism, or one or more deleted exons, or one or more duplicated exons) in the genomic DNA sample obtained in step a),

wherein each of the targeting MIPs in each target population comprises in sequence the following components:

first targeting polynucleotide arm—first unique targeting molecular tag—polynucleotide linker—second unique targeting molecular tag—second targeting polynucleotide arm;

wherein the pair of first and second targeting polynucleotide arms in each of the targeting MIPs in each target population are identical, and are substantially complementary to first and second regions in the genomic DNA that, respectively, flank each target sequence;

wherein the first and second unique targeting molecular tags in each of the targeting MIPs in each target population are distinct in each of the targeting MIPs and in each member of the target population;

wherein each control population of control MIPs is capable of amplifying a distinct control sequence in the genomic DNA sample obtained in step a),

wherein each of the control MIPs in each control population comprises in sequence the following components:

first control polynucleotide arm—first unique control molecular tag—polynucleotide linker—second unique control molecular tag—second control polynucleotide arm;

wherein the pair of first and second control polynucleotide arms in each of the control MIPs in each control population are identical, and are substantially complementary to first and second regions in the genomic DNA that, respectively, flank each control sequence;

wherein the first and second unique control molecular tags in each of the control MIPs in each control population are distinct in each of the control MIPs and in each member of the control population, and are different from the unique targeting molecular tags;

c) incubating the genomic DNA sample with the probe mixture for the targeting MIPs to capture the target sequence and for the control MIPs to capture the control sequences;

d) adding an extension/ligation mixture to the sample of c) for the targeting MIPs and the captured target sequence to form the targeting MIPs replicons and for the control MIPs and the captured control sequences to form the control MIPs replicons, wherein the extension/ligation mixture comprises a polymerase, a plurality of dNTPs, a ligase, and buffer;

e) adding an exonuclease mixture to the targeting and control MIPs replicons to remove excess probes or excess genomic DNA;

f) adding an indexing PCR mixture to the sample of e) to add a pair of indexing primers, a unique sample barcode and a pair of sequencing adaptors to the targeting and control MIPs replicons to produce the targeting and control MIPs amplicons;

g) using a massively parallel sequencing method to determine, for each target population, the number of the unique targeting molecular tags present in the barcoded targeting MIPs amplicons provided in step f);

h) using a massively parallel sequencing method to determine, for each control population, the number of the unique control molecular tags present in the barcoded control MIPs amplicons provided in step f);

i) determining the number of target capture events by the targeting MIPs based on the number of the unique targeting molecular tags determined in step g);

j) determining the numbers of control capture events by the control MIPs based on the numbers of the unique control molecular tags determined in step h);

k) computing a target probe capture metric for each target sequence based at least in part on the number of target capture events determined in step i) and a plurality of control probe capture metrics based at least in part on the numbers of the control capture events determined in step j);

l) identifying a subset of the control populations of control MIPs that have control probe capture metrics satisfying at least one criterion;

m) normalizing each target probe capture metric by a factor computed from the subset of control probe capture metrics satisfying the at least one criterion, to obtain a test normalized target probe capture metric for each target sequence;

n) comparing each test normalized target probe capture metric to a plurality of reference normalized target probe capture metrics that are computed based on reference genomic DNA samples obtained from reference subjects exhibiting known genotypes using the same target and control sequences, target population, one subset of control populations in steps b)-h); and

o) determining, based on the comparing in step n) and the known genotypes of reference subjects, the copy number variation for each target sequence.

In another aspect, the disclosure provides a method for producing a genotype cluster. In some embodiments, the method comprises:

a) receiving sequencing data obtained from a plurality of nucleic acid samples from a plurality of subsets of a plurality of subjects, each sample in the plurality of samples being obtained from a different subject, and each subset being characterized by subjects exhibiting a same known genotype for a gene of interest, wherein the sequencing data for the nucleic acid sample from each subject in the plurality of subsets is obtained by:
i) obtaining a nucleic acid sample isolated from the subject;
ii) capturing one or more target sequences of interest in the nucleic acid sample obtained in step a.i) by using one or more target populations of targeting molecular inversion probes (MIPs) to produce targeting MIPs replicons for each target sequence,
wherein each of the targeting MIPs in each of the target populations comprises in sequence the following components:
first targeting polynucleotide arm—first unique targeting molecular tag—polynucleotide linker—second unique targeting molecular tag—second targeting polynucleotide arm;
wherein the pair of first and second targeting polynucleotide arms in each of the targeting MIPs in each target population are identical, and are substantially complementary to first and second regions in the nucleic acid that, respectively, flank the target sequence of interest that is targeted by the one or more targeting MIPs;
wherein the first and second unique targeting molecular tags in each of the targeting MIPs in each target population are distinct in each of the targeting MIPs and in each member of the target population;
iii) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),
wherein each of the control MIPs in each control population comprises in sequence the following components:
first control polynucleotide arm—first unique control molecular tag—polynucleotide linker—second unique control molecular tag—second control polynucleotide arm;
wherein the pair of first and second control polynucleotide arms in each of the control MIPs in each control population are identical, and are substantially complementary to first and second regions in the nucleic acid that, respectively, flank each control sequence;
wherein the first and second unique control molecular tags in each of the control MIPs in each control population are distinct in each of the control MIPs and in each member of the control population, and are different from the unique targeting molecular tags;
iv) sequencing the targeting and control MIPs amplicons that are amplified from the targeting and control MIPs replicons obtained in steps a.ii) and a.iii);

b) for each respective sample obtained from a subset in the plurality of subsets:
i) determining, for each target population, the number of the unique targeting molecular tags present in the targeting MIPs amplicons sequenced in step a.iv);
ii) determining, for each control population, the number of the unique control molecular tags present in the control MIPs amplicons sequenced in step a.iv);
iii) computing a target probe capture metric, for each target sequence, based at least in part on the number of the unique targeting molecular tags determined in step b.i) and a plurality of control probe capture metrics based at least in part on the numbers of the unique control molecular tags determined in step b.ii);
iv) identifying a subset of the control populations of control MIPs that have control probe capture metrics satisfying at least one criterion;
v) normalizing each target probe capture metric by a factor computed from the control probe capture metrics satisfying the at least one criterion, to obtain a normalized target probe capture metric for each of the one or more target sites; and

c) grouping, across the samples obtained from each subset of subjects, the normalized target probe capture metrics to obtain the genotype cluster for the known genotype.

In some embodiments, computing the target probe capture metric comprises normalizing the number of the unique targeting molecular tags by a sum of the number of the unique targeting molecular tags and the numbers of the unique control molecular tags. In some embodiments, computing the plurality of control probe capture metrics comprises normalizing, for each control population, the number of unique control molecular tags by a sum of the number of the unique targeting molecular tags and the numbers of the unique control molecular tags.

In another aspect, the disclosure provides a method for producing a genotype cluster. In some embodiments, the method comprises:

a) receiving sequencing data obtained from a plurality of nucleic acid samples from a plurality of subsets of a plurality of subjects, each sample in the plurality of samples being obtained from a different subject, and each subset being characterized by subjects exhibiting a same known genotype for a gene of interest, wherein the sequencing data for the nucleic acid sample from each subject in the plurality of subsets is obtained by:
i) obtaining a nucleic acid sample isolated from the subject;
ii) capturing one or more target sequences of interest in the nucleic acid sample obtained in step a.i) by using one or more target populations of targeting molecular inversion probes (MIPs) to produce targeting MIPs replicons for each target sequence,
wherein each of the targeting MIPs in each of the target populations comprises in sequence the following components:
first targeting polynucleotide arm—first unique targeting molecular tag—polynucleotide linker—second unique targeting molecular tag—second targeting polynucleotide arm;
wherein the pair of first and second targeting polynucleotide arms in each of the targeting MIPs in each target population are identical, and are substantially complementary to first and second regions in the nucleic acid that, respectively, flank the target sequence of interest that is targeted by the one or more targeting MIPs;
wherein the first and second unique targeting molecular tags in each of the targeting MIPs in each target population are distinct in each of the targeting MIPs and in each member of the target population;
iii) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),
wherein each of the control MIPs in each control population comprises in sequence the following components:
first control polynucleotide arm—first unique control molecular tag—polynucleotide linker—second unique control molecular tag—second control polynucleotide arm;
wherein the pair of first and second control polynucleotide arms in each of the control MIPs in each control population are identical, and are substantially complementary to first and second regions in the nucleic acid that, respectively, flank each control sequence;
wherein the first and second unique control molecular tags in each of the control MIPs in each control population are distinct in each of the control MIPs and in each member of the control population, and are different from the unique targeting molecular tags;
iv) sequencing the targeting and control MIPs amplicons that are amplified from the targeting and control MIPs replicons obtained in steps a.ii) and a.iii);

b) for each respective sample obtained from a subset in the plurality of subsets:
i) determining, for each target population, the number of the target capture events by targeting MIPs based on the number of unique targeting molecular tags present in the targeting MIPs amplicons sequenced in step a.iv);
ii) determining, for each control population, the number of the control capture events by control MIPs based on the number of unique control molecular tags present in the control MIPs amplicons sequenced in step a.iv);
iii) computing a target probe capture metric, for each target sequence, based at least in part on the number of the target capture events determined in step b.i) and a plurality of control probe capture metrics based at least in part on the numbers of the control capture events determined in step b.ii);
iv) identifying a subset of the control populations of control MIPs that have control probe capture metrics satisfying at least one criterion;
v) normalizing each target probe capture metric by a factor computed from the control probe capture metrics satisfying the at least one criterion, to obtain a normalized target probe capture metric for each of the one or more target sites; and

c) grouping, across the samples obtained from each subset of subjects, the normalized target probe capture metrics to obtain the genotype cluster for the known genotype.

In another aspect, the disclosure provides a method of selecting a genotype for a test subject. In some embodiments, the method comprises:

a) receiving sequencing data obtained from a nucleic acid sample from the test subject, wherein the sequencing data for the nucleic acid sample is obtained by:
i) obtaining a nucleic acid sample isolated from the test subject;
ii) capturing one or more target sequences of interest in the nucleic acid sample obtained in step a) by using one or more target populations of targeting molecular inversion probes (MIPs) to produce a plurality of targeting MIPs replicons for each target sequence,
wherein each of the targeting MIPs in the target population comprises in sequence the following components:
first targeting polynucleotide arm—first unique targeting molecular tag—polynucleotide linker—second unique targeting molecular tag—second targeting polynucleotide arm;
wherein the pair of first and second targeting polynucleotide arms in each of the targeting MIPs in each target population are identical, and are substantially complementary to first and second regions in the nucleic acid that, respectively, flank the target sequence of interest that is targeted by the one or more targeting MIPs;
wherein the first and second unique targeting molecular tags in each of the targeting MIPs in each target population are distinct in each of the targeting MIPs and in each member of the target population;
iii) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),
wherein each of the control MIPs in each control population comprises in sequence the following components:
first control polynucleotide arm—first unique control molecular tag—polynucleotide linker—second unique control molecular tag—second control polynucleotide arm;
wherein the pair of first and second control polynucleotide arms in each of the control MIPs in each control population are identical, and are substantially complementary to first and second regions in the nucleic acid that, respectively, flank each control sequence;
wherein the first and second unique control molecular tags in each of the control MIPs in each control population are distinct in each of the control MIPs and in each member of the control population, and are different from the unique targeting molecular tags;
iv) sequencing the targeting and control MIPs amplicons that are amplified from the targeting and control MIPs replicons obtained in steps a.ii) and a.iii);

b) determining, for each target population, the number of the unique targeting molecular tags present in the targeting MIPs amplicons sequenced in step a.iv);

c) determining, for each control population, the number of the unique control molecular tags present in the control MIPs amplicons sequenced in step a.iv);

d) computing a target probe capture metric, for each target site, based at least in part on the number of the unique targeting molecular tags determined in step b) and a plurality of control probe capture metrics based at least in part on the numbers of the unique control molecular tags determined in step c);

e) identifying a subset of the control populations of control MIPs that have control probe capture metrics satisfying at least one criterion;

f) normalizing each of the one or more target probe capture metrics by a factor computed from the control probe capture metrics satisfying the at least one criterion, to obtain a normalized target probe capture metric for each of the one or more target sequences;

g) receiving a group of values corresponding to normalized target probe capture metrics computed from nucleic acid samples from a first plurality of reference subjects exhibiting a same known genotype for a gene of interest;

h) comparing each of the one or more normalized target probe capture metrics obtained in step f) to the group of values received in step g); and

i) determining, based on the comparing in step h), whether the test subject exhibits the same known genotype for the gene of interest in each of the one or more target sequences.

In another aspect, the disclosure provides a method of selecting a genotype for a test subject. In some embodiments, the method comprises:

a) receiving sequencing data obtained from a nucleic acid sample from the test subject, wherein the sequencing data for the nucleic acid sample is obtained by:
i) obtaining a nucleic acid sample isolated from the test subject;
ii) capturing one or more target sequences of interest in the nucleic acid sample obtained in step a) by using one or more target populations of targeting molecular inversion probes (MIPs) to produce a plurality of targeting MIPs replicons for each target sequence,
wherein each of the targeting MIPs in the target population comprises in sequence the following components:
first targeting polynucleotide arm—first unique targeting molecular tag—polynucleotide linker—second unique targeting molecular tag—second targeting polynucleotide arm;
wherein the pair of first and second targeting polynucleotide arms in each of the targeting MIPs in each target population are identical, and are substantially complementary to first and second regions in the nucleic acid that, respectively, flank the target sequence of interest that is targeted by the one or more targeting MIPs;
wherein the first and second unique targeting molecular tags in each of the targeting MIPs in each target population are distinct in each of the targeting MIPs and in each member of the target population;
iii) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),
wherein each of the control MIPs in each control population comprises in sequence the following components:
first control polynucleotide arm—first unique control molecular tag—polynucleotide linker—second unique control molecular tag—second control polynucleotide arm;
wherein the pair of first and second control polynucleotide arms in each of the control MIPs in each control population are identical, and are substantially complementary to first and second regions in the nucleic acid that, respectively, flank each control sequence;
   wherein the first and second unique control molecular tags in each of the control MIPs in each control population are distinct in each of the control MIPs and in each member of the control population, and are different from the unique targeting molecular tags;
   iv) sequencing the targeting and control MIPs amplicons that are amplified from the targeting and control MIPs replicons obtained in steps a.ii) and a.iii);

b) determining, for each target population, the number of the target capture events by the targeting MIPs based on the unique targeting molecular tags present in the targeting MIPs amplicons sequenced in step a.iv);

c) determining, for each control population, the number of the control capture events by the control MIPs based on the number of the unique control molecular tags present in the control MIPs amplicons sequenced in step a.iv);

d) computing a target probe capture metric, for each target site, based at least in part on the number of the target capture events determined in step b) and a plurality of control probe capture metrics based at least in part on the numbers of the control capture events determined in step c);

e) identifying a subset of the control populations of control MIPs that have control probe capture metrics satisfying at least one criterion;

f) normalizing each of the one or more target probe capture metrics by a factor computed from the control probe capture metrics satisfying the at least one criterion, to obtain a normalized target probe capture metric for each of the one or more target sequences;

g) receiving a group of values corresponding to normalized target probe capture metrics computed from nucleic acid samples from a first plurality of reference subjects exhibiting a same known genotype for a gene of interest;

h) comparing each of the one or more normalized target probe capture metrics obtained in step f) to the group of values received in step g); and

i) determining, based on the comparing in step h), whether the test subject exhibits the same known genotype for the gene of interest in each of the one or more target sequences.

In some embodiments, computing the target probe capture metric comprises normalizing the number of the target capture events by a sum of the number of the target capture events and the numbers of the control capture events. In some embodiments, computing the plurality of control probe capture metrics comprises normalizing, for each control population, the number of control capture events determined in step by a sum of the number of the target capture events and the numbers of the control capture events.

In some embodiments, the number of capture events (e.g., a probe capturing or hybridizing to, or binding to a sequence of interest, or a site of interest, or a gene of interest) may be determined without using or counting the number of unique control molecular tags.

In some embodiments of the methods of the disclosure, the nucleic acid sample is DNA or RNA. In some embodiments, the nucleic acid sample is genomic DNA. In some embodiments, the methods of the disclosure can be used to detect copy number variations of a plurality of subjects. For example, one or more nucleic acid samples are obtained from different subjects (test or reference subjects). A sample barcoding step, as described above, can be used to individually label each sample from a distinct subject. The sample barcode can be incorporated into MIPs replicons or amplicons using a well-known technique, such as a PCR reaction. After sample barcoding, samples from different subjects can be mixed together and then be sequenced together.

In some embodiments of the methods of the disclosure, the subject is a candidate for carrier screening. In some embodiments, the carrier status of a subject is determined for a plurality of genetic conditions or disorders. In some embodiments, the carrier screening is for one genetic condition or disorder. In some embodiments, the screening is for more than one genetic condition or disorder, such as, two, three, four, five, six, seven, eight, nine, ten, fifteen, twenty, thirty, forty, fifty, sixty, seventy, eighty, ninety, one hundred or more. In some embodiments, the subject is a candidate for a carrier screening of one or more autosomal recessive conditions or disorders. In some embodiments, the autosomal recessive condition or disorder is spinal muscular atrophy, cystic fibrosis, Bloom syndrome, Canavan disease, dihydrolipoyl dehydrogenase deficiency, Familial dysautonomia, Familial hyperinsulinemic hypoglycemia, Fanconi anemia, Gaucher disease, Glycogen storage disease type I (GSD1a), Joubert syndrome, Maple syrup urine disease, Mucolipidosis IV, nemaline myopathy, Niemann-Pick disease types A and B, Tay-Sachs disease, Usher syndrome, Walker-Warburg Syndrome, Congenital amegakaryocytic thrombocytopenia, Prothrombin-Related Thrombophilia, sickle cell anemia, Fragile X Syndrome, Ataxia telangiectasia, Krabbe's disease, Galactosemia, Charcot-Marie-Tooth Disease with Deafness, Wilson's disease, Ehlers Danlos syndrome, type VIIC, Sjorgren-Larsson Syndrome, Metachromatic Leukodystrophy, Sanfilippo, Type C. In some embodiments, the subject is a candidate for an SMA carrier screening. In some embodiments, the subject is a prospective parent (mother or father). In some embodiments, the subject is an expecting parent (e.g., a pregnant woman or an expecting father). In some embodiments, the subject is a fetus carrier by a pregnant woman. In these embodiments, a nucleic acid sample of a fetal subject is fetal nucleic acid present in the pregnant woman carrying the fetus, such as cell-free fetal nucleic acid (DNA or RNA).

In some embodiments, the subject is a candidate for pharmacogenomics testing. In some embodiments, the subject is a candidate for targeted tumor testing (e.g., targeted tumor sequencing or targeted tumor analysis). In some embodiments, the subject is a candidate for pediatric diagnostic testing, such as for Duchenne's muscular dystrophy. In some embodiments, the subject is a candidate for BRCA1 or BRCA2 exonic deletion screening or testing. In some embodiments, the subject is a candidate for DMD gene exonic deletion or duplication testing. In some embodiments, the subject is a candidate for p450 enzyme CYP2D6 copy count testing. In some embodiments, the subject is a candidate for p450 enzyme CYP2D6 copy count testing. In some embodiments, the subject is a candidate for a targeted tumor analysis of MYC gene duplication. In some embodiments, the subject is a candidate for a targeted tumor analysis of MYCN gene duplication. In some embodiments, the subject is a candidate for a targeted tumor analysis of RET gene duplication. In some embodiments, the subject is a candidate for a targeted tumor analysis of EGFR gene duplication.

In some embodiments of the methods of the disclosure, the targeting molecular inversion probes (or circular capture probes) are used to capture a target site or sequence (or a site or sequence of interest). A target site or sequence, as used herein, refers to a portion or region of a nucleic acid sequence that is sought to be sorted out from other nucleic acid sequences within a nucleic acid sample, which is informative for determining the presence or absence of a genetic disorder or condition (e.g., the presence or absence of mutations, polymorphisms, deletions, insertions, aneuploidy etc.). A control site or sequence, as used herein, refers to a site that has known or normal copy numbers of a particular control gene. In some embodiments, the targeting MIPs comprise in sequence the following components: first targeting polynucleotide arm—first unique targeting molecular tag—polynucleotide linker—second unique targeting molecular tag—second targeting polynucleotide arm. In some embodiments, a target population of the targeting MIPs are used in the methods of the disclosure. In the target population, the pair of the first and second targeting polynucleotide arms in each of the targeting MIPs are identical and are substantially complementary to first and second regions in the nucleic acid that, respectively, flank the target site.

In some embodiments, the length of each of the targeting polynucleotide arms is between 18 and 35 base pairs. In some embodiments, the length of each of the targeting polynucleotide arms is 18, 19, 20, 21, 22, 23, 24, 25, 26, 27, 28, 29, 30, 31, 32, 33, 34, or 35 base pairs, or any size ranges between 18 and 35 base pairs. In some embodiments, the length of each of the control polynucleotide arms is between 18 and 35 base pairs. In some embodiments, the length of each of the control polynucleotide arms is 18, 19, 20, 21, 22, 23, 24, 25, 26, 27, 28, 29, 30, 31, 32, 33, 34, or 35 base pairs, or any size ranges between 18 and 35 base pairs. In some embodiments, each of the targeting polynucleotide arms has a melting temperature between 57° C. and 63° C. In some embodiments, each of the targeting polynucleotide arms has a melting temperature at 57° C., 58° C., 59° C., 60° C., 61° C., 62° C., or 63° C., or any size ranges between 57° C. and 63° C. In some embodiments, each of the control polynucleotide arms has a melting temperature between 57° C. and 63° C. In some embodiments, each of the control polynucleotide arms has a melting temperature at 57° C., 58° C., 59° C., 60° C., 61° C., 62° C., or 63° C., or any size ranges between 57° C. and 63° C. In some embodiments, each of the targeting polynucleotide arms has a GC content between 30% and 70%. In some embodiments, each of the targeting polynucleotide arms has a GC content of 30-40%, or 30-50%, or 30-60%, or 40-50%, or 40-60%, or 40-70%, or 50-60%, or 50-70%, or any size ranges between 30% and 70%, or any specific percentage between 30% and 70%. In some embodiments, each of the control polynucleotide arms has a GC content between 30% and 70%. In some embodiments, each of the control polynucleotide arms has a GC content of 30-40%, or 30-50%, or 30-60%, or 40-50%, or 40-60%, or 40-70%, or 50-60%, or 50-70%, or any size ranges between 30% and 70%, or any specific percentage between 30% and 70%.

In some embodiments, the length of each of the unique targeting molecular tags is between 12 and 20 base pairs. In some embodiments, the length of each of the unique targeting molecular tags is 12, 13, 14, 15, 16, 17, 18, 19, or 20 base pairs, or any interval between 12 and 20 base pairs. In some embodiments, the length of each of the unique control molecular tags is between 12 and 20 base pairs. In some embodiments, the length of each of the unique control molecular tags is 12, 13, 14, 15, 16, 17, 18, 19, or 20 base pairs, or any interval between 12 and 20 base pairs. In some embodiments, each of the unique targeting or control molecular tags is not substantially complementary to any genomic region of the subject (e.g., a test subject or a reference subject). In some embodiments, each of the unique targeting or control molecular tags is a randomly generated short sequence.

In some embodiments, the polynucleotide linker is not substantially complementary to any genomic region of the subject. In some embodiments, the polynucleotide linker has a length of between 30 and 40 base pairs. In some embodiments, the polynucleotide linker has a length of 30, 31, 32, 33, 34, 35, 36, 37, 38, or 39 base pairs, or any interval between 30 and 40 base pairs. In some embodiments, the polynucleotide linker has a melting temperature of between 60° C. and 80° C. In some embodiments, the polynucleotide linker has a melting temperature of 60° C., 65° C., 70° C., 75° C., or 80° C., or any interval between 60° C. and 80° C., or any specific temperature between 60° C. and 80° C. In some embodiments, the polynucleotide linker has a GC content between 40% and 60%. In some embodiments, the polynucleotide linker has a GC content of 40%, 45%, 50%, 55%, or 60%, or any interval between 40% and 60%, or any specific percentage between 40% and 60%. In some embodiments, the polynucleotide linker comprises CTTCAGCTTCCCGATATCCGACGGTAGTGT (SEQ ID NO: 1).

In some embodiments, the target population of targeting MIPs and the plurality of control populations of control MIPs are in a probe mixture. In some embodiments, the probe mixture has a concentration between 1-100 pM. In some embodiments, the probe mixture has a concentration between 1-10 pM, 10-100 pM, 10-50 pM, or 50-100 pM, or any interval between 1-100pM. The concentration of the probe mixture can be adjusted based on the probe capture efficiency.

In some embodiments, each of the targeting MIPs replicons is a single-stranded circular nucleic acid molecule. In some embodiments, each of the control MIPs replicons is a single-stranded circular nucleic acid molecule.

In some embodiments, each of the targeting MIPs amplicons is a double-stranded nucleic acid molecule. In some embodiments, each of the control MIPs amplicons is a double-stranded nucleic acid molecule.

In some embodiments, a targeting MIPs replicons is produced by: i) the first and second targeting polynucleotide arms, respectively, hybridizing to the first and second regions in the nucleic acid that, respectively, flank the target site; and ii) after the hybridization, using a ligation/extension mixture to extend and ligate the gap region between the two targeting polynucleotide arms to form single-stranded circular nucleic acid molecules.

In some embodiments, each of the control MIPs replicons is produced by: i) the first and second control polynucleotide arms, respectively, hybridizing to the first and second regions in the nucleic acid that, respectively, flank the control site; and ii) after the hybridization, using a ligation/extension mixture to extend and ligate the gap region between the two control polynucleotide arms to form single-stranded circular nucleic acid molecules.

In some embodiments, the sequencing step comprises a next-generation sequencing method, for example, a massive parallel sequencing method, or a short read sequencing method, or a massive parallel short-read sequencing method. In some embodiments, sequencing may be by any method known in the art, for example, targeted sequencing, single molecule real-time sequencing, electron microscopy-based sequencing, transistor-mediated sequencing, direct sequencing, random shotgun sequencing, Sanger dideoxy termination sequencing, targeted sequencing, exon sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, ion semiconductor sequencing, nanoball sequencing, nanopore sequencing, 454 sequencing, Solexa Genome Analyzer sequencing, miSeq (Illumina), HiSeq 2000 (Illumina), HiSeq 2500 (Illumina), Illumina Genome Analyzer (Illumina), Ion Torrent PGM™ (Life Technologies), MinION™ (Oxford Nanopore Technologies), real-time SMIRT™ technology (Pacific Biosciences), the Probe-Anchor Ligation (cPAL™) (Complete Genomics/BGI), SOLiD® sequencing, MS-PET sequencing, mass spectrometry, and a combination thereof. In some embodiments, sequencing comprises an detecting the sequencing product using an instrument, for example but not limited to an ABI PRISM® 377 DNA Sequencer, an ABI PRISM® 310, 3100, 3100-Avant, 3730, or 373OxI Genetic Analyzer, an ABI PRISM® 3700 DNA Analyzer, or an Applied Biosystems SOLiD™ System (all from Applied Biosystems), a Genome Sequencer 20 System (Roche Applied Science), or a mass spectrometer. In certain embodiments, sequencing comprises emulsion PCR. In certain embodiments, sequencing comprises a high throughput sequencing technique, for example but not limited to, massively parallel signature sequencing (MPSS).

A sequencing technique that can be used in the methods of the disclosure includes, for example, Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. No. 7,960,120; U.S. Pat. No. 7,835,871; U.S. Pat. No. 7,232,656; U.S. Pat. No. 7,598,035; U.S. Pat. No. 6,911,345; U.S. Pat. No. 6,833,246; U.S. Pat. No. 6,828,100; U.S. Pat. No. 6,306,597; U.S. Pat. No. 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

In some embodiments, the method of the disclosure comprises before the sequencing step of d), a PCR reaction (or other convention reaction) to amplify the targeting and control MIPs replicons for sequencing. In some embodiments, the PCR or other reaction is an indexing PCR or other reaction. In some embodiments, the indexing PCR or other reaction introduces into each of the targeting MIPs replicons the following components: a pair of indexing primers, a unique sample barcode and a pair of sequencing adaptors, thereby producing the targeting or control MIPs amplicons.

In some embodiments, the barcoded targeting MIPs amplicons comprise in sequence the following components:
a first sequencing adaptor—a first sequencing primer—the first unique targeting molecular tag—the first targeting polynucleotide arm—captured target nucleic acid—the second targeting polynucleotide arm—the second unique targeting molecular tag—a unique sample barcode—a second sequencing primer—a second sequencing adaptor.

In some embodiments, the barcoded control MIPs amplicons comprise in sequence the following components:
a first sequencing adaptor—a first sequencing primer—the first unique control molecular tag—the first control polynucleotide arm—captured control nucleic acid—the second control polynucleotide arm—the second unique control molecular tag—a unique sample barcode—a second sequencing primer—a second sequencing adaptor.

In some embodiments, the target site and at least one of the control sites are on the same chromosome. In some embodiments, the target site and at least one of the control sites are on different chromosomes.

In some embodiments, the target site is SMN1 or SMN2. In some embodiments, the first and second targeting polynucleotide arms for SMN1/SMN2 are, respectively, 5′-AGG AGT AAG TCT GCC AGC ATT-3′ (SEQ ID NO: 2) and 5′-AAA TGT CTT GTG AAA CAA AAT GCT-3′ (SEQ ID NO: 3). In some embodiments, the first and second targeting polynucleotide arms for SMN1/SMN2 are, respectively, 5′-ACC ACC TCC CAT ATG TCC AGA-3′ (SEQ ID NO: 5) and 5′-ACC AGT CTG GGC AAC ATA GC-3′ (SEQ ID NO: 6).

In some embodiments, the MIPs are designed to capture the base change difference in exon 7 of the SMN1/SMN2 genes. In some embodiments, the MIP for detecting copy number variation of SMN1/SMN2 comprises the sequence of 5′-AGG AGT AAG TCT GCC AGC ATT NNN NNN NNN NCT TCA GCT TCC CGA TTA CGG GTA CGA TCC GAC GGT AGT GTN NNN NNN NNN AAA TGT CTT GTG AAA CAA AAT GCT-3.

In some embodiments, the control sites comprise one or more genes or sites selected from the group consisting of CFTR, HEXA, HFE, HBB, BLM, IDS, IDUA, LCA5, LPL, MEFV, GBA, MPL, PEX6, PCCB, ATM, NBN, FANCC, F8, CBS, CPT1, CPT2, FKTN, G6PD, GALC, ABCC8, ASPA, MCOLN1, SPMD1, CLRN1, NEB, G6PC, TMEM216, BCKDHA, BCKDHB, DLD, IKBKAP, PCDH15, TTN, GAMT, KCNJ11, IL2RG, and GLA.

In another aspect, The systems and methods of embodiments of this disclosure may be used for detecting deletions, such as BRCA1 exonic deletions, BRCA2 exonic deletions, or 1p36 deletion syndrome.

In certain embodiments, the methods described herein are used to detect exonic deletions or insertions or duplication. In some embodiments, the target site (or sequence) is a deletion or insertion or duplication in a gene of interest or a genomic region of interest. In some embodiments, the target site is a deletion or insertion or duplication in one or more exons of a gene of interest. In some embodiments, the target multiple exons are consecutive. In some embodiments, the target multiple exons are non-consecutive. In some embodiments, the first and second targeting polynucleotide arms of MIPs are designed to hybridize upstream and downstream of the deletion (or insertion, or duplication) or deleted (or inserted, or duplicated) genomic region (e.g., one or more exons) in a gene or a genomic region of interest. In some embodiments, the first or second targeting polynucleotide arm of MIPs comprises a sequence that is substantially complementary to the genomic region of a gene of interest that encompasses the target deletion or duplication site (e.g., exons or partial exons).

In certain embodiments, the gene of interest is BRCA1 or BRCA2. In some embodiments, the target site (or sequence) is a deletion (partial or full deletion) of one or more exons of a BRCA1 or BRCA2 gene (e.g., BRCA1 Exon 11). In some embodiments, the target site is an insertion within one or more exons of a BRCA1 or BRCA2 gene. In some embodiments, the target site is a duplication (partial or full duplication) of one or more exons of a BRCA1 or BRCA2 gene. In some embodiments, the deleted or duplicated multiple exons are consecutive. In some embodiments, the deleted or duplicated multiple exons are non-consecutive. In some embodiments, the first or second targeting polynucleotide arm of MIPs (but not both) comprises a sequence that is substantially complementary to the wild type sequence of a BRCA genomic region that is expected to exhibit the target exonic deletion or duplication. In some embodiments, the first and second targeting polynucleotide arms for detecting a partial deletion of BRCA exon 11 are, respectively, 5′-GTCTGAATCAAATGCCAAAGT-3′ (SEQ ID NO: 7) and 5′-TCCCCTGTGTGAGAGAAAAGA-3′ (SEQ ID NO: 8). In some embodiments, the MIP that is used in the methods described herein for detecting a partial deletion of BRCA exon 11 is/5Phos/GTCTGAATCAAATGCCAAAG CTTCAGCTTCCCG ATTACGGGTACGATCCGACGGTAGTGT TCCCCTGTGTG AGAGAAAAGA (SEQ ID NO: 9).

In some embodiments, the gene of interest is DMD. In some embodiments, the target site (or sequence) is a deletion (partial or full deletion) of one or more exons of a DMD gene. In some embodiments, the target site is an insertion within one or more exons of a DMD gene. In some embodiments, the target site is duplication (partial or full duplication) of one or more exons of a DMD gene. In some embodiments, the deleted or duplicated multiple exons are consecutive. In some embodiments, the deleted or duplicated multiple exons are non-consecutive. In some embodiments, the first or second targeting polynucleotide arm of MIPs (but not both) comprises a sequence that is substantially complementary to the wild type sequence of a DMD genomic region that is expected to exhibit the target exonic deletion or duplication. In some embodiments, the target deleted or duplicated exons of a DMD gene are listed in Table 4 or any known deletion or duplications in the DMD gene. In some embodiments, the MIP that is used in the methods described herein for detecting one or more exonic deletions (partial or full deletions) or duplications of a DMD gene is listed in Table 3.

In another aspect, the systems and methods of embodiments of this disclosure may be used for detecting chromosomal aneuploidies, such as diagnosis of down syndrome.

In another aspect, the systems and methods of embodiments of this disclosure may use PCR probes or primers to produce PCR amplicons instead of MIPs. In some embodiments, the disclosure provides a method for detecting copy number variations in a subject using PCR probes (or primers) and PCR amplicons. In some embodiments, the method comprises:

a) obtaining a nucleic acid sample isolated from, or derived from, or obtained from the subject;

b) amplifying one or more target sequences in the nucleic acid sample obtained in step a) by using one or more target populations of targeting polymerase reaction chain (PCR) forward and reverse probes to produce targeting PCR amplicons for each target sequence,

wherein each of the targeting PCR forward probes in each of the target populations comprises in sequence the following components:

5′-targeting PCR forward primer -unique targeting forward molecular tag-3′;

wherein each of the targeting PCR reverse probes in the target population comprises in sequence the following components:

5′-unique targeting reverse molecular tag-targeting PCR reverse primer-3′;

wherein the pair of targeting PCR forward and reserve probes in each of the targeting PCR probes in each of the target populations are identical, and are substantially complementary to first and second regions in the nucleic acid that, respectively, flank the target sequence that is targeted by the one or more targeting PCR forward and reverse probes; wherein the unique targeting forward and reverse molecular tags in each of the targeting PCR probes in the target population are distinct in each of the targeting PCR probes and in each member of the target population;

c) capturing a plurality of control sequences in the nucleic acid sample obtained in step a) by using a plurality of control populations of control PCR forward and reverse probes to produce a plurality of control PCR amplicons, each control population of control PCR forward and reverse probes being capable of amplifying a distinct control sequence in the nucleic acid sample obtained in step a),

wherein each of the control PCR forward probes in the control population comprises in sequence the following components:

5′-control PCR forward primer -unique control forward molecular tag-3′;

wherein each of the control PCR reverse probes in the control population comprises in sequence the following components:

5′-unique control reverse molecular tag—control PCR reverse primer-3′;

wherein the pair of control PCR forward and reserve probes in each of the control PCR probes in the target population are identical, and are substantially complementary to first and second regions in the nucleic acid that, respectively, flank the control sequence;

wherein the unique control forward and reverse molecular tags in each of the control PCR probes in the control population are distinct in each of the control PCR probes and in each member of the control population;

d) sequencing the targeting and control PCR amplicons obtained in steps b) and c);

e) determining, for each target population, the number of the unique targeting molecular tags present in the targeting PCR amplicons sequenced in step d);

f) determining, for each control population, the number of the unique control molecular tags present in the control PCR amplicons sequenced in step d);

g) computing a target probe capture metric, for each of the one or more targeted sequences, based at least in part on the number of the unique targeting molecular tags determined in step e) and a plurality of control probe capture metrics based at least in part on the numbers of the unique control molecular tags determined in step f);

h) identifying a subset of the control populations of control PCR probes that have control probe capture metrics satisfying at least one criterion;

i) normalizing each of the one or more target probe capture metrics by a factor computed from the subset of control probe capture metrics satisfying the at least one criterion, to obtain a test normalized target probe capture metric for each of the one or more target sequences;

j) comparing each of the one or more test normalized target probe capture metrics to a plurality of reference normalized target probe capture metrics that are computed based on reference nucleic acid samples obtained from reference subjects exhibiting known genotypes using the same target and control sequences, target population, one subset of control populations in steps b)-g) and i); and

k) determining, based on the comparing in step j) and the known genotypes of reference subjects, the copy number variation of each of the one or more target sequence of interest.

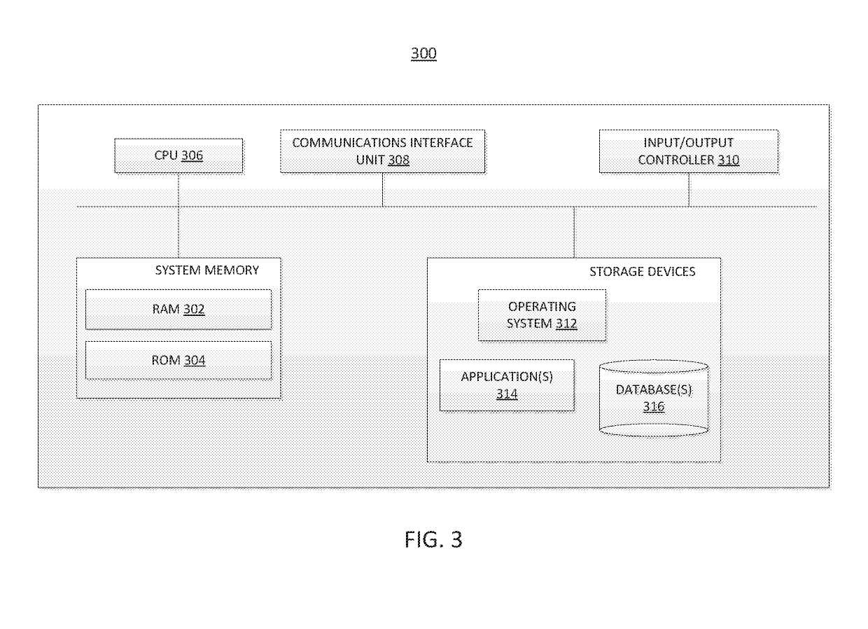

FIG. 3 is a block diagram of a computing device 300 for performing any of the processes described herein, including forming genotype clusters based on samples obtained from reference subjects exhibiting known genotypes, or computing a probe capture metric for a test subject and comparing the probe capture metric to a set of genotype clusters to select an appropriate genotype for the test subject. As used herein, the term “processor” or “computing device” refers to one or more computers, microprocessors, logic devices, servers, or other devices configured with hardware, firmware, and software to carry out one or more of the computerized techniques described herein. Processors and processing devices may also include one or more memory devices for storing inputs, outputs, and data that are currently being processed. The computing device 300 may include a “user interface,” which may include, without limitation, any suitable combination of one or more input devices (e.g., keypads, touch screens, trackballs, voice recognition systems, etc.) and/or one or more output devices (e.g., visual displays, speakers, tactile displays, printing devices, etc.). The computing device 300 may include, without limitation, any suitable combination of one or more devices configured with hardware, firmware, and software to carry out one or more of the computerized techniques described herein. Each of the components described herein may be implemented on one or more computing devices 300. In certain aspects, a plurality of the components of these systems may be included within one computing device 300. In certain implementations, a component and a storage device may be implemented across several computing devices 300.

The computing device 300 comprises at least one communications interface unit, an input/output controller 310, system memory, and one or more data storage devices. The system memory includes at least one random access memory (RAM 302) and at least one read-only memory (ROM 304). All of these elements are in communication with a central processing unit (CPU 306) to facilitate the operation of the computing device 300. The computing device 300 may be configured in many different ways. For example, the computing device 300 may be a conventional standalone computer or alternatively, the functions of computing device 300 may be distributed across multiple computer systems and architectures. In FIG. 3, the computing device 300 is linked, via network or local network, to other servers or systems.

The computing device 300 may be configured in a distributed architecture, wherein databases and processors are housed in separate units or locations. Some units perform primary processing functions and contain at a minimum a general controller or a processor and a system memory. In distributed architecture implementations, each of these units may be attached via the communications interface unit 308 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM and TCP/IP.

The CPU 306 comprises a processor, such as one or more conventional microprocessors and one or more supplementary co-processors such as math co-processors for offloading workload from the CPU 306. The CPU 306 is in communication with the communications interface unit 308 and the input/output controller 310, through which the CPU 306 communicates with other devices such as other servers, user terminals, or devices. The communications interface unit 308 and the input/output controller 310 may include multiple communication channels for simultaneous communication with, for example, other processors, servers or client terminals.

The CPU 306 is also in communication with the data storage device. The data storage device may comprise an appropriate combination of magnetic, optical or semiconductor memory, and may include, for example, RAM 302, ROM 304, flash drive, an optical disc such as a compact disc or a hard disk or drive. The CPU 306 and the data storage device each may be, for example, located entirely within a single computer or other computing device; or connected to each other by a communication medium, such as a USB port, serial port cable, a coaxial cable, an Ethernet cable, a telephone line, a radio frequency transceiver or other similar wireless or wired medium or combination of the foregoing. For example, the CPU 306 may be connected to the data storage device via the communications interface unit 308. The CPU 306 may be configured to perform one or more particular processing functions.

The data storage device may store, for example, (i) an operating system 312 for the computing device 300; (ii) one or more applications 314 (e.g., computer program code or a computer program product) adapted to direct the CPU 306 in accordance with the systems and methods described here, and particularly in accordance with the processes described in detail with regard to the CPU 306; or (iii) database(s) 316 adapted to store information that may be utilized to store information required by the program.

The operating system 312 and applications 314 may be stored, for example, in a compressed, an uncompiled and an encrypted format, and may include computer program code. The instructions of the program may be read into a main memory of the processor from a computer-readable medium other than the data storage device, such as from the ROM 304 or from the RAM 302. While execution of sequences of instructions in the program causes the CPU 306 to perform the process steps described herein, hard-wired circuitry may be used in place of, or in combination with, software instructions for implementation of the processes of the present disclosure. Thus, the systems and methods described are not limited to any specific combination of hardware and software.

Suitable computer program code may be provided for performing one or more functions as described herein. The program also may include program elements such as an operating system 312, a database management system and “device drivers” that allow the processor to interface with computer peripheral devices (e.g., a video display, a keyboard, a computer mouse, etc.) via the input/output controller 310.

The term “computer-readable medium” as used herein refers to any non-transitory medium that provides or participates in providing instructions to the processor of the computing device 300 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 306 (or any other processor of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 300 (e.g., a server) can receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information.

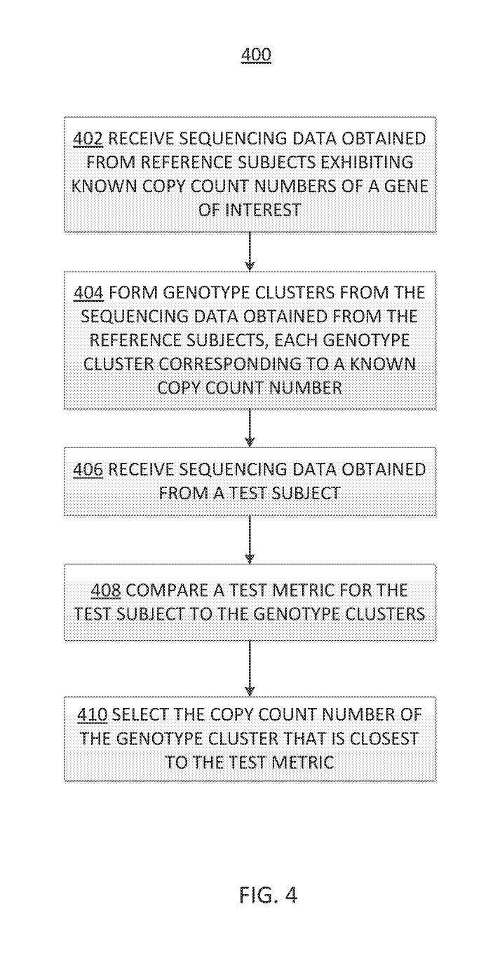

FIG. 4 is a flowchart of a process 400 for determining a copy count number/variation for a test subject, according to an illustrative embodiment. The process 400 includes the steps of receiving sequencing data obtained from reference subjects exhibiting known copy count numbers of a gene of interest (step 402), or a site of interest, or a sequence of interest, forming genotype clusters from the sequencing data obtained from the reference subjects, each genotype cluster corresponding to a known copy count number (step 404), receiving sequencing data obtained from a test subject (step 406), comparing a test metric for the test subject to the genotype clusters (step 408), and selecting the copy count number of the genotype cluster that is closest to the test metric (step 410).

At step 402, sequencing data is received. The received sequencing data is obtained from reference subjects exhibiting known copy count numbers of a gene of interest, or a site of interest, or a sequence of interest. In an example, the sequencing data is obtained by obtaining a nucleic acid sample from each reference subject and using one or more target populations of targeting MIPs and a set of control populations of control MIPs to capture one or more target sites and a set of control sites in each nucleic acid sample. As is described in detail in relation to FIG. 1, each targeting MIPs includes in sequence a first targeting polynucleotide arm, a first unique targeting molecular tag, a polynucleotide linker, a second unique targeting molecular tag, and a second targeting polynucleotide arm. The first and second targeting polynucleotide arms are the same across the targeting MIPs in the target population, while the first and second unique targeting molecular tags are distinct across the targeting MIPs in the target population. Targeting MIPs replicons and a set of control MIPs replicons result from the capture of the target site and the set of control sites, and further amplified to produce targeting or control MIPs amplicons. The amplicons are sequenced to obtain the sequencing data. The example described herein in relation to SMN1 and SMN2 copy number variation is described for illustrative purposes only. In general, one of ordinary skill in the art will understand that the systems and methods of the present disclosure are applicable to determining a genotype from sequencing data.

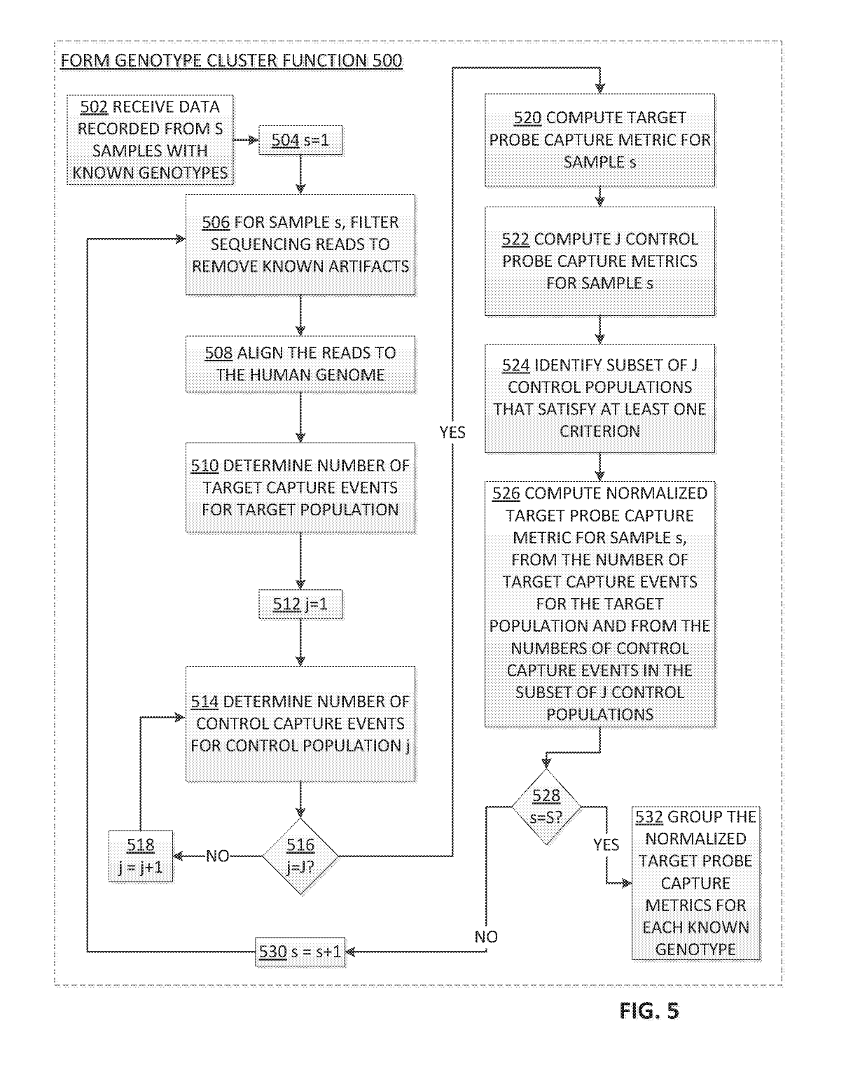

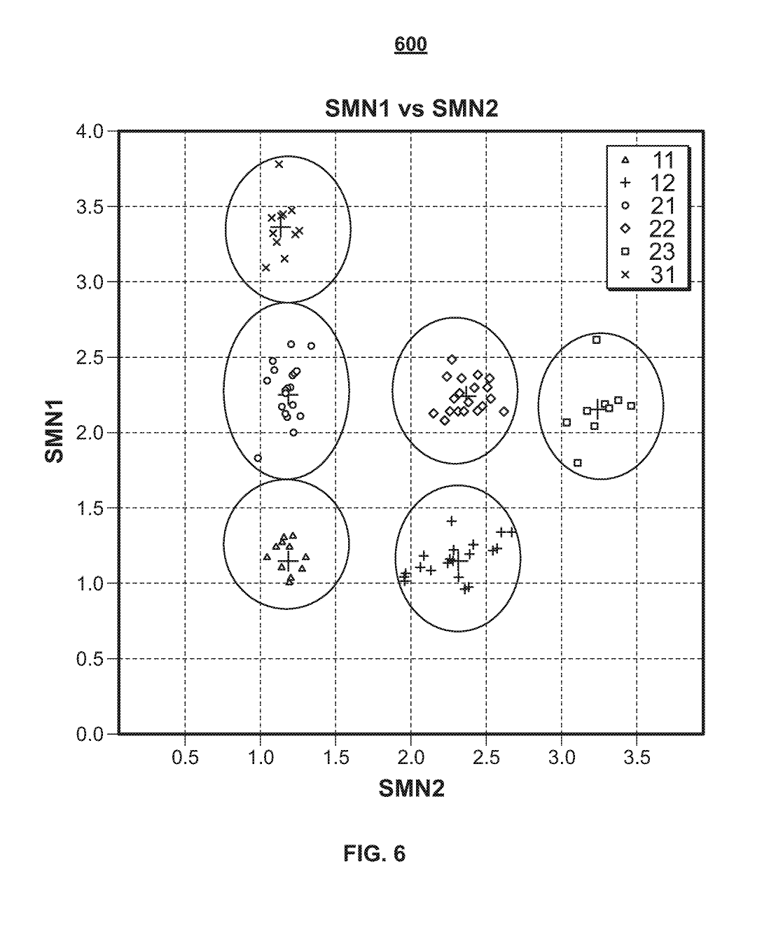

At step 404, genotype clusters are formed from the sequencing data obtained from the reference subjects. In an example, each genotype cluster corresponds to a set of data points (each data point corresponding to a sample obtained from a different reference subject) that quantitatively describe an observation from the samples. The set of data points in the same genotype cluster are computed from the sequencing data obtained from reference subjects exhibiting the same known genotype. Each genotype may correspond to a known copy count number for a gene of interest, such as for SMN1 or SMN2. One example of how the genotype clusters may be formed is described in relation to FIG. 5, and FIG. 6 is a scatter plot of six sets of data points forming six genotype clusters. As is described herein, the genotype clusters are used as references for comparing to a data point computed from a sample obtained from a test subject, for whom the genotype may not be known. In some implementations, steps 402 and 404 of the process 400 are collapsed into a single step, in which data indicative of the genotype clusters is received by a device.

At step 406, sequencing data that is obtained from a test subject is received. The genotype for the test subject may be unknown, and it may be desirable to provide a computational prediction of the test subject's genotype by using the genotype clusters as a reference. In particular, the test subject may exhibit an unknown copy count number of a particular gene of interest (site of interest or sequence of interest), and the systems and methods present disclosure may be used to compute a test metric for the test subject. For example, the test metric is computed in the same manner as the data points that form each genotype cluster, and may correspond to a normalized target probe capture metric. As is described in more detail in relation to FIG. 5, the normalized target probe capture metric is representative of a relative ability of a target population of targeting MIPs to hybridize to a target site on the gene of interest (or site of interest, or sequence of interest), compared to a set of control populations of control MIPs.

At step 408, the test metric for the test subject is compared to the genotype clusters. The test metric is computed in a similar manner as the set of data points that form the genotype clusters. In particular, as is described in relation to FIG. 5, the genotype clusters are formed by computing normalized target probe capture metrics for a set of reference subjects and grouping the resulting values for the normalized target probe capture metrics according to the different genotypes of the reference subjects. The test metric may be computed by determining a normalized target probe capture metric for the test subject in a similar manner as is outlined in steps 506-526 for the test sample.

At step 410, the copy count number of the genotype cluster that is closest to the test metric is selected. In one example, a distance metric is computed between the test metric and each of the genotype clusters, and the known genotype (e.g., the copy count number) of the genotype cluster having the shortest distance is selected. In particular, a Mahalanobis distance may be used to compute the distance between a data point and a distribution of data points on a two-dimensional grid, as is shown in FIG. 6.

FIG. 5 is a flowchart of a process 500 for forming a genotype cluster, according to an illustrative embodiment. In an example, the process 500 may be used to implement the step 404 of the process 400 shown and described in relation to FIG. 4. As was described in relation to FIG. 4, the function of forming a genotype cluster may be used to process data obtained from a set of samples having known genotypes for a particular gene of interest. The genotype cluster includes a set of data points (each corresponding to a different sample) that quantitatively describe an observation from the processed data, where each data point in a set corresponds to the same known genotype. In the example of copy count number variation, the genotype corresponds to a copy count number for a gene of interest, such as for SMN1 and/or SMN2.

The process 500 includes the steps of receiving data recorded from S samples with known genotypes (step 502) and initializing a sample iteration parameter s to 1 (step 504). For each sample s, the process 500 includes filtering the sequencing reads to remove known artifacts (step 506), aligning the reads to the human genome (step 508), determining a number of target capture events for a target population (step 510), determining numbers of control capture events for a set of control populations (steps 514, 516, and 518), computing a target probe capture metric (step 520), computing control probe capture metrics (step 522), identifying a subset of control populations that satisfy at least one criterion (step 524), and computing a normalized target probe capture metric (step 526). When all S samples have been considered, the normalized target probe capture metrics are then grouped according to the known genotypes (step 532).

In some embodiments, the number of target capture events corresponds to the number of unique targeting molecular tags present in the sequenced targeting MIPs amplicons. In some embodiments, the number of target capture events is determined based on the number of unique targeting molecular tags present in the sequenced targeting MIPs amplicons. In some embodiments, the number of control capture events corresponds to the number of unique control molecular tags present in the sequenced control MIPs amplicons. In some embodiments, the number of control capture events is determined based on the number of unique control molecular tags present in the sequenced control MIPs amplicons.

At step 502, data recorded from a set of S samples is received, where the S samples each corresponds to a known genotype. In particular, each of the S samples may be obtained from a reference subject exhibiting a known genotype for a gene of interest, where each of the S samples corresponds to a different reference subject. The samples may be nucleic acid samples isolated from, or derived from, or obtained from the reference subjects, and the data may include sequencing data obtained from the nucleic acid samples. In an example, the sequencing data is obtained by using a target population of targeting MIPs to amplify a target site (or sequence) of interest in the nucleic acid sample, and by using a set of control populations of control MIPs to amplify a set of control sites (or sequences) in the nucleic acid sample to produce target MIPs replicons and control MIPs replicons. The replicons may then be further amplified and subsequently be sequenced to obtain the sequencing data received at step 502.

At step 504, a sample iteration parameter s is initialized to 1. As the S samples are processed, the sample iteration parameter s is incremented until each of the S samples is processed to obtain a normalized target probe capture metric.

At step 506, the sequencing reads for sample s are filtered to remove known artifacts. In one example, the data received at step 502 may be processed to remove an effect of probe-to-probe interaction. For example, when an intervening MIP has polynucleotide arms that share high sequence identities with the targeting polynucleotide arms of a targeting MIP, due to the high ratio of probe to target in the reaction, this intervening capture event or reaction may dominate and produce a captured product of the intervening MIP which is a byproduct and needs to be removed. In some implementations, the ligation and extension targeting arms of all MIPs are matched to the paired-end sequence reads. Reads that failed to match both arms of the MIPs are determined to be invalid and discarded. The arm sequences for the remaining valid reads are removed, and the molecular tags from both ligation and extension ends may be also removed from the reads. The removed molecular tags may be kept separately for further processing at steps 510 and 514.

At step 508, the resulting trimmed reads are aligned to the human genome. In some embodiments, an alignment tool may be used to align the reads to a reference human genome. In particular, an alignment score may be assessed for representing how well does a specific read align to the reference. Reads with alignment scores above a threshold may be referred to herein as primary alignments, and are retained. In contrast, reads with alignment scores below the threshold may be referred to herein as secondary alignments, and are discarded. Any reads that aligned to multiple locations along the reference genome may be referred to herein as multi-alignments, and are discarded.

At step 510, the number of target capture events for the target population of targeting MIPs is determined. In particular, each targeting MIP in the target population may target the same target sequence on the gene of interest, but may include a different molecular tag from every other targeting MIP in the target population. The aligned reads may be examined to count the number of unique molecular tags for the targeted site (or sequence) on the gene of interest. These counts may correspond to the initial number of MIP-to-site hybridization events (e.g., MIP-to-site capture events) that were sequenced in a Next-Generation Sequencing (NGS) platform, such as the Illumina HiSeq 2500 flowcell.

At step 512, a control population iteration parameter j is initialized to 1. For the j-th control population, the number of control capture events for the j-th control population is determined at step 514. In particular, similar to the target population described in relation to step 510, each control MIP in the j-th control population may target the same control sequence on a reference gene that is different from the gene of interest, but may include a different molecular tag from every other control MIP in the j-th control population. For each j-th control population (and therefore the j-th control site), the aligned reads from step 508 are examined to count the number of unique molecular tags for the j-th control site on the associated reference gene. At decision block 516, the control population iteration parameter j is compared to the total number J of control populations. If j is less than J, then the process 500 proceeds to step 518 to increment j and returns to step 514 to determine the number of control capture events for the next control population.

In some embodiments, the number of target capture events corresponds to the number of unique targeting molecular tags present in the sequenced targeting MIPs amplicons. In some embodiments, the number of target capture events is determined based on the number of unique targeting molecular tags present in the sequenced targeting MIPs amplicons. In some embodiments, the number of control capture events corresponds to the number of unique control molecular tags present in the sequenced control MIPs amplicons. In some embodiments, the number of control capture events is determined based on the number of unique control molecular tags present in the sequenced control MIPs amplicons.

When all J control populations have been considered, the process 500 proceeds to step 520 to compute a target probe capture metric for the sample s. The target probe capture metric may correspond to a performance measure of how efficiently does the target population of targeting MIPs capture the target site (or sequence) on the gene of interest. In one example, the target probe capture metric for the sample s may be computed by dividing the number determined at step 510 by the sum of the numbers determined at steps 510 and 514 (e.g., numbers of unique molecular tags, or numbers of capture events). The resulting ratio may then be normalized by one or more normalizing factors to align the metric to a copy count number. In particular, the target probe capture metric (PC_{TARGET,s}) may be computed in accordance with EQ. 1 below, where J corresponds to the total number of control populations used in the sample s, u_{TARGET,s} corresponds to the number of target capture events determined at step 510, and each u_{CONTROL i,s} corresponds to the number of control capture events for the i-th control population determined at step 514.

$$\begin{matrix}{PC}_{TARGET , s} = 2 \times ( J + 1 ) \frac{{u}_{TARGET , s}}{{u}_{TARGET , s} + {\sum }_{i = 1}^{J} {u}_{CONTROL i , s}} & ( EQ . 1 ) \\ {PC}_{TARGET , s} = 2 \times ( J + 1 ) \frac{{u}_{TARGET , s}}{{u}_{TARGET , s} + {\sum }_{i = 1}^{J } {u}_{CONTROL i , s}} & \end{matrix}$$

As can be determined from EQ. 1, the target probe capture metric is representative of a relative performance efficiency of the target population's ability to capture or hybridize to the target site (or sequence) on the gene of interest, relative to all the populations, including the target population and the set of control populations. EQ. 1 for computing the target probe capture metric is shown for illustrative purposes only, and in general, other forms of performance efficiency metrics may be used to represent the relative capture efficiency of a population of MIPs, without departing from the scope of the present disclosure.

At step 522, J control probe capture metrics are computed for the sample s. Each of the J control probe capture metrics is computed in a similar manner as the target probe capture metric described in relation to step 520. In particular, the j-th control probe capture metric may correspond to a performance measure of how efficiently does the j-th control population of control MIPs capture the corresponding control site on the reference gene. In one example, the j-th control probe capture metric for the sample s may be computed by dividing the number of control capture events for the j-th control population by the sum of the numbers determined at step 510 and 514. The resulting ratio may then be normalized by one or more normalizing factors to align the metric to a copy count number. In particular, the control probe capture metric (PC_{CONTROL j,s} may be computed in accordance with EQ. 2 below, where J corresponds to the total number of control populations used in the sample s, u_{TARGET,s} corresponds to the number of target capture events determined at step 510, and each u_{CONTROL i,s} corresponds to the number of control capture events for the i-th control population determined at step 514.

$$\begin{matrix}{PC}_{CONTROL j , s} = 2 \times ( J + 1 ) \frac{{u}_{CONTROL j , s}}{{u}_{TARGET , s} + {\sum }_{i = 1}^{J} {u}_{CONTROL i , s}} & ( EQ . 2 ) \\ {PC}_{CONTROL j , s} = 2 \times ( J + 1 ) \frac{{u}_{CONTROL j , s}}{{u}_{TARGET , s} + {\sum }_{i = 1}^{J} {u}_{CONTROL i , s}} & \end{matrix}$$

As can be determined from EQ. 2, the control probe capture metric is representative of a relative performance efficiency of the j-th control population's ability to capture or hybridize to the control site on the reference gene, relative to all the populations, including the target population and the set of control populations. EQ. 2 for computing the control probe capture metric is shown for illustrative purposes only, and in general, other forms of performance efficiency metrics may be used to represent the relative capture efficiency of a population of MIPs, without departing from the scope of the present disclosure. However, in general, it may be desirable to use the same computational process to compute the target probe capture metric as the control probe capture metric, to allow for direct comparison between them.

At step 524, a subset of the J control populations is identified that satisfies at least one criterion. For example, the control probe capture metrics (PC_{CONTROL j,s}) computed at step 522 are evaluated, and those control probe capture metrics that do not meet the at least one criterion are discarded. The at least one criterion may include a requirement that the control probe capture metrics are all above a first threshold level, below a second threshold level, or both. The first threshold and/or second threshold may be predetermined values, or may be values that depend on the values of the probe capture metrics. For example, one or both thresholds may be determined from the set of J control probe capture metrics, such that the bottom X percentage and top Y percentage of the J control probe capture metrics are discarded, where X or Y may correspond to 5%, 10%, 15%, or any other suitable percentile. Moreover, the values for X and Y may be the same or different. In another example, one or both thresholds may be determined based on the target probe capture metric computed at step 520, and any of the J control populations with control probe capture metrics that fall outside a specific range around the target probe capture metric may be discarded.

In some embodiments, the at least one criterion used at step 524 includes a requirement that the subset of J control populations has a low sample-to-sample variation. In other words, the subset of J control populations may be required to include only those control populations that performed relatively consistently across the different S samples. In this case, the step 524 may be performed for each of the samples only after all the samples have been processed to compute the target probe capture metrics and the control probe capture metrics. To require a low sample-to-sample variation, the at least one criterion at step 524 may include computing a coefficient of variability of the control probe capture metrics for the j-th control population across the set of S samples. In an example, the coefficient of variability may be computed as the standard deviation divided by the mean of a set of values. Those control populations having high coefficients of variability may be discarded, and the remaining subset of the J control populations is identified as satisfying the at least one criterion.

In some embodiments, the at least one criterion used at step 524 includes a requirement that the subset of J control populations remains the same across the set of S samples. In some embodiments, the at least one criterion used at step 524 includes a requirement that the subset of J control populations is different across the set of S samples. In some embodiments, the subset of control populations are the same across different samples. In some embodiments, the subset of control populations are different for different samples. In this case, the steps 524 and 526 may follow the decision block 528.

At step 526, a normalized target probe capture metric is computed for the sample s. In an example, the normalized target probe capture metric corresponds to the target probe capture metric (computed at step 520) divided by the average of the control probe capture metrics for the subset of control populations (identified at step 524). The average of the control probe capture metrics for the subset of control populations is representative of the average control population, and may be referred to herein as a “composite control population.” By normalizing the target probe capture metric by the average control probe capture metrics for the subset of control populations, sample-to-sample probe performance variability is reduced by taking into account possible differences in the input quantity and quality of the DNA, and other possible experimental differences across the set of S samples. In general, the present disclosure is not limited to the average, and any suitable statistic may be used, including the median.

At decision block 528, the sample iteration parameter s is compared to the total number of samples S. If s is less than S, then the process 500 proceeds to step 530 to increment s and returns to step 506 to begin processing of the next sample. Otherwise, when all S samples have been processed, the process 500 proceeds to step 532 to group the normalized target probe capture metrics for each known genotype. In particular, the resulting set of S values for the normalized target probe capture metrics are separated according to the known genotypes of the corresponding S samples.

The order of the steps in FIG. 5 is shown for illustrative purposes only, and are not limiting. In particular, the order of steps 510 and 514 may be reversed, such that the numbers of control capture events are determined before the number of target capture events is determined. In general, the numbers of target capture events and control capture events may be determined in any order. Similarly, the order of steps 520 and 522 is shown in FIG. 5 as step 520 occurring before step 522. In general, the computation of the target probe capture metric may be performed after the computation of some or all of the J control probe capture metrics, without departing from the scope of the present disclosure.

Moreover, as is shown in FIG. 5, a sample s is completely processed before moving on to the next sample s+1. However, one of ordinary skill in the art will appreciate that one or more of the metrics described herein may be computed only after all the samples are partially processed. As an example, one of the metrics may involve a measure that spans across samples, such as a coefficient of variation statistic. In this case, a coefficient of variation may be computed based on the set of control probe capture metrics determined across the set of S samples. One of the at least one criterion used at step 524 may include a requirement for a low across-sample variation, and may involve computing a coefficient of variation for each control population of control MIPs. In this case, the coefficient of variation for a control population represents a variance of the performance of the control MIPs across the set of samples. A control population having a high coefficient of variation means that the control MIPs in that particular control population did not have a consistent performance across the set of samples, and so it may be undesirable to include those control populations that perform inconsistently in the set.

FIG. 6 is a plot 600 of six illustrative genotype clusters that are formed using the method described in relation to FIG. 5. In FIG. 6, the vertical axis corresponds to normalized target probe capture metrics for SMN1, and the horizontal axis corresponds to normalized target probe capture metrics for SMN2. Each circle surrounds a set of data points having two coordinates—the normalized target probe capture metric for SMN1 and the normalized target probe capture metric for SMN2. The example shown in FIG. 6 shows two different normalized target probe capture metrics (e.g., the normalized target probe capture metric for SMN1 and the normalized target probe capture metric for SMN2) that may be used simultaneously together to determine a proper genotype for a test subject. However, a single metric may be used to form a genotype cluster. In this case, a plot of the genotype cluster would be reduced to a set of values on a single axis. Moreover, depending on the application, three or more metrics may be used to form a genotype cluster. In this case, an N-dimensional array may be used to represent each data point in the cluster, where N corresponds to the number of metrics.

The genotype clusters shown in FIG. 6 correspond to a reference map that may be used to determine identify a predicted genotype exhibited by a test subject. This identification may be performed by performing steps 406, 408, and 410 of FIG. 4 to receiving sequencing data obtained from the test subject, comparing a test metric to the genotype clusters, and selecting the genotype cluster that is closest to the test metric. In this example, the test metric may correspond to a pair of coordinates on the map, and the genotype cluster that is nearest the test metric may be chosen. Then, the genotype of the chosen genotype cluster is used to predict the status of the test subject. The test described herein may be determined to be inconclusive if the test metric is outside any of the circles shown in FIG. 6, or too far away from any of the genotype clusters.

### EXAMPLES

### Example 1

### Determination of a Single Site or Single Gene Copy Number Variation Overview

In some embodiments, the methods of the disclosure use molecular inversion probes (MIPs) (e.g., 5′ phosphorylated single stranded DNA capture probes) to prepare targeted libraries for massive parallel sequencing. These MIPs are added together in a mixture at low concentrations (e.g., 1-100 pM), incubated with a genomic DNA, upon which a mixture of polymerase and ligase is added to form single-stranded DNA circles (MIP replicons). An exonuclease cocktail is then added to the mixture to remove the excess probe and genomic DNA which is then moved to an indexing PCR reaction to add unique sample barcodes and sequencing adaptors. Hence, an assay may be divided into three parts: 1) target enrichment; 2) sample barcoding for multiplexed sequencing; and 3) massive parallel sequencing.

### Target Enrichment

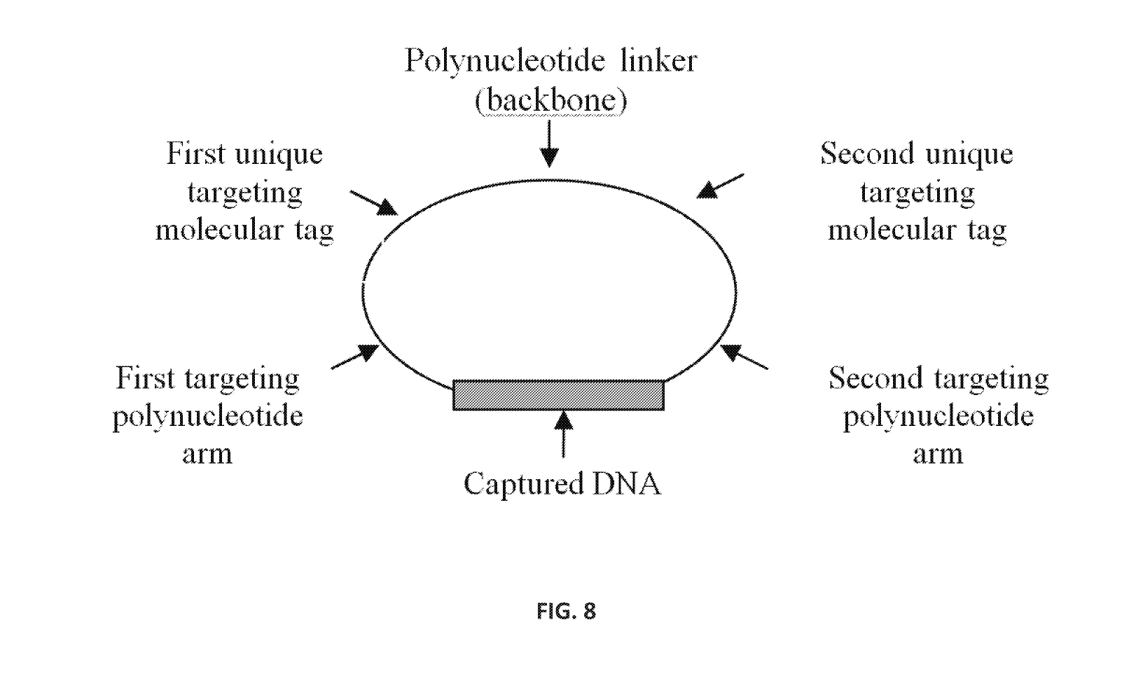

Target enrichment refers to the ability to select a specific region of interest (e.g., a target site or sequence) prior to sequencing. For example, if one is interested in examining 20 specific genes from a large cohort of individuals, it would be both wasteful and prohibitively expensive to sample the entire genome of each individual. Instead, target enrichment technologies allow selection of regions for amplification from each individual and thus only sequence the specific area of interest (e.g., a target site or sequence), such as the captured DNA depicted in FIG. 8.

### Sample Barcoding for Multiplexed Sequencing

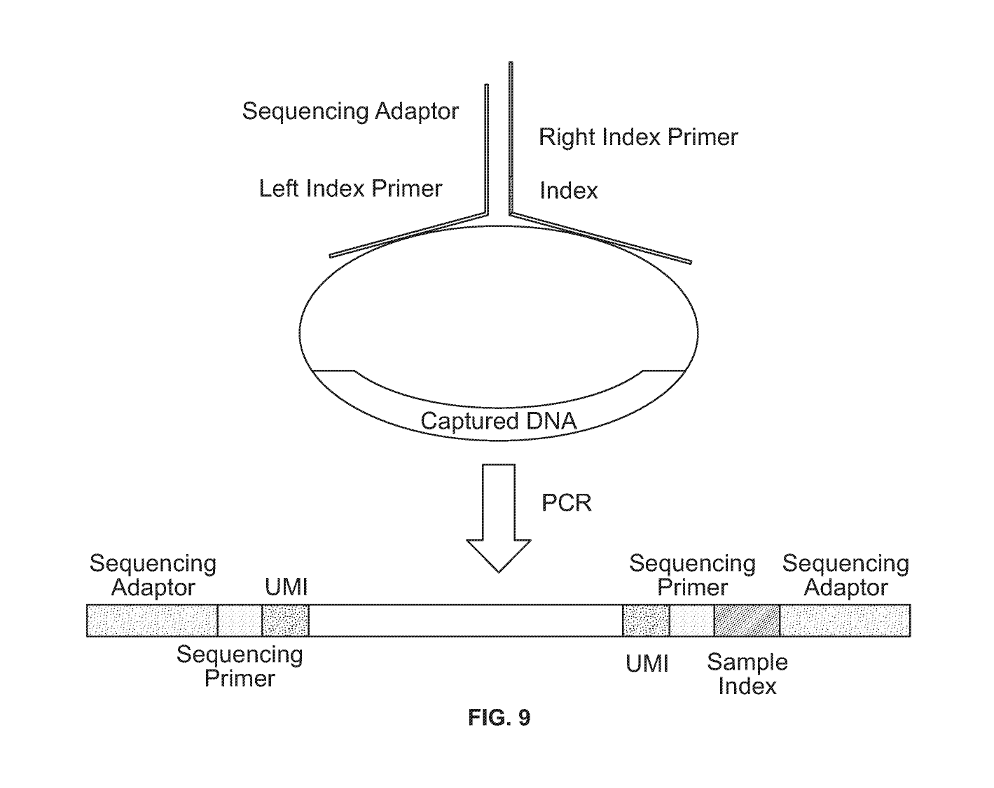

Barcoding samples during the target enrichment process enables one to pool multiple samples per sequencing run, and deconvolute the sample source during the data analysis step based on the barcode. The diagram in FIG. 9 illustrates an example MIP, where UMI refers to a unique molecular identifier, i.e., unique molecular tag, and sample index refers to a unique sample barcode for each individual subject.

### Library Preparation Using Amplicon Tagging

Library preparation for next-generation sequencing is by far the most time and labor consuming part of the entire next-generation sequencing process. While necessary for whole genome sequencing studies, the process can be essentially eliminated for re-sequencing projects by using the methods in some embodiments of this disclosure. By incorporating the adaptor sequences into the primer design, the MIP amplicon product is ready to go directly into clonal amplification since it already contains the necessary capture sequences.

### Massive Parallel Sequencing

The GCS LDT 8001 assay, a carrier screening assay developed in this disclosure, is designed to operate on the Illumina HiSeq™ 2500 device. After generation of the targeted DNA library with the MIPs, the library is analyzed using the Illumina HiSeq 2500 in rapid Run Mode.

Here, the DNA templates are hybridized via the adaptors to a planar surface, where each DNA template is clonally amplified by solid-phase PCR, also known as bridge amplification. This creates a surface with a high density of spatially distinct clusters, each cluster of which contains a unique DNA template. These are primed and sequenced by passing the four spectrally distinct reversible dye terminators in a flow of solution over the surface in the presence of a DNA polymerase. Only single base extensions are possible due to the 3′ modification of the chain-termination nucleotides, and each cluster incorporates only one type of nucleotide, as dictated by the DNA template forming the cluster. The incorporated base in all clusters is detected by fluorescence imaging of the surface before chemical removal of the dye and terminator, generating an extendable base that is ready for a new round of sequencing. The most common sequencing errors produced in reversible dye termination SBS are substitutions. This assay uses paired end reads as a variation.

In a specific example, blood or mouthwash/buccal samples are obtained from a human subject to determine a carrier status with respect to a target site (sequence) of interest. After accessioning, the blood and mouthwash/buccal samples are extracted for genomic DNA. The genomic DNA samples (4 μL) are added into “Probe mix” plates (96 well) holding the probe mix for capture (16 μL). The probe mixtures contain a mixture of targeting molecular inversion probes (MIPs) (e.g., for SMN1/SMN2) and a plurality of control MIPs. These probes are incubated on a thermocycler and placed back on the robotic system for addition of the Extension/ligation mixture. The Extension/ligation mixture (20 μL) is added and the plate is then incubated in the thermocycler again and subsequently placed back on the robotic system for addition of the exonuclease mixture. The exonuclease mixture is added (10 μL) and the plate is incubated on a thermocycler and subsequently stored or moved to the sequencing step. The plate containing targeting and control MIPs replicons is placed on the robotics liquid transfer station and 104, from the plate is transferred to an indexing PCR mixture in a 96-well format to attach indexing primers, massive parallel sequencing adaptors and unique sample barcodes. The plate is run in conjunction with another set of samples in a 96-well plate on the thermocycler. Barcoded samples are pooled at 54, each into a single vial. The pooled products are purified via AmPure beads, QC'd for size and contamination on a BioAnalyzer, Caliper or equivalent instrument (see the manuals). The pool is then quantified for DNA content with a Quibit broad range dye assay (see the manual). The library is then generated based on the estimation of DNA and gel sizes. This library is then combined with another 96 well-plate library (each well corresponding to a different sample). Once a 192-sample library is obtained, it is loaded onto the Illumina Rapid Run HiSeq 2500 flowcell (See the manual.) The Illumina HiSeq is then Run per instructions using a paired end 106 base pair kit for sequencing. Data are generated and sent to the Progenity Sequencing Drive and stored according to run number and date. Data are analyzed via a custom sequence analysis workflow, including alignment, variant calling, QC and sample reporting instructions.

The sequence of the SMN1/SMN2 MIP that are used to measure the PCE value is as follows:

/5Phos/AGG AGT AAG TCT GCC AGC ATT NNN NNN NNN NCT
TCA GCT TCC CGA TTA CGG GTA CGA TCC GAC GGT AGT
GTN NNN NNN NNN AAA TGT CTT GTG AAA CAA AAT GCT

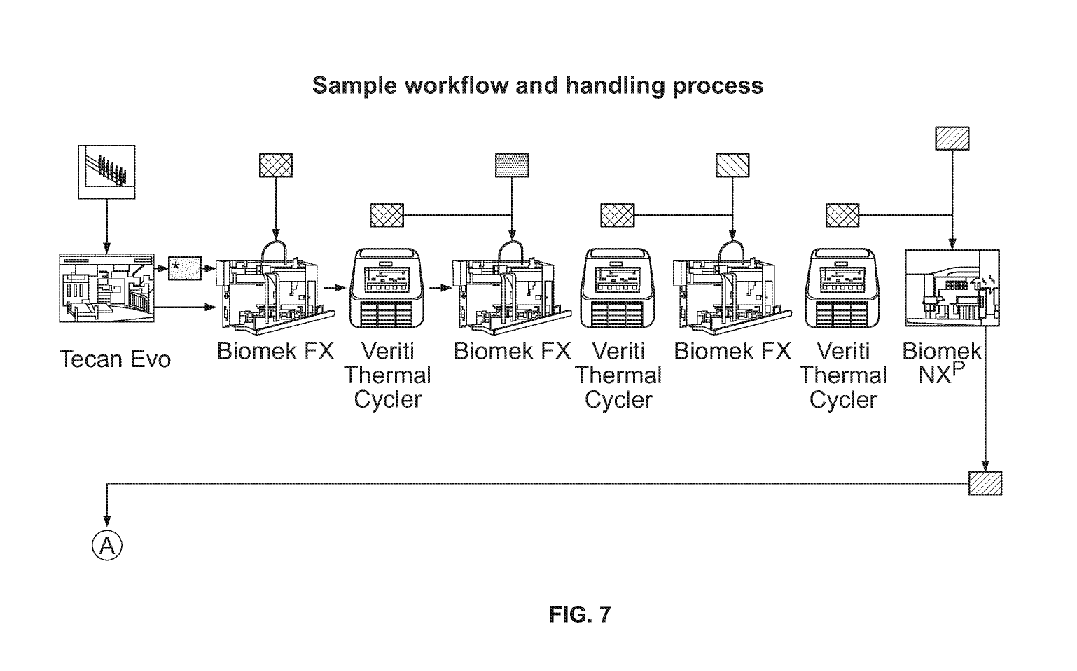

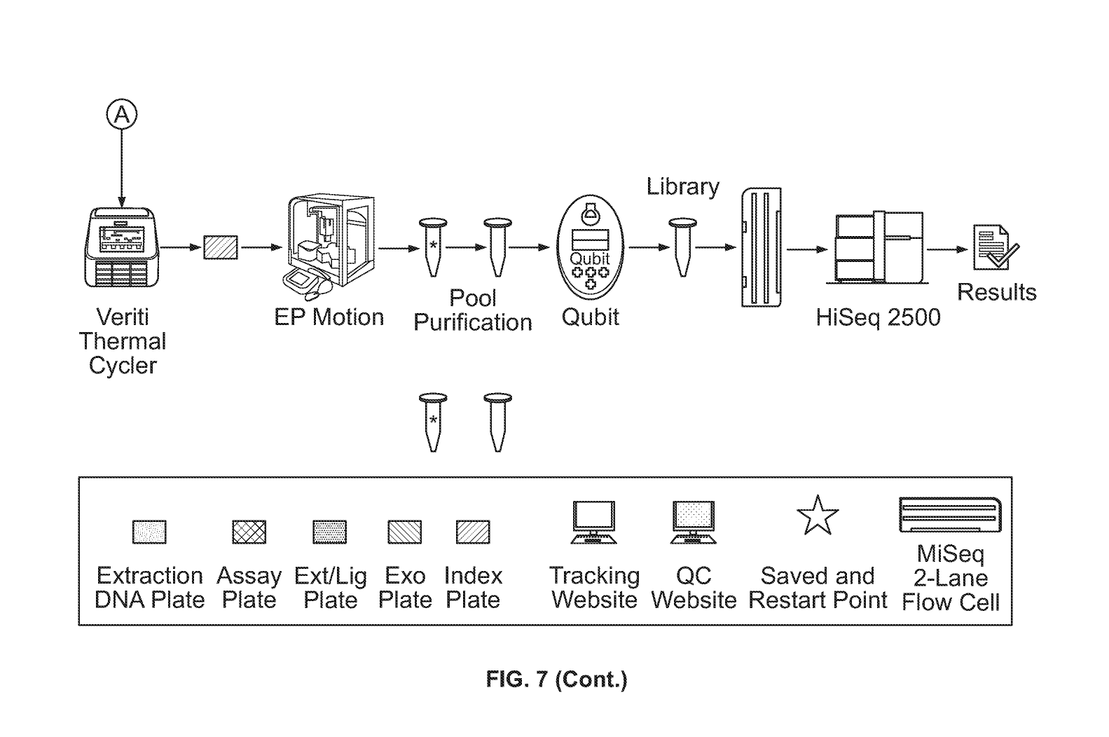

The workflow is outlined as follows (see also FIG. 7):
In the experiment, 96 DNA samples (the Optimization plate) run through the Global Carrier Screening (GCS) assay using the probe pool.
The probe pool in this experiment consists of 1471 unique probes.
Target Capture:
   1) The 1471 probes used for this experiment are from the GCS_G-W IDT plates (17 plates; each probe in 40 ul at 100 uM); 250 ng of DNA are used in each reaction; see Table 1 for sample details.
   2) Prepare target capture, master mix (see the Table below)

5 pM

| | | |
|---|---|---|
| Reagent | X1 | X112 |

| gDNA | 4 | ul | — | |
|---|---|---|---|---|
| 500 pM Probe Pool | 0.2 | ul | 22.4 | ul |
| 10X Ampligase Buffer | 2 | ul | 224 | ul |
| water | 13.8 | ul | 1545.6 | ul |

| | | | |
|---|---|---|---|
| Total vol | 20 | 1792 | ul |

3) Add 4 ul sample to 16 ul capture mix.
4) Thermocycler program: GCS MIP Capture (on Veriti thermocycler)

| | |
|---|---|
| 98° C. | 5 min |
| touchdown | ~90 min (2 mins/degree) |
| | (set ramp speed to 20$ for |
| | TD temps) |
| 56° C. | 120 min |

Extension/Ligation
   5) Prepare extension/ligation master mix (build plate was used):

| | | |
|---|---|---|
| Reagent | X1 | X106 |

| | | | | |
|---|---|---|---|---|
| 10 mM dNTP | .6 | ul | 63.6 | ul |
| 100X NAD | .8 | ul | 84.8 | ul |
| 5M Betaine | 3 | ul | 318 | ul |
| 10X Ampligase Buff | 2 | ul | 212 | ul |
| Ampligase, 5 U/ul | 2 | ul | 212 | ul |
| Phusion Pol HF, 2 U/ul | 0.5 | ul | 53 | ul |
| water | 11.1 | ul | 1176.6 | ul |
| Total vol | 20 | ul | 600 | ul |

6) Add 20 ul extension/ligation mix to each sample.
7) Thermocycler program: GCS MIP Ext/Lig (on Veriti thermocycler)

| | |
|---|---|
| 56° C. | 60 min |
| 72° C. | 20 min |
| 37° C. | hold |

8) Prepare Exonuclease master mix (build plate was used):

1X Enzyme + Buffer Master Mix

| | | |
|---|---|---|
| Reagent | X1 | X106 |

| | | | | |
|---|---|---|---|---|
| Exo I, 20 U/ul | 2 | ul | 212 | ul |
| Exo III, 100 U/ul | 2 | ul | 212 | ul |
| 10X NEBuffer I | 5 | ul | 530 | ul |
| Water | 1 | ul | 106 | ul |
| Total vol | 10 | ul | 1060 | ul |

9) Add 10 ul master mix to each reaction.
10) Thermocycler programs: GCS CCCP Exonuclease Digestion (on Veriti thermocycler)

| | |
|---|---|
| 37° C. | 45 min |
| 80° C. | 20 min |
| 4° C. | forever |

11) Cool samples on ice (can optionally store at −20° C.)
PCR Amplification
   12)Dilute primers 1:10 (100 uM to 10 uM)

| | |
|---|---|
| REV primer (100 uM) | 4 ul |
| water | 36 ul |

13) Circular CCCP amplification PCR master mix:

| | | |
|---|---|---|
| Reagent | X1 | X106 |

| CCCP circular DNA | 10 | ul | — | |
|---|---|---|---|---|
| 5X Phusion HF Buffer | 10 | ul | 1060 | ul |
| 10 mM dNTPs | 1 | ul | 106 | ul |
| Phusion Pol HS, 2 U/ul | 1 | ul | 106 | ul |
| FWD primer (100 uM) | 0.25 | ul | 26.5 | ul |
| Primers universal (REV; 10 uM) | 2.5 | ul | — | |
| water | 25.25 | ul | 2676.5 | ul |
| Total vol | 50 | ul | 3975 | ul |

14) Add lOul sample and 2.5 ul primer to 37.5 ul PCR mix
15) Thermocycler Programs: GCS CCCP PCR (on Veriti)

| | | | |
|---|---|---|---|
| | 95° C. | 2 | min |
| 24 Cycles | 98° C. | 15 | sec |
| | 65° C. | 15 | sec |
| | 72° C. | 15 | sec |
| | 72° C. | 5 | min |

| | |
|---|---|
| 4° C. | forever |

16) Purify amplified products using Ampure beads:
a. 5 uL of each sample is pooled and 50 ul of the pool is mixed with 50 ul Ampure beads. After 5 minutes, samples were washed twice with 170 ul 70% EtOH, dried for 5 minutes, and pellet was resuspended in 45 uL EB Buffer.
b. The purified pools were QC'd on the Qubit and Bioanalyzer.

TABLE 1

| | | Conc. | Vol of | Vol of | SMN1; | | |
|---|---|---|---|---|---|---|---|
| Well | GID | (ng/ul) | DNA | Water | SMN2 | CF Result | AJP Result |
| A1 | G191 | 81.6 | 23.0 | 7.0 | 2; 2 | | |
| B1 | G192 | 99.45 | 18.9 | 11.1 | 3; 1 | | |
| C1 | G193 | 61.34 | 30.6 | −0.6 | 2; 1 | | |
| D1 | G194 | 105.8 | 17.7 | 12.3 | 2; 1 | | |
| E1 | G195 | 71.25 | 26.3 | 3.7 | 2; 0 | | |
| F1 | G196 | 128.2 | 14.6 | 15.4 | 2; 2 | | |
| G1 | G197 | 81.34 | 23.1 | 6.9 | 2; 2 | | |
| H1 | G198 | 100.7 | 18.6 | 11.4 | 2; 1 | | |
| A2 | G199 | 88.2 | 21.3 | 8.7 | 2; 2 | | |
| B2 | G200 | 75.74 | 24.8 | 5.2 | 2; 2 | | |
| C2 | G201 | 68.98 | 27.2 | 2.8 | 2; 1 | | |
| D2 | G202 | 82.56 | 22.7 | 7.3 | 2; 2 | | |
| E2 | G203 | 70.64 | 26.5 | 3.5 | 2; atypical | | |
| | | | | | (between 0-1) | | |
| F2 | G204 | 69.05 | 27.2 | 2.8 | 3; 0 | | |
| G2 | G205 | 80.23 | 23.4 | 6.6 | 2; 0 | | |
| H2 | G206 | 150.9 | 12.4 | 17.6 | 3; 2 | | |
| A3 | G207 | 73.39 | 25.5 | 4.5 | 2; 2 | | |
| B3 | G208 | 92.04 | 20.4 | 9.6 | 3; 1 | | |
| C3 | G209 | 111 | 16.9 | 13.1 | 2; 1 | | |
| D3 | G210 | 70.39 | 26.6 | 3.4 | 1; 1 | | |
| E3 | G211 | 94.85 | 19.8 | 10.2 | 3; 2 | | |
| F3 | G212 | 87.9 | 21.3 | 8.7 | 2; 2 | | |
| G3 | G213 | 67.62 | 27.7 | 2.3 | 2; 1 | | |
| H3 | G214 | 86.16 | 21.8 | 8.2 | 2; 2 | | |
| A4 | G215 | 82.66 | 22.7 | 7.3 | 2; 2 | | |
| B4 | G216 | 99.69 | 18.8 | 11.2 | 2; 2 | | |
| C4 | G217 | 56.17 | 33.4 | −3.4 | 3; 1 | | |
| D4 | G218 | 88.39 | 21.2 | 8.8 | 2; 2 | | |
| E4 | G219 | 200.6 | 9.3 | 20.7 | 3; 0 | R1066H | |
| F4 | G220 | 87.19 | 21.5 | 8.5 | 2; 2 | R1162X | |
| G4 | G221 | 148.7 | 12.6 | 17.4 | | D1152H | |
| H4 | G222 | 123.3 | 15.2 | 14.8 | 2; 2 | R75X | |
| A5 | G223 | 90.67 | 20.7 | 9.3 | | 663delT | |
| B5 | G224 | 94.48 | 19.8 | 10.2 | 2; 2 | | p.N370S |
| C5 | G225 | 86.4 | 21.7 | 8.3 | | L206W | |
| D5 | G226 | 119.1 | 15.7 | 14.3 | | 3849 + 10kbC->T | |
| E5 | G227 | 60.67 | 30.9 | −0.9 | | R117C | |
| F5 | G228 | 80.35 | 23.3 | 6.7 | 2; 1 | S945L | |
| G5 | G229 | 108.2 | 17.3 | 12.7 | | L206W | |
| H5 | G230 | 72.48 | 25.9 | 4.1 | | G542X | |
| A6 | G231 | 67.31 | 27.9 | 2.1 | 2; 2 | G551D | |
| B6 | G232 | 111.6 | 16.8 | 13.2 | 2; 1 | R553X | |
| C6 | G233 | 73.5 | 25.5 | 4.5 | 2; 2 | W1282X | |
| D6 | G234 | 83.66 | 22.4 | 7.6 | 2; 2 | 3849 + 10kbC->T | p.N370S; |
| | | | | | | | p.R12L |
| E6 | G235 | 124.6 | 15.0 | 15.0 | | 3120 + 1G->A | p.L444P |
| F6 | G236 | 81.72 | 22.9 | 7.1 | 2; 0 | 2183delAA > G | |
| G6 | G237 | 78.51 | 23.9 | 6.1 | 2; 2 | 2789 + 5G > A | |
| H6 | G238 | 72.6 | 25.8 | 4.2 | | E1104X | |
| A7 | G239 | 114.9 | 16.3 | 13.7 | 2; 1 | G551D | |
| B7 | G240 | 53.06 | 35.3 | −5.3 | | W1204X | |
| C7 | G241 | 224.4 | 8.4 | 21.6 | 2; 2 | 1898 + 1G->A | |
| D7 | G242 | 66.96 | 28.0 | 2.0 | | D1152H | |
| E7 | G243 | 82.7 | 22.7 | 7.3 | | R560T | |
| F7 | G244 | 119 | 15.8 | 14.2 | 1; 2 | 3905insT | |
| G7 | G245 | 64.97 | 28.9 | 1.1 | 1; 0 | S945L | p.L444P |
| H7 | G246 | 135.5 | 13.8 | 16.2 | 2; 1 | 1717 − 1G->A | |
| A8 | G247 | 75.3 | 24.9 | 5.1 | 2; 1 | | |
| B8 | G248 | 88.93 | 21.1 | 8.9 | | P67L | |
| C8 | G249 | 75.45 | 24.9 | 5.1 | 2; 1 | 711 + 3A > G | |
| D8 | G250 | 94.97 | 19.7 | 10.3 | 2; 2 | G542X | |
| E8 | G251 | 70.18 | 26.7 | 3.3 | | D1152H | |
| F8 | G252 | 146.6 | 12.8 | 17.2 | 2; 1 | R553X | |
| G8 | G253 | 77.02 | 24.3 | 5.7 | 2; 1 | | p.N370S; |
| | | | | | | | p.R2478_D2512del |
| H8 | G254 | 89.1 | 21.0 | 9.0 | | G551D | del55bp |
| A9 | G255 | 87.68 | 21.4 | 8.6 | 2; 2 | W1282X | |
| B9 | G256 | 75.67 | 24.8 | 5.2 | 2; 2 | 3120G > A | |
| C9 | G257 | 67.66 | 27.7 | 2.3 | 2; 2 | R553X | |
| D9 | G258 | 73.14 | 25.6 | 4.4 | | R117C | |
| E9 | G259 | 82.53 | 22.7 | 7.3 | | G551D | |
| F9 | G260 | 81.96 | 22.9 | 7.1 | 2; 2 | | IVS3 − 2A > G |
| G9 | G261 | 89.04 | 21.1 | 8.9 | | N1303K | |
| H9 | G262 | 136.5 | 13.7 | 16.3 | | 3849 + 10kbC->T | |
| A10 | G263 | 57 | 32.9 | −2.9 | | 3120 + 1G->A | |
| B10 | G264 | 91.93 | 20.4 | 9.6 | 2; 0 | D1152H | 1278 + TATC |
| C10 | G265 | 104.6 | 17.9 | 12.1 | 3; 1 | 3791delC | |
| D10 | G266 | 81.11 | 23.1 | 6.9 | 2; 2 | | p.G229C |
| E10 | G267 | 91.94 | 20.4 | 9.6 | inconclusive; | | p.N370S |
| | | | | | inconclusive | | |
| F10 | G268 | 60.6 | 30.9 | −0.9 | 2; 2 | [delta]F508 | c.3992 − |
| | | | | | | | 9G > A |
| G10 | G269 | 134.6 | 13.9 | 16.1 | 2; 2 | | p.Q347X |
| H10 | G270 | 84.85 | 22.1 | 7.9 | | [delta]F508 | IVS4(+4)A > T |
| A11 | G271 | 67.82 | 27.6 | 2.4 | 2; 1 | | p.N370S |
| B11 | G272 | 106 | 17.7 | 12.3 | 2; 2 | | 1278 + TATC |
| C11 | G273 | 79.87 | 23.5 | 6.5 | 2; 0 | | p.A305E |
| D11 | G274 | 226.2 | 8.3 | 21.7 | 2; 2 | | |
| E11 | G275 | 96.09 | 19.5 | 10.5 | 2; 1 | | 1278 + TATC |
| F11 | G276 | 135.3 | 13.9 | 16.1 | 2; 1 | | 1278 + TATC |
| G11 | G277 | 51.82 | 36.2 | −6.2 | 2; 0 | | IVS1 + 2T > A |
| H11 | G278 | 149.9 | 12.5 | 17.5 | 2; 2 | | |
| A12 | G279 | 78.07 | 24.0 | 6.0 | 2; 0 | | p.R83C |
| B12 | G280 | 87.92 | 21.3 | 8.7 | 2; 1 | | del6.4kb |
| C12 | G281 | 112.7 | 16.6 | 13.4 | 2; 3 | | IVS12 + 1G > C |
| D12 | G282 | 77.97 | 24.0 | 6.0 | 2; 2 | | p.G229C |
| E12 | G283 | 90.55 | 20.7 | 9.3 | 3; 0 | | IVS12 + 1G > C |
| F12 | G284 | 103.6 | 18.1 | 11.9 | 2; 1 | | 2281Del6/Ins7 |
| G12 | G285 | 50.67 | 37.0 | −7.0 | 2; 2 | | p.N370S |
| H12 | | | | 30 | | | |

FIG. 6 is a plot of six illustrative genotype clusters (SMN1/SMN2) that are used for comparison to a test metric evaluated from a test subject, following the above-described workflow.

### Example 2

### Detection of Down Syndrome (Trisomy 21)

Down syndrome is a chromosomal condition that is associated with intellectual disability, a characteristic facial appearance and other symptoms.

The most common cause of Down syndrome is trisomy 21, i.e., each cell in the patient's body has three copies of chromosome 21. A number of N (e.g., N=5) sites that are distributed through chromosome 21 may be selected, for example, the first base of exon 1 for the following genes: TPTE, CHODL, CCT8, PSMG1 and PRMT2. A targeted probe (e.g. a targeting MIP) for each one of these sites as well as a collection of control sites on other chromosomes is designed. The copy counting method in some embodiments of this disclosure are then applied to each one of these five sites on Chr21. A T21 positive sample is expected to show a 50% increase in the probe capture efficiency (PCE) at all five sites.

The less common cause for Down syndrome is when part of the chromosome 21 becomes attached to another chromosome, resulting in three copies of a section of chr21 in each cell of the patient's body. To detect such conditions, the number of sites on Chr21 is increased from N=5 to a larger number. In this condition, a patient sample is expected to show 50% increase in the PCE value only in a fraction of these sites. Such sites correspond to the section of Chr21 that is attached to another chromosome.

### Example 3

### Detection of 1p36 Deletion Syndrome

1p36 deletion syndrome is a disorder that often causes severe intellectual disability together with certain typical craniofacial features. It affects between 1 in 5000 and 1 in 10000 newborns. In 1p36 patients, a section on the short arm of chromosome 1 is missing. To detect such conditions, a number of N (e.g. N=5) sites on the most distal band of the short arm of chromosome 1 (1p36) are selected. By applying the systems and methods of embodiments of this disclosure, the positive samples are expected to show a decreased PCE from those probes.

### Example 4

### Detection of Deletion in BRCA1/2

The present disclosure may be applied to detecting a deletion mutation in BRCA1 and/or BRCA2. In one example, a partial deletion of BRCA1 Exon 11 may be detected.

Blood samples are obtained from human subjects with known mutation status, and gDNA is extracted. Prior to proceeding with the assay, the gDNA may be sheared by sonication to a size within the range of 350-650 base pairs. Shearing of the DNA may greatly improve the assay efficiency by allowing access to regions of the genome that are traditionally difficult to access, such as GC rich regions.

A probe that spans the 40 bp deletion within BRCA1 exon 11 is selected and used at a concentration of 10 pM. As an example, the sequence of the MIP that is used to detect deletion is as follows:

(SEQ ID NO: 9)
/5Phos/GTCTGAATCAAATGCCAAAGTNNNNNNNNNNCTTCAGCTTCCC
GATTACGGGTACGATCCGACGGTAGTGTNNNNNNNNNNTCCCCTGTGTGA
GAGAAAAGA

96 DNA samples were run through a multiplexed assay using a probe pool that includes the above sequence. In particular, the probe pool may include 1, 2, 3, 4, 5, 10, 15, 20, 30, 40, 50, 60, 70, 80, 90, 100, 200, 300, 400, 500, 600, 700, 800, 900, 1000, 1500, 2000 other probes (or any other suitable number of probes) in a multiplexed assay to interrogate multiple genomic locations. In this example, 68 samples were tested for BRCA1 Exon 11 copy number variations.

The workflow is outlined as follows:

Target Capture:

1. Prepare target capture, master mix:

GCS Target Capture

| | |
|---|---|
| 98° C. | 5 min |

Touchdown 20% temp ramp speed, ~90 min

| | |
|---|---|
| 56° C. | 120 min |
| 4° C. | hold |

| Reagent | X1 | X112 |
|---|---|---|
| 250 ng gDNA | 5.0 | 560.0 |
| Probe Pool G | 0.2 | 22.4 |
| 10X Ampligase Buffer | 2.0 | 224.0 |
| Water | 11.3 | 1265.6 |
| 5M Betaine | 1.5 | 168.0 |
| Total vol | 20.0 | 2240.0 |

2. Add 5 ul sample to 15 ul capture mix

3. Thermocycler program: GCS Target Capture

Extension/ligation:

4. Prepare extension/ligation master mix:

GCS Extension Ligation

| | |
|---|---|
| 56 C. | 60 min |
| 72 C. | 20 min |
| 37 C. | hold |

| Reagent | X1 | X112 |
|---|---|---|
| 10 mM dNTP | 0.6 | 67.2 |
| 100X NAD | 0.8 | 89.6 |
| 5M Betaine | 0.0 | 0.0 |
| 10X Ampligase Buff | 2.0 | 224.0 |
| Ampligase, 5 U/ul | 2.0 | 224.0 |
| Phusion Pol HF, 2 U/ul | 0.5 | 56.0 |
| water | 14.1 | 1579.2 |
| Total vol | 20.0 | 2240.0 |

5. Add 20 ul extension/ligation mix to each sample.

6. Thermocycler program: GCS Extension Ligation

Exonuclease Digestion:

7. Prepare Exonuclease master mix:

| Reagent | X1 | X112 |
|---|---|---|
| Exo I, 20 U/ul | 2 | 224 |
| Exo III, 100 U/ul | 2 | 224 |
| 10X NEBuffer I | 5 | 560 |
| Water | 1 | 112 |
| Total vol | 10 | 1120 |

GCS Exonuclease Digestion

| | |
|---|---|
| 37 C. | 55 min |
| 90 C. | 40 min |
| 4 C. | forever |

8. Add 10 ul master mix to each reaction.

9. Thermocycler program: GCS Exonuclease Digestion

10. Cool samples on ice (optionally store at −20 C)

PCR Amplification:

11. Prepare circular amplification PCR master mix:

HCP PCR amplification

| | | |
|---|---|---|
| 95 C. | 2 min | |
| 98 C. | 15 sec | 24 Cycles |
| 65 C. | 15 sec | |
| 72 C. | 15 sec | |
| 72 C. | 5 min | |
| 4 C. | forever | |

| Reagent | X1 | X112 |
|---|---|---|
| CCCP circular DNA | 10 | 1120 |
| 5X Phusion HF Buffer | 10 | 1120 |
| 10 mM dNTPs | 1 | 112 |
| Phusion Pol HS, 2 U/ul | 1 | 112 |
| FW Primer (100 uM) | 0.25 | 28 |
| Universal Primers (REV, | 5 | 560 |
| 5 uM) | | |
| water | 22.75 | 2548 |
| Total vol | 50 | 5600 |

12. Add 10 ul sample and 5 ul primer to 35 ul PCR mix

13. Thermocycler program: HCP PCR amplification

14. Select samples were QC'd on tapestation after amplification.

15. Purify amplified products using Ampure beads. 5 ul from each sample is pooled and pool is mixed with 480 ul Ampure beads. After 5 minutes, samples are washed twice with 960 ul 70% EtOH, dried for 26 minutes, and the pellet is resuspended in 40 ul low TE buffer. The purified pool is QC'd on the Qubit.

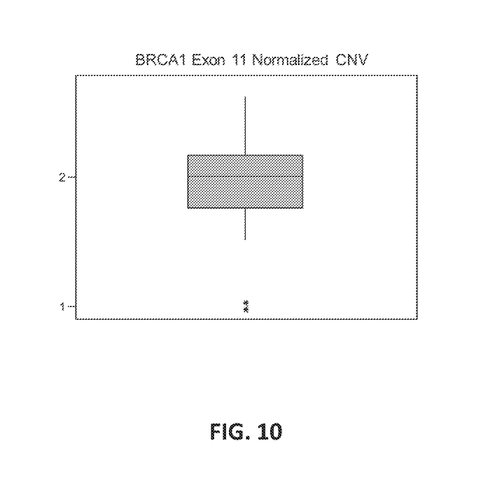

Following the above-described 15-step assay, the pooled 96 sample library is sequenced on an Illumina HiSeq 2500 instrument using 160 cycles of paired-end sequencing. Resultant reads are processed by trimming, filtering and flagging until they are aligned to the genome. The number of unique molecular tags (or number of capture events) originating from the selected MIP that aligned to the target region of BRCA1 exon 11 are counted, and may be referred to herein as u_{BRCA1}_{_}ₑₓₒₙ₁₁. To calculate a probe capture metric for BRCA1 Exon 11 for each sample, this number of unique molecular tags is normalized by a normalization factor that may include the total number of unique molecular tags across the entire sample. In an example, the normalization factor is represented by the denominator of EQ. 1. In another example, the normalization factor for normalizing U_{BRCA1}_{_}ₑₓₒₙ₁₁ may only include the sum of the control capture events in EQ. 1, or the sum of u_{CONTROL i,s} where i=1, 2 . . . . J, where J is the number of control populations used in the sample s. The resulting probe capture metric is then normalized again to reflect the presence of two copies in known normal samples. As an example, the probe capture metric may be normalized (to have a mean of one or two, for example) based on the status of the control population, or prior knowledge of the sample copy number in the known samples. In another example, if the copy number of the sample is unknown, then a normalization process similar to step 526 may be performed. In particular, the probe capture metric may be normalized by a composite control population. The results of the assay (where U_{BRCA}_{ₑₓₒₙ₁₁} is normalized by the sum of u_{CONTROL i,s}, and the resulting probe capture metrics are normalized based on the status of the control population) are shown in FIG. 10, which depicts a boxplot of the normalized BRCA1 exon 11 copy number. A total of 68 data points are represented, including 66 two-copy data points and two one-copy data points.

The normalized CNV for BRCA1 exon 11 as calculated using the UMI counts correctly identified the BRCA1 Exon 11 copy number of each of the 68 samples. In addition to correctly determining copy number, the normalized CNV score produced a clear separation between normal samples (2 copies) and those with the BRCA1 exon 11 partial deletion (1 copy).

Sample detail and results for the 68 samples tested for BRCA1 exon 11 deletion are shown in Table 2 below.

TABLE 2

| | | | BRCA1 | Result |
|---|---|---|---|---|
| | | | Exon 11 | consistent |
| | Known | Normalized | Copy | with known |
| Sample | Status | UMI | Number | status |
| A1 | Normal | 0.0213 | 2 | Yes |
| B1 | Normal | 0.0264 | 2 | Yes |
| MAXI1 | Normal | 0.0266 | 2 | Yes |
| MAXI10 | Normal | 0.0194 | 2 | Yes |
| MAXI12 | Normal | 0.0278 | 2 | Yes |
| MAXI16 | Normal | 0.0205 | 2 | Yes |
| MAXI17 | Normal | 0.0252 | 2 | Yes |
| MAXI18 | Normal | 0.0263 | 2 | Yes |
| MAXI19 | Normal | 0.0323 | 2 | Yes |
| MAXI2 | Normal | 0.0259 | 2 | Yes |
| MAXI20 | Normal | 0.0274 | 2 | Yes |
| MAXI21 | Normal | 0.0245 | 2 | Yes |
| MAXI3 | Normal | 0.0227 | 2 | Yes |
| MAXI4 | Normal | 0.0190 | 2 | Yes |
| MAXI6 | Normal | 0.0213 | 2 | Yes |
| MAXI7 | Normal | 0.0238 | 2 | Yes |
| MAXI8 | Normal | 0.0191 | 2 | Yes |
| NA00449 | Normal | 0.0241 | 2 | Yes |
| NA01526 | Normal | 0.0269 | 2 | Yes |
| NA02052 | Normal | 0.0246 | 2 | Yes |
| NA02633 | Normal | 0.0251 | 2 | Yes |
| NA02782 | Normal | 0.0206 | 2 | Yes |
| NA03189 | Normal | 0.0238 | 2 | Yes |
| NA03223 | Normal | 0.0274 | 2 | Yes |
| NA03332 | Normal | 0.0256 | 2 | Yes |
| NA04510 | Normal | 0.0280 | 2 | Yes |
| NA07499 | Normal | 0.0232 | 2 | Yes |
| NA08436 | Normal | 0.0303 | 2 | Yes |
| NA09587 | Normal | 0.0187 | 2 | Yes |
| NA10080 | Normal | 0.0237 | 2 | Yes |
| NA11254 | Normal | 0.0243 | 2 | Yes |
| NA11601 | Normal | 0.0288 | 2 | Yes |
| NA11602 | Normal | 0.0236 | 2 | Yes |
| NA11630 | Normal | 0.0289 | 2 | Yes |
| NA13021 | Normal | 0.0236 | 2 | Yes |
| NA13248 | Normal | 0.0193 | 2 | Yes |
| NA13250 | Normal | 0.0216 | 2 | Yes |
| NA13252 | Normal | 0.0244 | 2 | Yes |
| NA13255 | Normal | 0.0234 | 2 | Yes |
| NA13256 | Normal | 0.0301 | 2 | Yes |
| NA13328 | Normal | 0.0261 | 2 | Yes |
| NA13661 | Normal | 0.0268 | 2 | Yes |
| NA13691 | Normal | 0.0209 | 2 | Yes |
| NA13705 | Normal | 0.0213 | 2 | Yes |
| NA13707 | Known | 0.0093 | 1 | Yes |
| | Deletion | | | |
| NA13708 | Normal | 0.0198 | 2 | Yes |
| NA13712 | Normal | 0.0234 | 2 | Yes |
| NA13715 | Normal | 0.0198 | 2 | Yes |
| NA13792 | Normal | 0.0235 | 2 | Yes |
| NA13802 | Normal | 0.0186 | 2 | Yes |
| NA13862 | Normal | 0.0174 | 2 | Yes |
| NA13906 | Normal | 0.0254 | 2 | Yes |
| NA14090 | Normal | 0.0233 | 2 | Yes |
| NA14091 | Normal | 0.0238 | 2 | Yes |
| NA14092 | Normal | 0.0176 | 2 | Yes |
| NA14094 | Known | 0.0086 | 1 | Yes |
| | Deletion | | | |
| NA14170 | Normal | 0.0172 | 2 | Yes |
| NA14451 | Normal | 0.0194 | 2 | Yes |
| NA14471 | Normal | 0.0242 | 2 | Yes |
| NA14623 | Normal | 0.0267 | 2 | Yes |
| NA14626 | Normal | 0.0236 | 2 | Yes |
| NA14636 | Normal | 0.0193 | 2 | Yes |
| NA14637 | Normal | 0.0241 | 2 | Yes |
| NA14638 | Normal | 0.0227 | 2 | Yes |
| NA14639 | Normal | 0.0187 | 2 | Yes |
| NA14805 | Normal | 0.0254 | 2 | Yes |
| NA16533 | Normal | 0.0327 | 2 | Yes |
| NA21849 | Normal | 0.0165 | 2 | Yes |

### Example 5

### Detection of Exon Level Deletions and Duplications in the DMD Gene

The present disclosure may be applied to detecting exon level deletions and duplications in the DMD gene. DNA samples may be obtained from individuals with known DMD mutations to run an experiment. The probe pool may include 520 unique probes that range in concentration from 10 pM to 20 pM. All probes may span the intron/exon boundaries and tile 79 DMD exons. Table 3 lists a set of DMD MIPs or probes used for exon level copy counting.

TABLE 3

| | | SEQ |
|---|---|---|
| MIP | | ID |
| Probe | Sequence | NO |
| DMD1 | /5Phos/TCCGAAGGTAATTGCCTCCCNNNNN | 10 |
| | NNNNNCTTCAGCTTCCCGATTACGGGTACG | |
| | ATCCGACGGTAGTGTNNNNNNNNNNTACT | |
| | TCTTCCCACCAAAGCA | |
| DMD2 | /5Phos/ACGTTTAGTTTGTGACAAGCTCANN | 11 |
| | NNNNNNNNCTTCAGCTTCCCGATTACGGGT | |
| | ACGATCCGACGGTAGTGTNNNNNNNNNNT | |
| | GTTTTTAAGCCTACTGGAGCAA | |
| DMD3 | /5Phos/AGTCCTCTACTTCTTCCCACCANNNN | 12 |
| | NNNNNNCTTCAGCTTCCCGATTACGGGTAC | |
| | GATCCGACGGTAGTGTNNNNNNNNNNTGC | |
| | TTCTTTGCAAACTACTGT | |
| DMD4 | /5Phos/CAAAATGGACTATGTACCTGTGTNN | 13 |
| | NNNNNNNNCTTCAGCTTCCCGATTACGGGT | |
| | ACGATCCGACGGTAGTGTNNNNNNNNNNT | |
| | GCATTTTAGATGAAAGAGAAGATGT | |
| DMD5 | /5Phos/ACTTTCCATTATGATGTGTTAGTGTN | 14 |
| | NNNNNNNNNCTTCAGCTTCCCGATTACGG | |
| | GTACGATCCGACGGTAGTGTNNNNNNNNN | |
| | NACCTTAGAAAATTGTGCATTTACCC | |
| DMD6 | /5Phos/TGTGCATTTACCCATTTTGTGANNNN | 15 |
| | NNNNNNCTTCAGCTTCCCGATTACGGGTAC | |
| | GATCCGACGGTAGTGTNNNNNNNNNNATT | |
| | TCCAGATTTGCACAGCT | |
| DMD7 | /5Phos/ATGAAAGAGAAGATGTTCAAAAGA | 16 |
| | ANNNNNNNNNNCTTCAGCTTCCCGATTAC | |
| | GGGTACGATCCGACGGTAGTGTNNNNNNN | |
| | NNNCCCCAAACCAGCATCACTCA | |
| DMD8 | /5Phos/TGACCTACAGGATGGGAGGCNNNNN | 17 |
| | NNNNNCTTCAGCTTCCCGATTACGGGTACG | |
| | ATCCGACGGTAGTGTNNNNNNNNNNTCGG | |
| | CAGATTAATTATGCAC | |
| DMD9 | /5Phos/ACAAAGCACACTTCCAATGATACAN | 18 |
| | NNNNNNNNNCTTCAGCTTCCCGATTACGG | |
| | GTACGATCCGACGGTAGTGTNNNNNNNNN | |
| | NCCAGTTTTTGCCCTGTCAGG | |
| DMD10 | /5Phos/CAGGCCTTCGAGGAGGTCTANNNNN | 19 |
| | NNNNNCTTCAGCTTCCCGATTACGGGTACG | |
| | ATCCGACGGTAGTGTNNNNNNNNNNACGA | |
| | GGTTGCTTTACTAAGGA | |
| DMD11 | /5Phos/TCAGACCAGAAACTGACAACANNNN | 20 |
| | NNNNNNCTTCAGCTTCCCGATTACGGGTAC | |
| | GATCCGACGGTAGTGTNNNNNNNNNNTCA | |
| | GTGACCTACAGGATGGGA | |
| DMD12 | /5Phos/GGTCTGGATGCTGTGACACANNNNN | 21 |
| | NNNNNCTTCAGCTTCCCGATTACGGGTACG | |
| | ATCCGACGGTAGTGTNNNNNNNNNNCTCT | |
| | GCTGGTCAGTGAACACT | |
| DMD13 | /5Phos/AACGAACAGAGCCTGTGAGGNNNN | 22 |
| | NNNNNNCTTCAGCTTCCCGATTACGGGTAC | |
| | GATCCGACGGTAGTGTNNNNNNNNNNGGC | |
| | ATGAACTCTTGTGGATCC | |
| DMD14 | /5Phos/CGCAGTGCCTTGTTGACATTNNNNN | 23 |
| | NNNNNCTTCAGCTTCCCGATTACGGGTACG | |
| | ATCCGACGGTAGTGTNNNNNNNNNNTTTC | |
| | TCTGCATTTGGGGCCA | |
| DMD15 | /5Phos/CACTGACCAGCAGAGAGACCGACAA | 24 |
| | NNNNNNNNNNCTTCAGCTTCCCGATTACG | |
| | GGTACGATCCGACGGTAGTGTNNNNNNNN | |
| | NNCAAAGCCCTCACTCAAACATGAAGC | |
| DMD16 | /5Phos/ACCCTTGACGTGTGAAACAANNNNN | 25 |
| | NNNNNCTTCAGCTTCCCGATTACGGGTACG | |
| | ATCCGACGGTAGTGTNNNNNNNNNNACCC | |
| | CTTTCTTTAACAGGTTGA | |
| DMD17 | /5Phos/ACCAAGAGTCAGTTTATGATTTCCA | 26 |
| | NNNNNNNNNNCTTCAGCTTCCCGATTACG | |
| | GGTACGATCCGACGGTAGTGTNNNNNNNN | |
| | NNAAGCAGCACTATGGAGCAGG | |
| DMD18 | /5Phos/ATAATCCTCCACTGGCAGGTNNNNN | 27 |
| | NNNNNCTTCAGCTTCCCGATTACGGGTACG | |
| | ATCCGACGGTAGTGTNNNNNNNNNNAGCT | |
| | AAATGCAATTACCTTCACGT | |
| DMD19 | /5Phos/CGTGAAGGTAATTGCATTTAGCTNN | 28 |
| | NNNNNNNNCTTCAGCTTCCCGATTACGGGT | |
| | ACGATCCGACGGTAGTGTNNNNNNNNNNA | |
| | CCTGCCAGTGGAGGATTAT | |
| DMD20 | /5Phos/TCATGGCTGGATTGCAACAANNNNN | 29 |
| | NNNNNCTTCAGCTTCCCGATTACGGGTACG | |
| | ATCCGACGGTAGTGTNNNNNNNNNNTGTC | |
| | TCATTACTAATTGGCCCT | |
| DMD21 | /5Phos/TCCTTGAGCAAGAACCATGCANNNN | 30 |
| | NNNNNNCTTCAGCTTCCCGATTACGGGTAC | |
| | GATCCGACGGTAGTGTNNNNNNNNNNCCA | |
| | GCTGGTGGTGAAGTTGA | |
| DMD22 | /5Phos/GATTCTCCTGAGCTGGGTCCNNNNN | 31 |
| | NNNNNCTTCAGCTTCCCGATTACGGGTACG | |
| | ATCCGACGGTAGTGTNNNNNNNNNNGTTT | |
| | GCATGGTTCTTGCTCA | |
| DMD23 | /5Phos/ACGAGTTGATTGTCGGACCCNNNNN | 32 |
| | NNNNNCTTCAGCTTCCCGATTACGGGTACG | |
| | ATCCGACGGTAGTGTNNNNNNNNNNTGAT | |
| | CTGGAACCATACTGGGG | |
| DMD24 | /5Phos/GCCTGGCTTTGAATGCTCTCNNNNN | 33 |
| | NNNNNCTTCAGCTTCCCGATTACGGGTACG | |
| | ATCCGACGGTAGTGTNNNNNNNNNNGGCT | |
| | GGATTGCAACAAACCA | |
| DMD25 | /5Phos/TTCATTACATTTTTGACCTACATGTG | 34 |
| | GNNNNNNNNNNCTTCAGCTTCCCGATTAC | |
| | GGGTACGATCCGACGGTAGTGTNNNNNNN | |
| | NNNGTCTCAGTAATCTTCTTACCTATGACT | |
| | ATGG | |
| DMD26 | /5Phos/ACATGCATTCAACATCGCCANNNNN | 35 |
| | NNNNNCTTCAGCTTCCCGATTACGGGTACG | |
| | ATCCGACGGTAGTGTNNNNNNNNNNGACT | |
| | ATGGGCATTGGTTGTCAA | |
| DMD27 | /5Phos/ACCCTTTAAAATATTTCTATTTAAAC | 36 |
| | AAGTNNNNNNNNNNCTTCAGCTTCCCGAT | |
| | TACGGGTACGATCCGACGGTAGTGTNNNN | |
| | NNNNNNTTCCAGTCAAATAGGTCTGGC | |
| DMD28 | /5Phos/CCAGTCAAATAGGTCTGGCCNNNNN | 37 |
| | NNNNNCTTCAGCTTCCCGATTACGGGTACG | |
| | ATCCGACGGTAGTGTNNNNNNNNNNAAAA | |
| | GCAGTGGTAGTCCAGA | |
| DMD29 | /5Phos/GGATCGAGTAGTTTCTCTATGCCNN | 38 |
| | NNNNNNNNCTTCAGCTTCCCGATTACGGGT | |
| | ACGATCCGACGGTAGTGTNNNNNNNNNNT | |
| | CTTCACTGCAATTTTAGATACTGG | |
| DMD30 | /5Phos/TCTGAGACTTGTCATTTCTACACANN | 39 |
| | NNNNNNNNCTTCAGCTTCCCGATTACGGGT | |
| | ACGATCCGACGGTAGTGTNNNNNNNNNNA | |
| | GTCAGCCACACAACGACTG | |
| DMD31 | /5Phos/TGTCCATGAATGTCCTCCAGAGNNN | 40 |
| | NNNNNNNCTTCAGCTTCCCGATTACGGGTA | |
| | CGATCCGACGGTAGTGTNNNNNNNNNNGG | |
| | ACTTCTTATCTGGATAGGTGGT | |
| DMD32 | /5Phos/CACTTTAGGTGGCCTTGGCANNNNN | 41 |
| | NNNNNCTTCAGCTTCCCGATTACGGGTACG | |
| | ATCCGACGGTAGTGTNNNNNNNNNNAGGC | |
| | TTTGTATATATACACGTGT | |
| DMD33 | /5Phos/GAAGCCATCCAGGAAGTGGANNNN | 42 |
| | NNNNNNCTTCAGCTTCCCGATTACGGGTAC | |
| | GATCCGACGGTAGTGTNNNNNNNNNNTGA | |
| | TGTGTAGTGTTAATGTGCT | |
| DMD34 | /5Phos/GGACTTCTTATCTGGATAGGTGGTN | 43 |
| | NNNNNNNNNCTTCAGCTTCCCGATTACGG | |
| | GTACGATCCGACGGTAGTGTNNNNNNNNN | |
| | NTCACTTTAGGTGGCCTTGGC | |
| DMD35 | /5Phos/TGCATTTTTAGGTATTACGTGCACAN | 44 |
| | NNNNNNNNNCTTCAGCTTCCCGATTACGG | |
| | GTACGATCCGACGGTAGTGTNNNNNNNNN | |
| | NAGCATTGAAGCCATCCAGGA | |
| DMD36 | /5Phos/AGGAGGGGGAAAAACCATAANNNN | 45 |
| | NNNNNNCTTCAGCTTCCCGATTACGGGTAC | |
| | GATCCGACGGTAGTGTNNNNNNNNNNCGT | |
| | GTAGGGTCAGAGGTGGT | |
| DMD37 | /5Phos/CGGAGCCCATTTCCTTCACANNNNN | 46 |
| | NNNNNCTTCAGCTTCCCGATTACGGGTACG | |
| | ATCCGACGGTAGTGTNNNNNNNNNNGTCA | |
| | GTCTAGCACAGGGATATG | |
| DMD38 | /5Phos/AGGTGGTGACATAAGCAGCCNNNNN | 47 |
| | NNNNNCTTCAGCTTCCCGATTACGGGTACG | |
| | ATCCGACGGTAGTGTNNNNNNNNNNCAAA | |
| | CCAGCTCTTCACGAGG | |
| DMD39 | /5Phos/CAAACCAGAGAACTGCTTCCANNNN | 48 |
| | NNNNNNCTTCAGCTTCCCGATTACGGGTAC | |
| | GATCCGACGGTAGTGTNNNNNNNNNNCCC | |
| | TAAGCCTCGATTCAAGA | |
| DMD40 | /5Phos/AGAGAAGGGTTTGGGGGAGTNNNN | 49 |
| | NNNNNNCTTCAGCTTCCCGATTACGGGTAC | |
| | GATCCGACGGTAGTGTNNNNNNNNNNGGT | |
| | GGTGACATAAGCAGCCT | |
| DMD41 | /5Phos/GATGTGGAAGTGGTGAAAGACCNNN | 50 |
| | NNNNNNNCTTCAGCTTCCCGATTACGGGTA | |
| | CGATCCGACGGTAGTGTNNNNNNNNNNTT | |
| | GTGCAGCATTTGGAAGCT | |
| DMD42 | /5Phos/TCAGCAGAAAGAAGCCACGATNNN | 51 |
| | NNNNNNNCTTCAGCTTCCCGATTACGGGTA | |
| | CGATCCGACGGTAGTGTNNNNNNNNNNGA | |
| | GGAAAAAGGATGACTTGCCA | |
| DMD43 | /5Phos/GATTGTTCCAGTACATTAAATGATG | 52 |
| | AATCGNNNNNNNNNNCTTCAGCTTCCCGA | |
| | TTACGGGTACGATCCGACGGTAGTGTNNN | |
| | NNNNNNNACTCTCCATCAATGAACTGCCA | |
| DMD44 | /5Phos/CTATGATGTGCTTGGGATTCCANNN | 53 |
| | NNNNNNNCTTCAGCTTCCCGATTACGGGTA | |
| | CGATCCGACGGTAGTGTNNNNNNNNNNAT | |
| | GTGGAAGTGGTGAAAGACC | |
| DMD45 | /5Phos/TTTGATGTGGTTTGATGGTTAAGNN | 54 |
| | NNNNNNNNCTTCAGCTTCCCGATTACGGGT | |
| | ACGATCCGACGGTAGTGTNNNNNNNNNNC | |
| | TCCTAAATTCAAGATGGGAATG | |
| DMD46 | /5Phos/GGGCCGGGTTGGTAATATTCTNNNN | 55 |
| | NNNNNNCTTCAGCTTCCCGATTACGGGTAC | |
| | GATCCGACGGTAGTGTNNNNNNNNNNGGG | |
| | CCACAAGTTTAAAACTGCA | |
| DMD47 | /5Phos/ACCCTGAGGCATTCCCATCTNNNNN | 56 |
| | NNNNNCTTCAGCTTCCCGATTACGGGTACG | |
| | ATCCGACGGTAGTGTNNNNNNNNNNAAGA | |
| | AAGCTGTGTGCCTTGG | |
| DMD48 | /5Phos/ACCCCTGACAAAGAAGGAAGTTNNN | 57 |
| | NNNNNNNCTTCAGCTTCCCGATTACGGGTA | |
| | CGATCCGACGGTAGTGTNNNNNNNNNNAT | |
| | GCTAGCTACCCTGAGGCA | |
| DMD49 | /5Phos/TGCAGAATCCCAAAACCACTNNNNN | 58 |
| | NNNNNCTTCAGCTTCCCGATTACGGGTACG | |
| | ATCCGACGGTAGTGTNNNNNNNNNNTGGG | |
| | CTGTCAAATCCATCATGT | |
| DMD50 | /5Phos/GGAAAAACAAAGCAAGTAAGTCCN | 59 |
| | NNNNNNNNNCTTCAGCTTCCCGATTACGG | |
| | GTACGATCCGACGGTAGTGTNNNNNNNNN | |
| | NCAGGGCCGGGTTGGTAATAT | |
| DMD51 | /5Phos/TCGCATTTGGGGGCATCTATNNNNN | 60 |
| | NNNNNCTTCAGCTTCCCGATTACGGGTACG | |
| | ATCCGACGGTAGTGTNNNNNNNNNNGCCA | |
| | GTCATTCAACTCTTTCAGT | |
| DMD52 | /5Phos/GAAGAGCCTCTTGGACCTGANNNNN | 61 |
| | NNNNNCTTCAGCTTCCCGATTACGGGTACG | |
| | ATCCGACGGTAGTGTNNNNNNNNNNAGTT | |
| | GCTTTCAAAGAGGTCA | |
| DMD53 | /5Phos/CCTATACACAGTAACACAGATGACA | 62 |
| | TGNNNNNNNNNNCTTCAGCTTCCCGATTAC | |
| | GGGTACGATCCGACGGTAGTGTNNNNNNN | |
| | NNNCTTGAAGACCTAAAACGCCAAGT | |
| DMD54 | /5Phos/GCCAGTCATTCAACTCTTTCAGTNNN | 63 |
| | NNNNNNNCTTCAGCTTCCCGATTACGGGTA | |
| | CGATCCGACGGTAGTGTNNNNNNNNNNAA | |
| | GCACGCAACATAAGATACACC | |
| DMD55 | /5Phos/AGTGGAGATCACGCAACTGCNNNNN | 64 |
| | NNNNNCTTCAGCTTCCCGATTACGGGTACG | |
| | ATCCGACGGTAGTGTNNNNNNNNNNGCAA | |
| | ATCATTTCAACACACATGTAAGA | |
| DMD56 | /5Phos/CCACCACCATGTGAGTGAGANNNNN | 65 |
| | NNNNNCTTCAGCTTCCCGATTACGGGTACG | |
| | ATCCGACGGTAGTGTNNNNNNNNNNTTTT | |
| | CAAGTTATAGTTCTTTTAAAGGACA | |
| DMD57 | /5Phos/TCTGCTACATCTCAGGTACTCCNNN | 66 |
| | NNNNNNNCTTCAGCTTCCCGATTACGGGTA | |
| | CGATCCGACGGTAGTGTNNNNNNNNNNAC | |
| | CACCACCATGTGAGTGAG | |
| DMD58 | /5Phos/ACACACACTCATAATCAGCTGAANN | 67 |
| | NNNNNNNNCTTCAGCTTCCCGATTACGGGT | |
| | ACGATCCGACGGTAGTGTNNNNNNNNNNT | |
| | GGAGATCACGCAACTGCTG | |
| DMD59 | /5Phos/CCTTGGAATTCTTTAATGTCTTGCNN | 68 |
| | NNNNNNNNCTTCAGCTTCCCGATTACGGGT | |
| | ACGATCCGACGGTAGTGTNNNNNNNNNNC | |
| | CGCTGGGTTCTTTTACAAGAC | |
| DMD60 | /5Phos/AATGGCATGAATAATTTGCCNNNNN | 69 |
| | NNNNNCTTCAGCTTCCCGATTACGGGTACG | |
| | ATCCGACGGTAGTGTNNNNNNNNNNCGTT | |
| | GCCATTTGAGAAGGAT | |
| DMD61 | /5Phos/CGCTAGAAGTTGGAAGGGACANNN | 70 |
| | NNNNNNNCTTCAGCTTCCCGATTACGGGTA | |
| | CGATCCGACGGTAGTGTNNNNNNNNNNTT | |
| | TGCCCATCGATCTCCCAA | |
| DMD62 | /5Phos/AGCTGTAAAAGACACGGGGGNNNN | 71 |
| | NNNNNNCTTCAGCTTCCCGATTACGGGTAC | |
| | GATCCGACGGTAGTGTNNNNNNNNNNTGC | |
| | TGATGCTGTGCTTGATTG | |
| DMD63 | /5Phos/AAGCCATGCACTAAAAAGGCANNN | 72 |
| | NNNNNNNCTTCAGCTTCCCGATTACGGGTA | |
| | CGATCCGACGGTAGTGTNNNNNNNNNNTG | |
| | AAAGCTAGAAAGTACATACGGC | |
| DMD64 | /5Phos/AGCCAGTTGTGTGAATCTTGTNNNN | 73 |
| | NNNNNNCTTCAGCTTCCCGATTACGGGTAC | |
| | GATCCGACGGTAGTGTNNNNNNNNNNCCC | |
| | ACTTTAATTCAGAAAAGTAGCA | |
| DMD65 | /5Phos/ACAAGATTCACACAACTGGCTTTNN | 74 |
| | NNNNNNNNCTTCAGCTTCCCGATTACGGGT | |
| | ACGATCCGACGGTAGTGTNNNNNNNNNNA | |
| | GCTGTAAAAGACACGGGGG | |
| DMD66 | /5Phos/ACAGCACAGGTTAGTGATACCAANN | 75 |
| | NNNNNNNNCTTCAGCTTCCCGATTACGGGT | |
| | ACGATCCGACGGTAGTGTNNNNNNNNNNG | |
| | CAATCCATGGGCAAACTGT | |
| DMD67 | /5Phos/TAAGCCTGGGTTGCATTCCANNNNN | 76 |
| | NNNNNCTTCAGCTTCCCGATTACGGGTACG | |
| | ATCCGACGGTAGTGTNNNNNNNNNNTTAT | |
| | CCCAACACCGGGCAAA | |
| DMD68 | /5Phos/AAGCAATCCATGGGCAAACTGNNNN | 77 |
| | NNNNNNCTTCAGCTTCCCGATTACGGGTAC | |
| | GATCCGACGGTAGTGTNNNNNNNNNNTTT | |
| | TGATCCTTTGCGGGCAC | |
| DMD69 | /5Phos/TATCCAGCCATGCTTCCGTCNNNNN | 78 |
| | NNNNNCTTCAGCTTCCCGATTACGGGTACG | |
| | ATCCGACGGTAGTGTNNNNNNNNNNAGGG | |
| | CAAAAACTAATCTGGTTGC | |
| DMD70 | /5Phos/TGCTCAAGAGGAACTTCCACCNNNN | 79 |
| | NNNNNNCTTCAGCTTCCCGATTACGGGTAC | |
| | GATCCGACGGTAGTGTNNNNNNNNNNTGC | |
| | CACTCCAAGCAGTCTTT | |
| DMD71 | /5Phos/TGCCTCTTCTTTTGGGGAGGNNNNN | 80 |
| | NNNNNCTTCAGCTTCCCGATTACGGGTACG | |
| | ATCCGACGGTAGTGTNNNNNNNNNNCAGG | |
| | TACCCGAGGATTCTGG | |
| DMD72 | /5Phos/GCTTGTTGGTAGATTGACCTTCAGN | 81 |
| | NNNNNNNNNCTTCAGCTTCCCGATTACGG | |
| | GTACGATCCGACGGTAGTGTNNNNNNNNN | |
| | NGATGGCTGAGTGGTGGTGAC | |
| DMD73 | /5Phos/AGCAGTTTTGTTGGTGGTGTNNNNN | 82 |
| | NNNNNCTTCAGCTTCCCGATTACGGGTACG | |
| | ATCCGACGGTAGTGTNNNNNNNNNNTACG | |
| | GTGACCACAAGGGAAC | |
| DMD74 | /5Phos/GGTGGTGACAGCCTGTGAAANNNNN | 83 |
| | NNNNNCTTCAGCTTCCCGATTACGGGTACG | |
| | ATCCGACGGTAGTGTNNNNNNNNNNTGCC | |
| | TCTTCTTTTGGGGAGG | |
| DMD75 | /5Phos/TGCAGAGTCCTGAATTTGCANNNNN | 84 |
| | NNNNNCTTCAGCTTCCCGATTACGGGTACG | |
| | ATCCGACGGTAGTGTNNNNNNNNNNGTCA | |
| | GGCAGGAGTCTCAGAT | |
| DMD76 | /5Phos/TGAGCGAGTAATCCAGCTGTGNNNN | 85 |
| | NNNNNNCTTCAGCTTCCCGATTACGGGTAC | |
| | GATCCGACGGTAGTGTNNNNNNNNNNACT | |
| | AGTAGAATCACAGATAACAAAGCA | |
| DMD77 | /5Phos/AGATAGCAAGCAAAATCAAAGTTTA | 86 |
| | GNNNNNNNNNNCTTCAGCTTCCCGATTAC | |
| | GGGTACGATCCGACGGTAGTGTNNNNNNN | |
| | NNNGGCAACTTCTCAGACTTAAAAGAA | |
| DMD78 | /5Phos/AGCAGCACTATTTTCCCTGTNNNNN | 87 |
| | NNNNNCTTCAGCTTCCCGATTACGGGTACG | |
| | ATCCGACGGTAGTGTNNNNNNNNNNTCCA | |
| | GCTGTGAAGTTCAGTT | |
| DMD79 | /5Phos/GGTGAATGGTAATTACACGAGTTGA | 88 |
| | TNNNNNNNNNNCTTCAGCTTCCCGATTACG | |
| | GGTACGATCCGACGGTAGTGTNNNNNNNN | |
| | NNCTCTCATGCTGCAGGCCATA | |
| DMD80 | /5Phos/TCTACTTGCCCTTTCAAGCCTNNNNN | 89 |
| | NNNNNCTTCAGCTTCCCGATTACGGGTACG | |
| | ATCCGACGGTAGTGTNNNNNNNNNNCTGA | |
| | TCTGCTGGCATCTTGC | |
| DMD81 | /5Phos/ATCTGCTGGCATCTTGCAGTNNNNN | 90 |
| | NNNNNCTTCAGCTTCCCGATTACGGGTACG | |
| | ATCCGACGGTAGTGTNNNNNNNNNNAGTG | |
| | CTTGTCTGATATAATTCAGCT | |
| DMD82 | /5Phos/TGTCATCTGCTCCAATTGTTGNNNNN | 91 |
| | NNNNNCTTCAGCTTCCCGATTACGGGTACG | |
| | ATCCGACGGTAGTGTNNNNNNNNNNTTAT | |
| | GCTCCAAATGGAAGGAG | |
| DMD83 | /5Phos/ACCGGCTGTTCAGTTGTTCTNNNNN | 92 |
| | NNNNNCTTCAGCTTCCCGATTACGGGTACG | |
| | ATCCGACGGTAGTGTNNNNNNNNNNACTT | |
| | TTAATTGCTGTTGGCTCTGA | |
| DMD84 | /5Phos/GCCAGTTGCTAAGTGAGAGACTNNN | 93 |
| | NNNNNNNCTTCAGCTTCCCGATTACGGGTA | |
| | CGATCCGACGGTAGTGTNNNNNNNNNNTT | |
| | CAGTCTGTGGGTTCAGGG | |
| DMD85 | /5Phos/TGGCAATTTCCAAGAAGACAGTANN | 94 |
| | NNNNNNNNCTTCAGCTTCCCGATTACGGGT | |
| | ACGATCCGACGGTAGTGTNNNNNNNNNNA | |
| | AAATCCAACCCACCACCCC | |
| DMD86 | /5Phos/ACCACATGAATGATTTCAAACCAGA | 95 |
| | NNNNNNNNNNCTTCAGCTTCCCGATTACG | |
| | GGTACGATCCGACGGTAGTGTNNNNNNNN | |
| | NNACCGGCTGTTCAGTTGTTCT | |
| DMD87 | /5Phos/TTCTGATGTGCAGGCCAGAGNNNNN | 96 |
| | NNNNNCTTCAGCTTCCCGATTACGGGTACG | |
| | ATCCGACGGTAGTGTNNNNNNNNNNGCAC | |
| | AGGATGAAGTCAACCG | |
| DMD88 | /5Phos/AGCAGTAAGGCAAGTTTAGCTNNNN | 97 |
| | NNNNNNCTTCAGCTTCCCGATTACGGGTAC | |
| | GATCCGACGGTAGTGTNNNNNNNNNNAAC | |
| | ATGGGTCCTTGTCCTTTCT | |
| DMD89 | /5Phos/GGAACATGGGTCCTTGTCCTNNNNN | 98 |
| | NNNNNCTTCAGCTTCCCGATTACGGGTACG | |
| | ATCCGACGGTAGTGTNNNNNNNNNNACCT | |
| | TCTGGATTTCCCCACA | |
| DMD90 | /5Phos/ACCATTCTCCCTACAACCTGTNNNN | 99 |
| | NNNNNNCTTCAGCTTCCCGATTACGGGTAC | |
| | GATCCGACGGTAGTGTNNNNNNNNNNCAG | |
| | GCCAGAGAGAAAGAGCT | |
| DMD91 | /5Phos/TTGGTGGCAAAGTGTCAAAANNNNN | 100 |
| | NNNNNCTTCAGCTTCCCGATTACGGGTACG | |
| | ATCCGACGGTAGTGTNNNNNNNNNNGCTT | |
| | GATAAGCGTGCTTTATTG | |
| DMD92 | /5Phos/AGTCGGTGACACTAAGTTGAGGNNN | 101 |
| | NNNNNNNCTTCAGCTTCCCGATTACGGGTA | |
| | CGATCCGACGGTAGTGTNNNNNNNNNNTT | |
| | GCTCAATGGGCAAACTACC | |
| DMD93 | /5Phos/TTCACACTTTGCCATGTTTTCCTNNN | 102 |
| | NNNNNNNCTTCAGCTTCCCGATTACGGGTA | |
| | CGATCCGACGGTAGTGTNNNNNNNNNNTG | |
| | GTTTCTGACTGCTGGACC | |
| DMD94 | /5Phos/TGACACTTTGCCACCAATGCNNNNN | 103 |
| | NNNNNCTTCAGCTTCCCGATTACGGGTACG | |
| | ATCCGACGGTAGTGTNNNNNNNNNNAGCA | |
| | GGAATGTATCTTCATAATCAT | |
| DMD95 | /5Phos/GGGGAATTGCAGGTCTGTGANNNNN | 104 |
| | NNNNNCTTCAGCTTCCCGATTACGGGTACG | |
| | ATCCGACGGTAGTGTNNNNNNNNNNTGCG | |
| | CTATCAGGAGACCATG | |
| DMD96 | /5Phos/AGGAGCAAATGAATAAACTCCGANN | 105 |
| | NNNNNNNNCTTCAGCTTCCCGATTACGGGT | |
| | ACGATCCGACGGTAGTGTNNNNNNNNNNG | |
| | AGATGTCGAAGAAAGCGCC | |
| DMD97 | /5Phos/GGCCACTTTGTTGCTCTTGCNNNNN | 106 |
| | NNNNNCTTCAGCTTCCCGATTACGGGTACG | |
| | ATCCGACGGTAGTGTNNNNNNNNNNTCTT | |
| | CCAGCGTCCCTCAATT | |
| DMD98 | /5Phos/GCTGGGAGGAGAGCTTCTTCNNNNN | 107 |
| | NNNNNCTTCAGCTTCCCGATTACGGGTACG | |
| | ATCCGACGGTAGTGTNNNNNNNNNNAGAT | |
| | GCTGAAGGTCAAATGCTT | |
| DMD99 | /5Phos/GCCCTCTGAAATTAGCCGGANNNNN | 108 |
| | NNNNNCTTCAGCTTCCCGATTACGGGTACG | |
| | ATCCGACGGTAGTGTNNNNNNNNNNAGAT | |
| | TTCAAGTACAGTTAATTTCACT | |
| DMD100 | /5Phos/TCTATCAGTTATAAACTTCTAGTGGT | 109 |
| | AANNNNNNNNNNCTTCAGCTTCCCGATTA | |
| | CGGGTACGATCCGACGGTAGTGTNNNNNN | |
| | NNNNGGCCACTTTGTTGCTCTTGC | |
| DMD101 | /5Phos/CAGGCCCAAAAACAATTCCCANNNN | 110 |
| | NNNNNNCTTCAGCTTCCCGATTACGGGTAC | |
| | GATCCGACGGTAGTGTNNNNNNNNNNCAG | |
| | GCCATTCCTCCTTCAGAA | |
| DMD102 | /5Phos/GGCCATTCCTCCTTCAGAAANNNNN | 111 |
| | NNNNNCTTCAGCTTCCCGATTACGGGTACG | |
| | ATCCGACGGTAGTGTNNNNNNNNNNAGGA | |
| | GAGCAAAATCCACCCC | |
| DMD103 | /5Phos/CAGCTGAAACAGTGCAGAGTNNNNN | 112 |
| | NNNNNCTTCAGCTTCCCGATTACGGGTACG | |
| | ATCCGACGGTAGTGTNNNNNNNNNNTCAG | |
| | CACACCAGTAATGCCTT | |
| DMD104 | /5Phos/TGGGACTGATGGCATTGCATNNNNN | 113 |
| | NNNNNCTTCAGCTTCCCGATTACGGGTACG | |
| | ATCCGACGGTAGTGTNNNNNNNNNNCTGC | |
| | CCACCTTCATTGACACT | |
| DMD105 | /5Phos/CCTAATGTCTCCCTTCACCGNNNNN | 114 |
| | NNNNNCTTCAGCTTCCCGATTACGGGTACG | |
| | ATCCGACGGTAGTGTNNNNNNNNNNGCCA | |
| | GAGTTTGCTTCGAGAC | |
| DMD106 | /5Phos/TCAGTGGGATCACATGTGCCNNNNN | 115 |
| | NNNNNCTTCAGCTTCCCGATTACGGGTACG | |
| | ATCCGACGGTAGTGTNNNNNNNNNNTCAG | |
| | ACAATTCAGCCCAGTC | |
| DMD107 | /5Phos/GAAGCAAACTCTGGCTCTGCNNNNN | 116 |
| | NNNNNCTTCAGCTTCCCGATTACGGGTACG | |
| | ATCCGACGGTAGTGTNNNNNNNNNNAAGT | |
| | ACGTTGAGGCAAGCCA | |
| DMD108 | /5Phos/GGTGGGCAGAAGATAAAGAATGNN | 117 |
| | NNNNNNNNCTTCAGCTTCCCGATTACGGGT | |
| | ACGATCCGACGGTAGTGTNNNNNNNNNNG | |
| | CCATCAGTCCCAATTTTAC | |
| DMD109 | /5Phos/CCACAAAACAAACAAACAAAACAC | 118 |
| | GNNNNNNNNNNCTTCAGCTTCCCGATTAC | |
| | GGGTACGATCCGAGGTAGTGTNNNNNNN | |
| | NNNGCTTGTGTCATCCATTCGTGC | |
| DMD110 | /5Phos/TGCACGAATGGATGACACAAGNNNN | 119 |
| | NNNNNNCTTCAGCTTCCCGATTACGGGTAC | |
| | GATCCGACGGTAGTGTNNNNNNNNNNCGT | |
| | GTTTTGTTTGTTTGTTTTGTGG | |
| DMD111 | /5Phos/CATGGGGATCAGATACACTCAANNN | 120 |
| | NNNNNNNCTTCAGCTTCCCGATTACGGGTA | |
| | CGATCCGACGGTAGTGTNNNNNNNNNNTT | |
| | CAAGGCCTCCTTTCTGGC | |
| DMD112 | /5Phos/CCTCCTTTCTGGCATAGACCTNNNN | 121 |
| | NNNNNNCTTCAGCTTCCCGATTACGGGTAC | |
| | GATCCGACGGTAGTGTNNNNNNNNNNACC | |
| | TTCATCTCTTCAACTGCTT | |
| DMD113 | /5Phos/GCAGTTGAAGAGATGAAGGTNNNN | 122 |
| | NNNNNNCTTCAGCTTCCCGATTACGGGTAC | |
| | GATCCGACGGTAGTGTNNNNNNNNNNGCC | |
| | AGAAAGGAGGCCTTGAA | |
| DMD114 | /5Phos/GCCAGAAAGGAGGCCTTGAANNNN | 123 |
| | NNNNNNCTTCAGCTTCCCGATTACGGGTAC | |
| | GATCCGACGGTAGTGTNNNNNNNNNNTTG | |
| | AGTGTATCTGATCCCCATGAG | |
| DMD115 | /5Phos/GAAAGAAATGCAACAATGCTTGNNN | 124 |
| | NNNNNNNCTTCAGCTTCCCGATTACGGGTA | |
| | CGATCCGACGGTAGTGTNNNNNNNNNNCG | |
| | AATGGATGACACAAGCTG | |
| DMD116 | /5Phos/GGGCCATTTGCTTAACTTGTGTNNN | 125 |
| | NNNNNNNCTTCAGCTTCCCGATTACGGGTA | |
| | CGATCCGACGGTAGTGTNNNNNNNNNNGC | |
| | TGAATGGGAAATGCAAGACT | |
| DMD117 | /5Phos/TGAACTCCAGTCTCTTCCATNNNNN | 126 |
| | NNNNNCTTCAGCTTCCCGATTACGGGTACG | |
| | ATCCGACGGTAGTGTNNNNNNNNNNGCTT | |
| | CTTTTTGTTGGGCCTCT | |
| DMD118 | /5Phos/TGGTCATATGTGAGGCATAGTGGNN | 127 |
| | NNNNNNNNCTTCAGCTTCCCGATTACGGGT | |
| | ACGATCCGACGGTAGTGTNNNNNNNNNNC | |
| | TCAAGCTCCACCTGTAGCA | |
| DMD119 | /5Phos/TTCCCATTCAGCCTAGTGCANNNNN | 128 |
| | NNNNNCTTCAGCTTCCCGATTACGGGTACG | |
| | ATCCGACGGTAGTGTNNNNNNNNNNGCCA | |
| | AAGTTGTTTTGCACTGG | |
| DMD120 | /5Phos/GGGCCTCTTCTTTAGCTCTCTNNNNN | 129 |
| | NNNNNCTTCAGCTTCCCGATTACGGGTACG | |
| | ATCCGACGGTAGTGTNNNNNNNNNNGTGC | |
| | AGAGCCACTGGTAGTT | |
| DMD121 | /5Phos/CTCAAGCTCCACCTGTAGCANNNNN | 130 |
| | NNNNNCTTCAGCTTCCCGATTACGGGTACG | |
| | ATCCGACGGTAGTGTNNNNNNNNNNACTG | |
| | GGATGTTGTGAGAAAG | |
| DMD122 | /5Phos/CTAGCACCTCAGAGATTTCCTCANN | 131 |
| | NNNNNNNNCTTCAGCTTCCCGATTACGGGT | |
| | ACGATCCGACGGTAGTGTNNNNNNNNNNA | |
| | AGGTTATTAGGGGGAACAAAG | |
| DMD123 | /5Phos/CAGTATTAAAAGAGGTCAAGTACCA | 132 |
| | AATAGNNNNNNNNNNCTTCAGCTTCCCGA | |
| | TTACGGGTACGATCCGACGGTAGTGTNNN | |
| | NNNNNNNTAGAATTTAAACTTAAAACCAC | |
| | TGAAAACA | |
| DMD124 | /5Phos/GGTCACAAGATTTTGCAAAGGNNNN | 133 |
| | NNNNNNCTTCAGCTTCCCGATTACGGGTAC | |
| | GATCCGACGGTAGTGTNNNNNNNNNNGCA | |
| | AACAAGTGGCTAAATGAA | |
| DMD125 | /5Phos/GCAGCTAGACAGTTTCATCATCTNN | 134 |
| | NNNNNNNNCTTCAGCTTCCCGATTACGGGT | |
| | ACGATCCGACGGTAGTGTNNNNNNNNNNT | |
| | GCCAACATGCCCAAACTTC | |
| DMD126 | /5Phos/CCAACATGCCCAAACTTCCTNNNNN | 135 |
| | NNNNNCTTCAGCTTCCCGATTACGGGTACG | |
| | ATCCGACGGTAGTGTNNNNNNNNNNAGCA | |
| | CCTCAGAGATTTCCTCA | |
| DMD127 | /5Phos/GGAGAAAGCAAACAAGTGGCNNNN | 136 |
| | NNNNNNCTTCAGCTTCCCGATTACGGGTAC | |
| | GATCCGACGGTAGTGTNNNNNNNNNNACC | |
| | TGCTACAAAGTAAAGGTG | |
| DMD128 | /5Phos/AGGGTCTGTGCCAATATGCGNNNNN | 137 |
| | NNNNNCTTCAGCTTCCCGATTACGGGTACG | |
| | ATCCGACGGTAGTGTNNNNNNNNNNATCT | |
| | GAGAGGCCTGTATCTGC | |
| DMD129 | /5Phos/GCGGAGTCATGGATGAGCTANNNNN | 138 |
| | NNNNNCTTCAGCTTCCCGATTACGGGTACG | |
| | ATCCGACGGTAGTGTNNNNNNNNNNTCAG | |
| | AAGATACTGAGCATTTGC | |
| DMD130 | /5Phos/TGGATTATCAGCAAATGCTCANNNN | 139 |
| | NNNNNNCTTCAGCTTCCCGATTACGGGTAC | |
| | GATCCGACGGTAGTGTNNNNNNNNNNTCC | |
| | CTCCAACGAGAATTAAATG | |
| DMD131 | /5Phos/GTAGTTCCCTCCAACGAGAATNNNN | 140 |
| | NNNNNNCTTCAGCTTCCCGATTACGGGTAC | |
| | GATCCGACGGTAGTGTNNNNNNNNNNCAG | |
| | TGTCTGGCATTGGATTGT | |
| DMD132 | /5Phos/ACACCAAGGAGCATTTTTGCTNNNN | 141 |
| | NNNNNNCTTCAGCTTCCCGATTACGGGTAC | |
| | GATCCGACGGTAGTGTNNNNNNNNNNTCC | |
| | TCTGAATGTCGCATCAAAT | |
| DMD133 | /5Phos/GCTCAGCTTTCAGGTTTCAGANNNN | 142 |
| | NNNNNNCTTCAGCTTCCCGATTACGGGTAC | |
| | GATCCGACGGTAGTGTNNNNNNNNNNGGC | |
| | GGAGTCATGGATGAGCT | |
| DMD134 | /5Phos/AGACAGATTTCGCAGCTTCCTNNNN | 143 |
| | NNNNNNCTTCAGCTTCCCGATTACGGGTAC | |
| | GATCCGACGGTAGTGTNNNNNNNNNNTTC | |
| | AGTCTCCTGGGCAGACT | |
| DMD135 | /5Phos/GCAAGTACATCTGGGAATCAGCNNN | 144 |
| | NNNNNNNCTTCAGCTTCCCGATTACGGGTA | |
| | CGATCCGACGGTAGTGTNNNNNNNNNNAA | |
| | CAGAGCATCCAGTCTGCC | |
| DMD136 | /5Phos/GCTTGAACAGAGCATCCAGTCNNNN | 145 |
| | NNNNNNCTTCAGCTTCCCGATTACGGGTAC | |
| | GATCCGACGGTAGTGTNNNNNNNNNNGAG | |
| | CTGAATGAGTGCCAGGA | |
| DMD137 | /5Phos/ACTTTTGCCTCCTTACAGCCTNNNNN | 146 |
| | NNNNNCTTCAGCTTCCCGATTACGGGTACG | |
| | ATCCGACGGTAGTGTNNNNNNNNNNGCTT | |
| | CCTGAGGCATTTGAGC | |
| DMD138 | /5Phos/CATTGACAAGCAGTTGGCAGNNNNN | 147 |
| | NNNNNCTTCAGCTTCCCGATTACGGGTACG | |
| | ATCCGACGGTAGTGTNNNNNNNNNNACAT | |
| | TTAACTGATACACTCTTATTCCT | |
| DMD139 | /5Phos/CGTCCACCTTGTCTGCAATATAAGN | 148 |
| | NNNNNNNNNCTTCAGCTTCCCGATTACGG | |
| | GTACGATCCGACGGTAGTGTNNNNNNNNN | |
| | NAGACCCCCTTTTCTTCCTACC | |
| DMD140 | /5Phos/CCACCTCTACCATGTAGCTTCCNNN | 149 |
| | NNNNNNNCTTCAGCTTCCCGATTACGGGTA | |
| | CGATCCGACGGTAGTGTNNNNNNNNNNGC | |
| | CTCCTTCCCCTGATTATGT | |
| DMD141 | /5Phos/ACTCTTTGGGCAGCCTCCTTNNNNN | 150 |
| | NNNNNCTTCAGCTTCCCGATTACGGGTACG | |
| | ATCCGACGGTAGTGTNNNNNNNNNNTGTC | |
| | CTCAAATCCAATCTTGCC | |
| DMD142 | /5Phos/CGTTGGGCATTATACTCCAGTCTNN | 151 |
| | NNNNNNNNCTTCAGCTTCCCGATTACGGGT | |
| | ACGATCCGACGGTAGTGTNNNNNNNNNNT | |
| | CCTCCCAACAGAAAATCCA | |
| DMD143 | /5Phos/AGAC GCTGCTCAAAATTGGCNNNNN | 152 |
| | NNNNNCTTCAGCTTCCCGATTACGGGTACG | |
| | ATCCGACGGTAGTGTNNNNNNNNNNGGTA | |
| | CCTGCGTATTTGCCAC | |
| DMD144 | /5Phos/AGATCTGCCTTTATTTCTGAAGANN | 153 |
| | NNNNNNNNCTTCAGCTTCCCGATTACGGGT | |
| | ACGATCCGACGGTAGTGTNNNNNNNNNNG | |
| | CTGCTCAAAATTGGCTGGT | |
| DMD145 | /5Phos/GGACAGTGTAAAAAGGCACTGANN | 154 |
| | NNNNNNNNCTTCAGCTTCCCGATTACGGGT | |
| | ACGATCCGACGGTAGTGTNNNNNNNNNNG | |
| | TTTCCAATGCAGGCAAGTG | |
| DMD146 | /5Phos/CAGGTACCCCTTGACTTTCCNNNNN | 155 |
| | NNNNNCTTCAGCTTCCCGATTACGGGTACG | |
| | ATCCGACGGTAGTGTNNNNNNNNNNTCCA | |
| | GAAACCAGCCAATTTT | |
| DMD147 | /5Phos/TTTGCCTTTCAAACAATAACTGGTCN | 156 |
| | NNNNNNNNNCTTCAGCTTCCCGATTACGG | |
| | GTACGATCCGACGGTAGTGTNNNNNNNNN | |
| | NTTGCCACCAGAAATACATACCACACAAT | |
| | G | |
| DMD148 | /5Phos/GCACTTGCC TGCATTGGAAANNNNN | 157 |
| | NNNNNCTTCAGCTTCCCGATTACGGGTACG | |
| | ATCCGACGGTAGTGTNNNNNNNNNNAGGA | |
| | CCAGTTATTGTTTGAAAGGC | |
| DMD149 | /5Phos/TCTTTGTTTCCAATGCAGGCNNNNN | 158 |
| | NNNNNCTTCAGCTTCCCGATTACGGGTACG | |
| | ATCCGACGGTAGTGTNNNNNNNNNNGCCA | |
| | CAATACATGTGCCAAT | |
| DMD150 | /5Phos/TCTTTGGGATTTTCCGTCTGCNNNNN | 159 |
| | NNNNNCTTCAGCTTCCCGATTACGGGTACG | |
| | ATCCGACGGTAGTGTNNNNNNNNNNTTGC | |
| | CCGTTGCTTTACAATTT | |
| DMD151 | /5Phos/TCCACTTCAGACTTCACTTCACTNNN | 160 |
| | NNNNNNNCTTCAGCTTCCCGATTACGGGTA | |
| | CGATCCGACGGTAGTGTNNNNNNNNNNAC | |
| | CTTTGCTCCCAGCTCATT | |
| DMD152 | /5Phos/ACTGGACGTCAGATTGTACAGANNN | 161 |
| | NNNNNNNCTTCAGCTTCCCGATTACGGGTA | |
| | CGATCCGACGGTAGTGTNNNNNNNNNNAC | |
| | ATGGAATAGCAATTAAGGGG | |
| DMD153 | /5Phos/GTGGTCAATATCTAGCTTTTGCATTN | 162 |
| | NNNNNNNNNCTTCAGCTTCCCGATTACGG | |
| | GTACGATCCGACGGTAGTGTNNNNNNNNN | |
| | NTCCACTTCAGACTTCACTTCACT | |
| DMD154 | /5Phos/GCTGAGACCACAAACACTTCTNNNN | 163 |
| | NNNNNNCTTCAGCTTCCCGATTACGGGTAC | |
| | GATCCGACGGTAGTGTNNNNNNNNNNTGG | |
| | TGATAAAGACTGGACGTCA | |
| DMD155 | /5Phos/TTCTCCAACTGTTGCTTTCTTTCTGTT | 164 |
| | ACNNNNNNNNNNCTTCAGCTTCCCGATTA | |
| | CGGGTACGATCCGACGGTAGTGTNNNNNN | |
| | NNNNCTTTCCCCAGGCAACTTCAGAATCCA | |
| | AA | |
| DMD156 | /5Phos/CAGCAGTTGAAGGAATGCCTNNNNN | 165 |
| | NNNNNCTTCAGCTTCCCGATTACGGGTACG | |
| | ATCCGACGGTAGTGTNNNNNNNNNNAGCA | |
| | ACAGTTGGAGAAATGCT | |
| DMD157 | /5Phos/TGAAGGTTATTTTGAACATACGTGA | 166 |
| | NNNNNNNNNNCTTCAGCTTCCCGATTACG | |
| | GGTACGATCCGACGGTAGTGTNNNNNNNN | |
| | NNAGAATGGCTGGCAGCTACAG | |
| DMD158 | /5Phos/TTTCCCCAGGCAACTTCAGANNNNN | 167 |
| | NNNNNCTTCAGCTTCCCGATTACGGGTACG | |
| | ATCCGACGGTAGTGTNNNNNNNNNNTCAT | |
| | GGTCCTGAAAAGCACAGA | |
| DMD159 | /5Phos/CACTTATTTGGAACTTTTATATTTCT | 168 |
| | GTNNNNNNNNNNCTTCAGCTTCCCGATTAC | |
| | GGGTACGATCCGACGGTAGTGTNNNNNNN | |
| | NNNTCCTTTCGCATCTTACGGGAC | |
| DMD160 | /5Phos/GAACATACGTGAAAACACATAATAT | 169 |
| | GNNNNNNNNNNCTTCAGCTTCCCGATTAC | |
| | GGGTACGATCCGACGGTAGTGTNNNNNNN | |
| | NNNTTTCAGGTAACAGAAAGAAAGC | |
| DMD161 | /5Phos/CCTTTCGCATCTTACGGGACNNNNN | 170 |
| | NNNNNCTTCAGCTTCCCGATTACGGGTACG | |
| | ATCCGACGGTAGTGTNNNNNNNNNNGGTT | |
| | TTACCTTTCCCCAGGC | |
| DMD162 | /5Phos/GGCCTCTCCTACCTCTGTGANNNNN | 171 |
| | NNNNNCTTCAGCTTCCCGATTACGGGTACG | |
| | ATCCGACGGTAGTGTNNNNNNNNNNTTAA | |
| | CCACTCTTCTGCTCGGG | |
| DMD163 | /5Phos/CAAGAAGGAGACGTTGGTGGANNN | 172 |
| | NNNNNNNCTTCAGCTTCCCGATTACGGGTA | |
| | CGATCCGACGGTAGTGTNNNNNNNNNNTG | |
| | CTCTCCTTTTCACAGGCT | |
| DMD164 | /5Phos/ACACCCTTCTCTGTCACGAGNNNNN | 173 |
| | NNNNNCTTCAGCTTCCCGATTACGGGTACG | |
| | ATCCGACGGTAGTGTNNNNNNNNNNCAAG | |
| | AAGGAGACGTTGGTGGA | |
| DMD165 | /5Phos/TGAAACGGCTTTCTGTATGGNNNNN | 174 |
| | NNNNNCTTCAGCTTCCCGATTACGGGTACG | |
| | ATCCGACGGTAGTGTNNNNNNNNNNAGGC | |
| | CTCTCCTACCTCTGTG | |
| DMD166 | /5Phos/TGTACAGAGACATACCATGGCANNN | 175 |
| | NNNNNNNCTTCAGCTTCCCGATTACGGGTA | |
| | CGATCCGACGGTAGTGTNNNNNNNNNNAG | |
| | CACGTCTTCTTTTTGCTGG | |
| DMD167 | /5Phos/CAGGCTGACACACTTTTGGANNNNN | 176 |
| | NNNNNCTTCAGCTTCCCGATTACGGGTACG | |
| | ATCCGACGGTAGTGTNNNNNNNNNNTTCT | |
| | TTAAGAATATTGTCTAACCAATAATGC | |
| DMD168 | /5Phos/ACCAGTTACTTCAATCATCTTTGTCC | 177 |
| | NNNNNNNNNNCTTCAGCTTCCCGATTACG | |
| | GGTACGATCCGACGGTAGTGTNNNNNNNN | |
| | NNCACAAAGTGGATCATTCAGGC | |
| DMD169 | /5Phos/GTGGTATTTTCATATAGAATATTGCG | 178 |
| | TNNNNNNNNNNCTTCAGCTTCCCGATTACG | |
| | GGTACGATCCGACGGTAGTGTNNNNNNNN | |
| | NNTGTGGTCCACATTCTGGTCAA | |
| DMD170 | /5Phos/CACGTCTTCTTTTTGCTGGGGNNNNN | 179 |
| | NNNNNCTTCAGCTTCCCGATTACGGGTACG | |
| | ATCCGACGGTAGTGTNNNNNNNNNNTCCA | |
| | TTCAAAGGGGGAAGGA | |
| DMD171 | /5Phos/TGAGAGCAAGCACATGCAGANNNN | 180 |
| | NNNNNNCTTCAGCTTCCCGATTACGGGTAC | |
| | GATCCGACGGTAGTGTNNNNNNNNNNGCG | |
| | TATGTCATTCAGTTCTGCC | |
| DMD172 | /5Phos/CGGTGACCACTGCAGGAAATNNNNN | 181 |
| | NNNNNCTTCAGCTTCCCGATTACGGGTACG | |
| | ATCCGACGGTAGTGTNNNNNNNNNNCTCG | |
| | CTCTGTTTGGCTCTCT | |
| DMD173 | /5Phos/TGAGCTCTGAGATTTGGGGCNNNNN | 182 |
| | NNNNNCTTCAGCTTCCCGATTACGGGTACG | |
| | ATCCGACGGTAGTGTNNNNNNNNNNTGAA | |
| | AACCTGCTGTGGGGT | |
| DMD174 | /5Phos/GCAGTACTCTGAAAGGGGCANNNNN | 183 |
| | NNNNNCTTCAGCTTCCCGATTACGGGTACG | |
| | ATCCGACGGTAGTGTNNNNNNNNNNAGCA | |
| | AACTTGATGGCAAACC | |
| DMD175 | /5Phos/GGTCACGTGTAGAGTCCACCNNNNN | 184 |
| | NNNNNCTTCAGCTTCCCGATTACGGGTACG | |
| | ATCCGACGGTAGTGTNNNNNNNNNNCGCA | |
| | AGAGACCATTTAGCACA | |
| DMD176 | /5Phos/CCTCTTTCAGATTCACCCCCNNNNN | 185 |
| | NNNNNCTTCAGCTTCCCGATTACGGGTACG | |
| | ATCCGACGGTAGTGTNNNNNNNNNNAAGG | |
| | CCAAGAATATTCTGCAT | |
| DMD177 | /5Phos/TGGAAAGAACTTAGATAAGTCTCCA | 186 |
| | NNNNNNNNNNCTTCAGCTTCCCGATTACG | |
| | GGTACGATCCGACGGTAGTGTNNNNNNNN | |
| | NNCTTGAACCACTGGAGGCTGA | |
| DMD178 | /5Phos/CTTCAAAGGAATGGAGGCCTNNNNN | 187 |
| | NNNNNCTTCAGCTTCCCGATTACGGGTACG | |
| | ATCCGACGGTAGTGTNNNNNNNNNNTTTC | |
| | CACTCCTAGTTCATTCACA | |
| DMD179 | /5Phos/TTGCTTGAACCACTGGAGGCNNNNN | 188 |
| | NNNNNCTTCAGCTTCCCGATTACGGGTACG | |
| | ATCCGACGGTAGTGTNNNNNNNNNNTGTG | |
| | ATTAGTTTAGCAACAGGAGG | |
| DMD180 | /5Phos/CATTTATTCAACCTCCTGTTGCNNNN | 189 |
| | NNNNNNCTTCAGCTTCCCGATTACGGGTAC | |
| | GATCCGACGGTAGTGTNNNNNNNNNNTTT | |
| | CAGATTCACCCCCTGCT | |
| DMD181 | /5Phos/AGATGAGAGAAAGCGAGAGGANNN | 190 |
| | NNNNNNNCTTCAGCTTCCCGATTACGGGTA | |
| | CGATCCGACGGTAGTGTNNNNNNNNNNAC | |
| | CAAAATGAAGACTGTACTTGTTGT | |
| DMD182 | /5Phos/TTGTCTGTAACAGCTGCTGTNNNNN | 191 |
| | NNNNNCTTCAGCTTCCCGATTACGGGTACG | |
| | ATCCGACGGTAGTGTNNNNNNNNNNGAAC | |
| | AGAAAAAGTGAGTTTCTGATGA | |
| DMD183 | /5Phos/TGAGTGGTATTTGATTTTGAACGNN | 192 |
| | NNNNNNNNCTTCAGCTTCCCGATTACGGGT | |
| | ACGATCCGACGGTAGTGTNNNNNNNNNNT | |
| | GAGAGAAAGCGAGAGGAAA | |
| DMD184 | /5Phos/GCTCATAGCCTTTCTTTTACATTTGG | 193 |
| | NNNNNNNNNNCTTCAGCTTCCCGATTACG | |
| | GGTACGATCCGACGGTAGTGTNNNNNNNN | |
| | NNACAGTACCCTCATTGTCTTCATT | |
| DMD185 | /5Phos/CCCTCATTGTCTTCATTCTGATCANN | 194 |
| | NNNNNNNNCTTCAGCTTCCCGATTACGGGT | |
| | ACGATCCGACGGTAGTGTNNNNNNNNNNT | |
| | GTTTTGTCTGTAACAGCTGCTG | |
| DMD186 | /5Phos/TTGTTGCAAAGAGGAGACAACTNNN | 195 |
| | NNNNNNNCTTCAGCTTCCCGATTACGGGTA | |
| | CGATCCGACGGTAGTGTNNNNNNNNNNAG | |
| | CATTCCATGAAAGTTTTAAATTGG | |
| DMD187 | /5Phos/TTGATGTTCTTGTTTCTATTAACGTN | 196 |
| | NNNNNNNNNCTTCAGCTTCCCGATTACGG | |
| | GTACGATCCGACGGTAGTGTNNNNNNNNN | |
| | NGAGGCAGGCTGATGATCTCC | |
| DMD188 | /5Phos/CCTCAAATCCTGTTCATGGTGCNNN | 197 |
| | NNNNNNNCTTCAGCTTCCCGATTACGGGTA | |
| | CGATCCGACGGTAGTGTNNNNNNNNNNTG | |
| | GTATTGACATTCTAAAACAACATTACC | |
| DMD189 | /5Phos/TCAGTACAAGAGGCAGGCTGNNNNN | 198 |
| | NNNNNCTTCAGCTTCCCGATTACGGGTACG | |
| | ATCCGACGGTAGTGTNNNNNNNNNNTAAC | |
| | TGCAGCCAGAAGTGCA | |
| DMD190 | /5Phos/GCTCAGGTAGGCTGGCTAATNNNNN | 199 |
| | NNNNNCTTCAGCTTCCCGATTACGGGTACG | |
| | ATCCGACGGTAGTGTNNNNNNNNNNACAA | |
| | CACACAATACAAGGAAATGC | |
| DMD191 | /5Phos/TGTCATCCAAGCATTTCAGGNNNNN | 200 |
| | NNNNNCTTCAGCTTCCCGATTACGGGTACG | |
| | ATCCGACGGTAGTGTNNNNNNNNNNCAAC | |
| | ATTTTAAATATGATCTTCACAGG | |
| DMD192 | /5Phos/TTGTGCAAAGTTGAGTCTTCGANNN | 201 |
| | NNNNNNNCTTCAGCTTCCCGATTACGGGTA | |
| | CGATCCGACGGTAGTGTNNNNNNNNNNAG | |
| | TGTTACAGAAGCCCAAAGTGA | |
| DMD193 | /5Phos/GAGCTGGATCTGAGTTGGCTNNNNN | 202 |
| | NNNNNCTTCAGCTTCCCGATTACGGGTACG | |
| | ATCCGACGGTAGTGTNNNNNNNNNNAAAC | |
| | ACATACGTGGGTTTGC | |
| DMD194 | /5Phos/TTTGCTCTCAATTTCCCGCCNNNNNN | 203 |
| | NNNNCTTCAGCTTCCCGATTACGGGTACGA | |
| | TCCGACGGTAGTGTNNNNNNNNNNCCACT | |
| | CACTTTCAGAATGTACA | |
| DMD195 | /5Phos/CTGGCAAACCCACGTATGTGNNNNN | 204 |
| | NNNNNCTTCAGCTTCCCGATTACGGGTACG | |
| | ATCCGACGGTAGTGTNNNNNNNNNNGAGC | |
| | TGAATGCAGTGCGTAG | |
| DMD196 | /5Phos/GCAGTGGAGCCAACTCAGATNNNNN | 205 |
| | NNNNNCTTCAGCTTCCCGATTACGGGTACG | |
| | ATCCGACGGTAGTGTNNNNNNNNNNTGCT | |
| | TGCAAGTCGGTTGATG | |
| DMD197 | /5Phos/CCAGGGCAGTTAGCTAACCANNNNN | 206 |
| | NNNNNCTTCAGCTTCCCGATTACGGGTACG | |
| | ATCCGACGGTAGTGTNNNNNNNNNNTTGC | |
| | TCTCAATTTCCCGCCA | |
| DMD198 | /5Phos/TCAAAGGCTGTTGTCCCTTTNNNNN | 207 |
| | NNNNNCTTCAGCTTCCCGATTACGGGTACG | |
| | ATCCGACGGTAGTGTNNNNNNNNNNCCAT | |
| | CCTCAGACAAGCCCTC | |
| DMD199 | /5Phos/AATGCTCCTGACCTCTGTGCNNNNN | 208 |
| | NNNNNCTTCAGCTTCCCGATTACGGGTACG | |
| | ATCCGACGGTAGTGTNNNNNNNNNNTACC | |
| | AGCACACTGTCCGTGA | |
| DMD200 | /5Phos/CCATCATCGTTTCTTCACGGACAGTG | 209 |
| | TGNNNNNNNNNNCTTCAGCTTCCCGATTAC | |
| | GGGTACGATCCGACGGTAGTGTNNNNNNN | |
| | NNNCTTCAGAGACTCCTCTTGCTTAAAGAG | |
| | AT | |
| DMD201 | /5Phos/CCTAACAGTGAAACCTCCTCCATNN | 210 |
| | NNNNNNNNCTTCAGCTTCCCGATTACGGGT | |
| | ACGATCCGACGGTAGTGTNNNNNNNNNNG | |
| | GGCTTGTGAGACATGAGTGA | |
| DMD202 | /5Phos/TGCATCATGATGGCATTTTGACTNN | 211 |
| | NNNNNNNNCTTCAGCTTCCCGATTACGGGT | |
| | ACGATCCGACGGTAGTGTNNNNNNNNNNG | |
| | CTCCTGACCTCTGTGCTAA | |
| DMD203 | /5Phos/GGGCTTGTGAGACATGAGTGANNNN | 212 |
| | NNNNNNCTTCAGCTTCCCGATTACGGGTAC | |
| | GATCCGACGGTAGTGTNNNNNNNNNNGTG | |
| | CTTTGGTTTTACCTTCAGAGA | |
| DMD204 | /5Phos/TCTACAACAAAGCTCAGGTCGGNNN | 213 |
| | NNNNNNNCTTCAGCTTCCCGATTACGGGTA | |
| | CGATCCGACGGTAGTGTNNNNNNNNNNGT | |
| | CAATAATTAAGAATTGCAACACCA | |
| DMD205 | /5Phos/ACAAATCCCAAAGGTAGCAAATGGN | 214 |
| | NNNNNNNNNCTTCAGCTTCCCGATTACGG | |
| | GTACGATCCGACGGTAGTGTNNNNNNNNN | |
| | NTTCCACAGGCGTTGCACTTT | |
| DMD206 | /5Phos/GGGAGAGAGCTTCCTGTAGCNNNNN | 215 |
| | NNNNNCTTCAGCTTCCCGATTACGGGTACG | |
| | ATCCGACGGTAGTGTNNNNNNNNNNCTGA | |
| | AATAAATTCTACAGTTCCCTGAAAAC | |
| DMD207 | /5Phos/GGACCGACAAGGGTAGGTAACNNN | 216 |
| | NNNNNNNCTTCAGCTTCCCGATTACGGGTA | |
| | CGATCCGACGGTAGTGTNNNNNNNNNNAC | |
| | AACAAAGCTCAGGTCGGA | |
| DMD208 | /5Phos/ACTGTTCAGCTTCTGTTAGCCANNN | 217 |
| | NNNNNNNCTTCAGCTTCCCGATTACGGGTA | |
| | CGATCCGACGGTAGTGTNNNNNNNNNNTC | |
| | CATCACCCTTCAGAACCTG | |
| DMD209 | /5Phos/GGATCAAGAAAAATAGATGGATTAT | 218 |
| | GTNNNNNNNNNNCTTCAGCTTCCCGATTAC | |
| | GGGTACGATCCGACGGTAGTGTNNNNNNN | |
| | NNNCCCAATTCTCAGGAATTTGTGT | |
| DMD210 | /5Phos/GGTTATACTGACAAAGATATCACTC | 219 |
| | TGNNNNNNNNNNCTTCAGCTTCCCGATTAC | |
| | GGGTACGATCCGACGGTAGTGTNNNNNNN | |
| | NNNAGATCTGTCAAATCGCCTGC | |
| DMD211 | /5Phos/TTCCTGAGAATTGGGAACATGCNNN | 220 |
| | NNNNNNNCTTCAGCTTCCCGATTACGGGTA | |
| | CGATCCGACGGTAGTGTNNNNNNNNNNAT | |
| | GCTTTTACCTGCAGGCGA | |
| DMD212 | /5Phos/GGATCAAGAAAAATAGATGGATTAT | 221 |
| | GTNNNNNNNNNNCTTCAGCTTCCCGATTAC | |
| | GGGTACGATCCGACGGTAGTGTNNNNNNN | |
| | NNNCCCAATTCTCAGGAATTTGTGT | |
| DMD213 | /5Phos/TGCAGGTAAAAGCATATGGATCAAG | 222 |
| | NNNNNNNNNNCTTCAGCTTCCCGATTACG | |
| | GGTACGATCCGACGGTAGTGTNNNNNNN | |
| | NNTCCATCACCCTTCAGAACCTGATCT | |
| DMD214 | /5Phos/TTGGGAAGCCTGAATCTGCGNNNNN | 223 |
| | NNNNNCTTCAGCTTCCCGATTACGGGTACG | |
| | ATCCGACGGTAGTGTNNNNNNNNNNGGGG | |
| | CTTCATTTTTGTTTTGCC | |
| DMD215 | /5Phos/CCCAATGCCATCCTGGAGTTNNNNN | 224 |
| | NNNNNCTTCAGCTTCCCGATTACGGGTACG | |
| | ATCCGACGGTAGTGTNNNNNNNNNNTCTG | |
| | TCTGACAGCTGTTTGCA | |
| DMD216 | /5Phos/CAAAAATGAAGCCCCATGTCNNNNN | 225 |
| | NNNNNCTTCAGCTTCCCGATTACGGGTACG | |
| | ATCCGACGGTAGTGTNNNNNNNNNNTTTC | |
| | TTCCCCAGTTGCATTC | |
| DMD217 | /5Phos/TGACATGCCCATATCCAAAGGANNN | 226 |
| | NNNNNNNCTTCAGCTTCCCGATTACGGGTA | |
| | CGATCCGACGGTAGTGTNNNNNNNNNNCC | |
| | AATGCCATCCTGGAGTTC | |
| DMD218 | /5Phos/TGACAGCTGTTTGCAGACCTNNNNN | 227 |
| | NNNNNCTTCAGCTTCCCGATTACGGGTACG | |
| | ATCCGACGGTAGTGTNNNNNNNNNNGTTA | |
| | GTGCCTTTCACCCTGC | |
| DMD219 | /5Phos/AGAGGTAGGGCGACAGATCTNNNN | 228 |
| | NNNNNNCTTCAGCTTCCCGATTACGGGTAC | |
| | GATCCGACGGTAGTGTNNNNNNNNNNGGC | |
| | AAACTGTTGTCAGAACA | |
| DMD220 | /5Phos/AGCAATGTTATCTGCTTCCTCCANNN | 229 |
| | NNNNNNNCTTCAGCTTCCCGATTACGGGTA | |
| | CGATCCGACGGTAGTGTNNNNNNNNNNCT | |
| | TTATGCAAGCAGGCCCTG | |
| DMD221 | /5Phos/CTGGGACACAAACATGGCAANNNN | 230 |
| | NNNNNNCTTCAGCTTCCCGATTACGGGTAC | |
| | GATCCGACGGTAGTGTNNNNNNNNNNTGT | |
| | TATCTGCTTCCTCCAACCA | |
| DMD222 | /5Phos/ACCTGGAAAAGAGCAGCAACNNNN | 231 |
| | NNNNNNCTTCAGCTTCCCGATTACGGGTAC | |
| | GATCCGACGGTAGTGTNNNNNNNNNNTCT | |
| | TTCTCCAGGCTAGAAGAACA | |
| DMD223 | /5Phos/GACAAGATATTCTTTTGTTCTTCTAG | 232 |
| | CNNNNNNNNNNCTTCAGCTTCCCGATTAC | |
| | GGGTACGATCCGACGGTAGTGTNNNNNNN | |
| | NNNCTTGACTTGCTCAAGCTTTTCTTTTAG | |
| DMD224 | /5Phos/GTTTGAGAATTCCCTGGCGCNNNNN | 233 |
| | NNNNNCTTCAGCTTCCCGATTACGGGTACG | |
| | ATCCGACGGTAGTGTNNNNNNNNNNACAC | |
| | ATGTGACGGAAGAGATGG | |
| DMD225 | /5Phos/GGAGGCTGGTATGTGGATTGTNNNN | 234 |
| | NNNNNNCTTCAGCTTCCCGATTACGGGTAC | |
| | GATCCGACGGTAGTGTNNNNNNNNNNGTG | |
| | CTCCCATAAGCCCAGAA | |
| DMD226 | /5Phos/GGCCCAGTGGTACCTCAAATANNNN | 235 |
| | NNNNNNCTTCAGCTTCCCGATTACGGGTAC | |
| | GATCCGACGGTAGTGTNNNNNNNNNNGGG | |
| | CAACTCTTCCACCAGTAA | |
| DMD227 | /5Phos/AGGACCCGTGCTTGTAAGTGNNNNN | 236 |
| | NNNNNCTTCAGCTTCCCGATTACGGGTACG | |
| | ATCCGACGGTAGTGTNNNNNNNNNNCTCG | |
| | GTCAAGTCGCTTCATT | |
| DMD228 | /5Phos/TGGAGATTTGTCTGCTTGAGCTNNN | 237 |
| | NNNNNNNCTTCAGCTTCCCGATTACGGGTA | |
| | CGATCCGACGGTAGTGTNNNNNNNNNNTA | |
| | GCCAAAGCAAACGGTCAG | |
| DMD229 | /5Phos/GTAACTGAAACAGACAAATGCAACA | 238 |
| | ACGNNNNNNNNNNCTTCAGCTTCCCGATT | |
| | ACGGGTACGATCCGACGGTAGTGTNNNNN | |
| | NNNNNGTCTAACCTTTATCCACTGGAGATT | |
| | TG | |
| DMD230 | /5Phos/TGCTGCTGTGGTTATCTCCTNNNNNN | 239 |
| | NNNNCTTCAGCTTCCCGATTACGGGTACGA | |
| | TCCGACGGTAGTGTNNNNNNNNNNTTCCTT | |
| | TCAGGTTTCCAGAGCT | |
| DMD231 | /5Phos/GGCAATATCACTGAATTTTCTCATTT | 240 |
| | GGNNNNNNNNNNCTTCAGCTTCCCGATTA | |
| | CGGGTACGATCCGACGGTAGTGTNNNNNN | |
| | NNNNCTGCTGCTGTGGTTATCTCCT | |
| DMD232 | /5Phos/TTTCAAGCTGCCCAAGGTCTNNNNN | 241 |
| | NNNNNCTTCAGCTTCCCGATTACGGGTACG | |
| | ATCCGACGGTAGTGTNNNNNNNNNNAACG | |
| | TCAAATGGTCCTTCTTGG | |
| DMD233 | /5Phos/GGTAAATAATTCTCAAGGCATAAGC | 242 |
| | NNNNNNNNNNCTTCAGCTTCCCGATTACG | |
| | GGTACGATCCGACGGTAGTGTNNNNNNNN | |
| | NNTTTCAAGCTGCCCAAGGTCTT | |
| DMD234 | /5Phos/TCTCTTCCACATCCGGTTGTNNNNNN | 243 |
| | NNNNCTTCAGCTTCCCGATTACGGGTACGA | |
| | TCCGACGGTAGTGTNNNNNNNNNNGTCCA | |
| | CGTCAATGGCAAATGT | |
| DMD235 | /5Phos/TTCCTGGGGAAAAGAACCCANNNNN | 244 |
| | NNNNNCTTCAGCTTCCCGATTACGGGTACG | |
| | ATCCGACGGTAGTGTNNNNNNNNNNTGCT | |
| | TCATTACCTTCACTGGCT | |
| DMD236 | /5Phos/GGGCAGCATTTGTACAAGGANNNNN | 245 |
| | NNNNNCTTCAGCTTCCCGATTACGGGTACG | |
| | ATCCGACGGTAGTGTNNNNNNNNNNTCTG | |
| | CAATACATGTGGAGTCTCC | |
| DMD237 | /5Phos/GCCTGGTACATAAGGGCACANNNNN | 246 |
| | NNNNNCTTCAGCTTCCCGATTACGGGTACG | |
| | ATCCGACGGTAGTGTNNNNNNNNNNTCCA | |
| | CATCCGGTTGTTTAGCT | |
| DMD238 | /5Phos/AGCAGTTCAAGCTAAACAACCGNNN | 247 |
| | NNNNNNNCTTCAGCTTCCCGATTACGGGTA | |
| | CGATCCGACGGTAGTGTNNNNNNNNNNTT | |
| | CTCTGCACCAAAAGCTACA | |
| DMD239 | /5Phos/TGGATCCCATTCTCTTTGGCTNNNNN | 248 |
| | NNNNNCTTCAGCTTCCCGATTACGGGTACG | |
| | ATCCGACGGTAGTGTNNNNNNNNNNAGAG | |
| | GAAGTTAGAAGATCTGAGCT | |
| DMD240 | /5Phos/AGTGGGTAGAATTTCTTTTAAAGGN | 249 |
| | NNNNNNNNNCTTCAGCTTCCCGATTACGG | |
| | GTACGATCCGACGGTAGTGTNNNNNNNNN | |
| | NGGTTTACCGCCTTCCACTCA | |
| DMD241 | /5Phos/ACTTCAAGAGCTGAGGGCAANNNNN | 250 |
| | NNNNNCTTCAGCTTCCCGATTACGGGTACG | |
| | ATCCGACGGTAGTGTNNNNNNNNNNTTCA | |
| | CCAAATGGATTAAGATGTTC | |
| DMD242 | /5Phos/ATTCATGAACATCTTAATCCATTTGG | 251 |
| | TGNNNNNNNNNNCTTCAGCTTCCCGATTAC | |
| | GGGTACGATCCGACGGTAGTGTNNNNNNN | |
| | NNNTCTCTCTCACCCAGTCATCACTTCATA | |
| | G | |
| DMD243 | /5Phos/AGTCCAGGAGCTAGGTCAGGNNNNN | 252 |
| | NNNNNCTTCAGCTTCCCGATTACGGGTACG | |
| | ATCCGACGGTAGTGTNNNNNNNNNNTCTC | |
| | TCTCACCCAGTCATCAC | |
| DMD244 | /5Phos/GCAGATTTCAACCGGGCTTGNNNNN | 253 |
| | NNNNNCTTCAGCTTCCCGATTACGGGTACG | |
| | ATCCGACGGTAGTGTNNNNNNNNNNTTTC | |
| | CTTTTTGCAAAAACCCA | |
| DMD245 | /5Phos/AGCCAAACTCTTATTCATGACANNN | 254 |
| | NNNNNNNCTTCAGCTTCCCGATTACGGGTA | |
| | CGATCCGACGGTAGTGTNNNNNNNNNNAC | |
| | CACAGGTTGTGTCACCAG | |
| DMD246 | /5Phos/GTCACCCACCATCACCCTCTNNNNN | 255 |
| | NNNNNCTTCAGCTTCCCGATTACGGGTACG | |
| | ATCCGACGGTAGTGTNNNNNNNNNNAGTT | |
| | GCCTAAGAACTGGTGGG | |
| DMD247 | /5Phos/AATGAAGATTTTCCACCAATCACNN | 256 |
| | NNNNNNNNCTTCAGCTTCCCGATTACGGGT | |
| | ACGATCCGACGGTAGTGTNNNNNNNNNNT | |
| | ACCGACTGGCTTTCTCTGC | |
| DMD248 | /5Phos/TGTGTCACCAGAGTAACAGTCTGNN | 257 |
| | NNNNNNNNCTTCAGCTTCCCGATTACGGGT | |
| | ACGATCCGACGGTAGTGTNNNNNNNNNNA | |
| | AGCAGAGAAAGCCAGTCGG | |
| DMD249 | /5Phos/CGAGATGATCATCAAGCAGAAGGNN | 258 |
| | NNNNNNNNCTTCAGCTTCCCGATTACGGGT | |
| | ACGATCCGACGGTAGTGTNNNNNNNNNNG | |
| | TTGGAGGTACCTGCTCTGG | |
| DMD250 | /5Phos/TTGGGCAGCGGTAATGAGTTNNNNN | 259 |
| | NNNNNCTTCAGCTTCCCGATTACGGGTACG | |
| | ATCCGACGGTAGTGTNNNNNNNNNNTGAA | |
| | ACTTGTCATGCATCTTGC | |
| DMD251 | /5Phos/TGTGAGACCAGCCAAAACACTNNNN | 260 |
| | NNNNNNCTTCAGCTTCCCGATTACGGGTAC | |
| | GATCCGACGGTAGTGTNNNNNNNNNNTTC | |
| | AAATTTTGGGCAGCGGT | |
| DMD252 | /5Phos/AGACCAGCAATCAAGAGGCTNNNN | 261 |
| | NNNNNNCTTCAGCTTCCCGATTACGGGTAC | |
| | GATCCGACGGTAGTGTNNNNNNNNNNCAC | |
| | AACGCTGAAGAACCCTG | |
| DMD253 | /5Phos/CATCCCACTGATTCTGAATTCTTTCA | 262 |
| | ANNNNNNNNNNCTTCAGCTTCCCGATTAC | |
| | GGGTACGATCCGACGGTAGTGTNNNNNNN | |
| | NNNCTTGGTTTCTGTGATTTTCTTTTGGATT | |
| | G | |
| DMD254 | /5Phos/ATAGGGACCCTCCTTCCATGANNNN | 263 |
| | NNNNNNCTTCAGCTTCCCGATTACGGGTAC | |
| | GATCCGACGGTAGTGTNNNNNNNNNNACT | |
| | GTTCATTTCAGCTTTAACGTGA | |
| DMD255 | /5Phos/AAATGCTAGTCTGGAGGAGANNNNN | 264 |
| | NNNNNCTTCAGCTTCCCGATTACGGGTACG | |
| | ATCCGACGGTAGTGTNNNNNNNNNNGCCT | |
| | GTCCTAAGACCTGCTC | |
| DMD256 | /5Phos/CCAAAAGAAAATCACAGAAACCAA | 265 |
| | GGNNNNNNNNNNCTTCAGCTTCCCGATTA | |
| | CGGGTACGATCCGACGGTAGTGTNNNNNN | |
| | NNNNGAACCGGAGGCAACAGTTGA | |
| DMD257 | /5Phos/GGCTAGGATGATGAACAACAGGNN | 266 |
| | NNNNNNNNCTTCAGCTTCCCGATTACGGGT | |
| | ACGATCCGACGGTAGTGTNNNNNNNNNNG | |
| | GTGTTCTTGTACTTCATCCCAC | |
| DMD258 | /5Phos/ACCGGAGGCAACAGTTGAATNNNNN | 267 |
| | NNNNNCTTCAGCTTCCCGATTACGGGTACG | |
| | ATCCGACGGTAGTGTNNNNNNNNNNAGCA | |
| | ACATAAATGTGAGATAACGT | |
| DMD259 | /5Phos/TGGTGAAACTGGATGGACCANNNNN | 268 |
| | NNNNNCTTCAGCTTCCCGATTACGGGTACG | |
| | ATCCGACGGTAGTGTNNNNNNNNNNTTGG | |
| | CCCTGAAACTTCTCCG | |
| DMD260 | /5Phos/ATGTGGCAAATGACTTGGCCNNNNN | 269 |
| | NNNNNCTTCAGCTTCCCGATTACGGGTACG | |
| | ATCCGACGGTAGTGTNNNNNNNNNNTGAG | |
| | GATTCAGAAGCTGTTTACGA | |
| DMD261 | /5Phos/AGGTCTTTGGCCAACTGCTATNNNN | 270 |
| | NNNNNNCTTCAGCTTCCCGATTACGGGTAC | |
| | GATCCGACGGTAGTGTNNNNNNNNNNATG | |
| | AATGCTTCTCCAAGAGG | |
| DMD262 | /5Phos/TGAATGCTTCTCCAAGAGGCANNNN | 271 |
| | NNNNNNCTTCAGCTTCCCGATTACGGGTAC | |
| | GATCCGACGGTAGTGTNNNNNNNNNNAGA | |
| | AGTCTGAGCCAAGTCCG | |
| DMD263 | /5Phos/TACGGGTAGCATCCTGTAGGANNNN | 272 |
| | NNNNNNCTTCAGCTTCCCGATTACGGGTAC | |
| | GATCCGACGGTAGTGTNNNNNNNNNNTTT | |
| | GTCCCTGGCTTGTCAGT | |
| DMD264 | /5Phos/CACCCTGCAAAGGACCAAATGNNNN | 273 |
| | NNNNNNCTTCAGCTTCCCGATTACGGGTAC | |
| | GATCCGACGGTAGTGTNNNNNNNNNNGCC | |
| | TTTCCTTACGGGTAGCA | |
| DMD265 | /5Phos/GGGTGAGTTGTTGCTACAGCNNNNN | 274 |
| | NNNNNCTTCAGCTTCCCGATTACGGGTACG | |
| | ATCCGACGGTAGTGTNNNNNNNNNNTCTT | |
| | CCAAAGCAGCCTCTCG | |
| DMD266 | /5Phos/CCCCTGGACCTGGAAAAGTTNNNNN | 275 |
| | NNNNNCTTCAGCTTCCCGATTACGGGTACG | |
| | ATCCGACGGTAGTGTNNNNNNNNNNTGGA | |
| | GTTCACTAGGTGCACC | |
| DMD267 | /5Phos/TCAGGCATTTCCGCTTTAGCNNNNN | 276 |
| | NNNNNCTTCAGCTTCCCGATTACGGGTACG | |
| | ATCCGACGGTAGTGTNNNNNNNNNNTACT | |
| | GCAACAGTTCCCCCTG | |
| DMD268 | /5Phos/TCAAGTGGAGTGAACTTCGGANNNN | 277 |
| | NNNNNNCTTCAGCTTCCCGATTACGGTAC | |
| | GATCCGACGGTAGTGTNNNNNNNNNNTTC | |
| | TTCTTCCTGCTGTCCTGT | |
| DMD269 | /5Phos/ATGTGGAGCAAAAAGGCCACNNNN | 278 |
| | NNNNNNCTTCAGCTTCCCGATTACGGGTAC | |
| | GATCCGACGGTAGTGTNNNNNNNNNNTCC | |
| | TGAGATCCCTGGAAGGT | |
| DMD270 | /5Phos/TCCTACAGGACAGCAGGAAGANNN | 279 |
| | NNNNNNNCTTCAGCTTCCCGATTACGGGTA | |
| | CGATCCGACGGTAGTGTNNNNNNNNNNAA | |
| | CAGGACTGCATCATCGGA | |
| DMD271 | /5Phos/CGATGAATGTGAATTTGGAGAANNN | 280 |
| | NNNNNNNCTTCAGCTTCCCGATTACGGGTA | |
| | CGATCCGACGGTAGTGTNNNNNNNNNNTT | |
| | GGCTGTTTTCATCCAGGT | |
| DMD272 | /5Phos/AACAGGACTGCATCATCGGANNNNN | 281 |
| | NNNNNCTTCAGCTTCCCGATTACGGGTACG | |
| | ATCCGACGGTAGTGTNNNNNNNNNNTGTG | |
| | AGATACCAGTTACTTGTGCT | |
| DMD273 | /5Phos/CAAATCCCTTTTCTTGGCGTNNNNN | 282 |
| | NNNNNCTTCAGCTTCCCGATTACGGGTACG | |
| | ATCCGACGGTAGTGTNNNNNNNNNNAGCT | |
| | TCAATTTCACCTTGGAGG | |
| DMD274 | /5Phos/TGAGAGCCACAAAACAGAGGATNN | 283 |
| | NNNNNNNNCTTCAGCTTCCCGATTACGGGT | |
| | ACGATCCGACGGTAGTGTNNNNNNNNNNT | |
| | TCCACTGGTCAGAACTGGC | |
| DMD275 | /5Phos/AGCCACACCAGAAGTTCCTGNNNNN | 284 |
| | NNNNNCTTCAGCTTCCCGATTACGGGTACG | |
| | ATCCGACGGTAGTGTNNNNNNNNNNTGTG | |
| | CTTAACATGTGCAAGGC | |
| DMD276 | /5Phos/GAGGCGACTTTCCAGCAGTTNNNNN | 285 |
| | NNNNNCTTCAGCTTCCCGATTACGGGTACG | |
| | ATCCGACGGTAGTGTNNNNNNNNNNTCTG | |
| | ACATGGTACGCTGCTG | |
| DMD277 | /5Phos/CTCTTCTCACCCAAGGGTCANNNNN | 286 |
| | NNNNNCTTCAGCTTCCCGATTACGGGTACG | |
| | ATCCGACGGTAGTGTNNNNNNNNNNTCCA | |
| | GCAGTTCAGAAGCAGA | |
| DMD278 | /5Phos/CCCTCTTGAAGGCCTGTGAANNNNN | 287 |
| | NNNNNCTTCAGCTTCCCGATTACGGGTACG | |
| | ATCCGACGGTAGTGTNNNNNNNNNNCTGC | |
| | TCCGTCACCACTGATC | |
| DMD279 | /5Phos/ACCAGGAGCCCAGAGGTAATNNNN | 288 |
| | NNNNNNCTTCAGCTTCCCGATTACGGGTAC | |
| | GATCCGACGGTAGTGTNNNNNNNNNNTGA | |
| | GAAGAATGCCACAAGCCA | |
| DMD280 | /5Phos/CCTGGGTGCTCAGAACTTGTTNNNN | 289 |
| | NNNNNNCTTCAGCTTCCCGATTACGGGTAC | |
| | GATCCGACGGTAGTGTNNNNNNNNNNTCC | |
| | AAAGGCTGCTCTGTCAG | |
| DMD281 | /5Phos/CAGGGTCTGGATAGCTCTCANNNNN | 290 |
| | NNNNNCTTCAGCTTCCCGATTACGGGTACG | |
| | ATCCGACGGTAGTGTNNNNNNNNNNGAAA | |
| | CTCTACCAGGAGCCCAG | |
| DMD282 | /5Phos/TCAATGAGGAGATCGCCCACNNNNN | 291 |
| | NNNNNCTTCAGCTTCCCGATTACGGGTACG | |
| | ATCCGACGGTAGTGTNNNNNNNNNNTGTG | |
| | AAAGACGGACTGATTTCTCT | |
| DMD283 | /5Phos/AGGGCCCTTTGAGAGACTCANNNNN | 292 |
| | NNNNNCTTCAGCTTCCCGATTACGGGTACG | |
| | ATCCGACGGTAGTGTNNNNNNNNNNTGAG | |
| | ACCCTTGAAAGACTCC | |
| DMD284 | /5Phos/AAGCTGAGGTGATCAAGGGANNNN | 293 |
| | NNNNNNCTTCAGCTTCCCGATTACGGGTAC | |
| | GATCCGACGGTAGTGTNNNNNNNNNNAGA | |
| | GCCCAGAATGTCACTCG | |
| DMD285 | /5Phos/GGCATAAATTTTGATACAGCCCAGA | 294 |
| | NNNNNNNNNNCTTCAGCTTCCCGATTACG | |
| | GGTACGATCCGACGGTAGTGTNNNNNNNN | |
| | NNTTCTGGGCTCTCTCCTCAGG | |
| DMD286 | /5Phos/TTCTGGGCTCTCTCCTCAGGNNNNN | 295 |
| | NNNNNCTTCAGCTTCCCGATTACGGGTACG | |
| | ATCCGACGGTAGTGTNNNNNNNNNNCAGC | |
| | TTGAGGTCCAGCTCAT | |
| DMD287 | /5Phos/AAATTGAACCTGCACTCCGCNNNNN | 296 |
| | NNNNNCTTCAGCTTCCCGATTACGGGTACG | |
| | ATCCGACGGTAGTGTNNNNNNNNNNTGTG | |
| | GCCTAAAACCTTGTCA | |
| DMD288 | /5Phos/TCGAAGTGCCTGTGTGCAATNNNNN | 297 |
| | NNNNNCTTCAGCTTCCCGATTACGGGTACG | |
| | ATCCGACGGTAGTGTNNNNNNNNNNTGCA | |
| | GAAGCTTCCATCTGGT | |
| DMD289 | /5Phos/TGTTCATGGTAATATTTGTGAGGAN | 298 |
| | NNNNNNNNNCTTCAGCTTCCCGATTACGG | |
| | GTACGATCCGACGGTAGTGTNNNNNNNNN | |
| | NTCTGGAAGACCTGAACACCA | |
| DMD290 | /5Phos/AGCACATTGTAAACATTGTTGTCCTN | 299 |
| | NNNNNNNNNCTTCAGCTTCCCGATTACGG | |
| | GTACGATCCGACGGTAGTGTNNNNNNNNN | |
| | NCACGTCAATGACCTTGCTCG | |
| DMD291 | /5Phos/CACGTCAATGACCTTGCTCGNNNNN | 300 |
| | NNNNNCTTCAGCTTCCCGATTACGGGTACG | |
| | ATCCGACGGTAGTGTNNNNNNNNNNAGCA | |
| | AACATTACTGGCACTGC | |
| DMD292 | /5Phos/TGGTTGATAAGTTGAGAAGGTTAGG | 301 |
| | NNNNNNNNNNCTTCAGCTTCCCGATTACG | |
| | GGTACGATCCGACGGTAGTGTNNNNNNNN | |
| | NNATGAAGCCCACAGGGACTTT | |
| DMD293 | /5Phos/CCAGTAAGTCATTTTCAGCTTTTATC | 302 |
| | ACNNNNNNNNNNCTTCAGCTTCCCGATTA | |
| | CGGGTACGATCCGACGGTAGTGTNNNNNN | |
| | NNNNCTCCTTTTCCTCCCAGGTGG | |
| DMD294 | /5Phos/TGCTGAGATGCTGGACCAAANNNNN | 303 |
| | NNNNNCTTCAGCTTCCCGATTACGGGTACG | |
| | ATCCGACGGTAGTGTNNNNNNNNNNCAGG | |
| | ATGATTTATGCTTCTACTGC | |
| DMD295 | /5Phos/TCCAAGACTGAGAACACTAAAGCAN | 304 |
| | NNNNNNNNNCTTCAGCTTCCCGATTACGG | |
| | GTACGATCCGACGGTAGTGTNNNNNNNNN | |
| | NTTCATGCAGCTGCCTGACTC | |
| DMD296 | /5Phos/TCAAGTAAGTTGGAAGTATCACATT | 305 |
| | NNNNNNNNNNCTTCAGCTTCCCGATTACG | |
| | GGTACGATCCGACGGTAGTGTNNNNNNNN | |
| | NNAGCAAACAGACCAATATCAGTG | |
| DMD297 | /5Phos/GCCAAACAAAGTGCCCTACTNNNNN | 306 |
| | NNNNNCTTCAGCTTCCCGATTACGGGTACG | |
| | ATCCGACGGTAGTGTNNNNNNNNNNTGTC | |
| | TTCATGGGCAGCTGAG | |
| DMD298 | /5Phos/CCCTGGACAGACGCTGAAAANNNNN | 307 |
| | NNNNNCTTCAGCTTCCCGATTACGGGTACG | |
| | ATCCGACGGTAGTGTNNNNNNNNNNACAG | |
| | GTATTGTAGGCCAGGC | |
| DMD299 | /5Phos/CATCGCAAACAGGAAAGACANNNN | 308 |
| | NNNNNNCTTCAGCTTCCCGATTACGGGTAC | |
| | GATCCGACGGTAGTGTNNNNNNNNNNACA | |
| | GGTTAGTCACAATAAATGCTCT | |
| DMD300 | /5Phos/GCTTTTGAACCATTCGGAATNNNNN | 309 |
| | NNNNNCTTCAGCTTCCCGATTACGGGTACG | |
| | ATCCGACGGTAGTGTNNNNNNNNNNGCTC | |
| | TGTCATTTTGGGATGG | |
| DMD301 | /5Phos/TGCAGTGTGAAAGTTACTTGCTNNN | 310 |
| | NNNNNNNCTTCAGCTTCCCGATTACGGGTA | |
| | CGATCCGACGGTAGTGTNNNNNNNNNNTG | |
| | TGTTTTAGCCACGAGACT | |
| DMD302 | /5Phos/GGATGGTCCCAGCAAGTTGTNNNNN | 311 |
| | NNNNNCTTCAGCTTCCCGATTACGGGTACG | |
| | ATCCGACGGTAGTGTNNNNNNNNNNTGGA | |
| | TAGGAAGGTGCCACTG | |
| DMD303 | /5Phos/GCTGTCACAATTCCTGTTGCANNNN | 312 |
| | NNNNNNCTTCAGCTTCCCGATTACGGGTAC | |
| | GATCCGACGGTAGTGTNNNNNNNNNNAGG | |
| | ACTGCCATGAAACTCCG | |
| DMD304 | /5Phos/AGGACTGCCATGAAACTCCGNNNNN | 313 |
| | NNNNNCTTCAGCTTCCCGATTACGGGTACG | |
| | ATCCGACGGTAGTGTNNNNNNNNNNTATT | |
| | GGCAAATCACTGGGCG | |
| DMD305 | /5Phos/AAAGGGCCTTCTGCAGTCTTNNNNN | 314 |
| | NNNNNCTTCAGCTTCCCGATTACGGGTACG | |
| | ATCCGACGGTAGTGTNNNNNNNNNNAGGC | |
| | AAACTCTAGGCCAAGG | |
| DMD306 | /5Phos/AGGTCAGCTGAAAAGAGGGANNNN | 315 |
| | NNNNNNCTTCAGCTTCCCGATTACGGGTAC | |
| | GATCCGACGGTAGTGTNNNNNNNNNNTAC | |
| | ATTGCAACAGGAATTGTG | |
| DMD307 | /5Phos/ATAACAGACAACCCACCCCCNNNNN | 316 |
| | NNNNNCTTCAGCTTCCCGATTACGGGTACG | |
| | ATCCGACGGTAGTGTNNNNNNNNNNACTT | |
| | ACAGCAAAGGGCCTTCT | |
| DMD308 | /5Phos/ACCTTCCTTTCAGTGTCCTTNNNNNN | 317 |
| | NNNNCTTCAGCTTCCCGATTACGGGTACGA | |
| | TCCGACGGTAGTGTNNNNNNNNNNCTTGC | |
| | TCCAGGCGGTCATAA | |
| DMD309 | /5Phos/ACCACACTCTCTTTGAAAGGTGTNN | 318 |
| | NNNNNNNNCTTCAGCTTCCCGATTACGGGT | |
| | ACGATCCGACGGTAGTGTNNNNNNNNNNC | |
| | AGCTGACAGGCTCAAGAGA | |
| DMD310 | /5Phos/GCCCATGGATATCCTGCAGANNNNN | 319 |
| | NNNNNCTTCAGCTTCCCGATTACGGGTACG | |
| | ATCCGACGGTAGTGTNNNNNNNNNNAGGG | |
| | TATGAGAGAGTCCTAGCT | |
| DMD311 | /5Phos/TTCAGCAGCCAGTTCAGACANNNNN | 320 |
| | NNNNNCTTCAGCTTCCCGATTACGGGTACG | |
| | ATCCGACGGTAGTGTNNNNNNNNNNCTTC | |
| | CAGGGCCCTGTTGTAA | |
| DMD312 | /5Phos/ACAGGAGGCTTAGCGTACAGNNNNN | 321 |
| | NNNNNCTTCAGCTTCCCGATTACGGGTACG | |
| | ATCCGACGGTAGTGTNNNNNNNNNNTTAT | |
| | GACCGCCTGGAGCAAG | |
| DMD313 | /5Phos/TTGAGGTTGTGCTGGTCCAANNNNN | 322 |
| | NNNNNCTTCAGCTTCCCGATTACGGGTACG | |
| | ATCCGACGGTAGTGTNNNNNNNNNNTTCA | |
| | GCAGCCAGTTCAGACA | |
| DMD314 | /5Phos/CCTCCCTGTTCGTCCCCTAT | 323 |
| | NNNNCTTCAGCTTCCCGATTACGGGTACGA | |
| | TCCGACGGTAGTGTNNNNNNNNNNAAGAA | |
| | CAGTCTGTCATTTCCCATC | |
| DMD315 | /5Phos/ATCTGTACTTGTCTTCCAAATGTGCN | 324 |
| | NNNNNNNNNCTTCAGCTTCCCGATTACGG | |
| | GTACGATCCGACGGTAGTGTNNNNNNNNN | |
| | NTGACAAGGAATGGCACAAACC | |
| DMD316 | /5Phos/ACTGGCATCATTTCCCTGTGTNNNN | 325 |
| | NNNNNNCTTCAGCTTCCCGATTACGGGTAC | |
| | GATCCGACGGTAGTGTNNNNNNNNNNAGA | |
| | GTTCACACATCATTGAGCA | |
| DMD317 | /5Phos/TCATAAAATTTGGTTTGTGCCANNN | 326 |
| | NNNNNNNCTTCAGCTTCCCGATTACGGGTA | |
| | CGATCCGACGGTAGTGTNNNNNNNNNNTT | |
| | CATAATAGGGGACGAACAGG | |
| DMD318 | /5Phos/ACCACTGTTTTATTAAGATTGTTTTG | 327 |
| | ANNNNNNNNNNCTTCAGCTTCCCGATTAC | |
| | GGGTACGATCCGACGGTAGTGTNNNNNNN | |
| | NNNGACACGGATCCTCCCTGTTC | |
| DMD319 | /5Phos/ACAGCAGATTCCTCATGTAAGATGT | 328 |
| | NNNNNNNNNNCTTCAGCTTCCCGATTACG | |
| | GGTACGATCCGACGGTAGTGTNNNNNNNN | |
| | NNACTGGCATCATTTCCCTGTGT | |
| DMD320 | /5Phos/ACCCACAGAGCTTCGTTTTCTNNNN | 329 |
| | NNNNNNCTTCAGCTTCCCGATTACGGGTACG | |
| | ATCCGACGGTAGTGTNNNNNNNNNNACTTG | |
| | GCCTCCTTCTGCATGAT | |
| DMD321 | /5Phos/GGGCCTCCTTCTGCATGATTNNNNN | 330 |
| | NNNNNCTTCAGCTTCCCGATTACGGGTACG | |
| | ATCCGACGGTAGTGTNNNNNNNNNNACTG | |
| | GCTACTCTTGAGAATTGC | |
| DMD322 | /5Phos/AAATTGGAAGCAGCTCCGGANNNNN | 331 |
| | NNNNNCTTCAGCTTCCCGATTACGGGTACG | |
| | ATCCGACGGTAGTGTNNNNNNNNNNAACC | |
| | TAGAGTTCCAGAAGCTGC | |
| DMD323 | /5Phos/TGAACTTGCCACTTGCTTGANNNNN | 332 |
| | NNNNNCTTCAGCTTCCCGATTACGGGTACG | |
| | ATCCGACGGTAGTGTNNNNNNNNNNCTCC | |
| | GGACACTTGGCTCAAT | |
| DMD324 | /5Phos/GTGGGGTTACTTCTAATTTGTGCTNN | 333 |
| | NNNNNNNNCTTCAGCTTCCCGATTACGGGT | |
| | ACGATCCGACGGTAGTGTNNNNNNNNNNG | |
| | CGCTGGTCACAAAATCCTG | |
| DMD325 | /5Phos/CCAGCAGAACCTGACATCCANNNNN | 334 |
| | NNNNNCTTCAGCTTCCCGATTACGGGTACG | |
| | ATCCGACGGTAGTGTNNNNNNNNNNCCCC | |
| | CAAAGGATGCAACTTC | |
| DMD326 | /5Phos/GCTGGCTTTTCACAGCTTGTNNNNN | 335 |
| | NNNNNCTTCAGCTTCCCGATTACGGGTACG | |
| | ATCCGACGGTAGTGTNNNNNNNNNNCCGC | |
| | TTCGATCTCTGGCTTA | |
| DMD327 | /5Phos/GGAGAGAGAAGGAGGGCAAANNNN | 336 |
| | NNNNNNCTTCAGCTTCCCGATTACGGGTAC | |
| | GATCCGACGGTAGTGTNNNNNNNNNNCAT | |
| | TTGGCCTGATGCTTGGC | |
| DMD328 | /5Phos/ATCCAGTCTAGGAAGAGGGCCNNNN | 337 |
| | NNNNNNCTTCAGCTTCCCGATTACGGGTAC | |
| | GATCCGACGGTAGTGTNNNNNNNNNNTGG | |
| | ACACTCTTTGCAGATGTT | |
| DMD329 | /5Phos/GCCAGTTGCTGTTAGTTCGTACNNN | 338 |
| | NNNNNNNCTTCAGCTTCCCGATTACGGGTA | |
| | CGATCCGACGGTAGTGTNNNNNNNNNNCA | |
| | GAGTGGCTGCTGCAGAAA | |
| DMD330 | /5Phos/CAATGATTGGACACTCTTTGCANNN | 339 |
| | NNNNNNNCTTCAGCTTCCCGATTACGGGTA | |
| | CGATCCGACGGTAGTGTNNNNNNNNNNGG | |
| | AGGGTGACAGGAATGATCG | |
| DMD331 | /5Phos/TGGATGAGACTGGAACCCCANNNNN | 340 |
| | NNNNNCTTCAGCTTCCCGATTACGGGTACG | |
| | ATCCGACGGTAGTGTNNNNNNNNNNCACC | |
| | TCCTTTGCCATCTTGC | |
| DMD332 | /5Phos/ATGACATCTGCCAAAGCTGCNNNNN | 341 |
| | NNNNNCTTCAGCTTCCCGATTACGGGTACG | |
| | ATCCGACGGTAGTGTNNNNNNNNNNTGTG | |
| | GGACTAATGAACATTGCT | |
| DMD333 | /5Phos/GCACTATCCCATGGTGGAATNNNNN | 342 |
| | NNNNNCTTCAGCTTCCCGATTACGGGTACG | |
| | ATCCGACGGTAGTGTNNNNNNNNNNTTGG | |
| | GAATTTGATTCGAAGA | |
| DMD334 | /5Phos/GTGCTTTAGACTCCTGTACCTGANN | 343 |
| | NNNNNNNNCTTCAGCTTCCCGATTACGGGT | |
| | ACGATCCGACGGTAGTGTNNNNNNNNNNT | |
| | CAGGCTGGCGTCAAACTTA | |
| DMD335 | /5Phos/GCCTTTTGCAACTCGACCAGNNNNN | 344 |
| | NNNNNCTTCAGCTTCCCGATTACGGGTACG | |
| | ATCCGACGGTAGTGTNNNNNNNNNNTGAG | |
| | AGCCACTTTAGCTGGG | |
| DMD336 | /5Phos/GTGAGAGTTAGTTCACCTGGGANNN | 345 |
| | NNNNNNNCTTCAGCTTCCCGATTACGGGTA | |
| | CGATCCGACGGTAGTGTNNNNNNNNNNAT | |
| | GACATCTGCCAAAGCTGC | |
| DMD337 | /5Phos/TGTCCAGTTGCCACTTTCCCNNNNN | 346 |
| | NNNNNCTTCAGCTTCCCGATTACGGGTACG | |
| | ATCCGACGGTAGTGTNNNNNNNNNNAGAG | |
| | GGGGACAACATGGAAA | |
| DMD338 | /5Phos/CCTTGGCAAAGTCTCGAACANNNNN | 347 |
| | NNNNNCTTCAGCTTCCCGATTACGGGTACG | |
| | ATCCGACGGTAGTGTNNNNNNNNNNGGGT | |
| | GTTCAGCTGAGAGGAG | |
| DMD339 | /5Phos/TGGAATCAGACAAATGGGGCNNNN | 348 |
| | NNNNNNCTTCAGCTTCCCGATTACGGGTAC | |
| | GATCCGACGGTAGTGTNNNNNNNNNNACC | |
| | TTGGCAAAGTCTCGAAC | |
| DMD340 | /5Phos/ACGTTTCCATGTTGTCCCCCNNNNN | 349 |
| | NNNNNCTTCAGCTTCCCGATTACGGGTACG | |
| | ATCCGACGGTAGTGTNNNNNNNNNNGACG | |
| | TGGGAAAGTGGCAACT | |
| DMD341 | /5Phos/AGCAGAACACACTCTTGTTTGANNN | 350 |
| | NNNNNNNCTTCAGCTTCCCGATTACGGGTA | |
| | CGATCCGACGGTAGTGTNNNNNNNNNNTC | |
| | TCCCTTTTAGACTACATCAGGA | |
| DMD342 | /5Phos/ATTTTGCGAAGCATCCCCGANNNNN | 351 |
| | NNNNNCTTCAGCTTCCCGATTACGGGTACG | |
| | ATCCGACGGTAGTGTNNNNNNNNNNAACA | |
| | AGTGTCATGGGGCAGA | |
| DMD343 | /5Phos/TCTGGCCAGTAGATTCTGCGNNNNN | 352 |
| | NNNNNCTTCAGCTTCCCGATTACGGGTACG | |
| | ATCCGACGGTAGTGTNNNNNNNNNNACAC | |
| | CTTGGTTTGGCTATTGC | |
| DMD344 | /5Phos/TTTGCTGAAGGGTGCTGCTANNNNN | 353 |
| | NNNNNCTTCAGCTTCCCGATTACGGGTACG | |
| | ATCCGACGGTAGTGTNNNNNNNNNNTTTTT | |
| | GCGGCTGAGTTTGCG | |
| DMD345 | /5Phos/GCAATAGCCAAACCAAGGTGTNNNN | 354 |
| | NNNNNNCTTCAGCTTCCCGATTACGGGTAC | |
| | GATCCGACGGTAGTGTNNNNNNNNNNACG | |
| | CAGAATCTACTGGCCAG | |
| DMD346 | /5Phos/AGGAGACACACGCAAACTCANNNN | 355 |
| | NNNNNNCTTCAGCTTCCCGATTACGGGTAC | |
| | GATCCGACGGTAGTGTNNNNNNNNNNAAA | |
| | GAGAACCAAGCGAGCGA | |
| DMD347 | /5Phos/CCTCGTCCCCTCAGCTTTCANNNNN | 356 |
| | NNNNNCTTCAGCTTCCCGATTACGGGTACG | |
| | ATCCGACGGTAGTGTNNNNNNNNNNAGAA | |
| | TAAAAGCATTCTAGGCCA | |
| DMD348 | /5Phos/AACCCACCACACAGTTATGTTNNNN | 357 |
| | NNNNNNCTTCAGCTTCCCGATTACGGGTAC | |
| | GATCCGACGGTAGTGTNNNNNNNNNNTGC | |
| | CTGGCATACAACTAGTCT | |
| DMD349 | /5Phos/TGCGTGAATGAGTATCATCGTGNNN | 358 |
| | NNNNNNNCTTCAGCTTCCCGATTACGGGTA | |
| | CGATCCGACGGTAGTGTNNNNNNNNNNGA | |
| | ACGGCATGCACGTTAGAG | |
| DMD350 | /5Phos/CCCCAAACTTGTCTGATTCCTNNNN | 359 |
| | NNNNNNCTTCAGCTTCCCGATTACGGGTAC | |
| | GATCCGACGGTAGTGTNNNNNNNNNNCTT | |
| | ATAGGCCTGCCTCGTCC | |
| DMD351 | /5Phos/CCATTTGAGGCAGTGTGTGGNNNNN | 360 |
| | NNNNNCTTCAGCTTCCCGATTACGGGTACG | |
| | ATCCGACGGTAGTGTNNNNNNNNNNGCTG | |
| | TTTTCCATTTCTGCTAGC | |
| DMD52 | /5Phos/TTCCATTTCTGCTAGCCTGATNNNNN | 361 |
| | NNNNNCTTCAGCTTCCCGATTACGGGTACG | |
| | ATCCGACGGTAGTGTNNNNNNNNNNTCCT | |
| | GTGCTATCCTACCTCT | |
| DMD353 | /5Phos/TGAGAGCATGTAAGTATCCCANNNN | 362 |
| | NNNNNNCTTCAGCTTCCCGATTACGGGTAC | |
| | GATCCGACGGTAGTGTNNNNNNNNNNTCC | |
| | TTTCTCTTCTTGCCATGA | |
| DMD354 | /5Phos/GCTCCCCTCTTTCCTCACTCNNNNNN | 363 |
| | NNNNCTTCAGCTTCCCGATTACGGGTACGA | |
| | TCCGACGGTAGTGTNNNNNNNNNNCCTGG | |
| | CACTTTTCTATGTGTGC | |
| DMD355 | /5Phos/GGAAAGAGGGGAGCTAGAGAGNNN | 364 |
| | NNNNNNNCTTCAGCTTCCCGATTACGGGTA | |
| | CGATCCGACGGTAGTGTNNNNNNNNNNAC | |
| | CCCCAAAGCAAAATAAGG | |
| DMD356 | /5Phos/AAGTTTGAACCAGGACTCCCCNNNN | 365 |
| | NNNNNNCTTCAGCTTCCCGATTACGGGTAC | |
| | GATCCGACGGTAGTGTNNNNNNNNNNTCA | |
| | AATACACTCCTGAGTCCCT | |
| DMD357 | /5Phos/CCCCTTATTTTGCTTTGGGGGNNNNN | 366 |
| | NNNNNCTTCAGCTTCCCGATTACGGGTACG | |
| | ATCCGACGGTAGTGTNNNNNNNNNNAGCT | |
| | CCCCTCTTTCCTCACT | |
| DMD358 | /5Phos/TGTCATTGGTATGCAGAGTGCNNNN | 367 |
| | NNNNNNCTTCAGCTTCCCGATTACGGGTAC | |
| | GATCCGACGGTAGTGTNNNNNNNNNNCCT | |
| | CGTAGTCCTGCCCAGAT | |
| DMD359 | /5Phos/GCTTGCAGATTCCTATTGGCNNNNN | 368 |
| | NNNNNCTTCAGCTTCCCGATTACGGGTACG | |
| | ATCCGACGGTAGTGTNNNNNNNNNNCTCA | |
| | GCAATGAGCTCAGCAT | |
| DMD360 | /5Phos/GCAAGTGAGGAGAGAGATGGGNNN | 369 |
| | NNNNNNNCTTCAGCTTCCCGATTACGGGTA | |
| | CGATCCGACGGTAGTGTNNNNNNNNNNCC | |
| | CTCCTGAAATGATGCCCA | |
| DMD361 | /5Phos/GTGGGGACAGGCCTTTATGTNNNNN | 370 |
| | NNNNNCTTCAGCTTCCCGATTACGGGTACG | |
| | ATCCGACGGTAGTGTNNNNNNNNNNGCCT | |
| | GTGTAACTGTGACTCCA | |
| DMD362 | /5Phos/TGCTGCTGCTTTAGACGGTCNNNNN | 371 |
| | NNNNNCTTCAGCTTCCCGATTACGGGTACG | |
| | ATCCGACGGTAGTGTNNNNNNNNNNTGTG | |
| | GTCTTCCAGGATTTGCA | |
| DMD363 | /5Phos/AACCTCAGAGAGCACTTTTTATAGN | 372 |
| | NNNNNNNNNCTTCAGCTTCCCGATTACGG | |
| | GTACGATCCGACGGTAGTGTNNNNNNNNN | |
| | NCCAAGCTACTGCGTCAACAC | |
| DMD364 | /5Phos/AGCCTGTGTAACTGTGACTCCNNNN | 373 |
| | NNNNNNCTTCAGCTTCCCGATTACGGGTAC | |
| | GATCCGACGGTAGTGTNNNNNNNNNNCAC | |
| | TTTGCAGGCACATACCA | |
| DMD365 | /5Phos/CATCTGACTGCCACCGAAGANNNNN | 374 |
| | NNNNNCTTCAGCTTCCCGATTACGGGTACG | |
| | ATCCGACGGTAGTGTNNNNNNNNNNTGGG | |
| | GACAGGCCTTTATGTTC | |
| DMD366 | /5Phos/GGACATGAATATTTGGCCGTNNNNN | 375 |
| | NNNNNCTTCAGCTTCCCGATTACGGGTACG | |
| | ATCCGACGGTAGTGTNNNNNNNNNNTCCG | |
| | ACAGCAGTCAGCCTAT | |
| DMD367 | /5Phos/TGGCCGTAAGTGTTTGACTCANNNN | 376 |
| | NNNNNNCTTCAGCTTCCCGATTACGGGTAC | |
| | GATCCGACGGTAGTGTNNNNNNNNNNCAC | |
| | AACGGTGTCCTCTCCTT | |
| DMD368 | /5Phos/ACAACGGTGTCCTCTCCTTCNNNNN | 377 |
| | NNNNNCTTCAGCTTCCCGATTACGGGTACG | |
| | ATCCGACGGTAGTGTNNNNNNNNNNACAA | |
| | TCTTTGGGAGGGCTTCT | |
| DMD369 | /5Phos/GGGATATTTCACTGTTGATATAATCC | 378 |
| | ANNNNNNNNNNCTTCAGCTTCCCGATTAC | |
| | GGGTACGATCCGACGGTAGTGTNNNNNNN | |
| | NNNCCATTCACTTTGGCCTCTGC | |
| DMD370 | /5Phos/AGTCCGAAGTTTGACTGCCANNNNN | 379 |
| | NNNNNCTTCAGCTTCCCGATTACGGGTACG | |
| | ATCCGACGGTAGTGTNNNNNNNNNNTCAG | |
| | TGGCTCCCTGATACCA | |
| DMD371 | /5Phos/CCTGGGGCTAAGTCATCCAAANNNN | 380 |
| | NNNNNNCTTCAGCTTCCCGATTACGGGTAC | |
| | GATCCGACGGTAGTGTNNNNNNNNNNGTT | |
| | TGACTGCCAACCACTCG | |
| DMD372 | /5Phos/AACAAAGAAAACCCTCAAGCTTNNN | 381 |
| | NNNNNNNCTTCAGCTTCCCGATTACGGGTA | |
| | CGATCCGACGGTAGTGTNNNNNNNNNNCA | |
| | CCTCCTCTAACCCTGTGC | |
| DMD373 | /5Phos/GGAAGATCTTCTCAGTCCTCCCNNN | 382 |
| | NNNNNNNCTTCAGCTTCCCGATTACGGGTA | |
| | CGATCCGACGGTAGTGTNNNNNNNNNNTC | |
| | CCTTTAAAGAATTACTTCCTCA | |
| DMD374 | /5Phos/TGAGGAAGTAATTCTTTAAAGGGAN | 383 |
| | NNNNNNNNNCTTCAGCTTCCCGATTACGG | |
| | GTACGATCCGACGGTAGTGTNNNNNNNNN | |
| | NTGGGGAGGACTGAGAAGATCTT | |
| DMD375 | /5Phos/GAAAACAGATATTAAAGGGCCATGN | 384 |
| | NNNNNNNNNCTTCAGCTTCCCGATTACGG | |
| | GTACGATCCGACGGTAGTGTNNNNNNNNN | |
| | NGGAAGGAGTTGTTGAGTTGCTC | |
| DMD376 | /5Phos/GGAAGCCAACACGCAGTATCNNNNN | 385 |
| | NNNNNCTTCAGCTTCCCGATTACGGGTACG | |
| | ATCCGACGGTAGTGTNNNNNNNNNNTCTT | |
| | CTCAGTCCTCCCCAGG | |
| DMD377 | /5Phos/CCTGGGGAGGACTGAGAAGANNNN | 386 |
| | NNNNNNCTTCAGCTTCCCGATTACGGGTAC | |
| | GATCCGACGGTAGTGTNNNNNNNNNNTGG | |
| | CCTGATCCCAGCAAATC | |
| DMD378 | /5Phos/AGTTGCTCCATCACCTCCTCNNNNN | 387 |
| | NNNNNCTTCAGCTTCCCGATTACGGGTACG | |
| | ATCCGACGGTAGTGTNNNNNNNNNNCAAA | |
| | TCTTTTCACCATGGACCCA | |
| DMD379 | /5Phos/GGAGGTGATGGAGCAACTCANNNN | 388 |
| | NNNNNNCTTCAGCTTCCCGATTACGGGTAC | |
| | GATCCGACGGTAGTGTNNNNNNNNNNGGT | |
| | GTTAAAAATGTAATCATGGCCC | |
| DMD380 | /5Phos/ACGCGCATGTGTGTATTACANNNNN | 389 |
| | NNNNNCTTCAGCTTCCCGATTACGGGTACG | |
| | ATCCGACGGTAGTGTNNNNNNNNNNTCTC | |
| | TGCCTCTTCCTCTCTCT | |
| DMD381 | /5Phos/AGATGACCATTTATTCTCTGCTGGNN | 390 |
| | NNNNNNNNCTTCAGCTTCCCGATTACGGGT | |
| | ACGATCCGACGGTAGTGTNNNNNNNNNNC | |
| | TCATTGGCTTTCCAGGGGT | |
| DMD382 | /5Phos/CTCATTGGCTTTCCAGGGGTNNNNN | 391 |
| | NNNNNCTTCAGCTTCCCGATTACGGGTACG | |
| | ATCCGACGGTAGTGTNNNNNNNNNNTGTT | |
| | CCTCATGAGCTGCAAGT | |
| DMD383 | /5Phos/TCCACATGGCAGATGATTTGNNNNN | 392 |
| | NNNNNCTTCAGCTTCCCGATTACGGGTACG | |
| | ATCCGACGGTAGTGTNNNNNNNNNNCGAT | |
| | GCAGCTTCTGTGTTGT | |
| DMD384 | /5Phos/CTGTTTCTTTGCCATTTGGGANNNNN | 393 |
| | NNNNNCTTCAGCTTCCCGATTACGGGTACG | |
| | ATCCGACGGTAGTGTNNNNNNNNNNAACA | |
| | TTTATTCTGCTCCTTCTTCA | |
| DMD385 | /5Phos/GCATCACTCTGTTTCTTTGCCNNNNN | 394 |
| | NNNNNCTTCAGCTTCCCGATTACGGGTACG | |
| | ATCCGACGGTAGTGTNNNNNNNNNNTCTG | |
| | CTCCTTCTTCATCTGTCA | |
| DMD386 | /5Phos/TTACAAAAGGTGCAGATAGATAGCA | 395 |
| | TNNNNNNNNNNCTTCAGCTTCCCGATTACG | |
| | GGTACGATCCGACGGTAGTGTNNNNNNNN | |
| | NNGCGGGAATCAGGAGTTGTAA | |

In an experiment, 96 DNA samples are run through the DMD assay using the probe pool described in Table 3 and according to the following workflow. 31 of these samples are tested for DMD copy number variations, and the results of the 31 samples are shown in Table 4.

The workflow is outlined as follows:

Target Capture:

1. Prepare target capture, master mix:

Target Capture

| | |
|---|---|
| 98 C. | 5 min |

97 C.-57 C.
Touchdown
20% temp ramp speed
(~2 min/degree)

| | |
|---|---|
| 56 C. | 120 min |
| 4 C. | hold |

| Reagent | X1 | X110 |
|---|---|---|
| ~500-600 ng gDNA | 6.0 | — |
| Probe Pool v9.2 | 0.2 | 22 |
| 10X Ampligase Buffer | 2.0 | 220 |
| Water | 11.8 | 1298 |
| Total vol | 20.0 | 1540 |

2. Add 6 ul sample to 14 ul capture mix.

3. Thermocycler program: Target Capture

Extension/ligation:

4. Prepare extension/ligation master mix:

| Reagent | X1 | X110 |
|---|---|---|
| 10 mM dNTP | 0.6 | 72 |
| 100X NAD | 0.8 | 96 |
| 5M Betaine | 3.0 | 360 |
| 10X Ampligase Buff | 2.0 | 240 |
| Ampligase, 5 U/ul | 2.0 | 240 |
| Phusion Pol HF, 2 U/ul | 0.5 | 60 |
| water | 11.1 | 1332 |
| Total vol | 20.0 | 2400 |

Extension Ligation

| | |
|---|---|
| 56 C. | 60 min |
| 72 C. | 20 min |
| 37 C. | hold |

5. Add 20 ul extension/ligation mix to each sample.

6. Thermocycler program: Extension Ligation

Exonuclease Digestion:

7. Prepare Exonuclease master mix:

| Reagent | X1 | X110 |
|---|---|---|
| Exo I, 20 U/ul | 2 | 220 |
| Exo III, 100 U/ul | 2 | 220 |
| 10X NEBuffer 1.1 | 5 | 550 |
| Water | 1 | 110 |
| Total vol | 10 | 1100 |

Exonuclease Digestion

| | |
|---|---|
| 37 C. | 55 min |
| 90 C. | 40 min |
| 4 C. | forever |

8. Add 10 ul master mix to each reaction.

9. Thermocycler program: Exonuclease Digestion

10. Store samples at −20 C or proceed to PCR amplification.

PCT Amplification:

11. Prepare circular amplification PCR master mix:

| Reagent | X1 | X112 |
|---|---|---|
| CCCP circular DNA | 10 | — |
| 5X Phusion HF Buffer | 10 | 1200 |
| 10 mM dNTPs | 1 | 120 |
| Phusion Pol HS, 2 U/ul | 1 | 120 |
| FW Primer (100 uM) | 0.25 | 30 |
| REV Primers (5 uM) | 5 | — |
| water | 22.75 | 2730 |
| Total vol | 50 | 4200 |

PCR amplification

| | | |
|---|---|---|
| 95 C. | 2 min | |
| 98 C. | 15 sec | 24 Cycles |
| 65 C. | 15 sec | |
| 72 C. | 15 sec | |
| 72 C. | 5 min | |
| 4 C. | forever | |

12. Add 10 ul sample to 5 ul REV primer to 35 ul PCR mix

13. Thermocycler program: DMD PCR amplification

14. Purify amplified products using Ampure beads. 5 ul from each sample is pooled and 45 ul of the pool is mixed with 45 ul Ampure beads. After 5 minutes, samples are washed twice with 180 ul 70% EtOH, dried for 5 minutes, and the pellet is resuspended in 35 ul EB buffer. 32 ul supernatant is removed and transferred to a clean 1.5 ml LoBind DNA tube. This tube contains the final purified library. The purified pool is QC' d using the Qubit assay, before loading on to the MiSeq sequencing platform.

Following the above-described 14-step assay, the pooled 96 sample library is sequenced on an Illumina MiSeq instrument using 125 cycles of paired end sequencing. Resultant reads are processed by trimming, filtering and flagging the reads until they are aligned to the genome. The number of unique molecular tags originating from each DMD probe that aligned to the target region are counted, and may be referred to herein as u_{DMD}. To calculate a probe capture metric for each DMD probe, this number of unique molecular tags (u_{DMD}) is normalized by a normalization factor that may include the total number of unique molecular tags across the entire sample. In an example, the normalization factor is represented by the denominator of EQ. 1. In another example, the normalization factor that is used to normalize u_{DMD} may only include the sum of the control capture events in EQ. 1, or the sum of u_{CONTROL i,s} where i=1, 2 . . . . J, where J is the number of control populations used in the sample s. The resulting probe capture metric is then normalized again to reflect the presence of one or two copies in known normal samples. In particular, since DMD is on the X chromosome, normal male samples are expected to have one copy, and normal female samples are expected to have two copies. As an example, the probe capture metric may be normalized (to have a mean of one or two, for example) based on the status of the control population, or prior knowledge of the sample copy number in the known samples. In another example, if the copy number of the sample is unknown, then a normalization process similar to step 526 may be performed. In particular, the probe capture metric may be normalized by a composite control population.

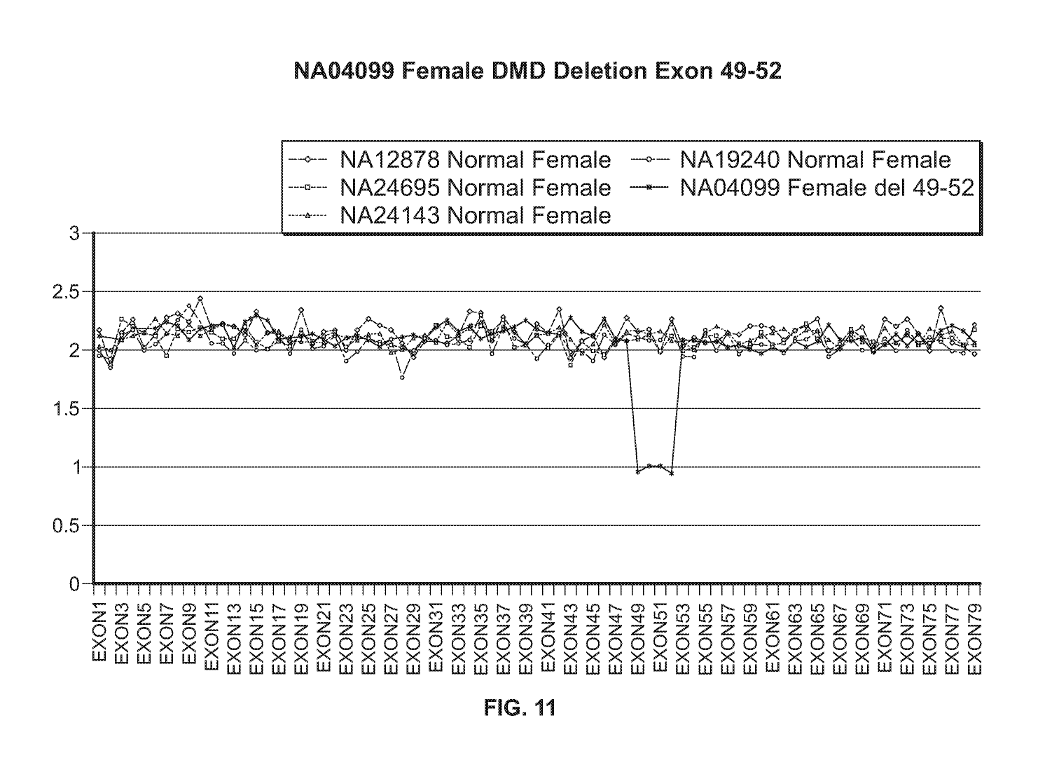

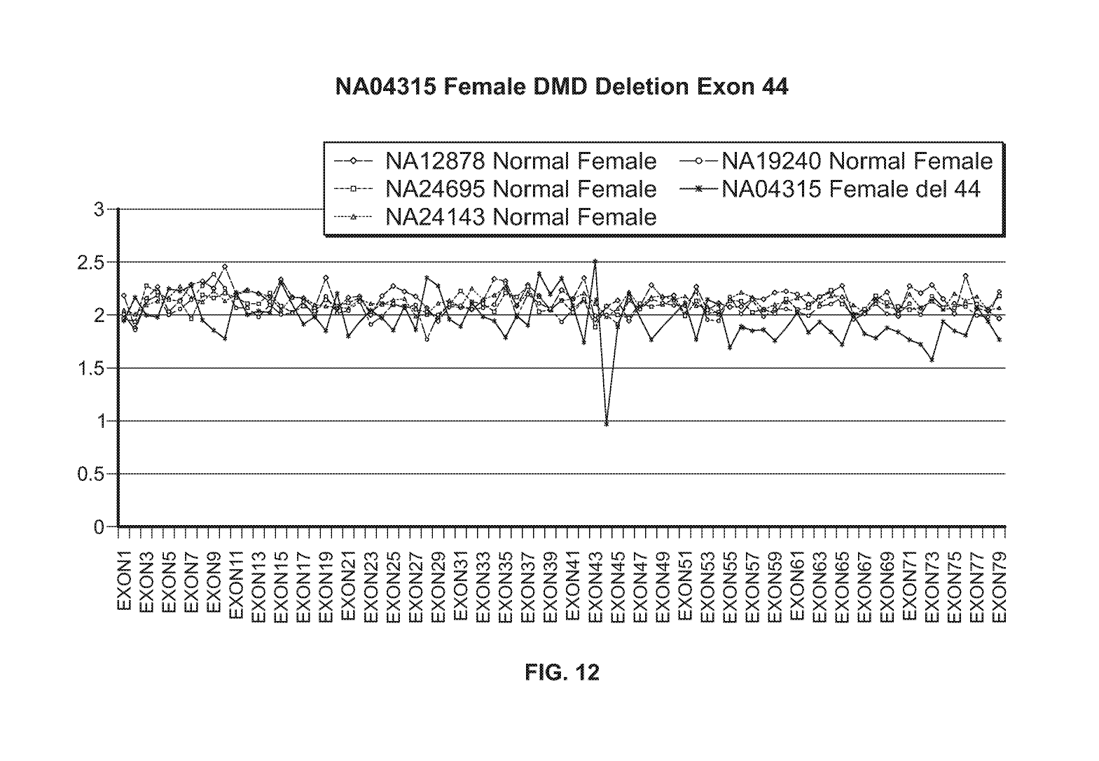

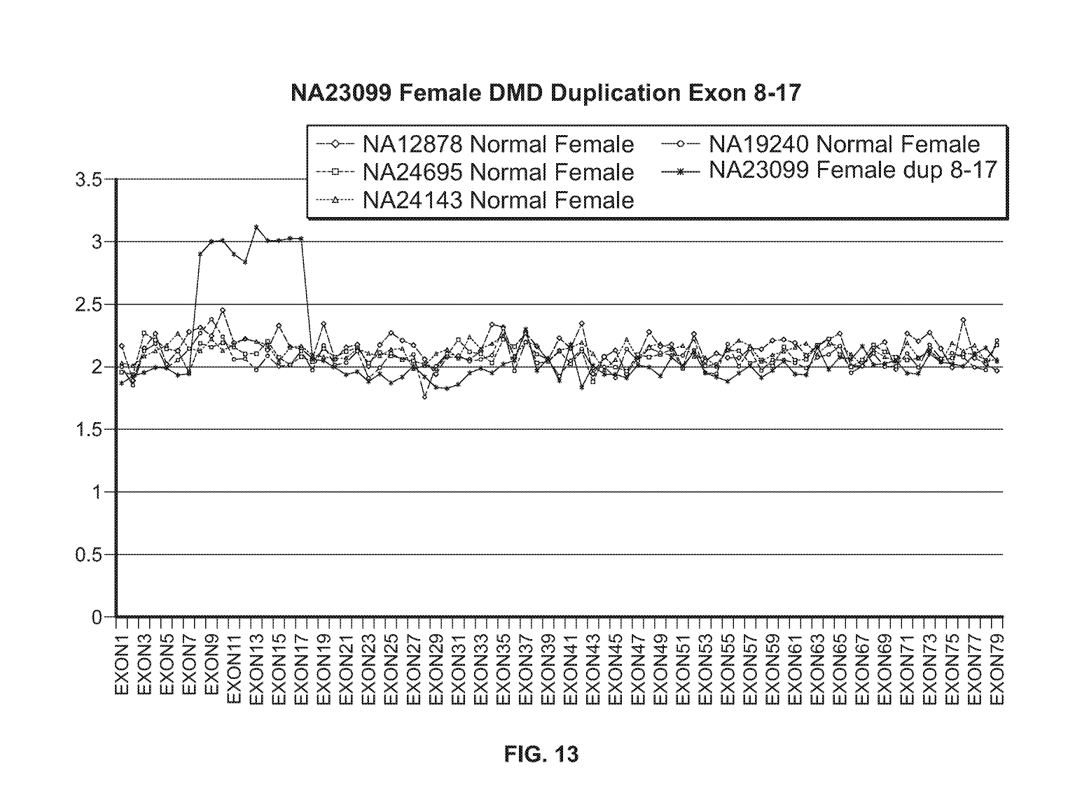

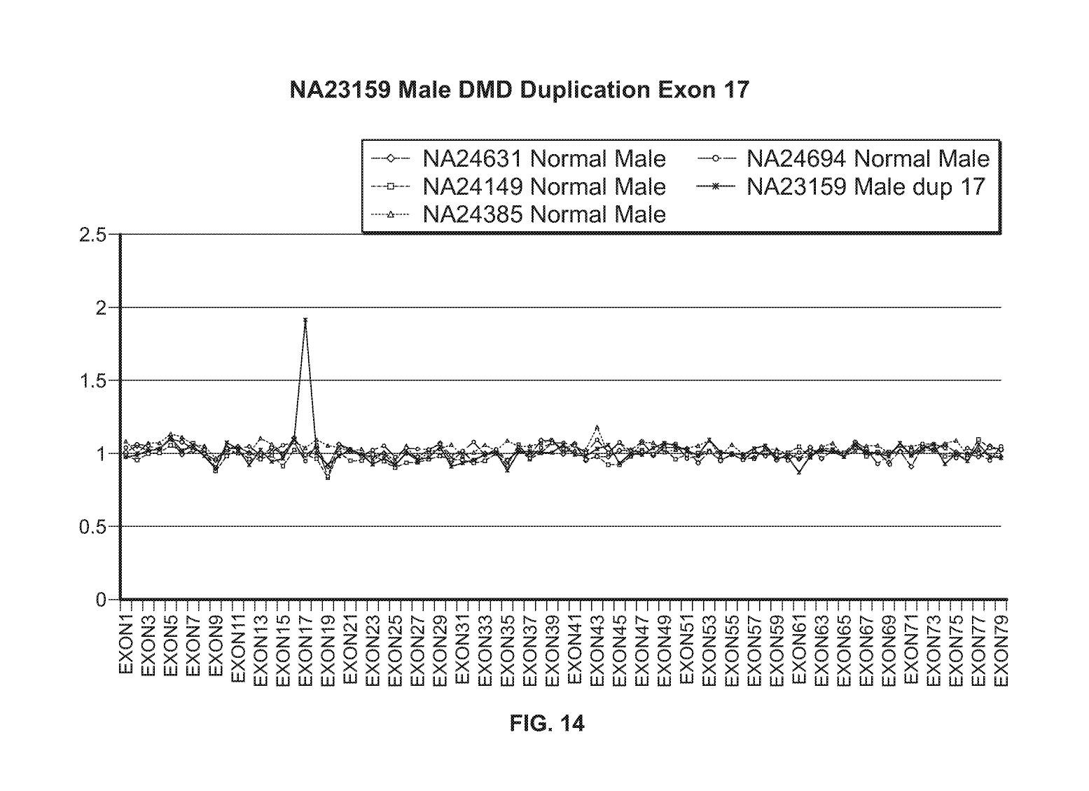

The resulting normalized probe capture metrics (where u_{DMD} was normalized by u_{CONTROL} and the resulting probe capture metrics were normalized based on the status of the control population) are averaged for each exon, and the averaged values are then plotted for all 79 exons in the DMD gene, as is shown in FIGS. 11-14. The results are displayed graphically, where the y-axis indicates the normalized probe capture metrics and the x-axis indicates the exon in the DMD gene. As a reference, each graph in FIGS. 11-14 includes four normal female samples (for FIGS. 11-13) or four normal male samples (for FIG. 14). A data point significantly higher than the reference values indicates a duplication for the corresponding exon, and a data point significantly lower than the reference values indicates a deletion for the corresponding exon. As is shown in FIG. 11, a female (sample NA04099) exhibits DMD deletion at multiple exons 49-52. As is shown in FIG. 12, a female (sample NA04315) exhibits DMD deletion at a single exon 44. As is shown in FIG. 13, a female (sample NA23099) exhibits DMD duplication at multiple exons 8-17. As is shown in FIG. 14, a male (sample NA23159) exhibits DMD duplication at a single exon 17. The assay correctly identifies exon level deletions/duplications in all 31 samples listed below in Table 4.

TABLE 4

| | | | DMD |
|---|---|---|---|
| | Sample | Gender | status |
| 1 | NA04099 | Female | del 49-52 |
| 2 | NA04315 | Female | del 44 |
| 3 | NA23099 | Female | dup 8-17 |
| 4 | NA05117 | Female | del 45 |
| 5 | NA05159 | Female | del 46-50 |
| 6 | NA05174 | Female | del 4-43 |
| 7 | NA09982 | Female | dup 2-4 |
| 8 | NA23087 | Female | dup 2-30 |
| 9 | NA23094 | Female | del 35-43 |
| 10 | NA07692 | Female | del 5′ end-18 |
| 11 | NA02339 | Male | del 31-43 |
| 12 | NA03604 | Male | del 18-41 |
| 13 | NA03780 | Male | del 3-17 |
| 14 | NA03929 | Male | del 46-50 |
| 15 | NA04100 | Male | del 49-52 |
| 16 | NA04327 | Male | dup 5-7 |
| 17 | NA04364 | Male | del 51-55 |
| 18 | NA04981 | Male | del 45-53 |
| 19 | NA05016 | Male | del 45-50 |
| 20 | NA05089 | Male | del 3-5 |
| 21 | NA05115 | Male | del 45 |
| 22 | NA05170 | Male | del 4-43 |
| 23 | NA05124 | Male | dup 45-62 |
| 24 | NA07691 | Male | del 5′ end-18 |
| 25 | NA07947 | Male | del 5′ end-30 |
| 26 | NA09981 | Male | dup 2-4 |
| 27 | NA10283 | Male | del 72-79 |
| 28 | NA23086 | Male | dup 2-30 |
| 29 | NA23096 | Male | del 35-43 |
| 30 | NA23127 | Male | dup 27-28 |
| 31 | NA23159 | Male | dup 17 |

For illustrative purposes, the examples provided by this disclosure focus primarily on a number of different example embodiments of systems and methods to determine copy number variations, chromosomal abnormalities, or micro-deletions. However, it is understood that variations in the general shape and design of one or more embodiments may be made without significantly changing the functions and operations of the present disclosure. Furthermore, it should be noted that the features and limitations described in any one embodiment may be applied to any other embodiment herein, and the descriptions and examples relating to one embodiment may be combined with any other embodiment in a suitable manner. Moreover, the figures and examples provided in disclosure are intended to be only exemplary, and not limiting. It should also be noted that the systems and/or methods described above may be applied to, or used in accordance with, other systems and/or methods, including systems and/or methods which may or may not be directly related to determining copy number variations.

What is claimed is:

1. A method of detecting copy number variation in a subject comprising:
a) obtaining a nucleic acid sample isolated from the subject;
b) capturing one or more target sequences in the nucleic acid sample obtained in step a) by using one or more target populations of targeting molecular inversion probes (MIPs) to produce a plurality of targeting MIPs replicons for each target sequence,
wherein each of the targeting MIPs in each of the target populations comprises in sequence the following components:
first targeting polynucleotide arm—first unique targeting molecular tag—polynucleotide linker—second unique targeting molecular tag—second targeting polynucleotide arm;
wherein the pair of first and second targeting polynucleotide arms in each of the targeting MIPs in each target population are identical, and are substantially complementary to first and second regions in the nucleic acid that, respectively, flank the target sequence that is targeted by the one or more targeting MIPs;
wherein the first and second unique targeting molecular tags in each of the targeting MIPs in each target population are distinct in each of the targeting MIPs, in each member of the target population, and in each of the target populations;
c) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),
wherein each of the control MIPs in each control population comprises in sequence the following components:
first control polynucleotide arm—first unique control molecular tag—polynucleotide linker—second unique control molecular tag—second control polynucleotide arm;
wherein the pair of first and second control polynucleotide arms in each of the control MIPs in each control population are identical, and are substantially complementary to first and second regions in the nucleic acid that, respectively, flank each control sequence;
wherein the first and second unique control molecular tags in each of the control MIPs in each control population are distinct in each of the control MIPs and in each member of the control population, and are different from the unique targeting molecular tags;
d) sequencing the targeting and control MIPs amplicons that are amplified from the targeting and control MIPs replicons obtained in steps b) and c);
e) determining, for each target population, the number of the unique targeting molecular tags present in the targeting MIPs amplicons sequenced in step d);
f) determining, for each control population, the number of the unique control molecular tags present in the control MIPs amplicons sequenced in step d);
g) computing a target probe capture metric, for each of the one or more target sequences, based at least in part on the number of the unique targeting molecular tags determined in step e) and a plurality of control probe capture metrics based at least in part on the numbers of the unique control molecular tags determined in step f);
h) identifying a subset of the control populations of control MIPs that have control probe capture metrics satisfying at least one criterion;
i) normalizing each of the one or more target probe capture metrics by a factor computed from the subset of control probe capture metrics satisfying the at least one criterion, to obtain a test normalized target probe capture metric for each of the one or more target sequences;
j) comparing each test normalized target probe capture metric obtained in step i) to a plurality of reference normalized target probe capture metrics that are computed based on reference nucleic acid samples obtained from reference subjects exhibiting known genotypes using the same target and control sequences, target population, one subset of control populations in steps b)-g) and i); and
k) determining, based on the comparing in step j) and the known genotypes of reference subjects, the copy number variation of each of the one or more target sequences of interest.

2. The method of claim 1, wherein the nucleic acid sample is DNA or RNA.

3. The method of claim 1 or 2, wherein the nucleic acid sample is genomic DNA.

4. The method of any one of claims 1-3, wherein the subject is a carrier screening candidate for one or more diseases or conditions.

5. The method of any one of claims 1-3, wherein the subject is a candidate for:
a) a pharmacogenomics test;
b) a targeted tumor test; or
c) an exonic deletion test.

6. The method of any one of claims 1-5, wherein the length of each of the targeting polynucleotide arms is between 18 and 35 base pairs.

7. The method of any one of claims 1-5, wherein the length of each of the control polynucleotide arms is between 18 and 35 base pairs.

8. The method of any one of claims 1-7, wherein each of the targeting polynucleotide arms has a melting temperature between 57° C. and 63° C.

9. The method of any one of claims 1-7, wherein each of the control polynucleotide arms has a melting temperature between 57° C. and 63° C.

10. The method of any one of claims 1-9, wherein each of the targeting polynucleotide arms has a GC content between 30% and 70%.

11. The method of any one of claims 1-9, wherein each of the control polynucleotide arms has a GC content between 30% and 70%.

12. The method of any one of claims 1-11, wherein the length of each of the unique targeting molecular tags is between 12 and 20 base pairs.

13. The method of any one of claims 1-11, wherein the length of each of the unique control molecular tags is between 12 and 20 base pairs.

14. The method of any one of claims 1-13, wherein each of the unique targeting or control molecular tags is not substantially complementary to any genomic region of the subject.

15. The method of any one of claims 1-13, wherein the polynucleotide linker is not substantially complementary to any genomic region of the subject.

16. The method of any one of claims 1-15, wherein the polynucleotide linker has a length of between 30 and 40 base pairs.

17. The method of any one of claims 1-15, wherein the polynucleotide linker has a melting temperature of between 60° C. and 80° C.

18. The method of any one of claims 1-15, wherein the polynucleotide linker has a GC content between 30% and 70%.

19. The method of any one of claims 1-15, wherein the polynucleotide linker comprises 5′-CTTCAGCTTCCCGATATCCGACGGTAGTGT-3′(SEQ ID NO: 1)

20. The method of any one of claims 1-19, wherein the plurality of target population of targeting MIPs and the plurality of control populations of control MIPs are in a probe mixture.

21. The method of claim 20, wherein the probe mixture has a concentration between 1-100 pM; 10-100 pM; 50-100 pM; or 10-50 pM.

22. The method of any one of claims 1-21, wherein each of the targeting MIPs replicons is a single-stranded circular nucleic acid molecule.

23. The method of claim 22, wherein each of the targeting MIPs replicons provided in step b) is produced by:
iii) the first and second targeting polynucleotide arms, respectively, hybridizing to the first and second regions in the nucleic acid that, respectively, flank the target sequence; and
iv) after the hybridization, using a ligation/extension mixture to extend and ligate the gap region between the two targeting polynucleotide arms to form single-stranded circular nucleic acid molecules.

24. The method of any one of claims 1-23, wherein each of the control MIPs replicons is a single-stranded circular nucleic acid molecule.

25. The method of claim 24, wherein each of the control MIPs replicons provided in step b) is produced by:
iii) the first and second control polynucleotide arms, respectively, hybridizing to the first and second regions in the nucleic acid that, respectively, flank the control sequence; and
iv) after the hybridization, using a ligation/extension mixture to extend and ligate the gap region between the two control polynucleotide arms to form single-stranded circular nucleic acid molecules.

26. The method of any one of claims 1-25, wherein the sequencing step of d) comprises a next-generation sequencing method.

27. The method of claim 26, wherein the next-generation sequencing method comprises a massive parallel sequencing method, or a massive parallel short-read sequencing method.

28. The method of any one of claims 1-27, wherein the method comprises, before the sequencing step of d), a PCR reaction to amplify the targeting and control MIPs replicons to produce the targeting and control MIPs amplicons for sequencing.

29. The method of claim 28, wherein the PCR reaction is an indexing PCR reaction.

30. The method of claim 29, wherein the indexing PCR reaction introduces, the following components: a pair of indexing primers, a unique sample barcode and a pair of sequencing adaptors, into each of the targeting or control MIPs replicons to produce barcoded targeting or control MIPs amplicons.

31. The method of claim 30, wherein the barcoded targeting MIPs amplicons comprise in sequence the following components:
a first sequencing adaptor—a first sequencing primer—the first unique targeting molecular tag—the first targeting polynucleotide arm—captured target nucleic acid—the second targeting polynucleotide arm—the second unique targeting molecular tag—a unique sample barcode—a second sequencing primer—a second sequencing adaptor; or
wherein the barcoded control MIPs amplicons comprise in sequence the following components:
a first sequencing adaptor—a first sequencing primer—the first unique control molecular tag—the first control polynucleotide arm—captured control nucleic acid—the second control polynucleotide arm—the second unique control molecular tag—a unique sample barcode—a second sequencing primer—a second sequencing adaptor.

32. The method of any one of claims 1-31, wherein at least one of the one or more target sequences and at least one of the control sequences are on the same chromosome.

33. The method of any one of claims 1-31, wherein at least one of the one or more target sequences and at least one of the control sequences are on different chromosomes.

34. The method of any one of claims 1-33, wherein the target sequence is SMN1/SMN2.

35. The method of claim 34, wherein the first targeting polynucleotide primer for the target sequence of SMN1/SMN2 comprises the sequence of 5′-AGG AGT AAG TCT GCC AGC ATT-3′ (SEQ ID NO: 2).

36. The method of claim 34 or 35, wherein the second targeting polynucleotide primer for the target sequence of SMN1/SMN2 comprises the sequence of 5′-AAA TGT CTT GTG AAA CAA AAT GCT-3′ (SEQ ID NO: 3).

37. The method of any one of claims 34-36, wherein the polynucleotide linker comprises 5′-CTT CAG CTT CCC GAT ATC CGA CGG TAG TGT-3′ (SEQ ID NO: 1).

38. The method of any one of claims 34-37, wherein the MIP for the target sequence of SMN1/SMN2 comprises the sequence of 5′-AGG AGT AAG TCT GCC AGC ATT NNN NNN NNN NCT TCA GCT TCC CGA TTA CGG GTA CGA TCC GAC GGT AGT GTN NNN NNN NNN AAA TGT CTT GTG AAA CAA AAT GCT-3′ (SEQ ID NO: 4).

39. The method of any one of claims 1-38, wherein the control sequences comprise one or more genes or sequences selected from the group consisting of CFTR, HEXA, HFE, HBB, BLM, IDS, IDUA, LCA5, LPL, MEFV, GBA, MPL, PEX6, PCCB, ATM, NBN, FANCC, F8, CBS, CPT1, CPT2, FKTN, G6PD, GALC, ABCC8, ASPA, MCOLN1, SPMD1, CLRN1, NEB, G6PC, TMEM216, BCKDHA, BCKDHB, DLD, IKBKAP, PCDH15, TTN, GAMT, KCNJ11, IL2RG, and GLA.

40. A method of detecting copy number variation in a subject comprising:
a) isolating a genomic DNA sample from the subject;
b) adding the genomic DNA sample into each well of a multi-well plate, wherein each well of the multi-well plate comprises a probe mixture, wherein the probe mixture comprises a plurality of target populations of targeting molecular inversion probes (MIPs), a plurality of control populations of control MIPs and buffer;
wherein each targeting population of targeting MIPs is capable of amplifying a distinct target sequence in the genomic DNA sample obtained in step a),
wherein each of the targeting MIPs in each target population comprises in sequence the following components:
first targeting polynucleotide arm—first unique targeting molecular tag—polynucleotide linker—second unique targeting molecular tag—second targeting polynucleotide arm;
wherein the pair of first and second targeting polynucleotide arms in each of the targeting MIPs in each target population are identical, and are substantially complementary to first and second regions in the genomic DNA that, respectively, flank each target sequence;
wherein the first and second unique targeting molecular tags in each of the targeting MIPs in each target population are distinct in each of the targeting MIPs and in each member of the target population;
wherein each control population of control MIPs is capable of amplifying a distinct control sequence in the genomic DNA sample obtained in step a),
wherein each of the control MIPs in each control population comprises in sequence the following components:
first control polynucleotide arm—first unique control molecular tag—polynucleotide linker—second unique control molecular tag—second control polynucleotide arm;
wherein the pair of first and second control polynucleotide arms in each of the control MIPs in each control population are identical, and are substantially complementary to first and second regions in the genomic DNA that, respectively, flank each control sequence;
wherein the first and second unique control molecular tags in each of the control MIPs in each control population are distinct in each of the control MIPs and in each member of the control population, and are different from the unique targeting molecular tags;
c) incubating the genomic DNA sample with the probe mixture for the targeting MIPs to capture the target sequence and for the control MIPs to capture the control sequences;
d) adding an extension/ligation mixture to the sample of c) for the targeting MIPs and the captured target sequence to form the targeting MIPs replicons and for the control MIPs and the captured control sequences to form the control MIPs replicons, wherein the extension/ligation mixture comprises a polymerase, a plurality of dNTPs, a ligase, and buffer;
e) adding an exonuclease mixture to the targeting and control MIPs replicons to remove excess probes or excess genomic DNA;
f) adding an indexing PCR mixture to the sample of e) to add a pair of indexing primers, a unique sample barcode and a pair of sequencing adaptors to the targeting and control MIPs replicons to produce the targeting and control MIPs amplicons;
g) using a massively parallel sequencing method to determine, for each target population, the number of the unique targeting molecular tags present in the barcoded targeting MIPs amplicons provided in step f);
h) using a massively parallel sequencing method to determine, for each control population, the number of the unique control molecular tags present in the barcoded control MIPs amplicons provided in step f);
i) computing a target probe capture metric for each target sequence based at least in part on the number of the unique targeting molecular tags determined in step g) and a plurality of control probe capture metrics based at least in part on the numbers of the unique control molecular tags determined in step h);
j) identifying a subset of the control populations of control MIPs that have control probe capture metrics satisfying at least one criterion;
k) normalizing each target probe capture metric by a factor computed from the subset of control probe capture metrics satisfying the at least one criterion, to obtain a test normalized target probe capture metric for each target sequence;
l) comparing each test normalized target probe capture metric to a plurality of reference normalized target probe capture metrics that are computed based on reference genomic DNA samples obtained from reference subjects exhibiting known genotypes using the same target and control sequences, target population, one subset of control populations in steps b)-h); and
m) determining, based on the comparing in step l) and the known genotypes of reference subjects, the copy number variation for each target sequence.

41. A nucleic acid molecule comprising the sequence of:
(SEQ ID NO: 4)
5′-AGG AGT AAG TCT GCC AGC ATT NNN NNN NNN NCT
TCA GCT TCC CGA TTA CGG GTA CGA TCC GAC GGT AGT
GTN NNN NNN NNN AAA TGT CTT GTG AAA CAA AAT
GCT-3′.

42. The nucleic acid molecule of claim 41, wherein the nucleic acid is 5′ phosphorylated.

43. A method for producing a genotype cluster, the method comprising:
a) receiving sequencing data obtained from a plurality of nucleic acid samples from a plurality of subsets of a plurality of subjects, each sample in the plurality of samples being obtained from a different subject, and each subset being characterized by subjects exhibiting a same known genotype for a gene of interest, wherein the sequencing data for the nucleic acid sample from each subject in the plurality of subsets is obtained by:
i) obtaining a nucleic acid sample isolated from the subject;
ii) capturing one or more target sequences of interest in the nucleic acid sample obtained in step a.i) by using one or more target populations of targeting molecular inversion probes (MIPs) to produce targeting MIPs replicons for each target sequence,
wherein each of the targeting MIPs in each of the target populations comprises in sequence the following components:
first targeting polynucleotide arm—first unique targeting molecular tag—polynucleotide linker—second unique targeting molecular tag—second targeting polynucleotide arm;
wherein the pair of first and second targeting polynucleotide arms in each of the targeting MIPs in each target population are identical, and are substantially complementary to first and second regions in the nucleic acid that, respectively, flank the target sequence of interest that is targeted by the one or more targeting MIPs;
wherein the first and second unique targeting molecular tags in each of the targeting MIPs in each target population are distinct in each of the targeting MIPs and in each member of the target population;
iii) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),
wherein each of the control MIPs in each control population comprises in sequence the following components:
first control polynucleotide arm—first unique control molecular tag—polynucleotide linker—second unique control molecular tag—second control polynucleotide arm;
wherein the pair of first and second control polynucleotide arms in each of the control MIPs in each control population are identical, and are substantially complementary to first and second regions in the nucleic acid that, respectively, flank each control sequence;
wherein the first and second unique control molecular tags in each of the control MIPs in each control population are distinct in each of the control MIPs and in each member of the control population, and are different from the unique targeting molecular tags;
iv) sequencing the targeting and control MIPs amplicons that are amplified from the targeting and control MIPs replicons obtained in steps a.ii) and a.iii);
b) for each respective sample obtained from a subset in the plurality of subsets:
i) determining, for each target population, the number of the unique targeting molecular tags present in the targeting MIPs amplicons sequenced in step a.iv);
ii) determining, for each control population, the number of the unique control molecular tags present in the control MIPs amplicons sequenced in step a.iv);
iii) computing a target probe capture metric, for each target sequence, based at least in part on the number of the unique targeting molecular tags determined in step b.i) and a plurality of control probe capture metrics based at least in part on the numbers of the unique control molecular tags determined in step b.ii);
iv) identifying a subset of the control populations of control MIPs that have control probe capture metrics satisfying at least one criterion;
v) normalizing each target probe capture metric by a factor computed from the control probe capture metrics satisfying the at least one criterion, to obtain a normalized target probe capture metric for each of the one or more target sites; and
c) grouping, across the samples obtained from each subset of subjects, the normalized target probe capture metrics to obtain the genotype cluster for the known genotype.

44. The method of claim 43, wherein computing the target probe capture metric at step b.iii) comprises normalizing the number of the unique targeting molecular tags determined in step b.i) by a sum of the number of the unique targeting molecular tags and the numbers of the unique control molecular tags.

45. The method of claim 43, wherein computing the plurality of control probe capture metrics at step b.iii) comprises normalizing, for each control population, the number of unique control molecular tags determined in step b.ii) by a sum of the number of the unique targeting molecular tags and the numbers of the unique control molecular tags.

46. The method of any of claims 43-45, wherein the target probe capture metric for the target population is indicative of the target population's ability to hybridize to the target sequence of interest, relative to the abilities of the plurality of control populations to hybridize to the distinct control sequences.

47. The method of any of claims 43-46, wherein each control probe capture metric for a respective control population is indicative of the respective control population's ability to hybridize to one of the control sequences, relative to the abilities of 1) the target population to hybridize to the target sequence and 2) remaining control populations to hybridize to respective control sequences.

48. The method of any of claims 43-47, wherein the target sequence of interest is located on the gene of interest, and the control sequences correspond to one or more reference genes that are different from the gene of interest.

49. The method of any of claims 43-48, wherein the gene of interest is a survival of motor neuron 1 (SMN1) gene and/or a survival of motor neuron 2 (SMN2) gene.

50. The method of any of claims 43-49, wherein the at least one criterion includes a requirement that the control probe capture metric is above a first threshold and below a second threshold.

51. The method of claim 50, further comprising determining the first threshold and the second threshold based at least in part on the target probe capture metric computed at step b.iii).

52. The method of claim 51, wherein the first threshold and the second threshold are determined further based at least in part on the plurality of control probe capture metrics computed at step b.iii).

53. The method of any of claims 43-52, further comprising, for each control population, computing a variability coefficient for the control probe capture metrics computed at step b.iii) across the samples obtained from each subset in the plurality of subsets.

54. The method of claim 53, wherein the at least one criterion includes a requirement that the variability coefficient is below a threshold.

55. The method of any of claims 43-54, wherein the factor computed at step b.v) is an average of the control probe capture metrics satisfying the at least one criterion.

56. The method of any of claims 43-55, wherein a first subset is characterized by subjects exhibiting a known copy count of a survival of motor neuron 1 (SMN1) gene, and a second subset is characterized by subjects exhibiting a known copy count of a survival motor neuron 2 (SMN2) gene.

57. The method of any of claims 43-56, wherein the known genotype corresponds to a known copy count of a survival of motor neuron 1 (SMN1) gene or of a survival of motor neuron 2 (SMN2) gene.

58. The method of any of claims 43-57, wherein the first and second unique targeting molecular tags and the first and second unique control molecular tags are generated randomly for each MIP in the targeting population of targeting MIPS and in the control populations of control MIPs.

59. A system configured to perform the method of any of claims 43-58.

60. A computer program product comprising computer-readable instructions that, when executed in a computerized system comprising at least one processor, cause the processor to carry out one or more steps of the method of any of claims 43-58.

61. A method of selecting a genotype for a test subject, the method comprising:
a) receiving sequencing data obtained from a nucleic acid sample from the test subject, wherein the sequencing data for the nucleic acid sample is obtained by:
i) obtaining a nucleic acid sample isolated from the test subject;
ii) capturing one or more target sequences of interest in the nucleic acid sample obtained in step a) by using one or more target populations of targeting molecular inversion probes (MIPs) to produce a plurality of targeting MIPs replicons for each target sequence,
wherein each of the targeting MIPs in the target population comprises in sequence the following components:
first targeting polynucleotide arm—first unique targeting molecular tag—polynucleotide linker—second unique targeting molecular tag—second targeting polynucleotide arm;
wherein the pair of first and second targeting polynucleotide arms in each of the targeting MIPs in each target population are identical, and are substantially complementary to first and second regions in the nucleic acid that, respectively, flank the target sequence of interest that is targeted by the one or more targeting MIPs;
wherein the first and second unique targeting molecular tags in each of the targeting MIPs in each target population are distinct in each of the targeting MIPs and in each member of the target population;
iii) capturing a plurality of control sequences in the nucleic acid sample obtained in step a) by using a plurality of control populations of control MIPs to produce a plurality of control MIPs replicons, each control population of control MIPs being capable of amplifying a distinct control sequence in the nucleic acid sample obtained in step a),
wherein each of the control MIPs in each control population comprises in sequence the following components:
first control polynucleotide arm—first unique control molecular tag—polynucleotide linker—second unique control molecular tag—second control polynucleotide arm;
wherein the pair of first and second control polynucleotide arms in each of the control MIPs in each control population are identical, and are substantially complementary to first and second regions in the nucleic acid that, respectively, flank each control sequence;
wherein the first and second unique control molecular tags in each of the control MIPs in each control population are distinct in each of the control MIPs and in each member of the control population, and are different from the unique targeting molecular tags;
iv) sequencing the targeting and control MIPs amplicons that are amplified from the targeting and control MIPs replicons obtained in steps a.ii) and a.iii);
b) determining, for each target population, the number of the unique targeting molecular tags present in the targeting MIPs amplicons sequenced in step a.iv);
c) determining, for each control population, the number of the unique control molecular tags present in the control MIPs amplicons sequenced in step a.iv);
d) computing a target probe capture metric, for each target site, based at least in part on the number of the unique targeting molecular tags determined in step b) and a plurality of control probe capture metrics based at least in part on the numbers of the unique control molecular tags determined in step c);
e) identifying a subset of the control populations of control MIPs that have control probe capture metrics satisfying at least one criterion;
f) normalizing each of the one or more target probe capture metrics by a factor computed from the control probe capture metrics satisfying the at least one criterion, to obtain a normalized target probe capture metric for each of the one or more target sequences;
g) receiving a group of values corresponding to normalized target probe capture metrics computed from nucleic acid samples from a first plurality of reference subjects exhibiting a same known genotype for a gene of interest;
h) comparing each of the one or more normalized target probe capture metrics obtained in step f) to the group of values received in step g); and
i) determining, based on the comparing in step h), whether the test subject exhibits the same known genotype for the gene of interest in each of the one or more target sequences.

62. The method of claim 61, wherein the group of values is a first group of values, the same known genotype is a first copy number of the target sequence of interest, the method further comprising:
j) receiving a second group of values corresponding to normalized target probe capture metrics computed from nucleic acid samples from a second plurality of reference subjects exhibiting a second copy number of the target sequence of interest; and
k) comparing the normalized target probe capture metric obtained in step f) to the second group of values, wherein the determining in step i) comprises selecting between the first copy number and the second copy number for the test subj ect.

63. The method of claim 62, wherein:
the comparing in step h) comprises computing a first distance metric between the normalized probe capture metric obtained in step f) and the first group of values;
the comparing in step k) comprises computing a second distance metric between the normalized probe capture metric obtained in step f) and the second group of values; and
the selecting between the first copy number and second copy number comprises selecting the first copy number if the first distance metric is less than the second distance metric, and selecting the second copy number if the first distance metric exceeds the second distance metric.

64. The method of any of claims 63, wherein the first group of values and the second group of values are computed by:
repeating steps a-f) for each subject in the first and second pluralities of reference subjects;
grouping the normalized target probe capture metrics for the first plurality of reference subjects to obtain the first group of values; and
grouping the normalized target probe capture metrics for the second plurality of reference subjects to obtain the second group of values.

65. The method of any of claims 61-64, wherein the computing the target probe capture metric at step d) comprises normalizing the number of the unique targeting molecular tags determined in step b) by a sum of the number of the unique targeting molecular tags and the numbers of the unique control molecular tags.

66. The method of any of claims 61-65, wherein computing the plurality of control probe capture metrics at step d) comprises normalizing, for each control population, the number of the unique control molecular tags determined in step c) by a sum of the unique targeting molecular tags and the numbers of the unique control molecular tags.

67. The method of any of claims 61-66, wherein the target probe capture metric for the target population is indicative of the target population's ability to hybridize to the target sequence of interest, relative to the abilities of the plurality of control populations to hybridize to the control sequences.

68. The method of any of claims 61-67, wherein the target sequence of interest is on the gene of interest, and the control sequences correspond to one or more reference genes that are different from the gene of interest.

69. The method of any of claims 61-68, wherein the gene of interest is a survival of motor neuron 1 (SMN1) gene and/or a survival of motor neuron 2 (SMN2) gene.

70. The method of any of claims 61-69, wherein the at least one criterion includes a requirement that the control probe capture metric are above a first threshold and below a second threshold.

71. The method of claim 70, further comprising determining the first threshold and the second threshold based at least in part on the target probe capture metric computed at step d).

72. The method of claim 71, wherein the first threshold and the second threshold are determined further based at least in part on the plurality of control probe capture metrics computed at step d).

73. The method of any of claims 61-72, further comprising, for each control population, computing a variability coefficient for the control probe capture metrics computed at step d).

74. The method of claim 73, wherein the at least one criterion includes a requirement that the variability coefficient is below a threshold.

75. The method of any of claims 61-74, wherein the factor computed at step f) is an average of the control probe capture metrics satisfying the at least one criterion.

76. The method of any of claims 61-75, wherein the target sequence of interest is on a survival of motor neuron 1 (SMN1) gene and/or a survival of motor neuron 2 (SMN2) gene.

77. The method of claim 76, wherein the same known genotype corresponds to a known copy count of an SMN1 gene or an SMN2 gene.

78. A system configured to perform the method of any of claims 61-77.

79. A computer program product comprising computer-readable instructions that, when executed in a computerized system comprising at least one processor, cause the processor to carry out one or more steps of the method of any of claims 61-77.

80. The method of any one of claims 41-55, 58, and 61-75, wherein the subject or the test subject is a candidate for carrier screening of one or more diseases or conditions.

81. The method of any one of claims 41-55, 58, and 61-75, wherein the subject or the test subject is a candidate for:
a) a pharmacogenomics test;
b) a targeted tumor test; or
c) an exonic deletion test.
